(19) United States
(12) Patent Application Publication
(10) Pub. No.: US 2016/0159774 A1
(43) Pub. Date: Jun. 9, 2016

(54) HETEROARYL COMPOUNDS AND USES THEREOF

(71) Applicant: Celgene Avilomics Research, Inc., Bedford, MA (US)

(72) Inventors: C. Eric Schwartz, Lynnfield, MA (US); Sekhar S. Surapaneni, Warren, NJ (US); Karin Irmgard Worm, Jackson, NJ (US)

(21) Appl. No.: 14/957,761

(22) Filed: Dec. 3, 2015

Related U.S. Application Data
(60) Provisional application No. 62/088,377, filed on Dec. 5, 2014.

(51) Int. Cl. C07D 401/14; C07D 403/14; C07D 487/08; C07D 405/14

(52) U.S. Cl. CPC C07D 401/14; C07D 405/14; C07D 403/14; C07D 487/08

(57) ABSTRACT

The present invention provides compounds, pharmaceutically acceptable compositions thereof, and methods of using the same.

### CROSS REFERENCE TO RELATED APPLICATIONS

The present application claims priority to U.S. provisional application Ser. No. 62/088,377, filed Dec. 5, 2014, the entirety of which is hereby incorporated by reference.

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there remains a need to find protein kinase inhibitors useful as therapeutic agents.

### SUMMARY OF THE INVENTION

It has now been found that compounds of this invention, and pharmaceutically acceptable compositions thereof, are effective as inhibitors of one or more protein kinases. Such compounds have general formula I:

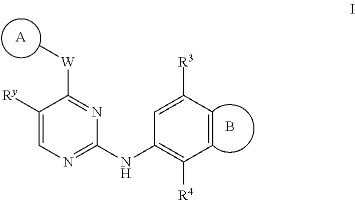

or a pharmaceutically acceptable salt thereof, wherein Ring A, Ring B, W, R^{y}, R³ and R⁴ are as defined herein.

Compounds of the present invention, and pharmaceutically acceptable compositions thereof, are useful for treating a variety of diseases, disorders or conditions associated with abnormal cellular responses triggered by protein kinase-mediated events. Such diseases, disorders, or conditions include those described herein.

Compounds provided by this invention are also useful for the study of kinases in biological and pathological phenomena; the study of intracellular signal transduction pathways mediated by such kinases; and the comparative evaluation of new kinase inhibitors.

### DETAILED DESCRIPTION OF CERTAIN EMBODIMENTS

### 1. General Description of Compounds of the Invention

In certain embodiments, the present invention provides a compound of formula I:

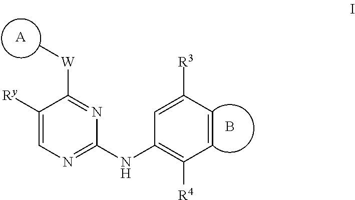

or a pharmaceutically acceptable salt thereof, wherein:
Ring A is a saturated 4-8 membered monocyclic or bridged heterocyclic ring having one —N(R¹)—, a saturated 7-11 membered spirofused heterocyclic ring having one —N(R¹)—, or a saturated 8-10 membered bicyclic heterocyclic ring having one —N(R¹)—, wherein Ring A is substituted with 0-3 R^{v} groups;
R¹ is -L-Y, wherein:
   L is an optionally substituted bivalent C₂₋₈ unsaturated, straight or branched, hydrocarbon chain, wherein one, two, or three methylene units of L are optionally and independently replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—; and
   Y is hydrogen, halogen, —CN, C₁₋₆ aliphatic optionally substituted with oxo, halogen, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 groups independently selected from -Q-Z, oxo, —NO₂, halogen, —CN, and C₁₋₆ aliphatic, wherein:
      Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —SO—, or —SO₂—; and
      Z is hydrogen or C₁₋₆ aliphatic optionally substituted with oxo, halogen, or CN;
Ring B is a saturated 5-7-membered heterocyclo ring having 1-2 nitrogen atoms, wherein Ring B is substituted with 0-5 R^{x} groups;
W is —N(R²)CH₂— or —NH—;
R² is selected from hydrogen, C₁₋₆ aliphatic or —C(O)R;
R³ and R⁴ are each independently selected from hydrogen or halogen;
each R group is independently hydrogen or an optionally substituted group selected from C₁₋₆ aliphatic, phenyl, a 3-7 membered saturated or partially unsaturated carbocyclic ring, a 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
R^{y} is hydrogen, halogen, —CF₃, or C₁₋₄ aliphatic;
each R^{x} is independently oxo, halogen, —OR, —N(R)₂, —S(O)ₓR, —N(R)(CH₂)_{q}N(R)₂, —N(R)(CH₂)_{q}OR, —O(CH₂)_{q}OR, —O(CH₂)_{q}N(R)₂, an optionally substituted C₂₋₆ saturated, straight or branched, hydrocarbon chain wherein one or two methylene units are independently replaced by —O—, —N(R)— or —S(O)ₓ—, or an optionally substituted group selected from C₁₋₆ aliphatic, phenyl, a 3-7 membered saturated or partially unsaturated carbocyclic ring, a 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
each R^{v} is independently selected from halogen or C₁₋₆ aliphatic;
q is 1 or 2; and
each x is 0, 1 or 2.

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “heteroatom” means one or more of oxygen, sulfur, nitrogen, phosphorus, or silicon (including any oxidized form of nitrogen, sulfur, phosphorus, or silicon; and the quaternized form of any basic nitrogen or a substitutable nitrogen of a heterocyclic ring, for example N (as in 3,4-dihydro-2H-pyrrolyl-

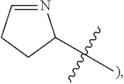 [SMILES: *C1CCC=N1]

NH (as in pyrrolidinyl-

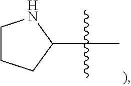 [SMILES: *C1CCCN1]

NR̂ (as in N-substituted 2-pyrrolidinyl-

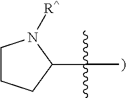

or ⁺NR̂ (as in N-substituted 1-pyrrolidinyl-

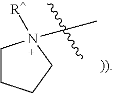

The term “unsaturated”, as used herein, means that a moiety has one or more units of unsaturation. In some embodiments, a unit of unsaturation is a carbon-carbon double bond (i.e., —C═C—). In some embodiments, a unit of unsaturation is a carbon-carbon triple bond (i.e., —C≡C—).

As used herein, the term “bivalent C₂₋₈ (or C₂₋₆) unsaturated, straight or branched, hydrocarbon chain” refers to bivalent alkenylene and alkynylene chains that are straight or branched as defined herein and have one or more units of unsaturation.

The term “alkylene” refers to a straight or branched bivalent alkyl group. Exemplary alkylenes include —CH₂—, —CH₂CH₂—, —CH(CH₃)—, —CH₂CH(CH₃)—, —CH(CH₃)CH₂—, etc. In some embodiments, an “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a bivalent alkyl group in which one or more hydrogen atoms are replaced with a substituent. Suitable substituents include those described below for a substituted aliphatic group.

The term “alkenylene” refers to a bivalent alkenyl group. A substituted alkenylene chain is a bivalent alkenyl group containing at least one double bond in which one or more hydrogen atoms are optionally replaced with a substituent. Suitable substituents include those described below for a substituted aliphatic group.

As used herein, the term “cyclopropylene” refers to a bivalent cyclopropyl group of the following structure:

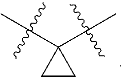 [SMILES: *C1(*)CC1]

The term “halogen” means F, Cl, Br, or I.

The term “aryl” refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. A heteroaryl group may be mono- or bicyclic. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. When a heteroaryl ring is fused to an aryl ring, the term “heteroaro” is used to refer to the heteroaryl ring that is fused to the aryl ring. The term “heteroaryl” may be used interchangeably with the terms “heteroaryl ring”, “heteroaryl group”, or “heteroaromatic”, any of which terms include rings that are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl-

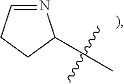 [SMILES: *C1CCC=N1]

NH (as in pyrrolidinyl-

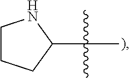 [SMILES: *C1CCCN1]

NR̂ (as in N-substituted 2-pyrrolidinyl-

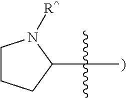

or ⁺NR̂ (as in N-substituted 1-pyrrolidinyl-

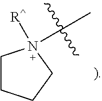

A heterocyclic ring can be attached to its pendant group at any heteroatom or carbon atom that results in a stable structure and any of the ring atoms can be optionally substituted. A heterocyclyl group may be mono- or bicyclic. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. For purposes of clarity, a “heterocyclic” ring includes a saturated or partially unsaturated ring having one or more heteroatoms, wherein the ring is either monocyclic or fused to one or more aryl, heteroaryl, or cycloaliphatic rings. When a heterocyclic ring is fused to an aryl ring, the term “heterocyclo” is used to refer to the heterocyclic ring that is fused to the aryl ring. A “saturated heterocyclic ring” refers to a saturated ring having one or more heteroatoms, wherein the ring is monocyclic or fused to one or more saturated cycloaliphatic rings.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘)}₂; SiR^{∘}₃; —(C₁₋₄ straight or branched alkylene)O—N(R^{∘})₂; or —(C₁₋₄ straight or branched)alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂— (5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In some embodiments, the R¹ group of formula I comprises one or more deuterium atoms.

As used herein, the term “irreversible” or “irreversible inhibitor” refers to an inhibitor (i.e. a compound) that is able to be covalently bonded to a target protein kinase in a substantially non-reversible manner. That is, whereas a reversible inhibitor is able to bind to (but is generally unable to form a covalent bond) the target protein kinase, and therefore can become dissociated from the target protein kinase, an irreversible inhibitor will remain substantially bound to the target protein kinase once covalent bond formation has occurred. Irreversible inhibitors usually display time dependency, whereby the degree of inhibition increases with the time with which the inhibitor is in contact with the enzyme. Methods for identifying if a compound is acting as an irreversible inhibitor are known to one of ordinary skill in the art. Such methods include, but are not limited to, enzyme kinetic analysis of the inhibition profile of the compound with the protein kinase target, the use of mass spectrometry of the protein drug target modified in the presence of the inhibitor compound, discontinuous exposure, also known as “washout,” experiments, and the use of labeling, such as radiolabelled inhibitor, to show covalent modification of the enzyme, as well as other methods known to one of skill in the art.

It will be appreciated that certain reactive functional groups can act as “warheads.” As used herein, the term “warhead” or “warhead group” refers to a functional group present on a compound of the present invention wherein that functional group is capable of covalently binding to an amino acid residue (such as cysteine, lysine, histidine, or other residues capable of being covalently modified) present in the binding pocket of the target protein, thereby irreversibly inhibiting the protein. In some embodiments, the -L-Y group, as defined and described herein, provides such warhead groups for covalently, and irreversibly, inhibiting the protein.

As used herein, the term “inhibitor” is defined as a compound that binds to and/or inhibits the target protein kinase with measurable affinity. In certain embodiments, an inhibitor has an IC₅₀ and/or binding constant of less about 50 μM, less than about 1 μM, less than about 500 nM, less than about 100 nM, or less than about 10 nM.

The terms “measurable affinity” and “measurably inhibit,” as used herein, means a measurable change in the activity of at least one kinase selected from TEC, BTK, ITK, BMX, BLK, EGFR, ErbB2, ErbB4, JAK3, MAP2K7, and/or TXK between a sample comprising a compound of the present invention, or composition thereof, and at least one kinase selected from TEC, BTK, ITK, BMX, BLK, EGFR, ErbB2, ErbB4, JAK3, MAP2K7, and/or TXK, and an equivalent sample comprising at least one kinase selected from TEC, BTK, ITK, BMX, BLK, EGFR, ErbB2, ErbB4, JAK3, MAP2K7, and/or TXK, in the absence of said compound, or composition thereof.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent, delay or lessen the severity of their recurrence.

### 3. Description of Exemplary Compounds

According to one aspect, the present invention provides a compound of formula I:

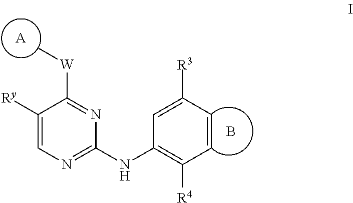

or a pharmaceutically acceptable salt thereof, wherein:
Ring A is a saturated 4-8 membered monocyclic or bridged heterocyclic ring having one —N(R¹)—, a saturated 7-11 membered spirofused heterocyclic ring having one —N(R¹)—, or a saturated 8-10 membered bicyclic heterocyclic ring having one —N(R¹)—, wherein Ring A is substituted with 0-3 R^{v} groups;
R¹ is -L-Y, wherein:
   L is an optionally substituted bivalent C₂₋₈ unsaturated, straight or branched, hydrocarbon chain, wherein one, two, or three methylene units of L are optionally and independently replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—; and
   Y is hydrogen, halogen, —CN, C₁₋₆ aliphatic optionally substituted with oxo, halogen, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 groups independently selected from -Q-Z, oxo, —NO₂, halogen, —CN, and C₁₋₆ aliphatic, wherein:
      Q is a covalent bond or a bivalent C₁₋₆ saturated or unsaturated, straight or branched, hydrocarbon chain, wherein one or two methylene units of Q are optionally and independently replaced by —NR—, —S—, —O—, —C(O)—, —SO—, or —SO₂—; and
      Z is hydrogen or C₁₋₆ aliphatic optionally substituted with oxo, halogen, or CN;
Ring B is a saturated 5-7-membered heterocyclo ring having 1-2 nitrogen atoms, wherein Ring B is substituted with 0-5 R^{x} groups;
W is —N(R²)CH₂— or —NH—;
R² is selected from hydrogen, C₁₋₆ aliphatic or —C(O)R;
R³ and R⁴ are each independently selected from hydrogen or halogen;
each R group is independently hydrogen or an optionally substituted group selected from C₁₋₆ aliphatic, phenyl, a 3-7 membered saturated or partially unsaturated carbocyclic ring, a 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
R^{y} is hydrogen, halogen, —CF₃, or C₁₋₄ aliphatic;
each R^{x} is independently oxo, halogen, —OR, —N(R)₂, —S(O)ₓR, —N(R)(CH₂)_{q}N(R)₂, —N(R)(CH₂)_{q}OR, —O(CH₂)_{q}OR, —O(CH₂)_{q}N(R)₂, an optionally substituted C₂₋₆ saturated, straight or branched, hydrocarbon chain wherein one or two methylene units are independently replaced by —O—, —N(R)— or —S(O)ₓ—, or an optionally substituted group selected from C₁₋₆ aliphatic, phenyl, a 3-7 membered saturated or partially unsaturated carbocyclic ring, a 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
each R^{v} is independently selected from halogen or C₁₋₆ aliphatic;
q is 1 or 2; and
each x is 0, 1 or 2.

As defined generally above, Ring A is a saturated 4-8 membered monocyclic or bridged bicyclic heterocyclic ring having one —N(R¹)—, a saturated 7-11 membered spirofused heterocyclic ring having one —N(R¹)—, or a saturated 8-10 membered bicyclic heterocyclic ring having one —N(R¹)—, wherein Ring A is substituted with 0-3 R^{v} groups. In certain embodiments, Ring A is a saturated 4-8 membered monocyclic or bridged bicyclic heterocyclic ring having one —N(R¹)—. In some embodiments, Ring A is a saturated 4-membered monocyclic heterocyclic ring having one —N(R¹)—. In some embodiments, Ring A is a saturated 5-membered monocyclic heterocyclic ring having one —N(R¹)—. In some embodiments, Ring A is a saturated 6-membered monocyclic heterocyclic ring having one —N(R¹)—. In some embodiments, Ring A is a saturated 7-membered monocyclic heterocyclic ring having one —N(R¹)—. In some embodiments, Ring A is a saturated 8-membered monocyclic heterocyclic ring having one —N(R¹)—. In some embodiments, Ring A is a saturated 7-membered bridged bicyclic heterocyclic ring having one —N(R¹)—. In some such embodiments, Ring A is an azabicyclo[2.2.1]heptane. In some embodiments, Ring A is a saturated 8-membered bridged bicyclic heterocyclic ring having one —N(R¹)—. In some such embodiments, Ring A is an azabicyclo[2.2.2]octane.

It will be appreciated that, when Ring A is a saturated 4-8 membered monocyclic or bridged bicyclic heterocyclic ring, —N(R¹)— can be located at a position adjacent to, or one or more atoms away from, the atom to which W is attached. For example, in some embodiments, —N(R¹)— is located at the alpha (α)-position of Ring A relative to the atom to which W is attached. In certain embodiments, —N(R¹)— is located at the beta (β)-, gamma (γ)- or delta (δ)-position of Ring A relative to the atom to which W is attached, according to the following convention:

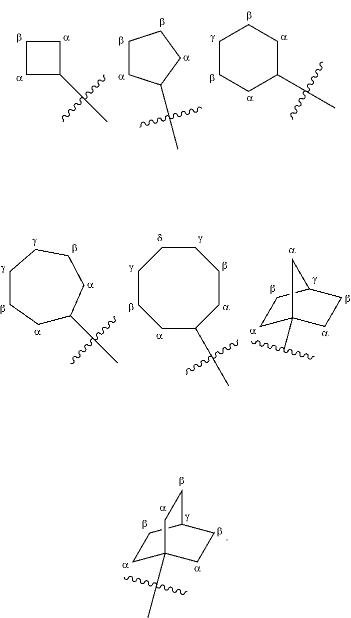

In certain preferred embodiments, —N(R¹)— is located at the beta (β)-position of Ring A relative to the atom to which W is attached. In some embodiments, Ring A is selected from those groups in Table 1A:

TABLE 1A
Exemplary Ring A Groups

| | |
|---|---|
| 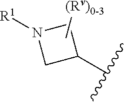 | 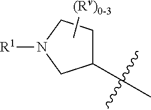 |
| 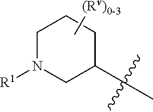 | 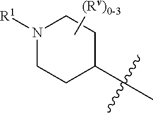 |
| 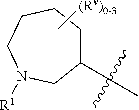 | 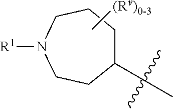 |
| 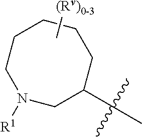 | 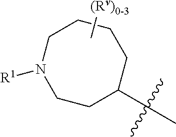 |
| 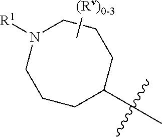 | 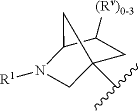 |
| 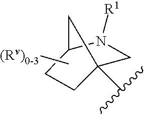 | 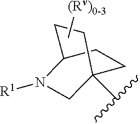 |

In certain preferred embodiments, Ring A is selected from those groups in Table 1B:

TABLE 1B
Exemplary Ring A Groups

| | |
|---|---|
| 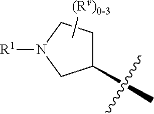 | 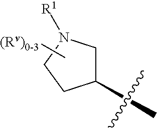 |
| 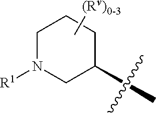 | 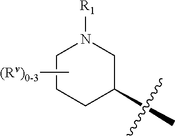 |
| 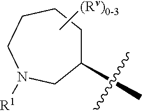 | 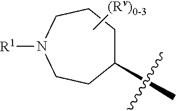 |
| 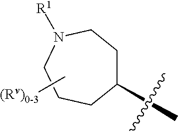 | 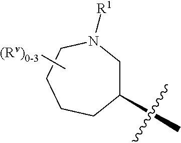 |
| 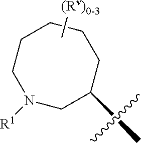 | 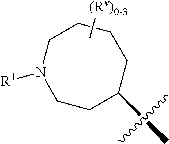 |
| 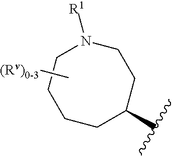 | 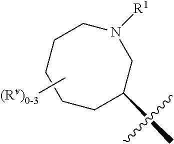 |

In some embodiments, Ring A is a saturated 7-11 membered spirofused heterocyclic ring having one —N(R¹)—. In some embodiments, Ring A is a saturated 7-membered spirofused heterocyclic ring having one —N(R¹)—. In some embodiments, Ring A is a saturated 8-membered spirofused heterocyclic ring having one —N(R¹)—. In some embodiments, Ring A is a saturated 9-membered spirofused heterocyclic ring having one —N(R¹)—. In some embodiments, Ring A is a saturated 10-membered spirofused heterocyclic ring having one —N(R¹)—. In some embodiments, Ring A is a saturated 11-membered spirofused heterocyclic ring having one —N(R¹)—.

It will be appreciated that a spirofused ring system consists of a proximal ring (i.e., the ring to which the remainder of the molecule is directly attached) and a distal ring (i.e., the ring spirofused to the proximal ring). It will be further appreciated that, when Ring A is a saturated 7-11 membered spirofused heterocyclic ring, —N(R¹)— can be located in the ring to which W is attached (i.e., the proximal ring of the spirofused ring system). Alternatively, —N(R¹)— can be located in the ring to which W is not attached (i.e., the distal ring of the spirofused ring system).

In some embodiments, —N(R¹)— is located at the alpha (α)-, alpha′ (α′)-, beta (β)-, beta′ (β′)-, gamma (γ)-, gamma′ (γ′)-, delta (δ)-, epsilon (ε)-, zeta (ζ)- or eta (η)-position of the proximal ring of the spirofused ring system according to the following convention:

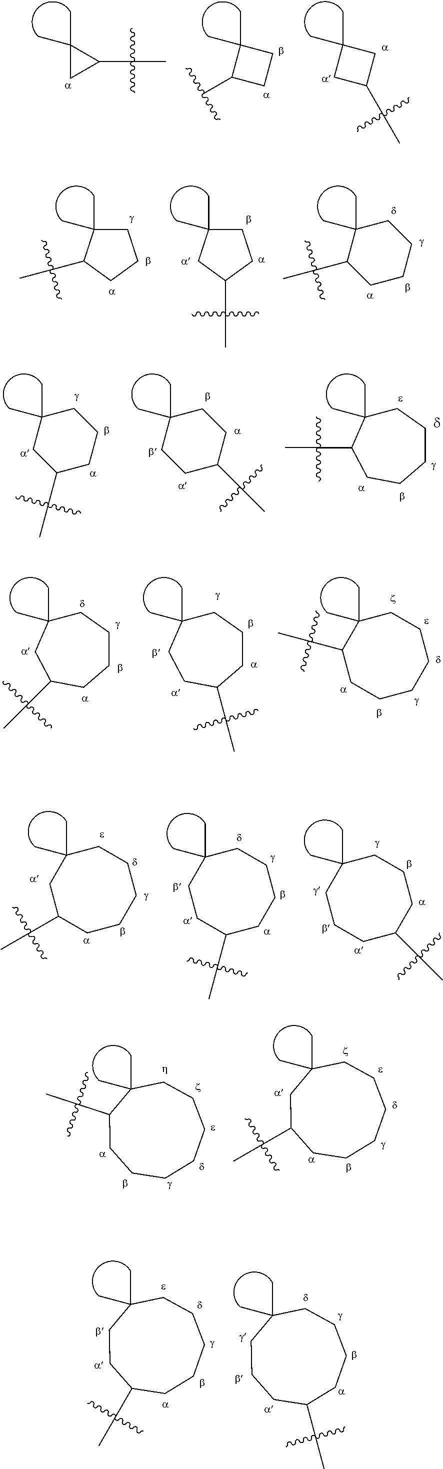

In some embodiments, Ring A is a saturated 7-membered spirofused heterocyclic ring, wherein —N(R¹)— is at the α-position of the proximal spirofused ring. In some embodiments, Ring A is a saturated 7-membered spirofused heterocyclic ring, wherein —N(R¹)— is at the α′-position of the proximal spirofused ring. In some embodiments, Ring A is a saturated 7-membered spirofused heterocyclic ring, wherein —N(R¹)— is at the β-position of the proximal spirofused ring. In some embodiments, Ring A is a saturated 7-membered spirofused heterocyclic ring, wherein —N(R¹)— is at the γ-position of the proximal spirofused ring.

In some embodiments, Ring A is a saturated 8-membered spirofused heterocyclic ring, wherein —N(R¹)— is at the α-position of the proximal spirofused ring. In some embodiments, Ring A is a saturated 8-membered spirofused heterocyclic ring, wherein —N(R¹)— is at the α′-position of the proximal spirofused ring. In some embodiments, Ring A is a saturated 8-membered spirofused heterocyclic ring, wherein —N(R¹)— is at the β-position of the proximal spirofused ring. In some embodiments, Ring A is a saturated 8-membered spirofused heterocyclic ring, wherein —N(R¹)— is at the β′-position of the proximal spirofused ring. In some embodiments, Ring A is a saturated 8-membered spirofused heterocyclic ring, wherein —N(R¹)— is at the γ-position of the proximal spirofused ring. In some embodiments, Ring A is a saturated 8-membered spirofused heterocyclic ring, wherein —N(R¹)— is at the δ-position of the proximal spirofused ring.

In some embodiments, Ring A is a saturated 9-membered spirofused heterocyclic ring, wherein —N(R¹)— is at the α-position of the proximal spirofused ring. In some embodiments, Ring A is a saturated 9-membered spirofused heterocyclic ring, wherein —N(R¹)— is at the α′-position of the proximal spirofused ring. In some embodiments, Ring A is a saturated 9-membered spirofused heterocyclic ring, wherein —N(R¹)— is at the β-position of the proximal spirofused ring. In some embodiments, Ring A is a saturated 9-membered spirofused heterocyclic ring, wherein —N(R¹)— is at the β′-position of the proximal spirofused ring. In some embodiments, Ring A is a saturated 9-membered spirofused heterocyclic ring, wherein —N(R¹)— is at the γ-position of the proximal spirofused ring. In some embodiments, Ring A is a saturated 9-membered spirofused heterocyclic ring, wherein —N(R¹)— is at the δ-position of the proximal spirofused ring. In some embodiments, Ring A is a saturated 9-membered spirofused heterocyclic ring, wherein —N(R¹)— is at the ε-position of the proximal spirofused ring.

In some embodiments, Ring A is a saturated 10-membered spirofused heterocyclic ring, wherein —N(R¹)— is at the α-position of the proximal spirofused ring. In some embodiments, Ring A is a saturated 10-membered spirofused heterocyclic ring, wherein —N(R¹)— is at the α′-position of the proximal spirofused ring. In some embodiments, Ring A is a saturated 10-membered spirofused heterocyclic ring, wherein —N(R¹)— is at the β-position of the proximal spirofused ring. In some embodiments, Ring A is a saturated 10-membered spirofused heterocyclic ring, wherein —N(R¹)— is at the β′-position of the proximal spirofused ring. In some embodiments, Ring A is a saturated 10-membered spirofused heterocyclic ring, wherein —N(R¹)— is at the γ-position of the proximal spirofused ring. In some embodiments, Ring A is a saturated 10-membered spirofused heterocyclic ring, wherein —N(R¹)— is at the γ′-position of the proximal spirofused ring. In some embodiments, Ring A is a saturated 10-membered spirofused heterocyclic ring, wherein —N(R¹)— is at the δ-position of the proximal spirofused ring. In some embodiments, Ring A is a saturated 10-membered spirofused heterocyclic ring, wherein —N(R¹)— is at the ε-position of the proximal spirofused ring. In some embodiments, Ring A is a saturated 10-membered spirofused heterocyclic ring, wherein —N(R¹)— is at the ζ-position of the proximal spirofused ring.

In some embodiments, Ring A is a saturated 11-membered spirofused heterocyclic ring, wherein —N(R¹)— is at the α-position of the proximal spirofused ring. In some embodiments, Ring A is a saturated 11-membered spirofused heterocyclic ring, wherein —N(R¹)— is at the α′-position of the proximal spirofused ring. In some embodiments, Ring A is a saturated 11-membered spirofused heterocyclic ring, wherein —N(R¹)— is at the β-position of the proximal spirofused ring. In some embodiments, Ring A is a saturated 11-membered spirofused heterocyclic ring, wherein —N(R¹)— is at the β-position of the proximal spirofused ring. In some embodiments, Ring A is a saturated 11-membered spirofused heterocyclic ring, wherein —N(R¹)— is at the γ-position of the proximal spirofused ring. In some embodiments, Ring A is a saturated 11-membered spirofused heterocyclic ring, wherein —N(R¹)— is at the γ′-position of the proximal spirofused ring. In some embodiments, Ring A is a saturated 11-membered spirofused heterocyclic ring, wherein —N(R¹)— is at the δ-position of the proximal spirofused ring. In some embodiments, Ring A is a saturated 11-membered spirofused heterocyclic ring, wherein —N(R¹)— is at the ε-position of the proximal spirofused ring. In some embodiments, Ring A is a saturated 11-membered spirofused heterocyclic ring, wherein —N(R¹)— is at the ζ-position of the proximal spirofused ring. In some embodiments, Ring A is a saturated 11-membered spirofused heterocyclic ring, wherein —N(R¹)— is at the n-position of the proximal spirofused ring.

In some embodiments, —N(R¹)— is located at the alpha (α)-, alpha′ (α′)-, beta (β)-, beta′ (β′)—, gamma (γ)-, gamma′ (γ′)—, delta (δ)—, delta′ (δ′)-position of the distal ring of the spirofused ring system according to the following convention:

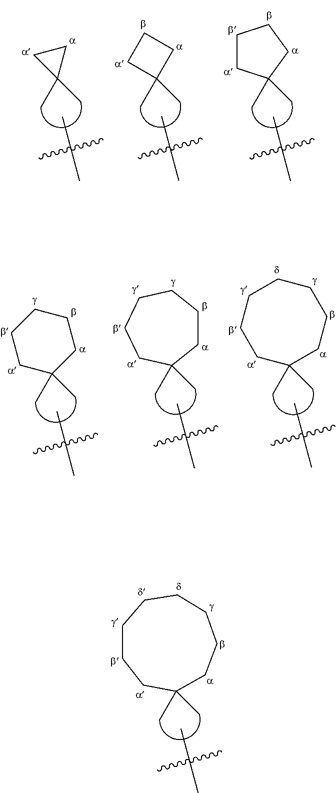

In some embodiments, Ring A is a saturated 7-membered spirofused heterocyclic ring, wherein —N(R¹)— is at the α- or α′-position of the distal spirofused ring. In some embodiments, Ring A is a saturated 7-membered spirofused heterocyclic ring, wherein —N(R¹)— is at the β- or β′-position of the distal spirofused ring.

In some embodiments, Ring A is a saturated 8-membered spirofused heterocyclic ring, wherein —N(R¹)— is at the α- or α′-position of the distal spirofused ring. In some embodiments, Ring A is a saturated 8-membered spirofused heterocyclic ring, wherein —N(R¹)— is at the β- or β′-position of the distal spirofused ring. In some embodiments, Ring A is a saturated 8-membered spirofused heterocyclic ring, wherein —N(R¹)— is at the γ-position of the distal spirofused ring.

In some embodiments, Ring A is a saturated 9-membered spirofused heterocyclic ring, wherein —N(R¹)— is at the α- or α′-position of the distal spirofused ring. In some embodiments, Ring A is a saturated 9-membered spirofused heterocyclic ring, wherein —N(R¹)— is at the β- or β′-position of the distal spirofused ring. In some embodiments, Ring A is a saturated 9-membered spirofused heterocyclic ring, wherein —N(R¹)— is at the γ- or γ′-position of the distal spirofused ring.

In some embodiments, Ring A is a saturated 10-membered spirofused heterocyclic ring, wherein —N(R¹)— is at the α- or α′-position of the distal spirofused ring. In some embodiments, Ring A is a saturated 10-membered spirofused heterocyclic ring, wherein —N(R¹)— is at the β- or β′-position of the distal spirofused ring. In some embodiments, Ring A is a saturated 10-membered spirofused heterocyclic ring, wherein —N(R¹)— is at the γ- or γ′-position of the distal spirofused ring. In some embodiments, Ring A is a saturated 10-membered spirofused heterocyclic ring, wherein —N(R¹)— is at the δ-position of the distal spirofused ring.

In some embodiments, Ring A is a saturated 11-membered spirofused heterocyclic ring, wherein —N(R¹)— is at the α- or α′-position of the distal spirofused ring. In some embodiments, Ring A is a saturated 11-membered spirofused heterocyclic ring, wherein —N(R¹)— is at the β- or β′-position of the distal spirofused ring. In some embodiments, Ring A is a saturated 11-membered spirofused heterocyclic ring, wherein —N(R¹)— is at the γ- or γ′-position of the distal spirofused ring. In some embodiments, Ring A is a saturated 11-membered spirofused heterocyclic ring, wherein —N(R¹)— is at the δ- or δ′-position of the distal spirofused ring.

In some embodiments, Ring A is substituted with 0-3 R^{v} groups and is selected from azaspiro[2.4]heptane, azaspiro[3.3]heptane, azaspiro[2.5]octane, azaspiro[3.4]octane, azaspiro[2.6]nonane, azaspiro[3.5]nonane, azaspiro[4.4]nonane, azaspiro[2.7]decane, azaspiro[3.6]decane, azaspiro[4.5]decane, azaspiro[2.8]undecane, azaspiro[3.7]undecane, azaspiro[4.6]undecane and azaspiro[5.5]undecane.

In some embodiments, Ring A is substituted with 0-3 R^{v} groups and is selected from 1-azaspiro[2.4]heptane, 4-azaspiro[2.4]heptane, 5-azaspiro[2.4]heptane, 1-azaspiro[3.3]heptane, 2-azaspiro[3.3]heptane, 1-azaspiro[2.5]octane, 4-azaspiro[2.5]octane, 5-azaspiro[2.5]octane, 6-azaspiro[2.5]octane, 1-azaspiro[3.4]octane, 2-azaspiro[3.4]octane, 5-azaspiro[3.4]octane, 6-azaspiro[3.4]octane, 1-azaspiro[2.6]nonane, 4-azaspiro[2.6]nonane, 5-azaspiro[2.6]nonane, 6-azaspiro[2.6]nonane, 1-azaspiro[3.5]nonane, 2-azaspiro[3.5]nonane, 5-azaspiro[3.5]nonane, 6-azaspiro[3.5]nonane, 7-azaspiro[3.5]nonane, 1-azaspiro[4.4]nonane, 2-azaspiro[4.4]nonane, 1-azaspiro[2.7]decane, 4-azaspiro[2.7]decane, 5-azaspiro[2.7]decane, 6-azaspiro[2.7]decane, 7-azaspiro[2.7]decane, 1-azaspiro[3.6]decane, 2-azaspiro[3.6]decane, 5-azaspiro[3.6]decane, 6-azaspiro[3.6]decane, 7-azaspiro[3.6]decane, 1-azaspiro[4.5]decane, 2-azaspiro[4.5]decane, 6-azaspiro[4.5]decane, 7-azaspiro[4.5]decane, 8-azaspiro[4.5]decane, 1-azaspiro[2.8]undecane, 4-azaspiro[2.8]undecane, 5-azaspiro[2.8]undecane, 6-azaspiro[2.8]undecane, 7-azaspiro[2.8]undecane, 1-azaspiro[3.7]undecane, 2-azaspiro[3.7]undecane, 5-azaspiro[3.7]undecane, 6-azaspiro[3.7]undecane, 7-azaspiro[3.7]undecane, 8-azaspiro[3.7]undecane, 1-azaspiro[4.6]undecane, 2-azaspiro[4.6]undecane, 6-azaspiro[4.6]undecane, 7-azaspiro[4.6]undecane, 8-azaspiro[4.6]undecane, 1-azaspiro[5.5]undecane, 2-azaspiro[5.5]undecane, and 3-azaspiro[5.5]undecane.

In some embodiments, Ring A is substituted with 0-3 R^{v} groups and is selected from any of the groups in Table 1C, Table 1D, Table 1E, Table 1F or Table 1G:

TABLE 1C

| | |
|---|---|
| 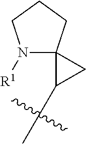 | i |
| 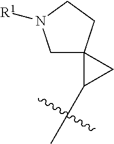 | ii |
| 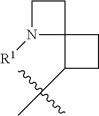 | iii |
| 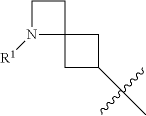 | iv |
| 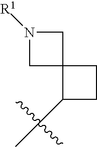 | v |
| 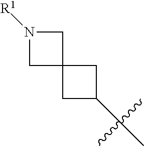 | vi |
| 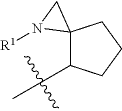 | vii |
| 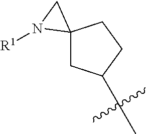 | viii |
| 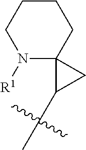 | ix |
| 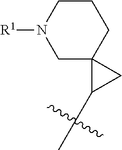 | x |
| 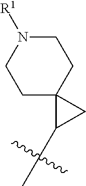 | xi |
| 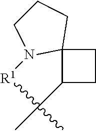 | xii |
| 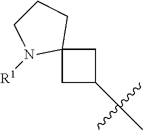 | xiii |
| 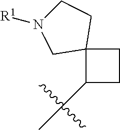 | xiv |
| 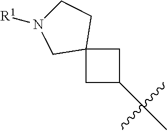 | xv |
| 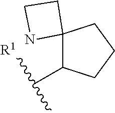 | xvi |
| 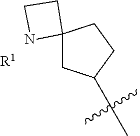 | xvii |
| 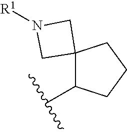 | xviii |
| 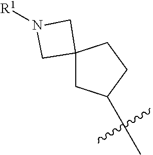 | xix |
| 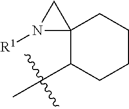 | xx |
| 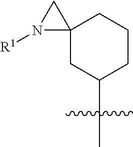 | xxi |
| 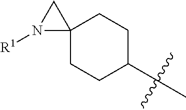 | xxii |
| 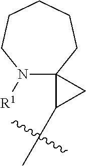 | xxiii |
| 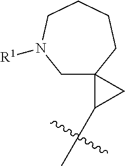 | xxiv |
| 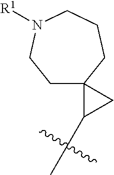 | xxv |
| 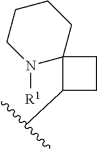 | xxvi |
| 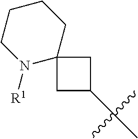 | xxvii |
| 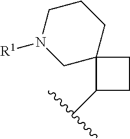 | xxviii |
| 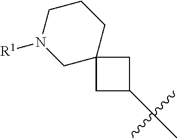 | xxix |
| 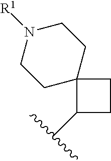 | xxx |
| 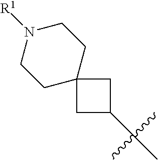 | xxxi |
| 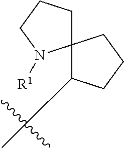 | xxxii |
| 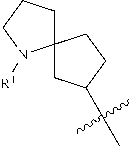 | xxxiii |
| 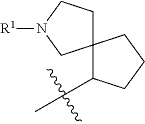 | xxxiv |
| 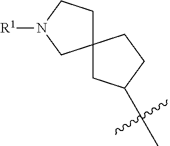 | xxxv |
| 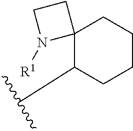 | xxxvi |
| 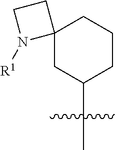 | xxxvii |
| 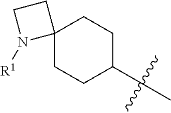 | xxxviii |
| 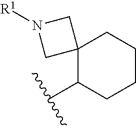 | xxxix |
| 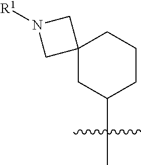 | xl |
| 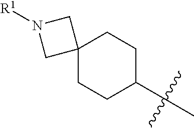 | xli |
| 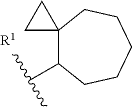 | xlii |
| 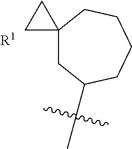 | xliii |
| 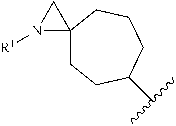 | xliv |
| 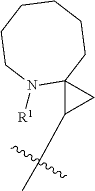 | xlv |
| 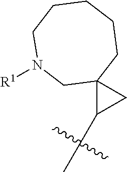 | xlvi |
| 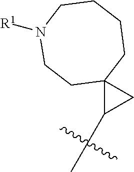 | xlvii |
| 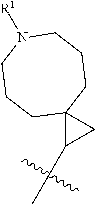 | xlviii |
| 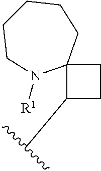 | xlix |
| 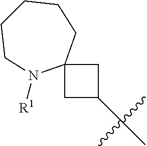 | l |
| 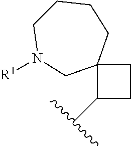 | li |
| 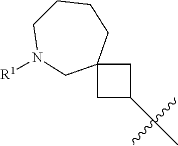 | lii |
| 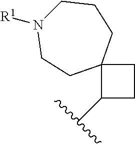 | liii |
| 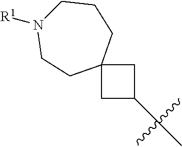 | liv |
| 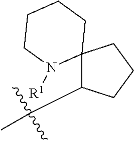 | lv |
| 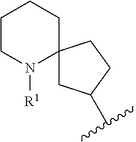 | lvi |
| 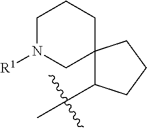 | lvii |
| 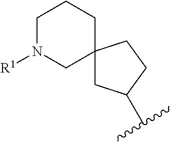 | lviii |
| 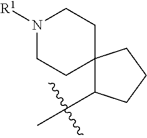 | lix |
| 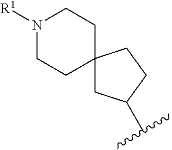 | lx |
| 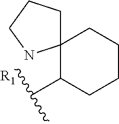 | lxi |
| 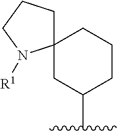 | lxii |
| 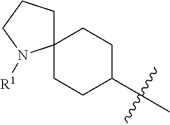 | lxiii |
| 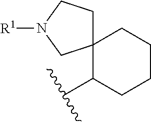 | lxiv |
| 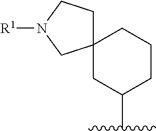 | lxv |
| 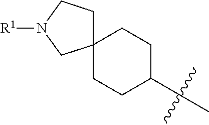 | lxvi |
| 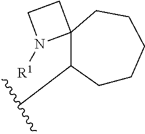 | lxvii |
| 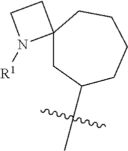 | lxviii |
| 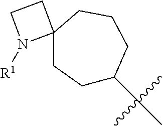 | lxix |
| 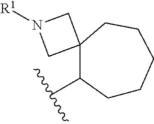 | lxx |
| 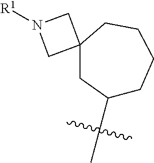 | lxxi |
| 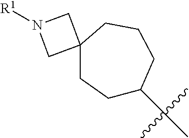 | lxxii |
| 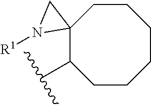 | lxxiii |
| 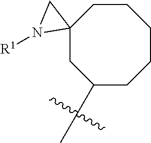 | lxxiv |
| 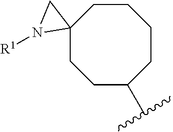 | lxxv |
| 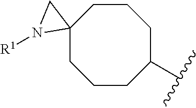 | lxxvi |
| 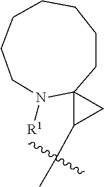 | lxxvii |
| 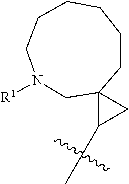 | lxxviii |
| 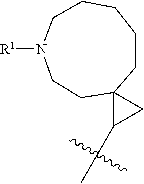 | lxxix |
| 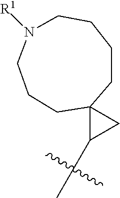 | lxxx |
| 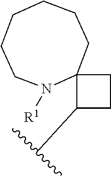 | lxxxi |
| 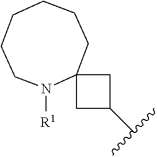 | lxxxii |
| 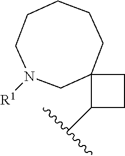 | lxxxiii |
| 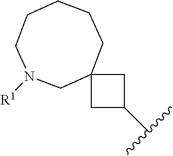 | lxxxiv |
| 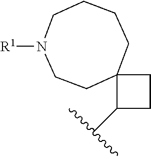 | lxxxv |
| 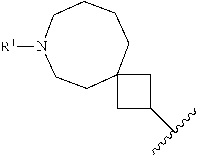 | lxxxvi |
| 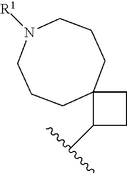 | lxxxvii |
| 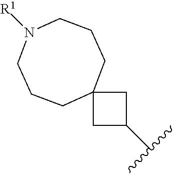 | lxxxviii |
| 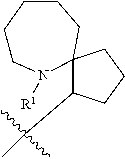 | lxxxix |
| 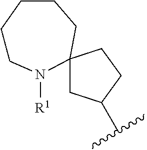 | xc |
| 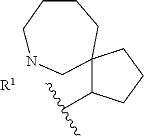 | xci |
| 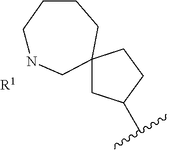 | xcii |
| 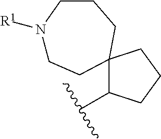 | xciii |
| 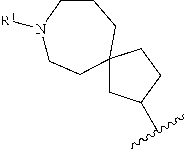 | xciv |
| 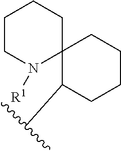 | xcv |
| 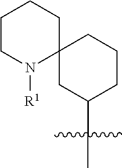 | xcvi |
| 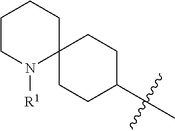 | xcvii |
| 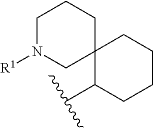 | xcviii |
| 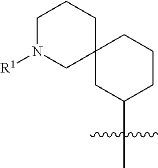 | xcix |
| 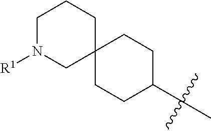 | c |
| 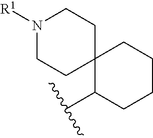 | ci |
| 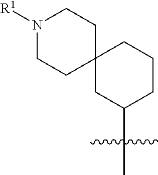 | cii |
| 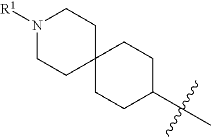 | ciii |
| 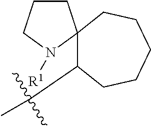 | civ |
| 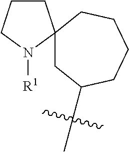 | cv |
| 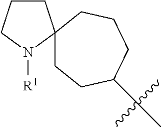 | cvi |
| 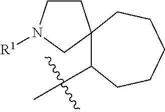 | cvii |
| 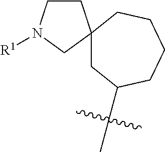 | cviii |
| 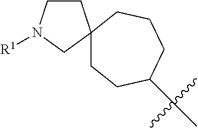 | cix |
| 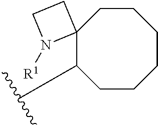 | cx |
| 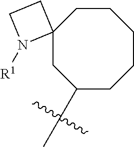 | cxi |
| 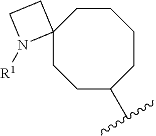 | cxii |
| 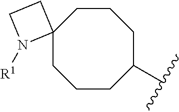 | cxiii |
| 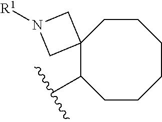 | cxiv |
| 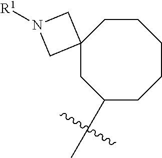 | cxv |
| 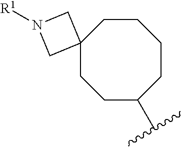 | cxvi |
| 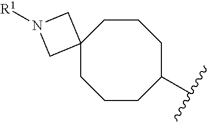 | cxvii |
| 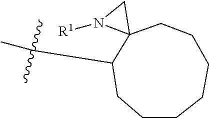 | cxviii |
| 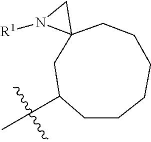 | cxix |
| 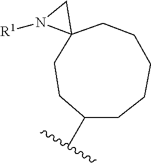 | cxx |
| 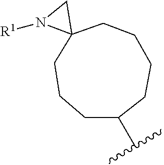 | cxxi |

TABLE 1D

| | |
|---|---|
| 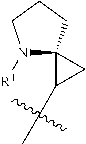 | i-a |
| 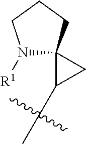 | i-b |
| 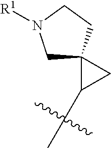 | ii-a |
| 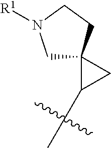 | ii-b |
| 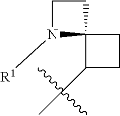 | iii-a |
| 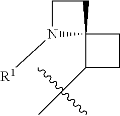 | iii-b |
| 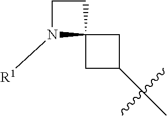 | iv-a |
| 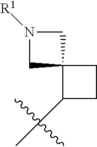 | v-a |
| 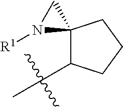 | vii-a |
| 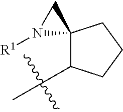 | vii-b |
| 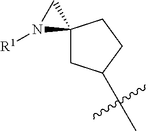 | viii-a |
| 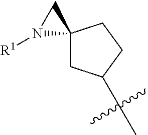 | viii-b |
| 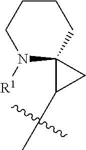 | ix-a |
| 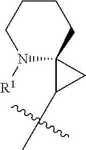 | ix-b |
| 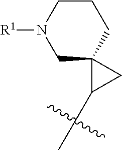 | x-a |
| 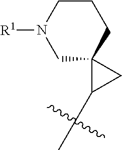 | x-b |
| 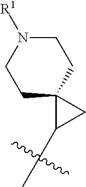 | xi-a |
| 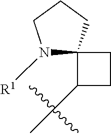 | xii-a |
| 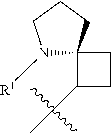 | xii-b |
| 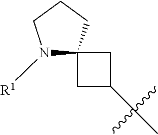 | xiii-a |
| 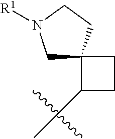 | xiv-a |
| 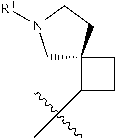 | xiv-b |
| 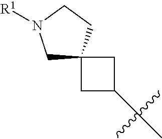 | xv-a |
| 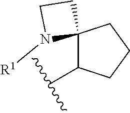 | xvi-a |
| 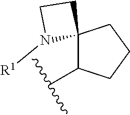 | xvi-b |
| 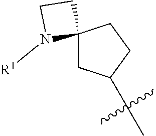 | xvii-a |
| 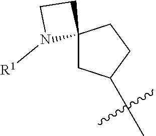 | xvii-b |
| 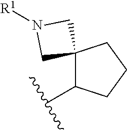 | xviii-a |
| 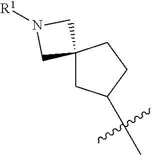 | xix-a |
| 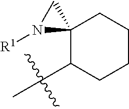 | xx-a |
| 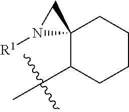 | xx-b |
| 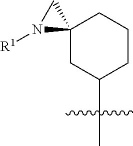 | xxi-a |
| 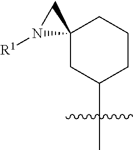 | xxi-b |
| 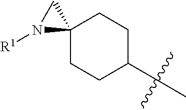 | xxii-a |
| 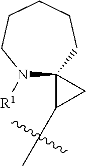 | xxiii-a |
| 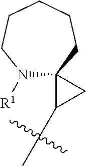 | xxiii-b |
| 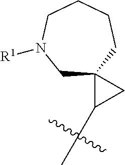 | xxiv-a |
| 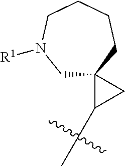 | xxiv-b |
| 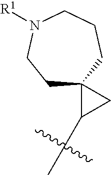 | xxv-a |
| 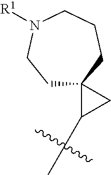 | xxv-b |
| 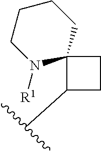 | xxvi-a |
| 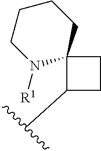 | xxvi-b |
| 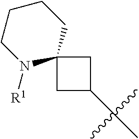 | xxvii-a |
| 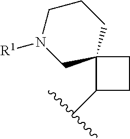 | xxviii-a |
| 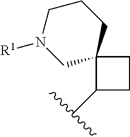 | xxviii-b |
| 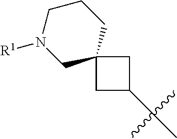 | xxix-a |
| 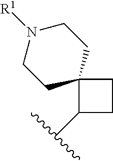 | xxx-a |
| 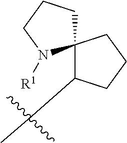 | xxxii-a |
| 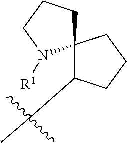 | xxxii-b |
| 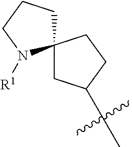 | xxxiii-a |
| 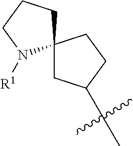 | xxxiii-b |
| 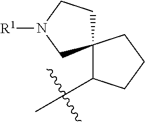 | xxxiv-a |
| 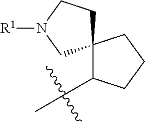 | xxxiv-b |
| 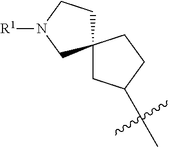 | xxxv-a |
| 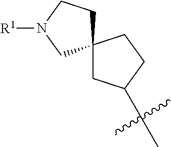 | xxxv-b |
| 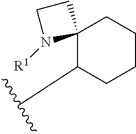 | xxxvi-a |
| 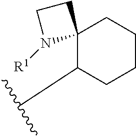 | xxxvi-b |
| 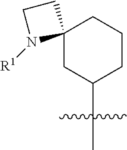 | xxxvii-a |
| 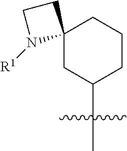 | xxxvii-b |
| 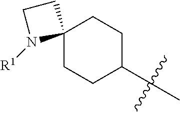 | xxxviii-a |
| 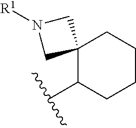 | xxxix-a |
| 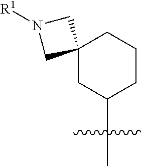 | xl-a |
| 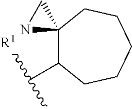 | xlii-a |
| 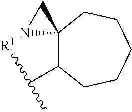 | xlii-b |
| 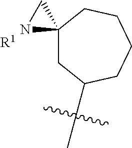 | xliii-a |
| 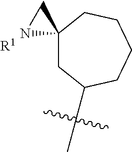 | xliii-b |
| 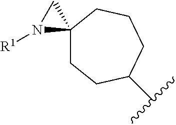 | xliv-a |
| 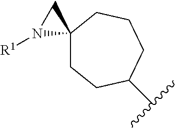 | xliv-b |
| 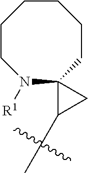 | xlv-a |
| 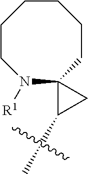 | xlv-b |
| 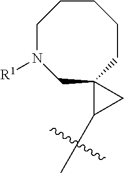 | xlvi-a |
| 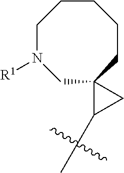 | xlvi-b |
| 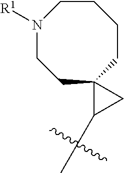 | xlvii-a |
| 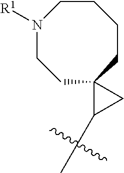 | xlvii-b |
| 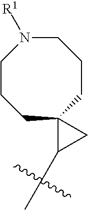 | xlviii-a |
| 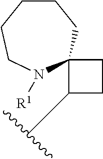 | xlix-a |
| 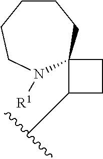 | xlix-b |
| 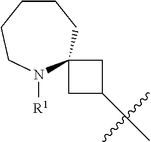 | l-a |
| 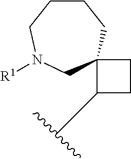 | li-a |
| 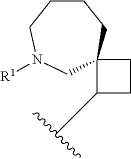 | li-b |
| 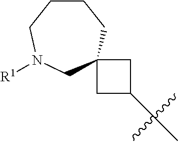 | lii-a |
| 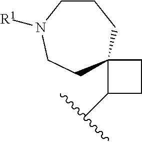 | liii-a |
| 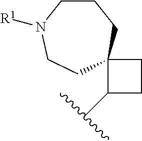 | liii-b |
| 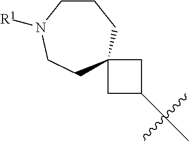 | liv-a |
| 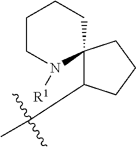 | lv-a |
| 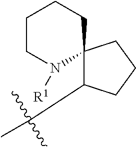 | lv-b |
| 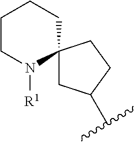 | lvi-a |
| 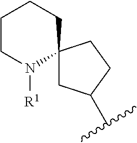 | lvi-b |
| 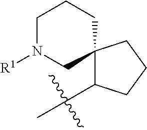 | lvii-a |
| 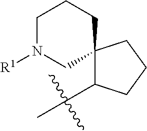 | lvii-b |
| 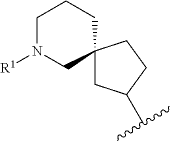 | lviii-a |
| 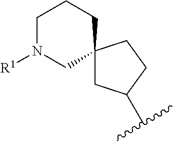 | lviii-b |
| 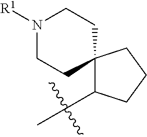 | lix-a |
| 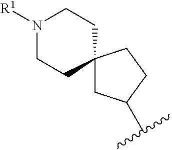 | lx-a |
| 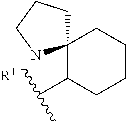 | lxi-a |
| 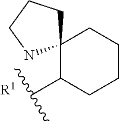 | lxi-b |
| 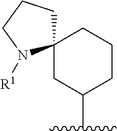 | lxii-a |
| 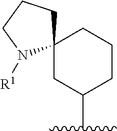 | lxii-b |
| 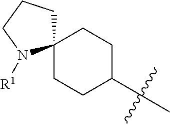 | lxiii-a |
| 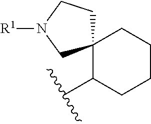 | lxiv-a |
| 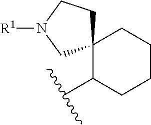 | lxiv-b |
| 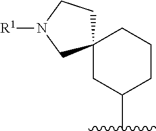 | lxv-a |
| 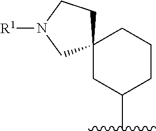 | lxv-b |
| 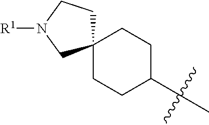 | lxvi-a |
| 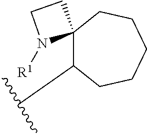 | lxvii-a |
| 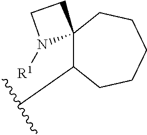 | lxvii-b |
| 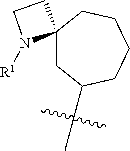 | lxviii-a |
| 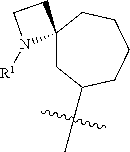 | lxviii-b |
| 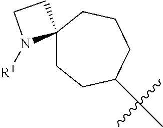 | lxix-a |
| 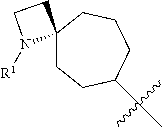 | lxix-b |
| 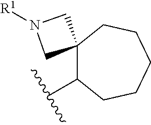 | lxx-a |
| 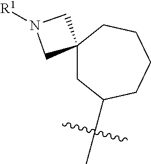 | lxxi-a |
| 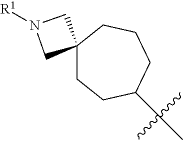 | lxxii-a |
| 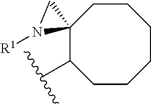 | lxxiii-a |
| 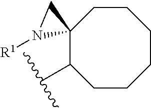 | lxxiii-b |
| 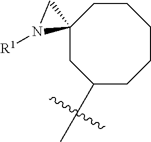 | lxxiv-a |
| 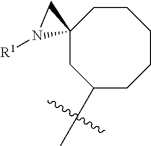 | lxxiv-b |
| 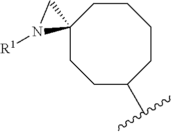 | lxxv-a |
| 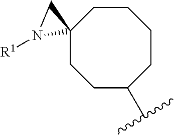 | lxxv-b |
| 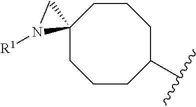 | lxxvi-a |
| 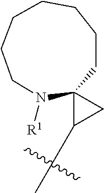 | lxxvii-a |
| 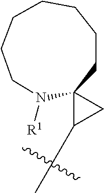 | lxxvii-b |
| 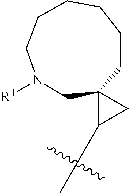 | lxxviii-a |
| 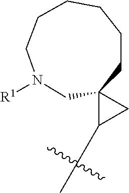 | lxxviii-b |
| 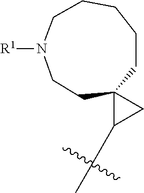 | lxxix-a |
| 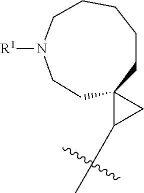 | lxxix-b |
| 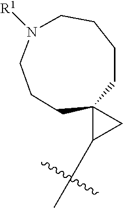 | lxxx-a |
| 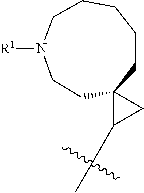 | lxxx-b |
| 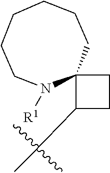 | lxxxi-a |
| 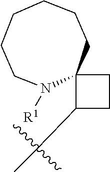 | lxxxi-b |
| 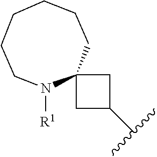 | lxxxii-a |
| 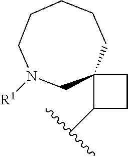 | lxxxiii-a |
| 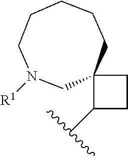 | lxxxiii-b |
| 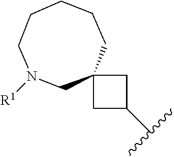 | lxxxiv-a |
| 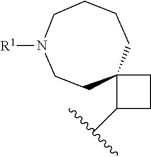 | lxxxv-a |
| 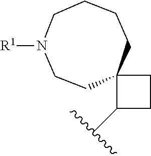 | lxxxv-b |
| 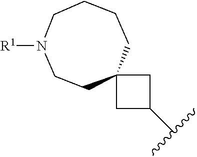 | lxxxvi-a |
| 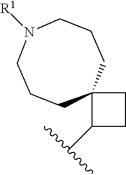 | lxxxvii-a |
| 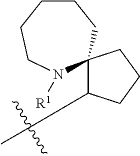 | lxxxix-a |
| 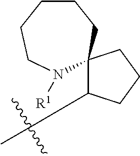 | lxxxix-b |
| 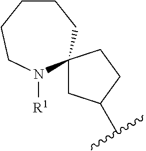 | xc-a |
| 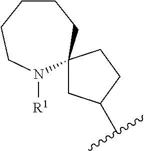 | xc-b |
| 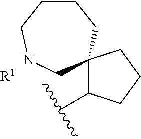 | xci-a |
| 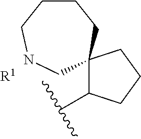 | xci-b |
| 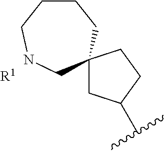 | xcii-a |
| 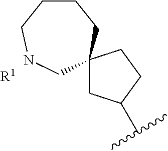 | xcii-b |
| 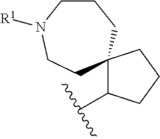 | xciii-a |
| 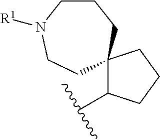 | xciii-b |
| 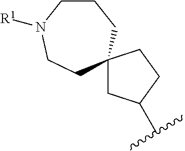 | xciv-a |
| 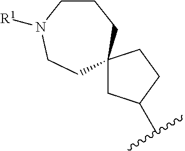 | xciv-b |
| 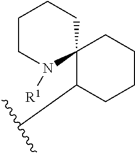 | xcv-a |
| 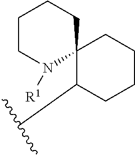 | xcv-b |
| 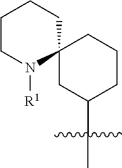 | xcvi-a |
| 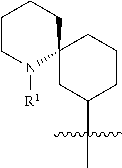 | xcvi-b |
| 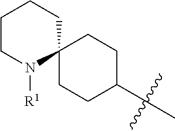 | xcvii-a |
| 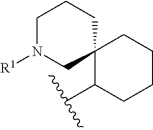 | xcviii-a |
| 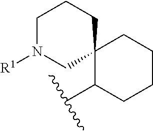 | xcviii-b |
| 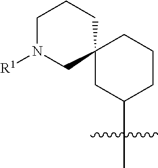 | xcix-a |
| 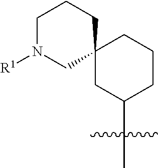 | xcix-b |
| 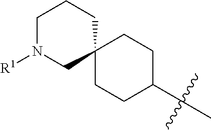 | c-a |
| 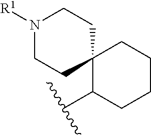 | ci-a |
| 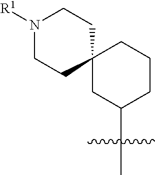 | cii-a |
| 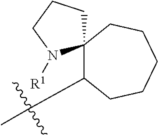 | civ-a |
| 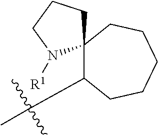 | civ-b |
| 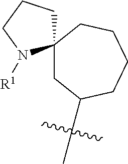 | cv-a |
| 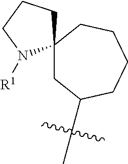 | cv-b |
| 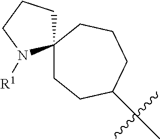 | cvi-a |
| 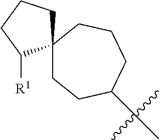 | cvi-b |
| 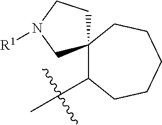 | cvii-a |
| 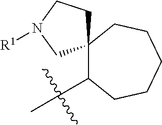 | cvii-b |
| 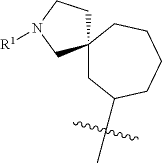 | cviii-a |
| 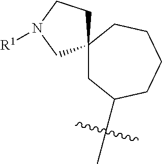 | cviii-b |
| 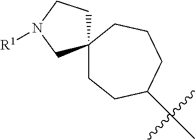 | cix-a |
| 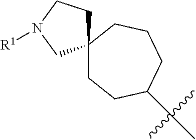 | cix-b |
| 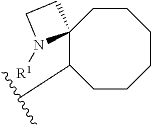 | cx-a |
| 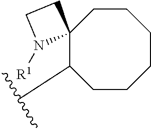 | cx-b |
| 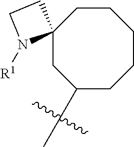 | cxi-a |
| 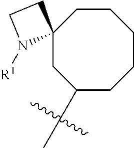 | cxi-b |
| 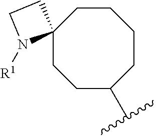 | cxii-a |
| 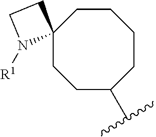 | cxii-b |
| 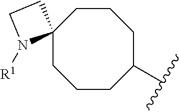 | cxiii-a |
| 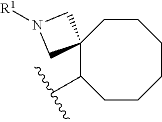 | cxiv-a |
| 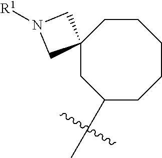 | cxv-a |
| 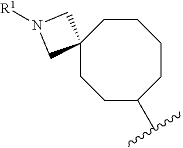 | cxvi-a |
| 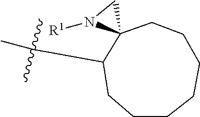 | cxviii-a |
| 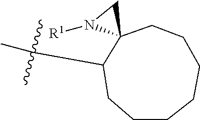 | cxviii-b |
| 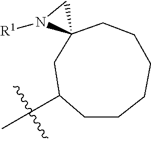 | cxix-a |
| 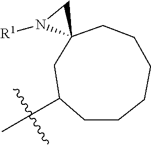 | cxix-b |
| 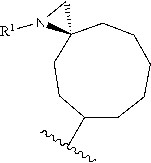 | cxx-a |
| 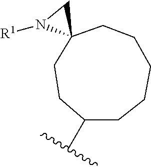 | cxx-b |
| 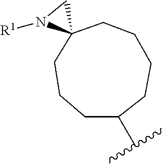 | cxxi-a |
| 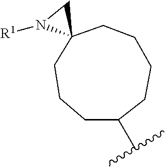 | cxxi-b |

TABLE 1E

| | |
|---|---|
| 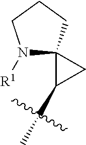 | i-a-i |
| 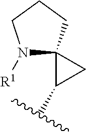 | i-a-ii |
| 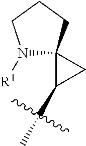 | i-b-i |
| 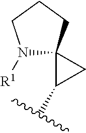 | i-b-ii |
| 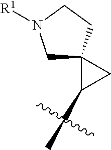 | ii-a-i |
| 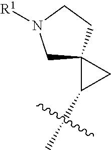 | ii-a-ii |
| 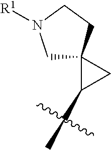 | ii-b-i |
| 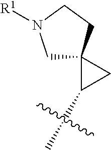 | ii-b-ii |
| 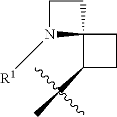 | iii-a-i |
| 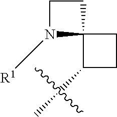 | iii-a-ii |
| 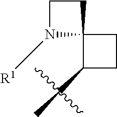 | iii-b-i |
| 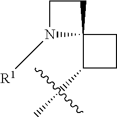 | iii-b-ii |
| 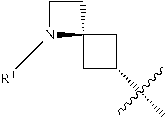 | iv-a-i |
| 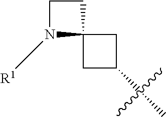 | iv-a-ii |
| 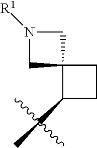 | v-a-i |
| 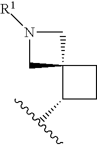 | v-a-ii |
| 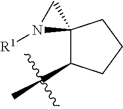 | vii-a-i |
| 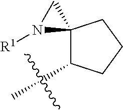 | vii-a-ii |
| 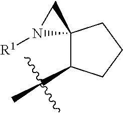 | vii-b-i |
| 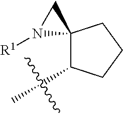 | vii-b-ii |
| 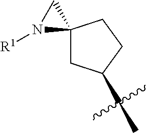 | viii-a-i |
| 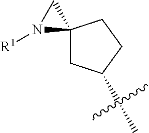 | viii-a-ii |
| 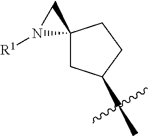 | viii-b-i |
| 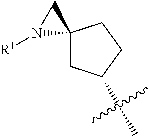 | viii-b-ii |
| 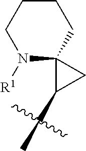 | ix-a-i |
| 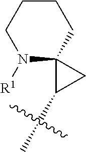 | ix-a-ii |
| 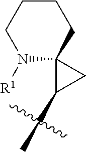 | ix-b-i |
| 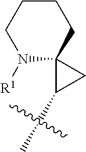 | ix-b-ii |
| 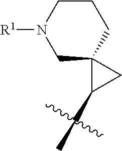 | x-a-i |
| 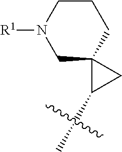 | x-a-ii |
| 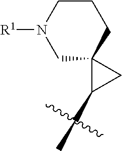 | x-b-i |
| 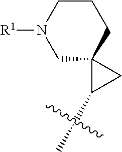 | x-b-ii |
| 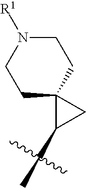 | xi-a-i |
| 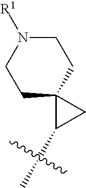 | xi-a-ii |
| 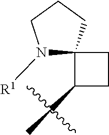 | xii-a-i |
| 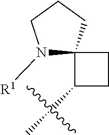 | xii-a-ii |
| 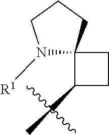 | xii-b-i |
| 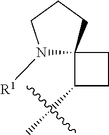 | xii-b-ii |
| 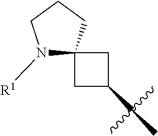 | xiii-a-i |
| 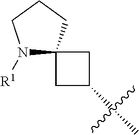 | xiii-a-ii |
| 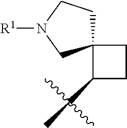 | xiv-a-i |
| 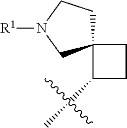 | xiv-a-ii |
| 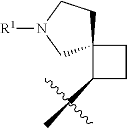 | xiv-b-i |
| 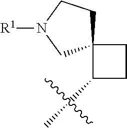 | xiv-b-ii |
| 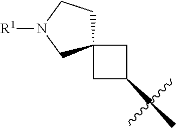 | xv-a-i |
| 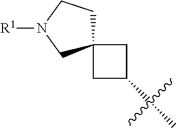 | xv-a-ii |
| 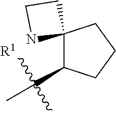 | xvi-a-i |
| 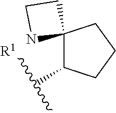 | xvi-a-ii |
| 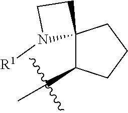 | xvi-b-i |
| 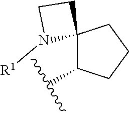 | xvi-b-ii |
| 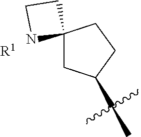 | xvii-a-i |
| 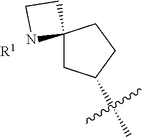 | xvii-a-ii |
| 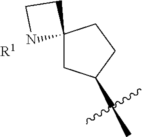 | xvii-b-i |
| 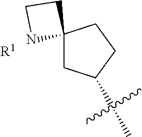 | xvii-b-ii |
| 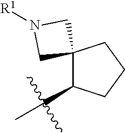 | xviii-a-i |
| 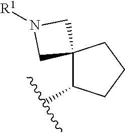 | xviii-a-ii |
| 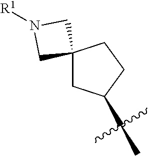 | xix-a-i |
| 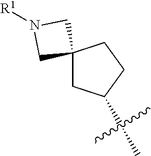 | xix-a-ii |
| 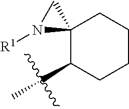 | xx-a-i |
| 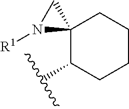 | xx-a-ii |
| 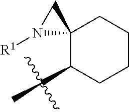 | xx-b-i |
| 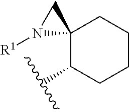 | xx-b-ii |
| 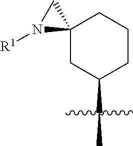 | xxi-a-i |
| 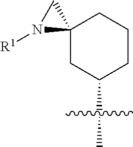 | xxi-a-ii |
| 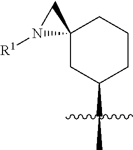 | xxi-b-i |
| 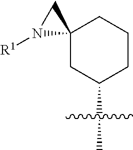 | xxi-b-ii |
| 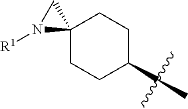 | xxii-a-i |
| 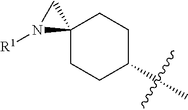 | xxii-a-ii |
| 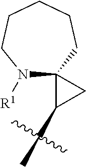 | xxiii-a-i |
| 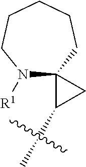 | xxiii-a-ii |
| 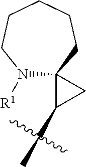 | xxiii-b |
| 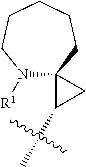 | xxiii-b |
| 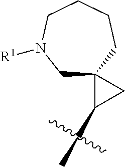 | xxiv-a-i |
| 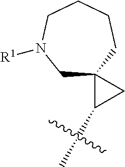 | xxiv-a-ii |
| 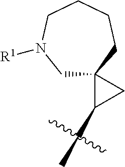 | xxiv-b-i |
| 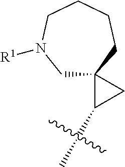 | xxiv-b-ii |
| 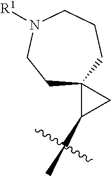 | xxv-a-i |
| 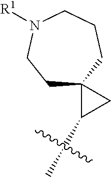 | xxv-a-ii |
| 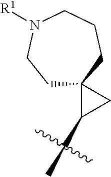 | xxv-b-i |
| 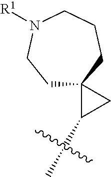 | xxv-b-ii |
| 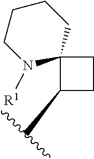 | xxvi-a-i |
| 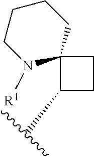 | xxvi-a-ii |
| 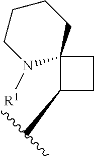 | xxvi-b-i |
| 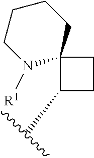 | xxvi-b-ii |
| 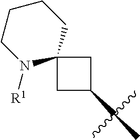 | xxvii-a-i |
| 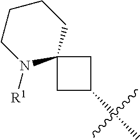 | xxvii-a-ii |
| 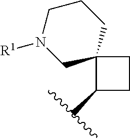 | xxviii-a-i |
| 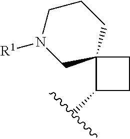 | xxviii-a-ii |
| 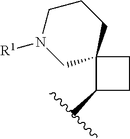 | xxviii-b-i |
| 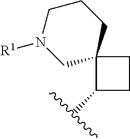 | xxviii-b-ii |
| 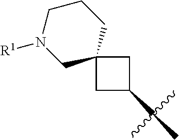 | xxix-a-i |
| 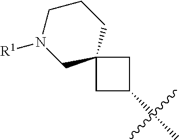 | xxix-a-ii |
| 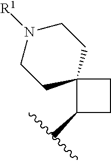 | xxx-a-i |
| 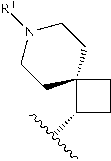 | xxx-a-ii |
| 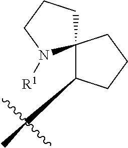 | xxxii-a-i |
| 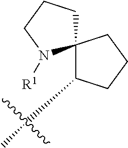 | xxxii-a-ii |
| 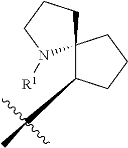 | xxxii-b-i |
| 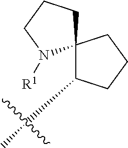 | xxxii-b-ii |
| 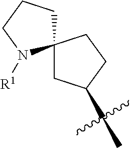 | xxxiii-a-i |
| 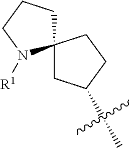 | xxxiii-a-ii |
| 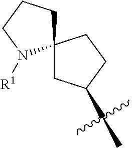 | xxxiii-b-i |
| 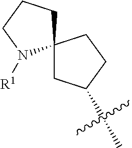 | xxxiii-b-ii |
| 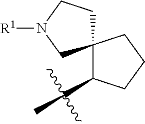 | xxxiv-a-i |
| 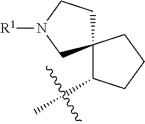 | xxxiv-a-ii |
| 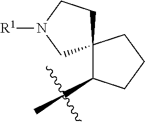 | xxxiv-b-i |
| 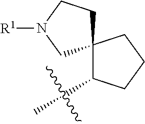 | xxxiv-b-ii |
| 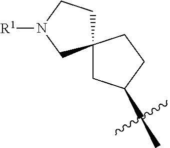 | xxxv-a-i |
| 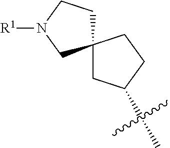 | xxxv-a-ii |
| 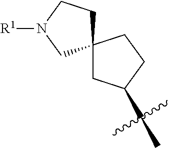 | xxxv-b-i |
| 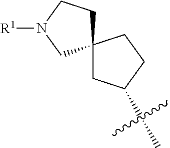 | xxxv-b-ii |
| 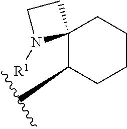 | xxxvi-a-i |
| 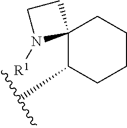 | xxxvi-a-ii |
| 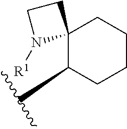 | xxxvi-b-i |
| 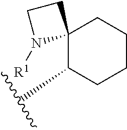 | xxxvi-b-ii |
| 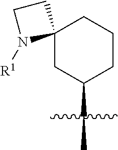 | xxxvii-a-i |
| 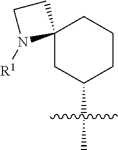 | xxxvii-a-ii |
| 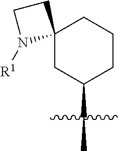 | xxxvii-b-i |
| 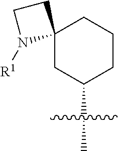 | xxxvii-b-ii |
| 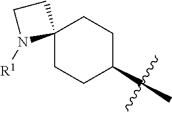 | xxxviii-a-i |
| 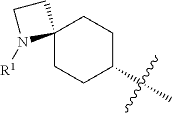 | xxxviii-a-ii |
| 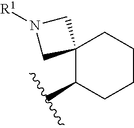 | xxxix-a-i |
| 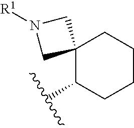 | xxxix-a-ii |
| 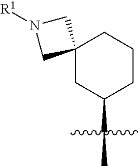 | xxxx-a-i |
| 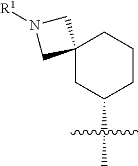 | xxxx-a-ii |
| 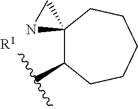 | xxxxii-a-i |
| 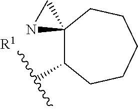 | xxxxii-a-ii |
| 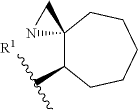 | xxxxii-b-i |
| 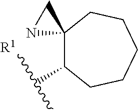 | xxxxii-b-ii |
| 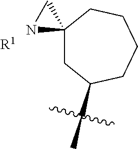 | xxxxiii-a-i |
| 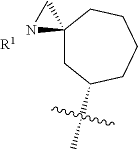 | xxxxiii-a-ii |
| 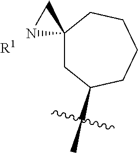 | xxxxiii-b-i |
| 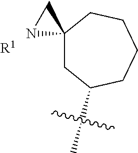 | xxxxiii-b-ii |
| 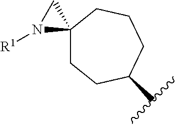 | xxxxiv-a-i |
| 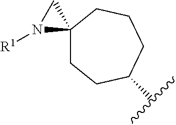 | xxxxiv-a-ii |
| 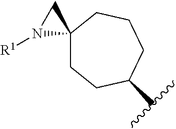 | xxxxiv-b-i |
| 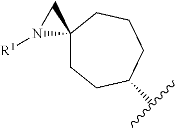 | xxxxiv-b-ii |
| 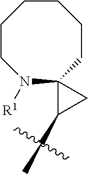 | xxxxv-a-i |
| 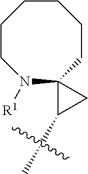 | xxxxv-a-ii |
| 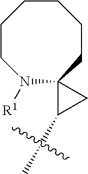 | xxxxv-b-i |
| 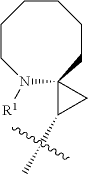 | xxxxv-b-ii |
| 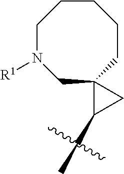 | xxxxvi-a-i |
| 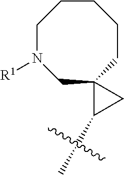 | xxxxvi-a-ii |
| 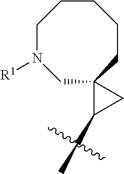 | xxxxvi-b-i |
| 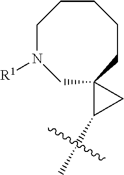 | xxxxvi-b-ii |
| 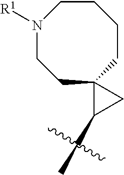 | xxxxvii-a-i |
| 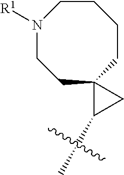 | xxxxvii-a-ii |
| 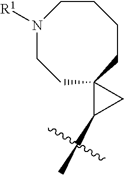 | xxxxvii-b-i |
| 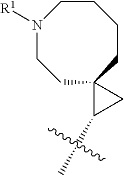 | xxxxvii-b-ii |
| 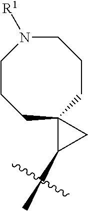 | xxxxviii-a-i |
| 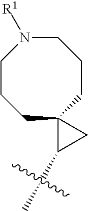 | xxxxviii-a-ii |
| 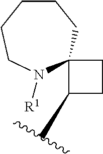 | xxxxix-a-i |
| 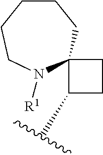 | xxxxix-a-ii |
| 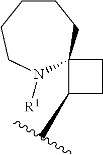 | xxxxix-b-i |
| 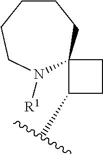 | xxxxix-b-ii |
| 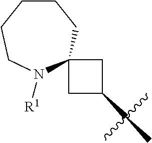 | l-a-i |
| 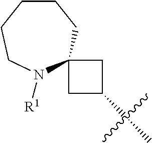 | l-a-ii |
| 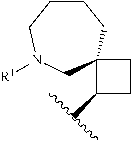 | li-a-i |
| 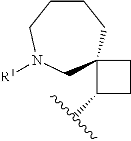 | li-a-ii |
| 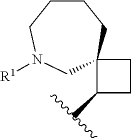 | li-b-i |
| 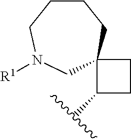 | li-b-ii |
| 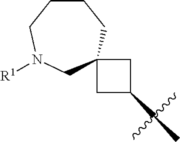 | lii-a-i |
| 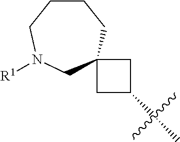 | lii-a-ii |
| 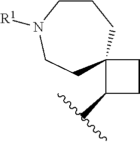 | liii-a-i |
| 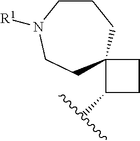 | liii-a-ii |
| 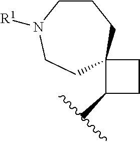 | liii-b-i |
| 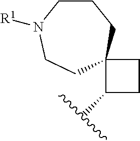 | liii-b-ii |
| 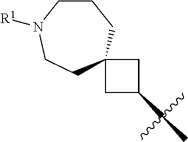 | liv-a-i |
| 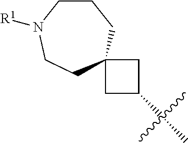 | liv-a-ii |
| 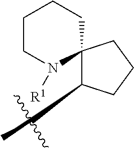 | lv-a-i |
| 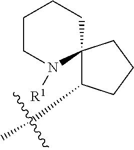 | lv-a-ii |
| 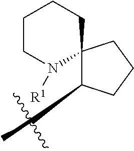 | lv-b-i |
| 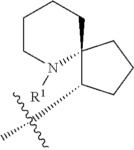 | lv-b-ii |
| 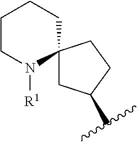 | lvi-a-i |
| 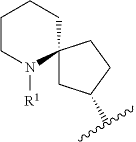 | lvi-a-ii |
| 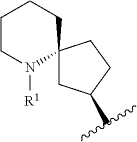 | lvi-b-i |
| 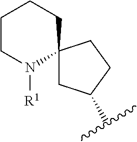 | lvi-b-ii |
| 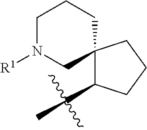 | lvii-a-i |
| 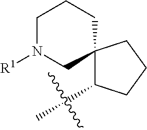 | lvii-a-ii |
| 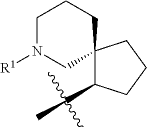 | lvii-b-i |
| 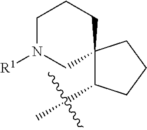 | lvii-b-ii |
| 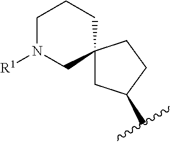 | lviii-a-i |
| 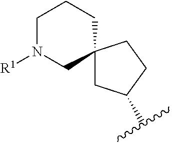 | lviii-a-ii |
| 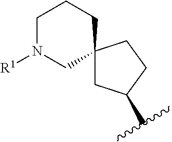 | lviii-b-i |
| 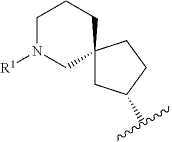 | lviii-b-ii |
| 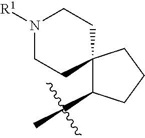 | lix-a-i |
| 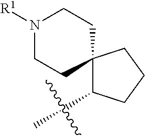 | lix-a-ii |
| 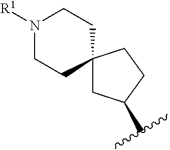 | lx-a-i |
| 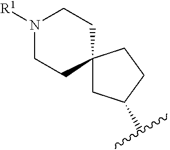 | lx-a-ii |
| 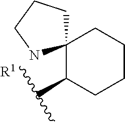 | lxi-a-i |
| 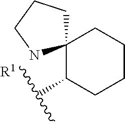 | lxi-a-ii |
| 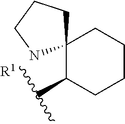 | lxi-b-i |
| 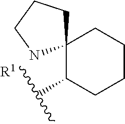 | lxi-b-ii |
| 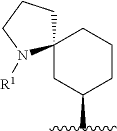 | lxii-a-i |
| 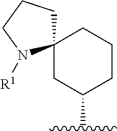 | lxii-a-ii |
| 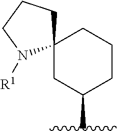 | lxii-b-i |
| 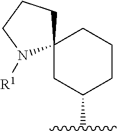 | lxii-b-ii |
| 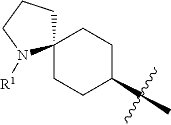 | lxiii-a-i |
| 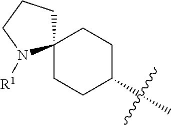 | lxiii-a-ii |
| 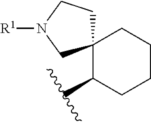 | lxiv-a-i |
| 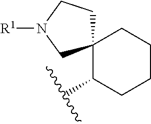 | lxiv-a-ii |
| 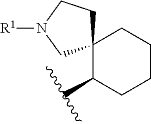 | lxiv-b-i |
| 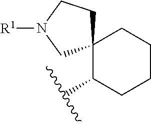 | lxiv-b-ii |
| 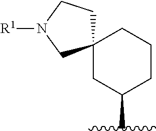 | lxv-a-i |
| 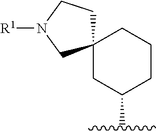 | lxv-a-ii |
| 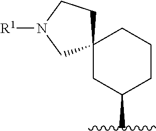 | lxv-b-i |
| 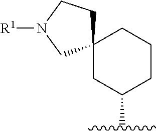 | lxv-b-ii |
| 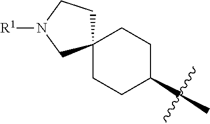 | lxvi-a-i |
| 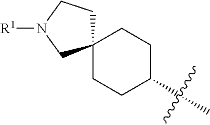 | lxvi-a-ii |
| 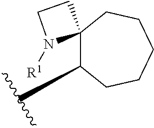 | lxvii-a-i |
| 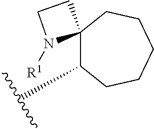 | lxvii-a-ii |
| 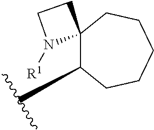 | lxvii-b-i |
| 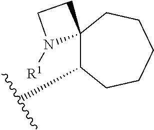 | lxvii-b-ii |
| 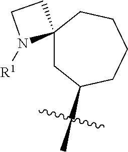 | lxviii-a-i |
| 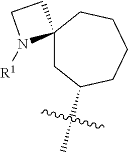 | lxviii-a-ii |
| 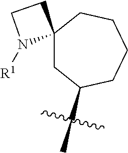 | lxviii-b-i |
| 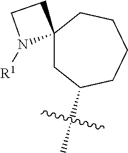 | lxviii-b-ii |
| 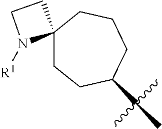 | lxix-a-i |
| 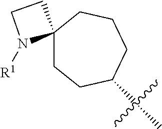 | lxix-a-ii |
| 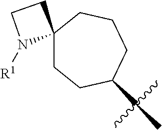 | lxix-b-i |
| 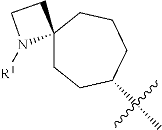 | lxix-b-ii |
| 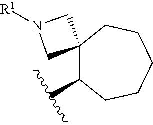 | lxx-a-i |
| 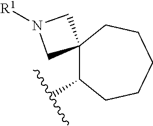 | lxx-a-ii |
| 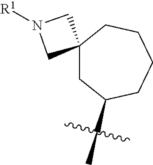 | lxxi-a-i |
| 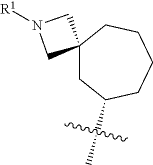 | lxxi-a-ii |
| 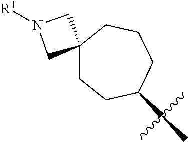 | lxxii-a-i |
| 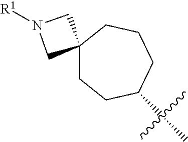 | lxxii-a-ii |
| 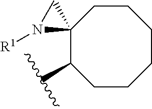 | lxxiii-a-i |
| 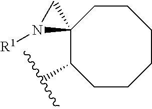 | lxxiii-a-ii |
| 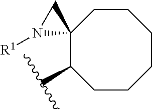 | lxxiii-b-i |
| 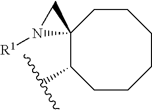 | lxxiii-b-ii |
| 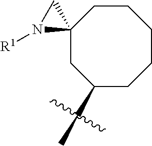 | lxxiv-a-i |
| 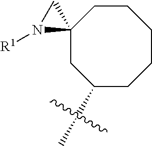 | lxxiv-a-ii |
| 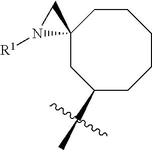 | lxxiv-b-i |
| 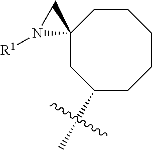 | lxxiv-b-ii |
| 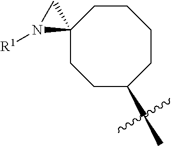 | lxxv-a-i |
| 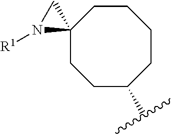 | lxxv-a-ii |
| 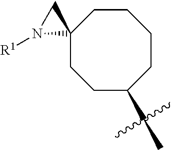 | lxxv-b-i |
| 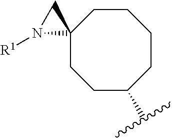 | lxxv-b-ii |
| 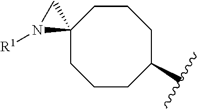 | lxxvi-a-i |
| 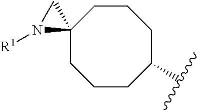 | lxxvi-a-ii |
| 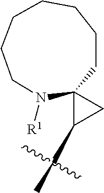 | lxxvii-a-i |
| 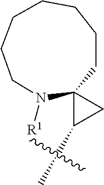 | lxxvii-a-ii |
| 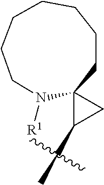 | lxxvii-b-i |
| 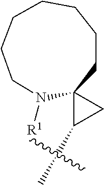 | lxxvii-b-ii |
| 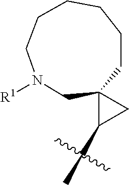 | lxxviii-a-i |
| 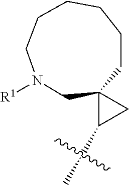 | lxxviii-a-ii |
| 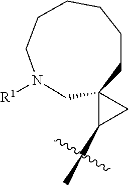 | lxxviii-b-i |
| 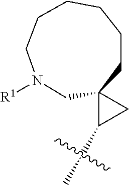 | lxxviii-b-ii |
| 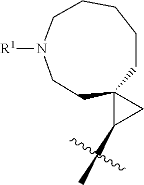 | lxxix-a-i |
| 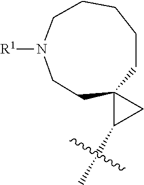 | lxxix-a-ii |
| 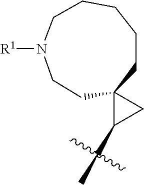 | lxxix-b-i |
| 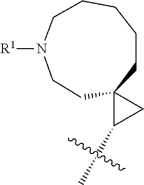 | lxxix-b-ii |
| 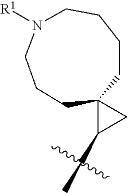 | lxxx-a-i |
| 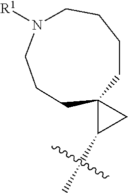 | lxxx-a-ii |
| 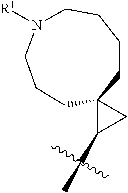 | lxxx-b-i |
| 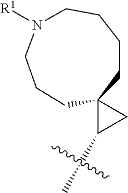 | lxxx-b-ii |
| 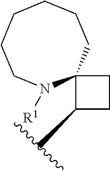 | lxxxi-a-i |
| 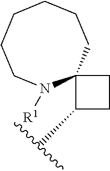 | lxxxi-a-ii |
| 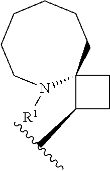 | lxxxi-b-i |
| 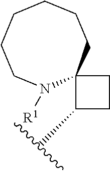 | lxxxi-b-ii |
| 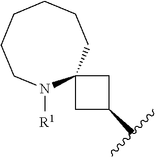 | lxxxii-a-i |
| 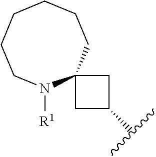 | lxxxii-a-ii |
| 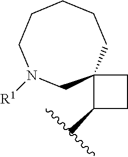 | lxxxiii-a-i |
| 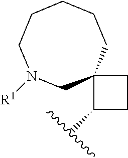 | lxxxiii-a-ii |
| 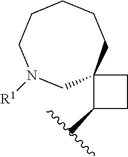 | lxxxiii-b-i |
| 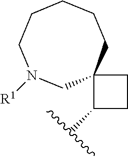 | lxxxiii-b-ii |
| 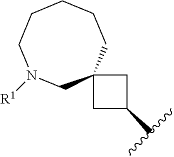 | lxxxiv-a-i |
| 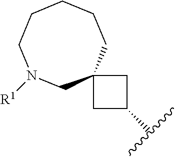 | lxxxiv-a-ii |
| 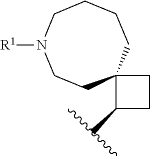 | lxxxv-a-i |
| 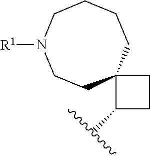 | lxxxv-a-ii |
| 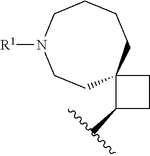 | lxxxv-b-i |
| 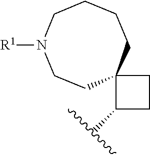 | lxxxv-b-ii |
| 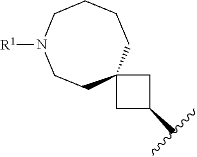 | lxxxvi-a-i |
| 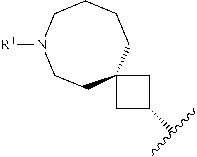 | lxxxvi-a-ii |
| 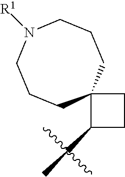 | lxxxvii-a-i |
| 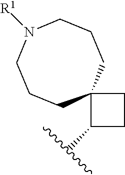 | lxxxvii-a-ii |
| 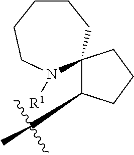 | lxxxix-a-i |
| 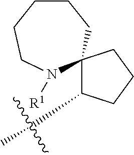 | lxxxix-a-ii |
| 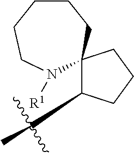 | lxxxix-b-i |
| 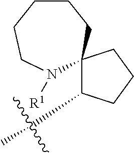 | lxxxix-b-ii |
| 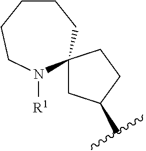 | xc-a-i |
| 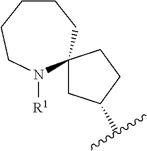 | xc-a-ii |
| 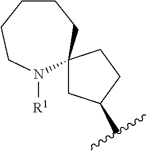 | xc-b-i |
| 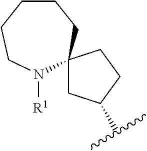 | xc-b-ii |
| 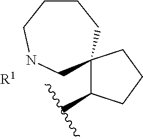 | xci-a-i |
| 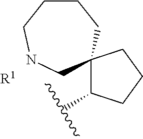 | xci-a-ii |
| 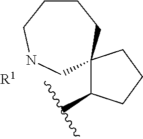 | xci-b-i |
| 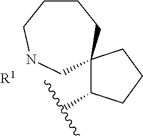 | xci-b-ii |
| 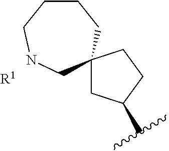 | xcii-a-i |
| 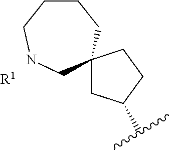 | xcii-a-ii |
| 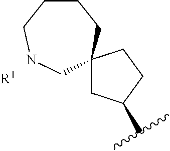 | xcii-b-i |
| 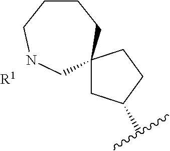 | xcii-b-ii |
| 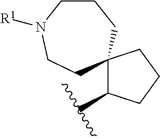 | xciii-a-i |
| 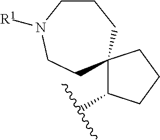 | xciii-a-ii |
| 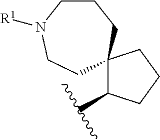 | xciii-b-i |
| 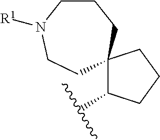 | xciii-b-ii |
| 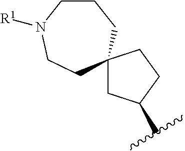 | xciv-a-i |
| 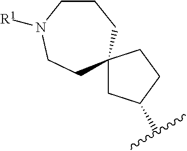 | xciv-a-ii |
| 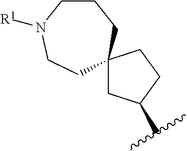 | xciv-b-i |
| 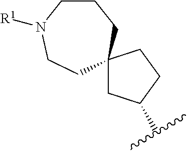 | xciv-b-ii |
| 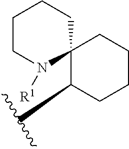 | xcv-a-i |
| 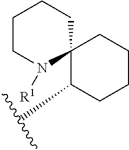 | xcv-a-ii |
| 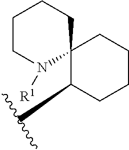 | xcv-b-i |
| 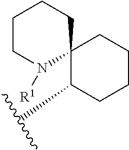 | xcv-b-ii |
| 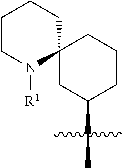 | xcvi-a-i |
| 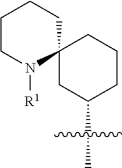 | xcvi-a-ii |
| 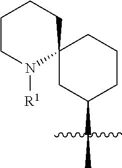 | xcvi-b-i |
| 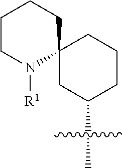 | xcvi-b-ii |
| 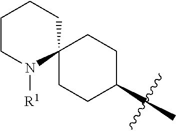 | xcvii-a-ii |
| 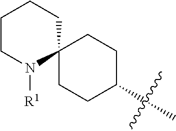 | xcvii-a-ii |
| 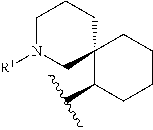 | xcviii-a-i |
| 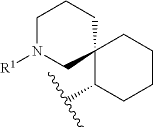 | xcviii-a-ii |
| 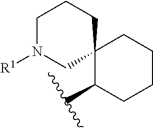 | xcviii-b-i |
| 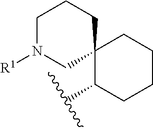 | xcviii-b-ii |
| 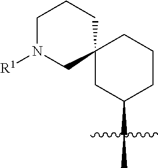 | xcix-a-i |
| 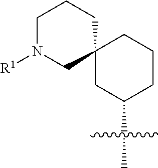 | xcix-a-ii |
| 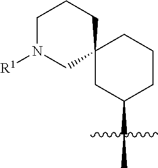 | xcix-b-i |
| 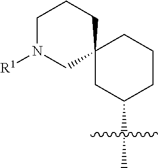 | xcix-b-ii |
| 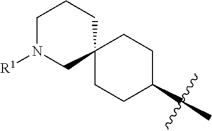 | c-a-i |
| 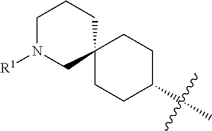 | c-a-ii |
| 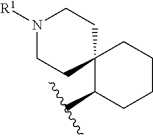 | ci-a-i |
| 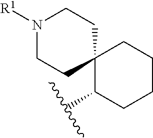 | ci-a-ii |
| 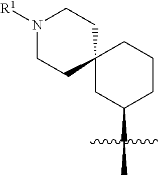 | cii-a-i |
| 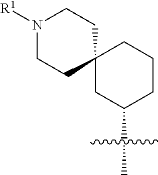 | cii-a-ii |
| 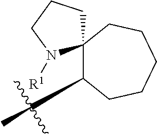 | civ-a-i |
| 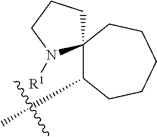 | civ-a-ii |
| 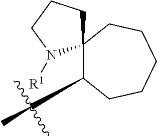 | civ-b-i |
| 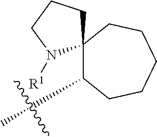 | civ-b-ii |
| 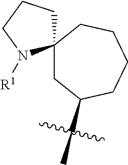 | cv-a-i |
| 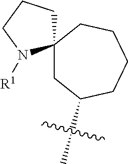 | cv-a-ii |
| 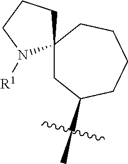 | cv-b-i |
| 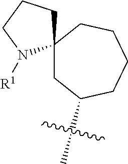 | cv-b-ii |
| 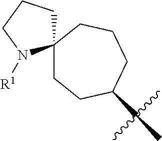 | cvi-a-i |
| 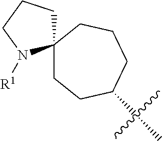 | cvi-a-ii |
| 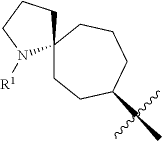 | cvi-b-i |
| 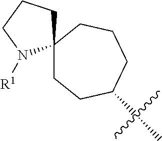 | cvi-b-ii |
| 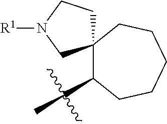 | cvii-a-i |
| 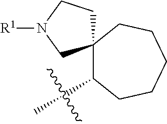 | cvii-a-ii |
| 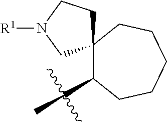 | cvii-b-i |
| 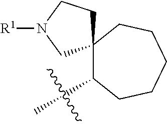 | cvii-b-ii |
| 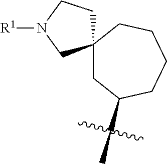 | cviii-a-i |
| 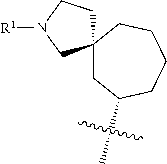 | cviii-a-ii |
| 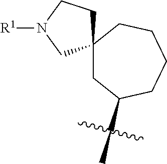 | cviii-b-i |
| 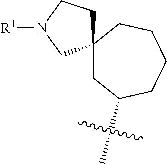 | cviii-b-ii |
| 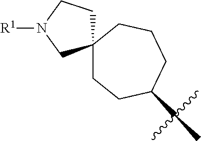 | cix-a-i |
| 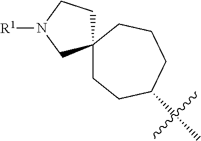 | cix-a-ii |
| 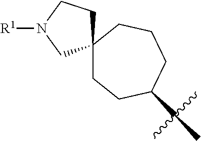 | cix-b-i |
| 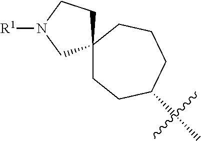 | cix-b-ii |
| 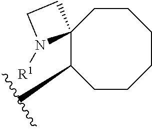 | cx-a-i |
| 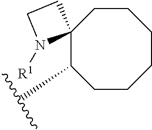 | cx-a-ii |
| 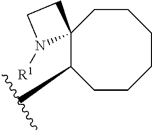 | cx-b-i |
| 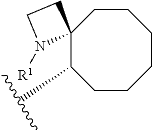 | cx-b-ii |
| 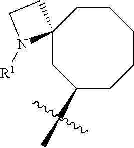 | cxi-a-i |
| 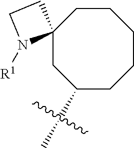 | cxi-a-ii |
| 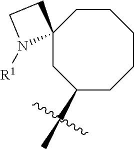 | cxi-b-i |
| 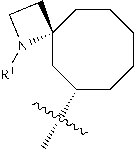 | cxi-b-ii |
| 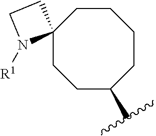 | cxii-a-i |
| 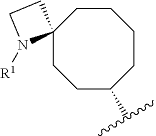 | cxii-a-ii |
| 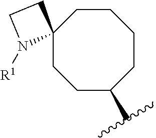 | cxii-b-i |
| 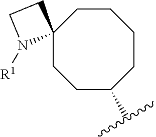 | cxii-b-ii |
| 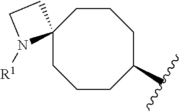 | cxiii-a-i |
| 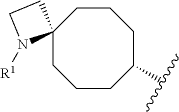 | cxiii-a-ii |
| 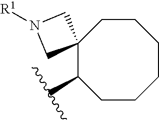 | cxiv-a-i |
| 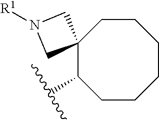 | cxiv-a-ii |
| 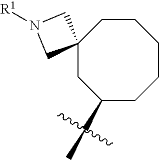 | cxv-a-i |
| 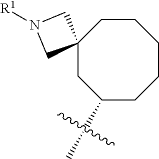 | cxv-a-ii |
| 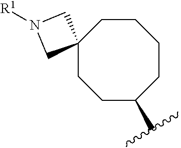 | cxvi-a-i |
| 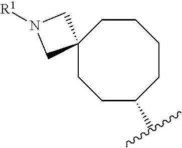 | cxvi-a-ii |
| 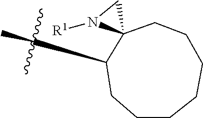 | cxviii-a-i |
| 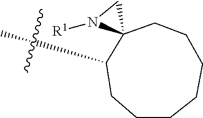 | cxviii-a-ii |
| 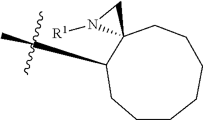 | cxviii-b-i |
| 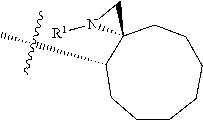 | cxviii-b-ii |
| 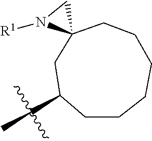 | cxix-a-i |
| 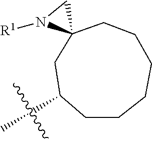 | cxix-a-ii |
| 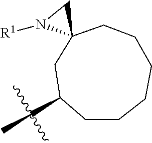 | cxix-b-i |
| 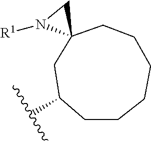 | cxix-b-ii |
| 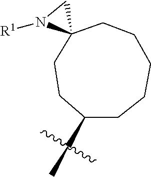 | cxx-a-i |
| 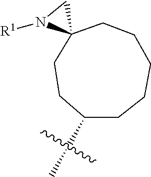 | cxx-a-ii |
| 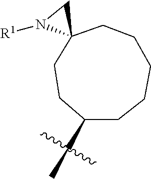 | cxx-b-i |
| 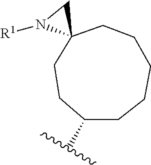 | cxx-b-ii |
| 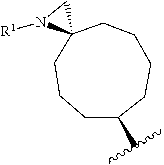 | cxxi-a-i |
| 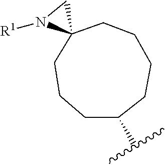 | cxxi-a-ii |
| 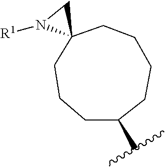 | cxxi-b-i |
| 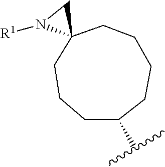 | cxxi-b-ii |

TABLE 1F

| | |
|---|---|
| 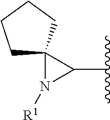 | cxxii |
| 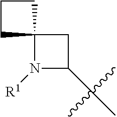 | cxxiiii |
| 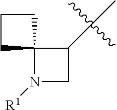 | cxxiv |
| 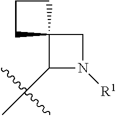 | cxxv |
| 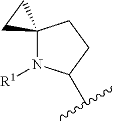 | cxxvi |
| 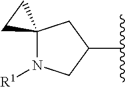 | cxxvii |
| 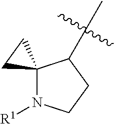 | cxxviii |
| 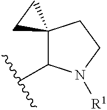 | cxxix |
| 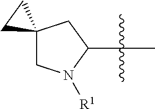 | cxxx |
| 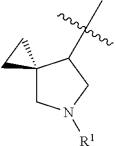 | cxxxi |
| 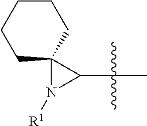 | cxxxii |
| 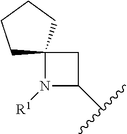 | cxxxiii |
| 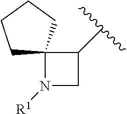 | cxxxiv |
| 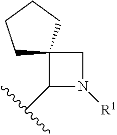 | cxxxv |
| 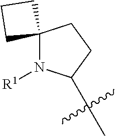 | cxxxvi |
| 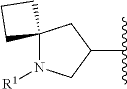 | cxxxvii |
| 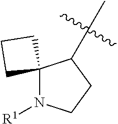 | cxxxviii |
| 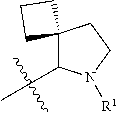 | cxxxix |
| 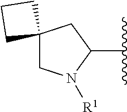 | cxl |
| 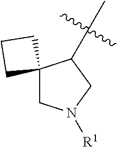 | cxli |
| 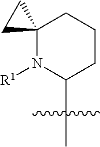 | cxlii |
| 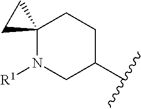 | cxliii |
| 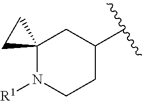 | cxliv |
| 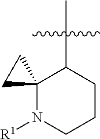 | cxlv |
| 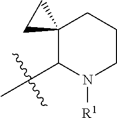 | cxlvi |
| 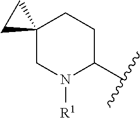 | cxlvii |
| 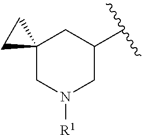 | cxlviii |
| 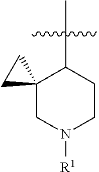 | cxlix |
| 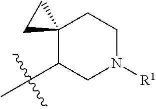 | cl |
| 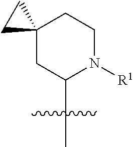 | cli |
| 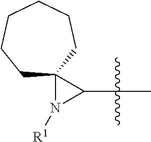 | clii |
| 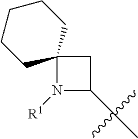 | cliii |
| 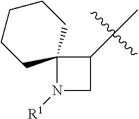 | cliv |
| 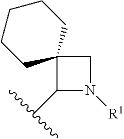 | clv |
| 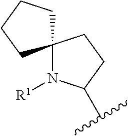 | clvi |
| 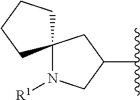 | clvii |
| 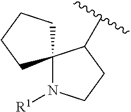 | clviii |
| 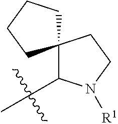 | clix |
| 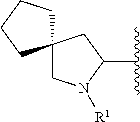 | clx |
| 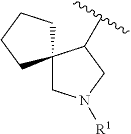 | clxi |
| 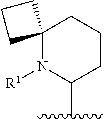 | clxii |
| 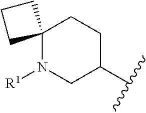 | clxiii |
| 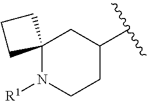 | clxiv |
| 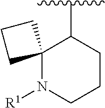 | clxv |
| 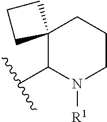 | clxvi |
| 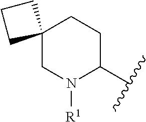 | clxvii |
| 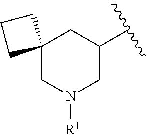 | clxviii |
| 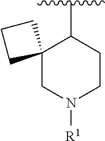 | clxix |
| 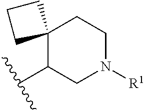 | clxx |
| 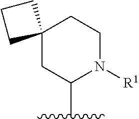 | clxxi |
| 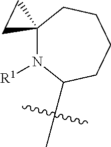 | clxxii |
| 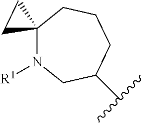 | clxxiii |
| 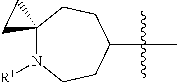 | clxxiv |
| 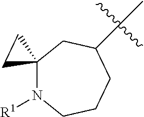 | clxxv |
| 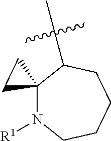 | clxxvi |
| 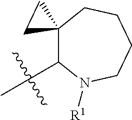 | clxxvii |
| 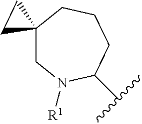 | clxxviii |
| 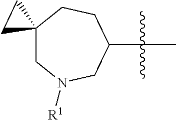 | clxxix |
| 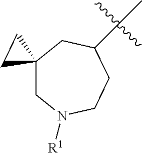 | clxxx |
| 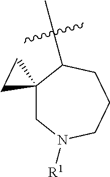 | clxxxi |
| 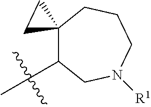 | clxxxii |
| 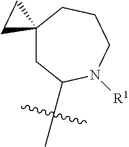 | clxxxiii |
| 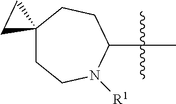 | clxxxiv |
| 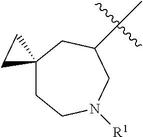 | clxxxv |
| 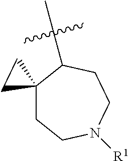 | clxxxvi |
| 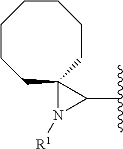 | clxxxvii |
| 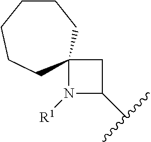 | clxxxviii |
| 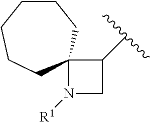 | clxxxix |
| 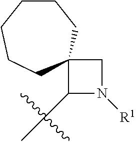 | cxc |
| 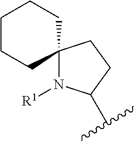 | cxci |
| 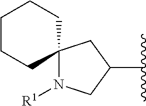 | cxcii |
| 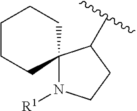 | cxciii |
| 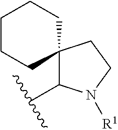 | cxciv |
| 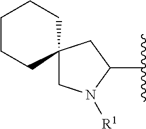 | cxcv |
| 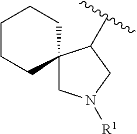 | cxcvi |
| 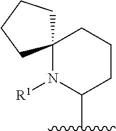 | cxcvii |
| 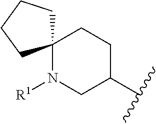 | cxcviii |
| 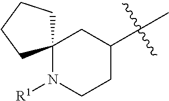 | cxcix |
| 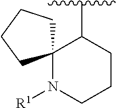 | cc |
| 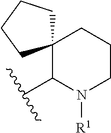 | cci |
| 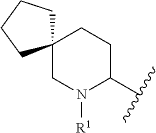 | ccii |
| 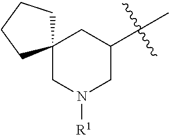 | cciii |
| 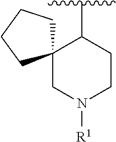 | cciv |
| 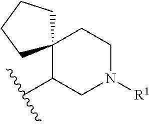 | ccv |
| 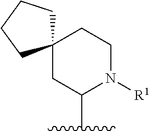 | ccvi |
| 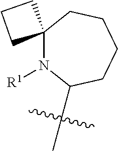 | ccvii |
| 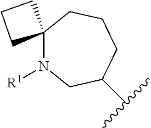 | ccviii |
| 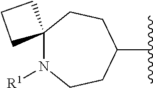 | ccix |
| 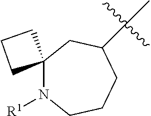 | ccx |
| 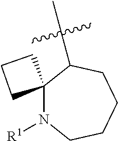 | ccxi |
| 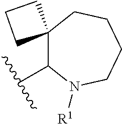 | ccxii |
| 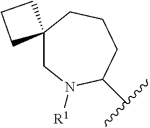 | ccxiii |
| 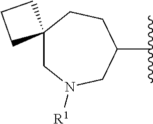 | ccxiv |
| 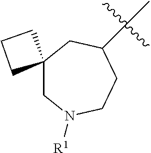 | ccxv |
| 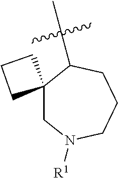 | ccxvi |
| 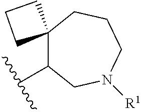 | ccxvii |
| 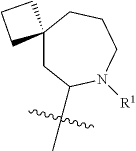 | ccxviii |
| 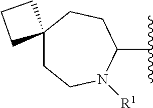 | ccxix |
| 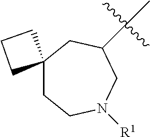 | ccxx |
| 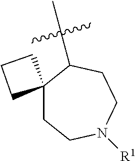 | ccxxi |
| 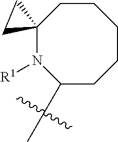 | ccxxii |
| 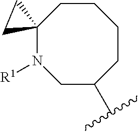 | ccxxiii |
| 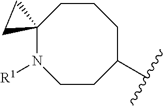 | ccxxiv |
| 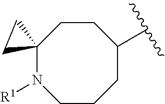 | ccxxv |
| 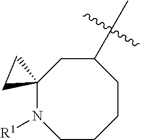 | ccxxvi |
| 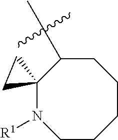 | ccxxvii |
| 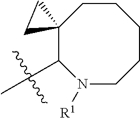 | ccxxviii |
| 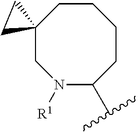 | ccxxix |
| 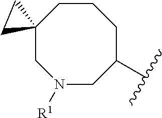 | ccxxx |
| 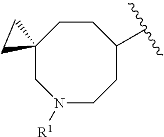 | ccxxxi |
| 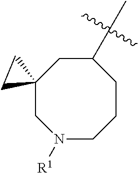 | ccxxxii |
| 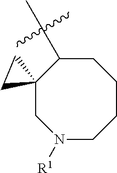 | ccxxxiii |
| 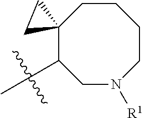 | ccxxxiv |
| 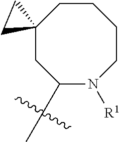 | ccxxxv |
| 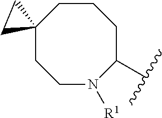 | ccxxxvi |
| 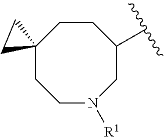 | ccxxxvii |
| 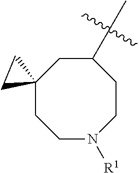 | ccxxxviii |
| 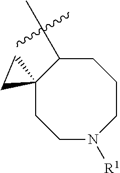 | ccxxxix |
| 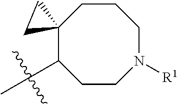 | ccxl |
| 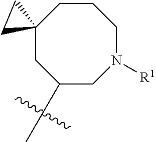 | ccxli |
| 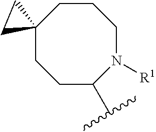 | ccxlii |
| 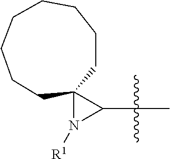 | ccxliii |
| 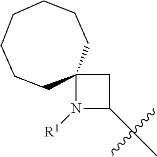 | ccxliv |
| 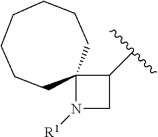 | ccxlv |
| 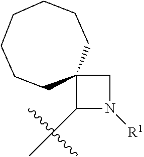 | ccxlvi |
| 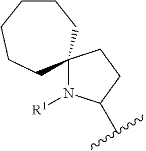 | ccxlvii |
| 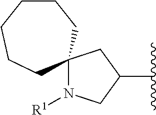 | ccxlviii |
| 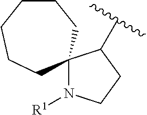 | ccxlix |
| 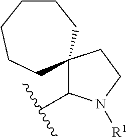 | ccl |
| 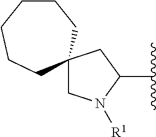 | ccli |
| 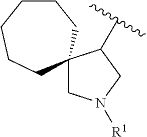 | cclii |
| 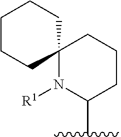 | ccliii |
| 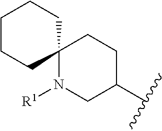 | ccliv |
| 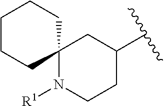 | cclv |
| 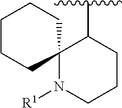 | cclvi |
| 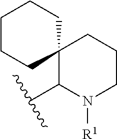 | cclvii |
| 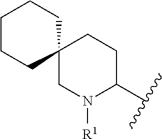 | cclviii |
| 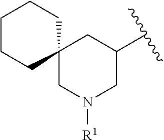 | cclix |
| 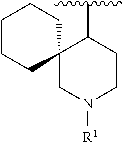 | cclx |
| 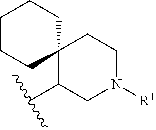 | cclxi |
| 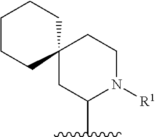 | cclxii |
| 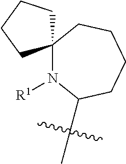 | cclxiii |
| 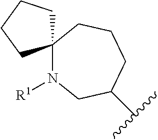 | cclxiv |
| 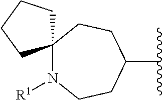 | cclxv |
| 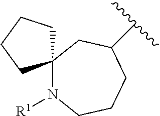 | cclxvi |
| 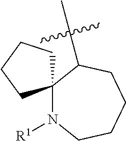 | cclxvii |
| 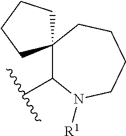 | cclxviii |
| 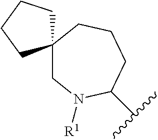 | cclxix |
| 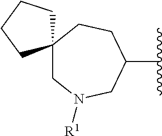 | cclxx |
| 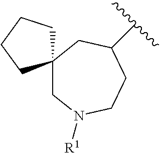 | cclxxi |
| 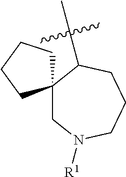 | cclxxii |
| 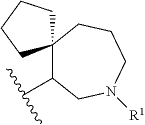 | cclxxiii |
| 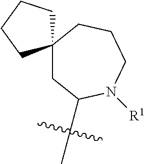 | cclxxiv |
| 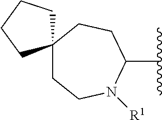 | cclxxv |
| 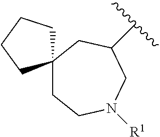 | cclxxvi |
| 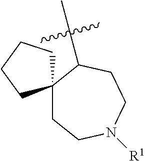 | cclxxvii |
| 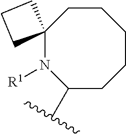 | cclxxviii |
| 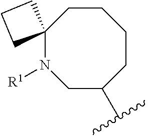 | cclxxix |
| 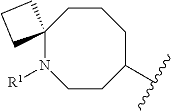 | cclxxx |
| 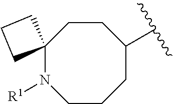 | cclxxxi |
| 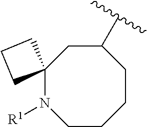 | cclxxxii |
| 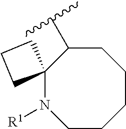 | cclxxxiii |
| 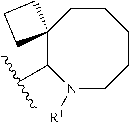 | cclxxxiv |
| 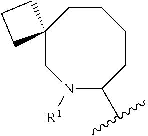 | cclxxxv |
| 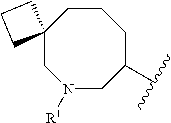 | cclxxxvi |
| 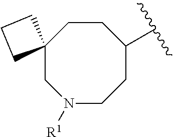 | cclxxxvii |
| 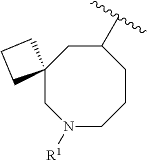 | cclxxxviii |
| 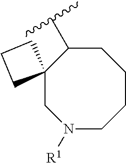 | cclxxxix |
| 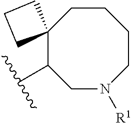 | ccxc |
| 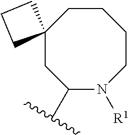 | ccxci |
| 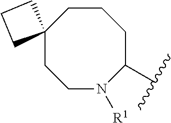 | ccxcii |
| 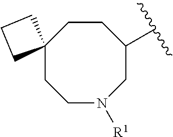 | ccxciii |
| 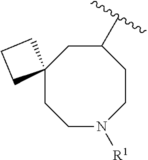 | ccxciv |
| 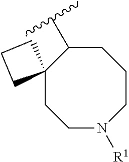 | ccxcv |
| 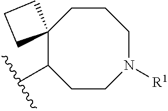 | ccxcvi |
| 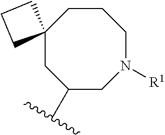 | ccxcvii |
| 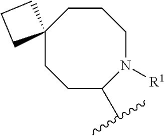 | ccxcviii |
| 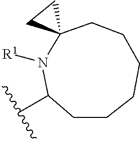 | ccxcix |
| 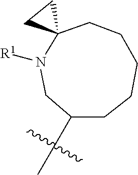 | ccc |
| 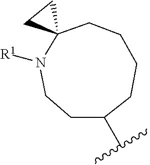 | ccci |
| 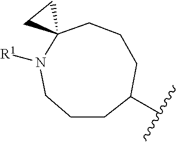 | cccii |
| 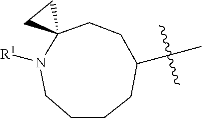 | ccciii |
| 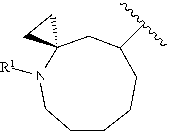 | ccciv |
| 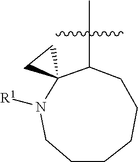 | cccv |
| 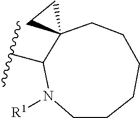 | cccvi |
| 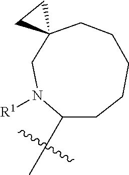 | cccvii |
| 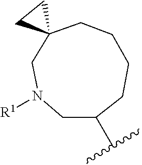 | cccviii |
| 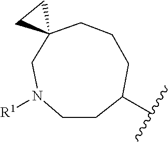 | cccix |
| 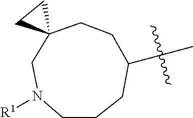 | cccx |
| 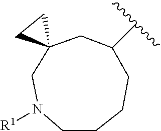 | cccxi |
| 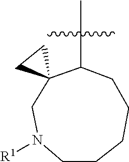 | cccxii |
| 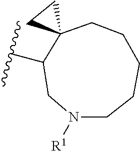 | cccxiii |
| 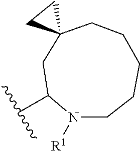 | cccxiv |
| 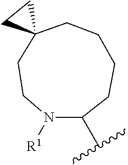 | cccxv |
| 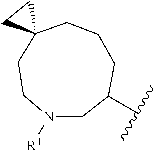 | cccxvi |
| 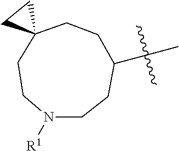 | cccxvii |
| 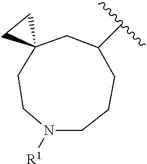 | cccxviii |
| 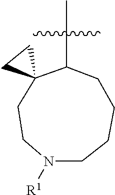 | cccxix |
| 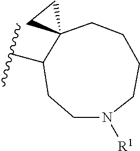 | cccxx |
| 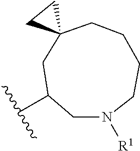 | cccxxi |
| 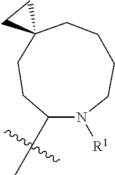 | cccxxii |
| 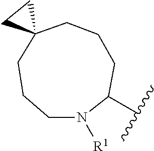 | cccxxiii |
| 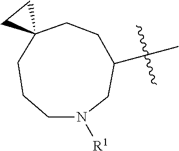 | cccxxiv |
| 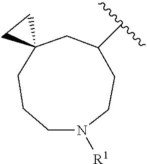 | cccxxv |
| 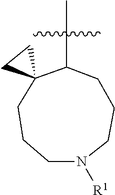 | cccxxvi |

TABLE 1G

| | |
|---|---|
| 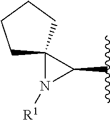 | cxxii-a |
| 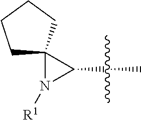 | cxxii-b |
| 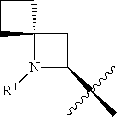 | cxxiii-a |
| 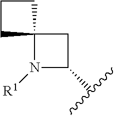 | cxxiii-b |
| 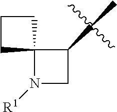 | cxxiv-a |
| 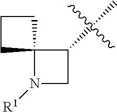 | cxxiv-b |
| 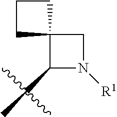 | cxxv-a |
| 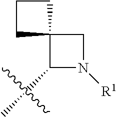 | cxxv-b |
| 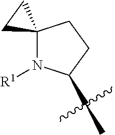 | cxxvi-a |
| 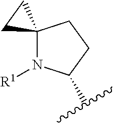 | cxxvi-b |
| 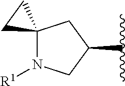 | cxxvii-a |
| 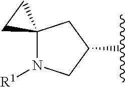 | cxxvii-b |
| 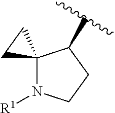 | cxxviii-a |
| 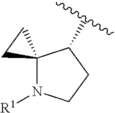 | cxxviii-b |
| 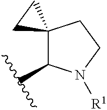 | cxxix-a |
| 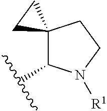 | cxxix-b |
| 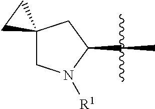 | cxxx-a |
| 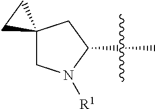 | cxxx-b |
| 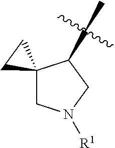 | cxxxi-a |
| 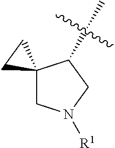 | cxxxi-b |
| 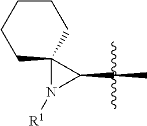 | cxxxii-a |
| 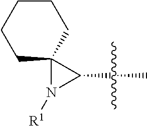 | cxxxii-b |
| 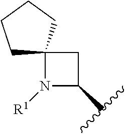 | cxxxiii-a |
| 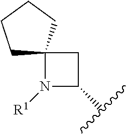 | cxxxiii-b |
| 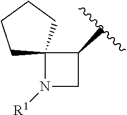 | cxxxiv-a |
| 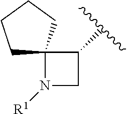 | cxxxiv-b |
| 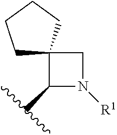 | cxxxv-a |
| 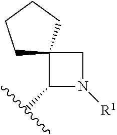 | cxxxv-b |
| 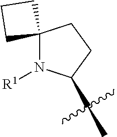 | cxxxvi-a |
| 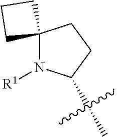 | cxxxvi-b |
| 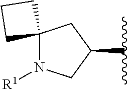 | cxxxvii-a |
| 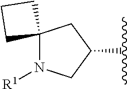 | cxxxvii-b |
| 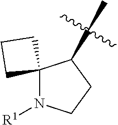 | cxxxviii-a |
| 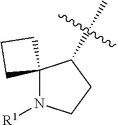 | cxxxviii-b |
| 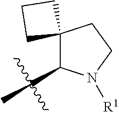 | cxxxix-a |
| 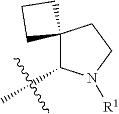 | cxxxix-b |
| 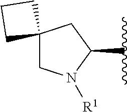 | cxl-a |
| 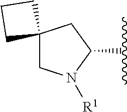 | cxl-b |
| 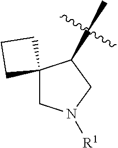 | cxli-a |
| 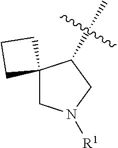 | cxli-b |
| 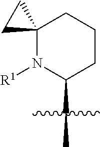 | cxlii-a |
| 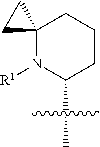 | cxlii-b |
| 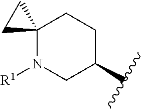 | cxliii-a |
| 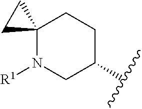 | cxliii-b |
| 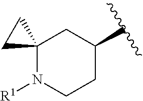 | cxliv-a |
| 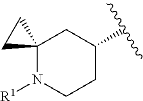 | cxliv-b |
| 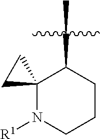 | cxlv-a |
| 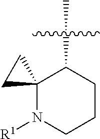 | cxlv-b |
| 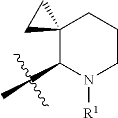 | cxlvi-a |
| 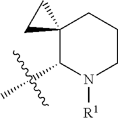 | cxlvi-b |
| 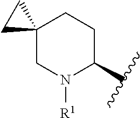 | cxlvii-a |
| 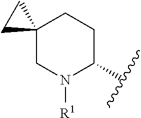 | cxlvii-b |
| 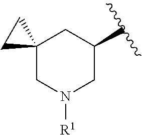 | cxlviii-a |
| 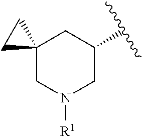 | cxlviii-b |
| 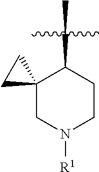 | cxlix-a |
| 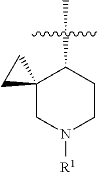 | cxlix-b |
| 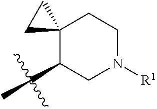 | cl-a |
| 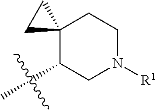 | cl-b |
| 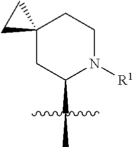 | cli-a |
| 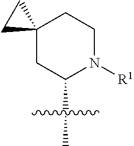 | cli-b |
| 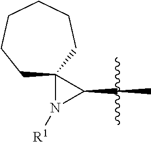 | clii-a |
| 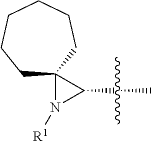 | clii-b |
| 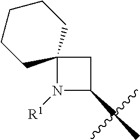 | cliii-a |
| 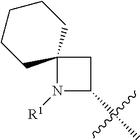 | cliii-b |
| 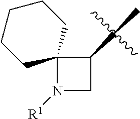 | cliv-a |
| 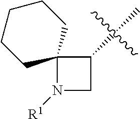 | cliv-b |
| 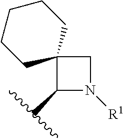 | clv-a |
| 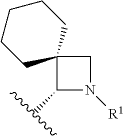 | clv-b |
| 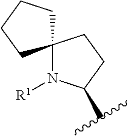 | clvi-a |
| 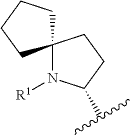 | clvi-b |
| 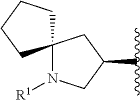 | clvii-a |
| 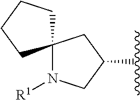 | clvii-b |
| 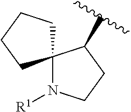 | clviii-a |
| 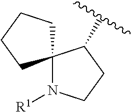 | clviii-b |
| 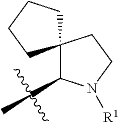 | clix-a |
| 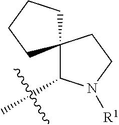 | clix-b |
| 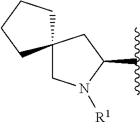 | clx-a |
| 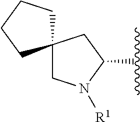 | clx-b |
| 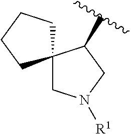 | clxi-a |
| 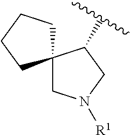 | clxi-a |
| 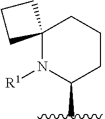 | clxii-a |
| 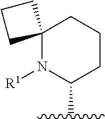 | clxii-b |
| 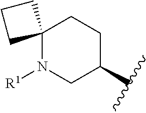 | clxiii-a |
| 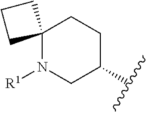 | clxiii-b |
| 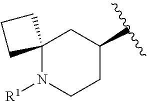 | clxiv-a |
| 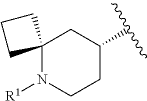 | clxiv-b |
| 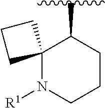 | clxv-a |
| 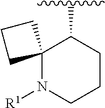 | clxv-b |
| 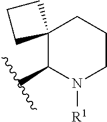 | clxvi-a |
| 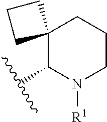 | clxvi-b |
| 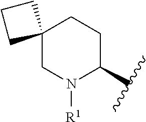 | clxvii-a |
| 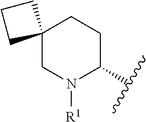 | clxvii-b |
| 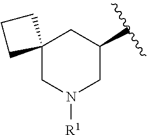 | clxviii-a |
| 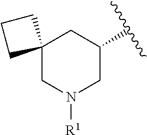 | clxviii-b |
| 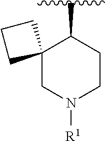 | clxix-a |
| 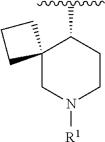 | clxix-b |
| 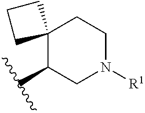 | clxx-a |
| 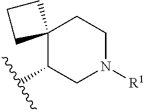 | clxx-b |
| 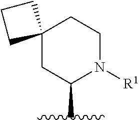 | clxxi-a |
| 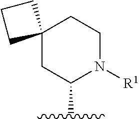 | clxxi-b |
| 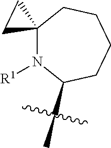 | clxxii-a |
| 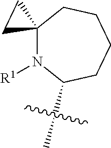 | clxxii-b |
| 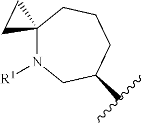 | clxxiii-a |
| 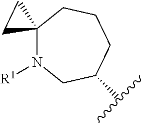 | clxxiii-b |
| 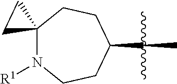 | clxxiv-a |
| 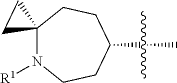 | clxxiv-b |
| 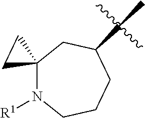 | clxxv-a |
| 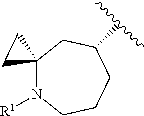 | clxxv-b |
| 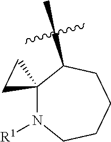 | clxxvi-a |
| 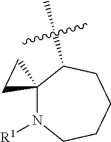 | clxxvi-b |
| 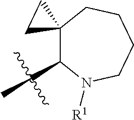 | clxxvii-a |
| 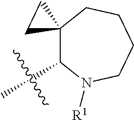 | clxxvii-b |
| 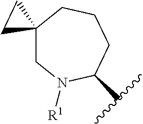 | clxxviii-a |
| 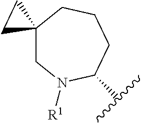 | clxxviii-b |
| 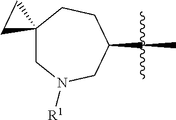 | clxxix-a |
| 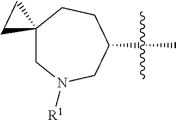 | clxxix-b |
| 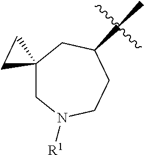 | clxxx-a |
| 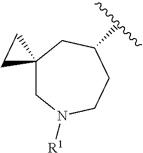 | clxxx-b |
| 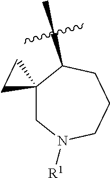 | clxxxi-a |
| 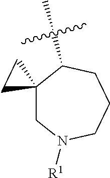 | clxxxi-b |
| 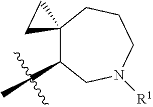 | clxxxii-a |
| 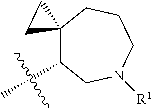 | clxxxii-b |
| 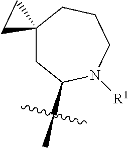 | clxxxiii-a |
| 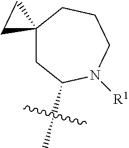 | clxxxiii-b |
| 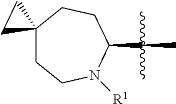 | clxxxiv-a |
| 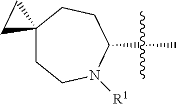 | clxxxiv-b |
| 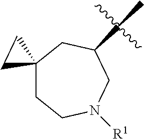 | clxxxv-a |
| 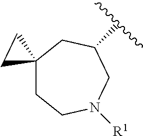 | clxxxv-b |
| 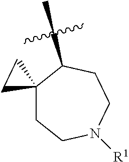 | clxxxvi-a |
| 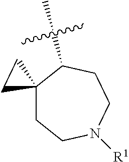 | clxxxvi-b |
| 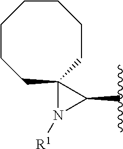 | clxxxvii-a |
| 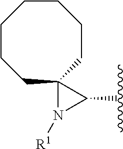 | clxxxvii-b |
| 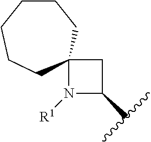 | clxxxviii-a |
| 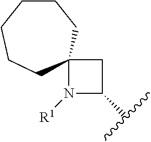 | clxxxviii-b |
| 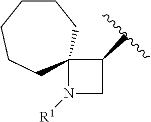 | clxxxix-a |
| 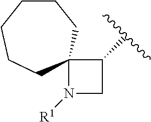 | clxxxix-b |
| 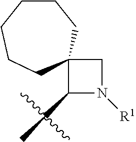 | cxc-a |
| 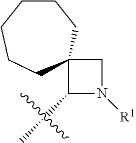 | cxc-b |
| 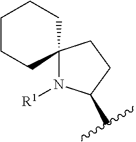 | cxci-a |
| 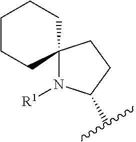 | cxci-b |
| 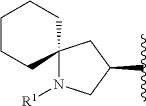 | cxcii-a |
| 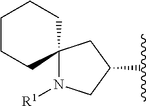 | cxcii-b |
| 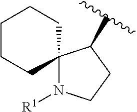 | cxciii-a |
| 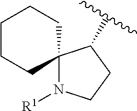 | cxciii-b |
| 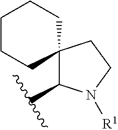 | cxciv-a |
| 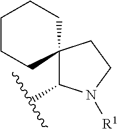 | cxciv-b |
| 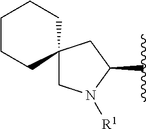 | cxcv-a |
| 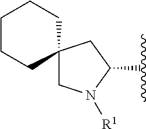 | cxcv-b |
| 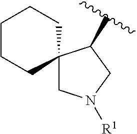 | cxcvi-a |
| 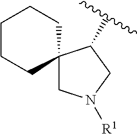 | cxcvi-b |
| 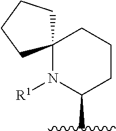 | cxcvii-a |
| 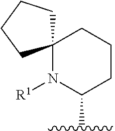 | cxcvii-b |
| 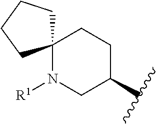 | cxcviii-a |
| 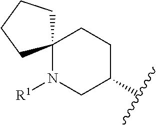 | cxcviii-b |
| 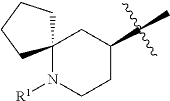 | cxcix-a |
| 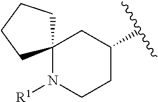 | cxcix-b |
| 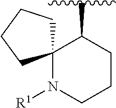 | cc-a |
| 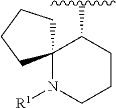 | cc-b |
| 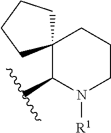 | cci-a |
| 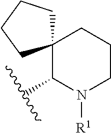 | cci-b |
| 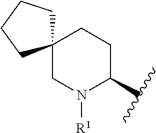 | ccii-a |
| 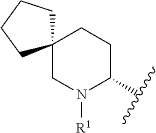 | ccii-b |
| 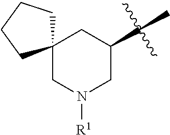 | cciii-a |
| 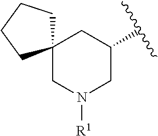 | cciii-b |
| 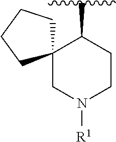 | cciv-a |
| 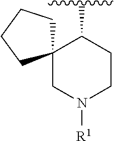 | cciv-b |
| 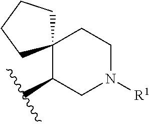 | ccv-a |
| 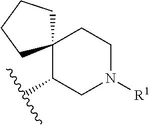 | ccv-b |
| 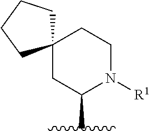 | ccvi-a |
| 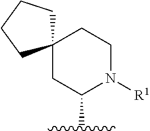 | ccvi-b |
| 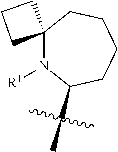 | ccvii-a |
| 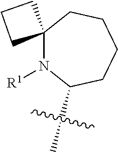 | ccvii-b |
| 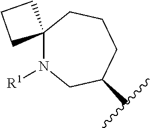 | ccviii-a |
| 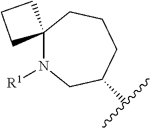 | ccviii-b |
| 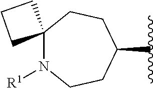 | ccix-a |
| 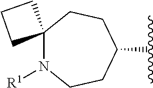 | ccix-b |
| 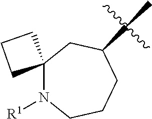 | ccx-a |
| 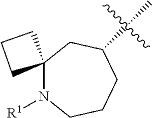 | ccx-b |
| 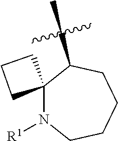 | ccxi-a |
| 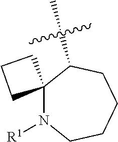 | ccxi-b |
| 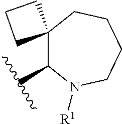 | ccxii-a |
| 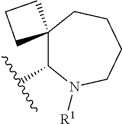 | ccxii-b |
| 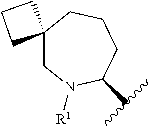 | ccxiii-a |
| 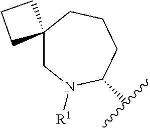 | ccxiii-b |
| 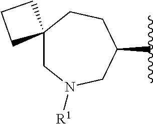 | ccxiv-a |
| 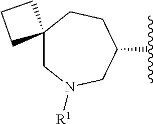 | ccxiv-b |
| 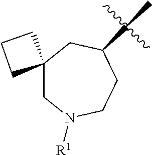 | ccxv-a |
| 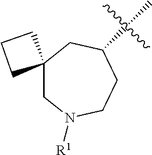 | ccxv-b |
| 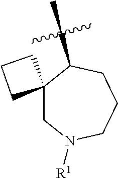 | ccxvi-a |
| 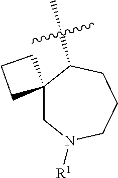 | ccxvi-b |
| 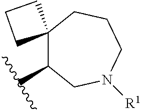 | ccxvii-a |
| 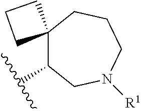 | ccxvii-b |
| 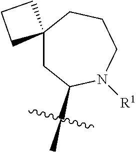 | ccxviii-a |
| 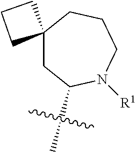 | ccxviii-b |
| 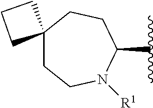 | ccxix-a |
| 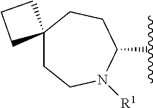 | ccxix-b |
| 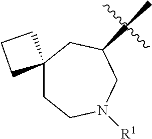 | ccxx-a |
| 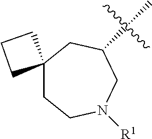 | ccxx-b |
| 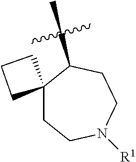 | ccxxi-a |
| 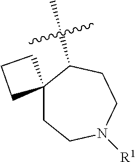 | ccxxi-b |
| 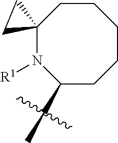 | ccxxii-a |
| 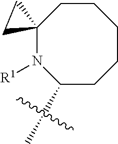 | ccxxii-b |
| 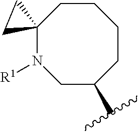 | ccxxiii-a |
| 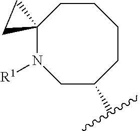 | ccxxiii-b |
| 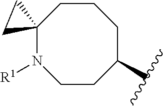 | ccxxiv-a |
| 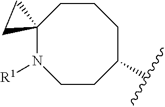 | ccxxiv-b |
| 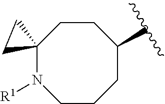 | ccxxv-a |
| 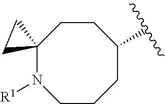 | ccxxv-b |
| 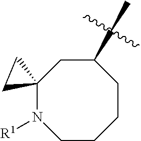 | ccxxvi-a |
| 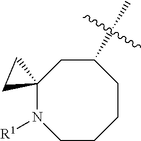 | ccxxvi-b |
| 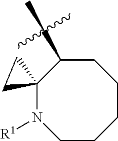 | ccxxvii-a |
| 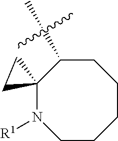 | ccxxvii-b |
| 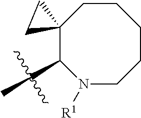 | ccxxviii-a |
| 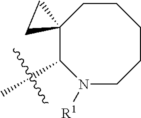 | ccxxviii-b |
| 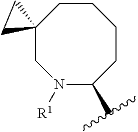 | ccxxix-a |
| 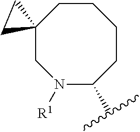 | ccxxix-b |
| 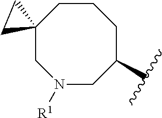 | ccxxx-a |
| 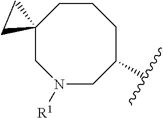 | ccxxx-b |
| 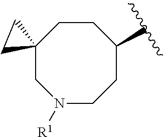 | ccxxi-a |
| 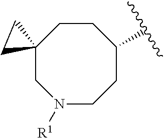 | ccxxxi-b |
| 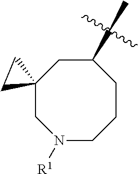 | ccxxxii-a |
| 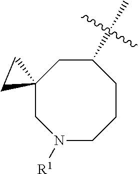 | ccxxxii-b |
| 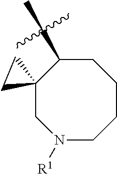 | ccxxxiii-a |
| 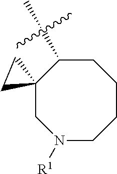 | ccxxxiii-b |
| 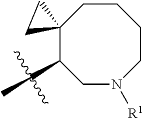 | ccxxxiv-a |
| 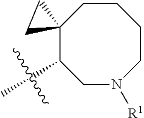 | ccxxxiv-b |
| 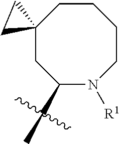 | ccxxxv-a |
| 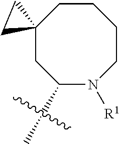 | ccxxxv-b |
| 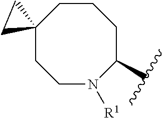 | ccxxxvi-a |
| 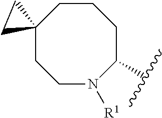 | ccxxxvi-b |
| 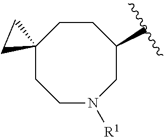 | ccxxxvii-a |
| 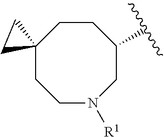 | ccxxxvii-b |
| 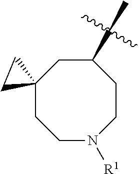 | ccxxxviii-a |
| 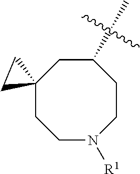 | ccxxxviii-b |
| 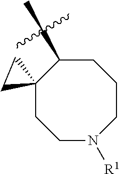 | ccxxxix-a |
| 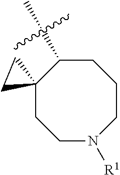 | ccxxxix-b |
| 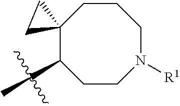 | ccxl-a |
| 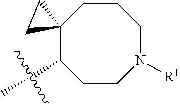 | ccxl-b |
| 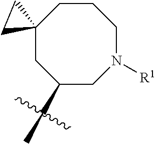 | ccxli-a |
| 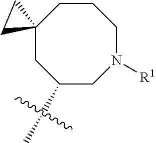 | ccxli-b |
| 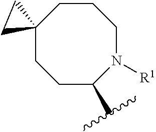 | ccxlii-a |
| 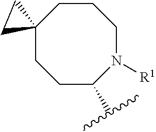 | ccxlii-b |
| 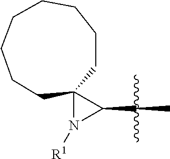 | ccxliii-a |
| 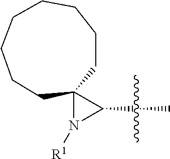 | ccxliii-b |
| 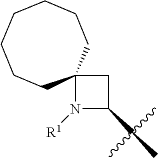 | ccxliv-a |
| 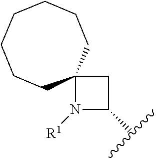 | ccxliv-b |
| 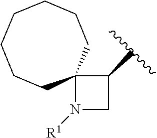 | ccxlv-a |
| 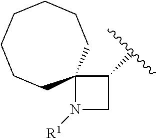 | ccxlv-b |
| 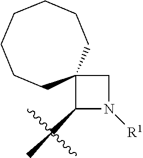 | ccxlvi-a |
| 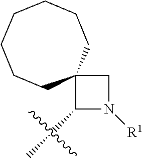 | ccxlvi-b |
| 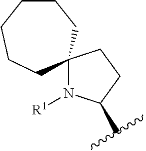 | ccxlvii-a |
| 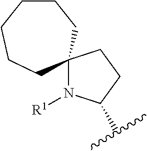 | ccxlvii-b |
| 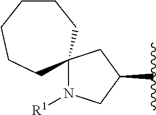 | ccxlviii-a |
| 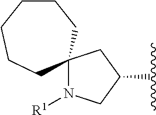 | ccxlviii-b |
| 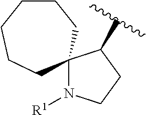 | ccxlix-a |
| 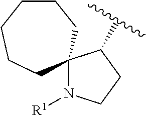 | ccxlix-b |
| 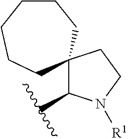 | ccl-a |
| 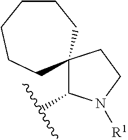 | ccl-b |
| 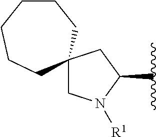 | ccli-a |
| 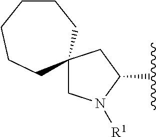 | ccli-b |
| 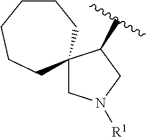 | cclii-a |
| 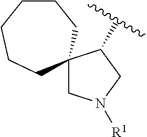 | cclii-b |
| 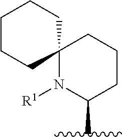 | ccliii-a |
| 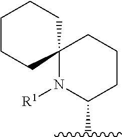 | ccliii-b |
| 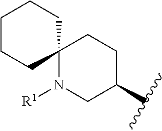 | ccliv-a |
| 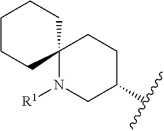 | ccliv-b |
| 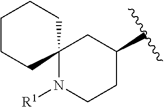 | cclv-a |
| 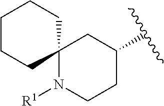 | cclv-b |
| 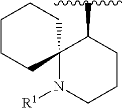 | cclvi-a |
| 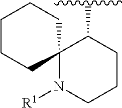 | cclvi-b |
| 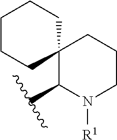 | cclvii-a |
| 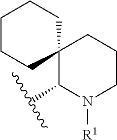 | cclvii-b |
| 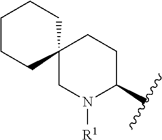 | cclviii-a |
| 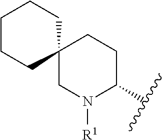 | cclviii-b |
| 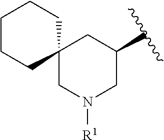 | cclix-a |
| 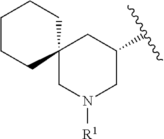 | cclix-b |
| 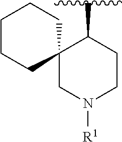 | cclx-a |
| 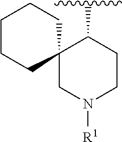 | cclx-b |
| 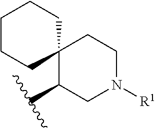 | cclxi-a |
| 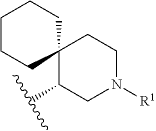 | cclxi-b |
| 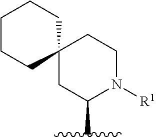 | cclxii-a |
| 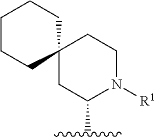 | cclxii-b |
| 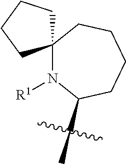 | cclxiii-a |
| 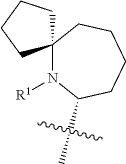 | cclxiii-b |
| 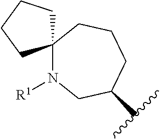 | cclxiv-a |
| 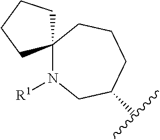 | cclxiv-b |
| 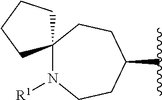 | cclxv-a |
| 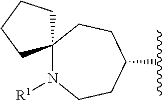 | cclxv-b |
| 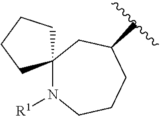 | cclxvi-a |
| 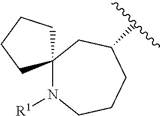 | cclxvi-b |
| 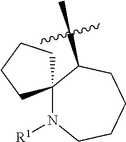 | cclxvii-a |
| 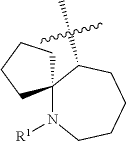 | cclxvii-b |
| 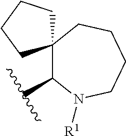 | cclxviii-a |
| 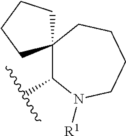 | cclxviii-b |
| 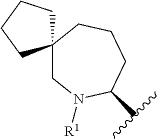 | cclxix-a |
| 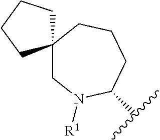 | cclxix-b |
| 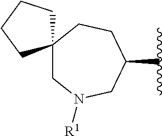 | cclxx-a |
| 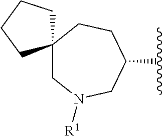 | cclxx-b |
| 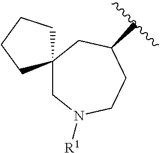 | cclxxi-a |
| 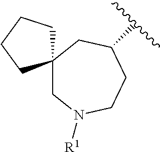 | cclxxi-b |
| 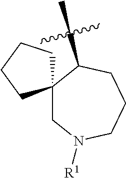 | cclxxii-a |
| 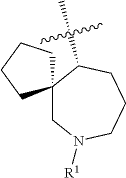 | cclxxii-b |
| 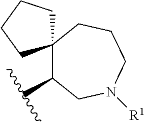 | cclxxiii-a |
| 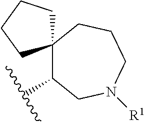 | cclxxiii-b |
| 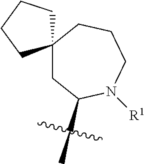 | cclxxiv-a |
| 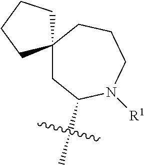 | cclxxiv-b |
| 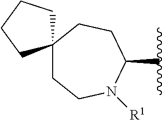 | cclxxv-a |
| 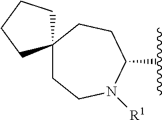 | cclxxv-b |
| 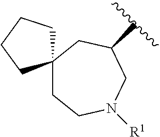 | cclxxvi-a |
| 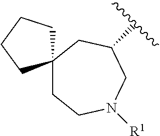 | cclxxvi-b |
| 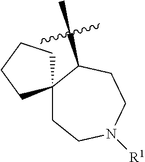 | cclxxvii-a |
| 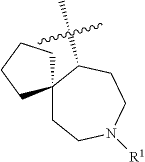 | cclxxvii-b |
| 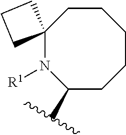 | cclxxviii-a |
| 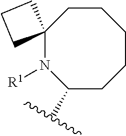 | cclxxviii-b |
| 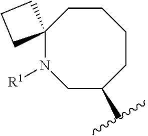 | cclxxix-a |
| 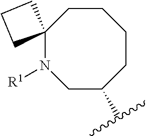 | cclxxix-b |
| 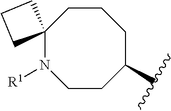 | cclxxx-a |
| 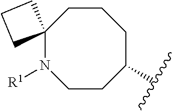 | cclxxx-b |
| 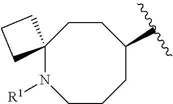 | cclxxxi-a |
| 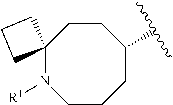 | cclxxxi-b |
| 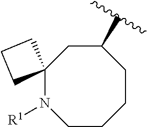 | cclxxxii-a |
| 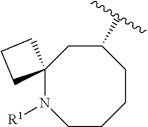 | cclxxxii-b |
| 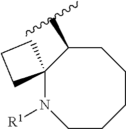 | cclxxxiii-a |
| 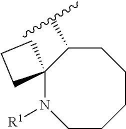 | cclxxxiii-b |
| 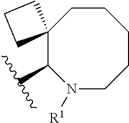 | cclxxxiv-a |
| 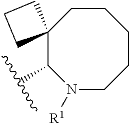 | cclxxxiv-b |
| 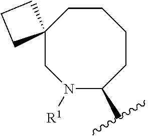 | cclxxxv-a |
| 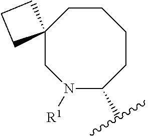 | cclxxxv-b |
| 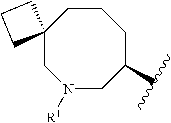 | cclxxxvi-a |
| 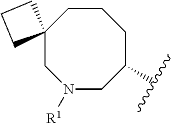 | cclxxxvi-b |
| 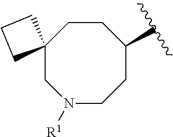 | cclxxxvii-a |
| 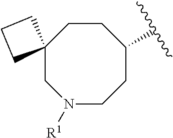 | cclxxxvii-b |
| 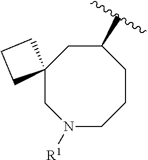 | cclxxxviii-a |
| 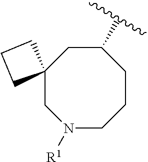 | cclxxxviii-b |
| 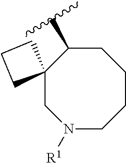 | cclxxxix-a |
| 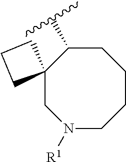 | cclxxxix-b |
| 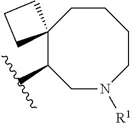 | ccxc-a |
| 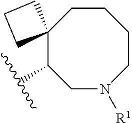 | ccxc-b |
| 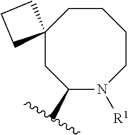 | ccxci-a |
| 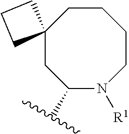 | ccxci-b |
| 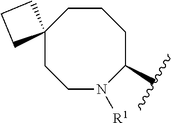 | ccxcii-a |
| 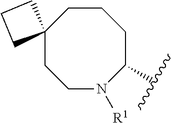 | ccxcii-b |
| 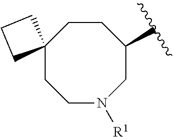 | ccxciii-a |
| 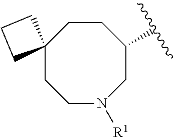 | ccxciii-b |
| 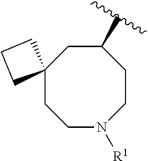 | ccxciv-a |
| 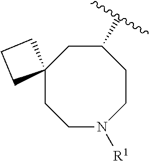 | ccxciv-b |
| 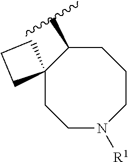 | ccxcv-a |
| 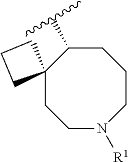 | ccxcv-b |
| 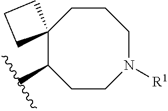 | ccxcvi-a |
| 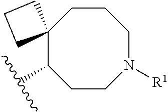 | ccxcvi-b |
| 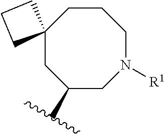 | ccxcvii-a |
| 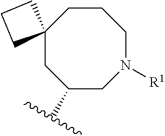 | ccxcvii-b |
| 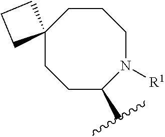 | ccxcviii-a |
| 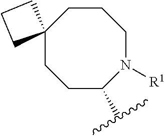 | ccxcviii-b |
| 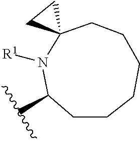 | ccxcix-a |
| 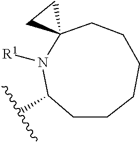 | ccxcix-b |
| 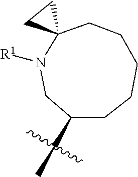 | ccc-a |
| 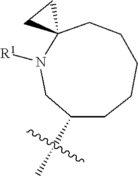 | ccc-b |
| 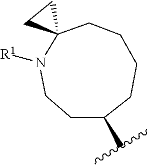 | ccci-a |
| 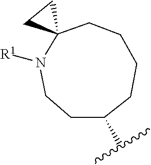 | ccci-b |
| 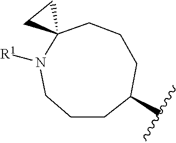 | cccii-a |
| 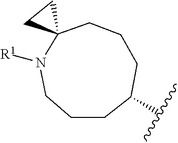 | cccii-b |
| 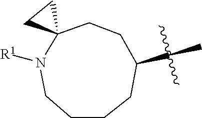 | ccciii-a |
| 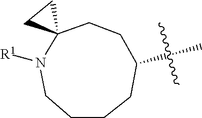 | ccciii-b |
| 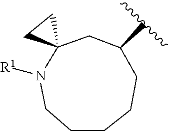 | ccciv-a |
| 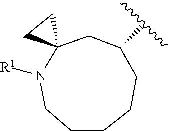 | ccciv-b |
| 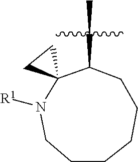 | cccv-a |
| 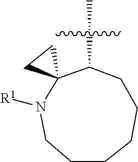 | cccv-b |
| 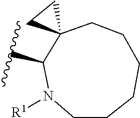 | cccvi-a |
| 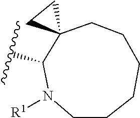 | cccvi-b |
| 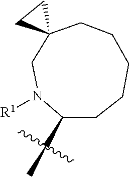 | cccvii-a |
| 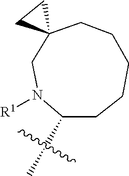 | cccvii-b |
| 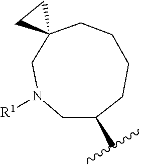 | cccviii-a |
| 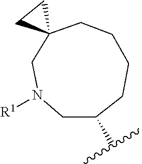 | cccviii-b |
| 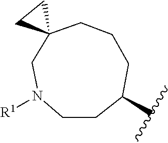 | cccix-a |
| 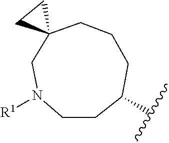 | cccix-b |
| 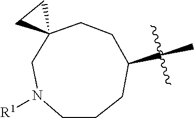 | cccx-a |
| 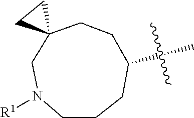 | cccx-b |
| 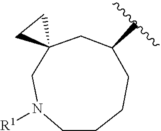 | cccxi-a |
| 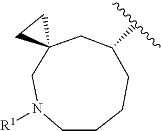 | cccxi-b |
| 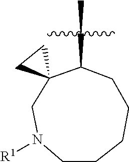 | cccxii-a |
| 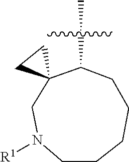 | cccxii-b |
| 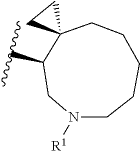 | cccxiii-a |
| 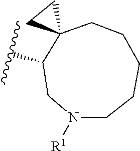 | cccxiii-b |
| 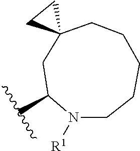 | cccxiv-a |
| 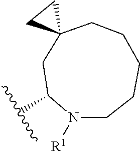 | cccxiv-b |
| 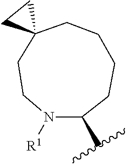 | cccxv-a |
| 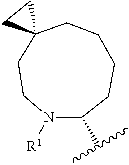 | cccxv-b |
| 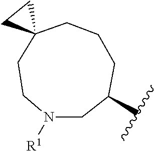 | cccxvi-a |
| 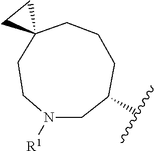 | cccxvi-b |
| 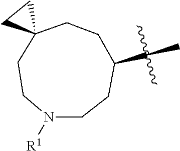 | cccxvii-a |
| 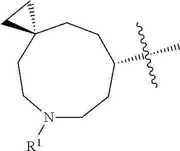 | cccxvii-b |
| 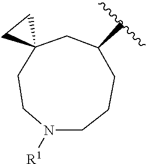 | cccxviii-a |
| 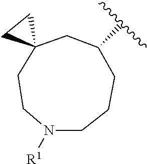 | cccxviii-b |
| 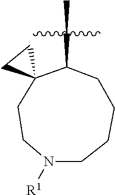 | cccxix-a |
| 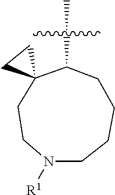 | cccxix-b |
| 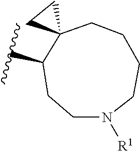 | cccxx-a |
| 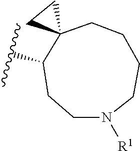 | cccxx-b |
| 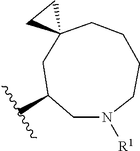 | cccxxi-a |
| 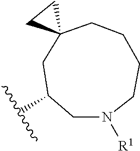 | cccxxi-b |
| 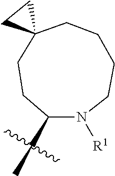 | cccxxii-a |
| 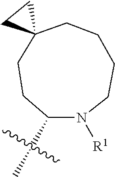 | cccxxii-b |
| 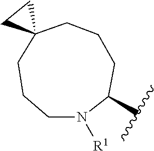 | cccxxiii-a |
| 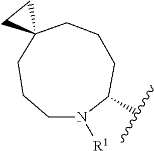 | cccxxiii-b |
| 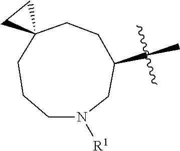 | cccxxiv-a |
| 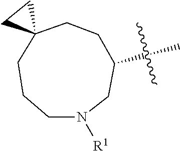 | cccxxiv-b |
| 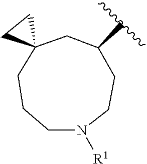 | cccxxv-a |
| 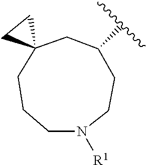 | cccxxv-b |
| 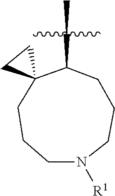 | cccxxvi-a |
| 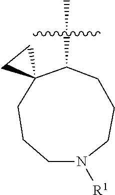 | cccxxvi-b |

In certain embodiments, Ring A is substituted with 0-3 R^{v} groups and is selected from:

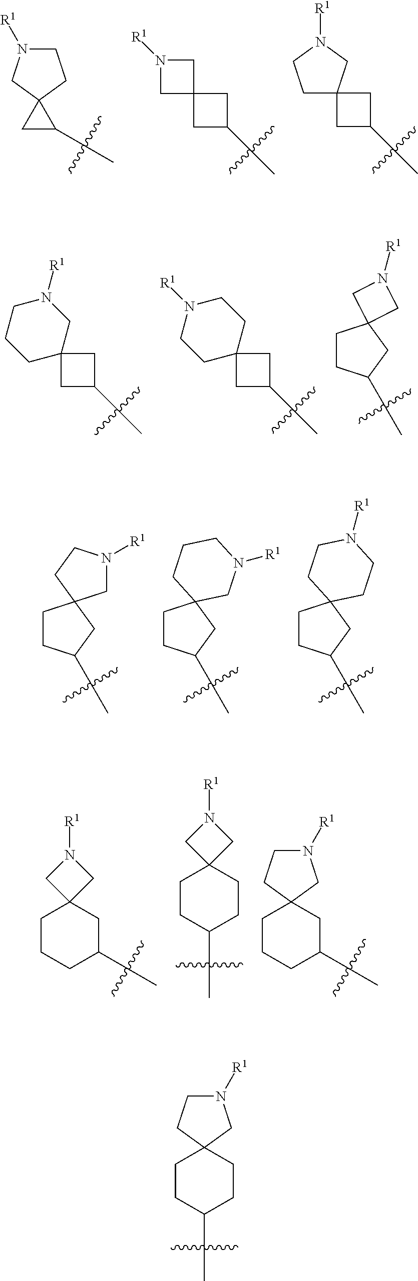

As described generally above, in some embodiments, Ring B is a saturated 5-7-membered heterocyclo ring having 1-2 nitrogen atoms, wherein Ring B is substituted with 0-5 R^{x} groups. In some embodiments, Ring B is unsubstituted. In some embodiments, Ring B is substituted with at least one R^{x} group. In some embodiments, Ring B is substituted with one R^{x} group. In some embodiments, Ring B is substituted with two R^{x} groups. In some embodiments, Ring B is substituted with three R^{x} groups. In some embodiments, Ring B is substituted with four R^{x} groups. In some embodiments, Ring B is substituted with five R^{x} groups.

In some embodiments, Ring B is a saturated 5-membered heterocyclo ring having 1-2 nitrogen atoms, wherein Ring B is substituted with 0-5 R^{x} groups. In some such embodiments, at least one nitrogen in Ring B is substituted with R^{x}. In some embodiments, Ring B is a saturated 5-membered heterocyclo ring having 1-2 nitrogen atoms, wherein Ring B is unsubstituted. In some embodiments, Ring B is a saturated 5-membered heterocyclo ring having 1 nitrogen atom, wherein Ring B is unsubstituted. In some embodiments, Ring B is a saturated 5-membered heterocyclo ring having 1 nitrogen atom, wherein Ring B is substituted with 1-3 R^{x} groups. In some embodiments, Ring B is a saturated 5-membered heterocyclo ring having 1 nitrogen atom, wherein Ring B is substituted with 1 R^{x} group. In some embodiments, Ring B is a saturated 5-membered heterocyclo ring having 1 nitrogen atom, wherein Ring B is substituted with 2 R^{x} groups. In some embodiments, Ring B is a saturated 5-membered heterocyclo ring having 1 nitrogen atom, wherein Ring B is substituted with 3 R^{x} groups.

In some embodiments, Ring B is a saturated 5-membered heterocyclo ring having 1 nitrogen atom. In some embodiments, Ring B is a saturated 5-membered heterocyclo ring having 1 nitrogen atom, wherein the 1 nitrogen atom is substituted with R^{x}. In some embodiments, Ring B is a saturated 5-membered heterocyclo ring having 2 nitrogen atoms.

In some embodiments, Ring B is a saturated 6-membered heterocyclo ring having 1-2 nitrogen atoms, wherein Ring B is substituted with 0-5 R^{x} groups. In some such embodiments, at least one nitrogen in Ring B is substituted with R^{x}. In some embodiments, Ring B is a saturated 6-membered heterocyclo ring having 1-2 nitrogen atoms, wherein Ring B is unsubstituted.

In some embodiments, Ring B is a saturated 6-membered heterocyclo ring having 1 nitrogen atom. In some such embodiments, the 1 nitrogen atom is substituted with R^{x}. In some embodiments, Ring B is a saturated 6-membered heterocyclo ring having 2 nitrogen atoms.

In some embodiments, Ring B is a saturated 7-membered heterocyclo ring having 1-2 nitrogen atoms, wherein Ring B is substituted with 0-5 R^{x} groups. In some such embodiments, at least one nitrogen in Ring B is substituted with R^{x}. In some embodiments, Ring B is a saturated 7-membered heterocyclo ring having 1-2 nitrogen atoms, wherein Ring B is unsubstituted. In some embodiments, Ring B is a saturated 7-membered heterocyclo ring having 1 nitrogen atom, wherein Ring B is unsubstituted. In some embodiments, Ring B is a saturated 7-membered heterocyclo ring having 1 nitrogen atom, wherein Ring B is substituted with 1-3 R^{x} groups. In some embodiments, Ring B is a saturated 7-membered heterocyclo ring having 1 nitrogen atom, wherein Ring B is substituted with 1 R^{x} group. In some embodiments, Ring B is a saturated 7-membered heterocyclo ring having 1 nitrogen atom, wherein Ring B is substituted with 2 R^{x} groups.

In some embodiments, Ring B is a saturated 7-membered heterocyclo ring having 1 nitrogen atom. In some such embodiments, the 1 nitrogen atom is substituted with R^{x}. In some embodiments, Ring B is a saturated 7-membered heterocyclo ring having 2 nitrogen atoms.

In some embodiments, Ring B is selected from those in Table 2A, where the dotted line represents the π-bond of the aryl ring to which Ring B is fused:

TABLE 2A

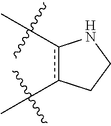

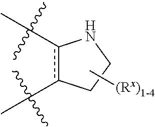

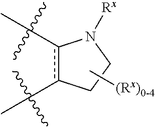

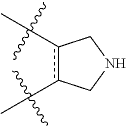

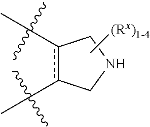

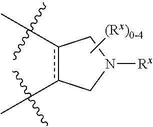

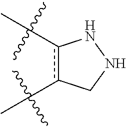

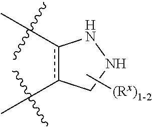

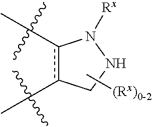

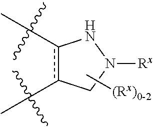

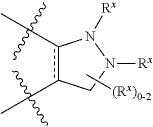

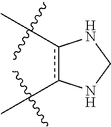

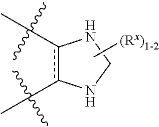

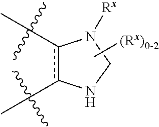

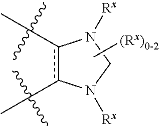

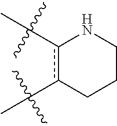

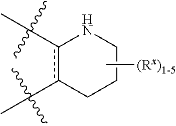

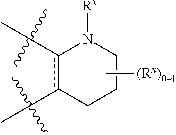

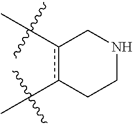

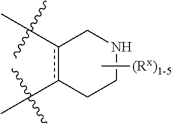

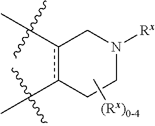

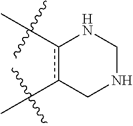

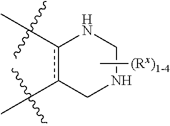

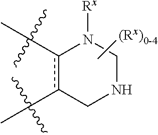

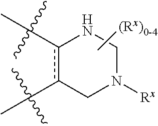

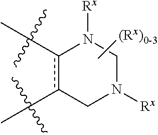

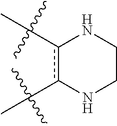

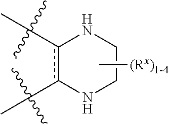

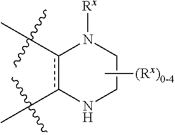

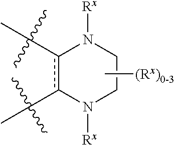

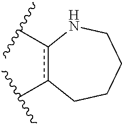

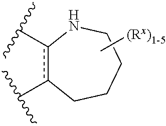

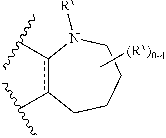

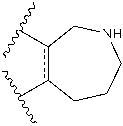

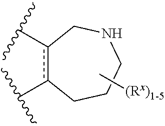

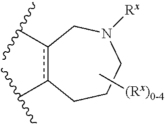

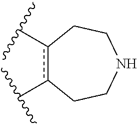

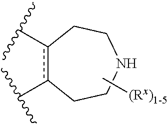

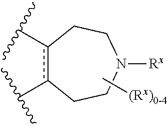

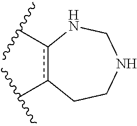

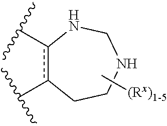

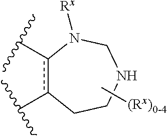

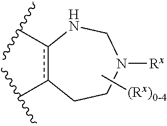

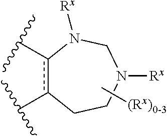

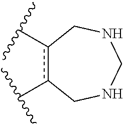

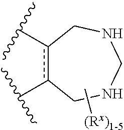

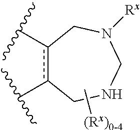

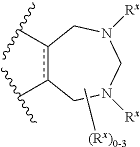

wherein each R^{x} is independently as defined above and described herein.

In some embodiments, Ring B is selected from those in Table 2B, where the dotted line represents the π-bond of the aryl ring to which Ring B is fused:

TABLE 2B

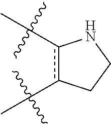

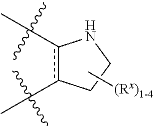

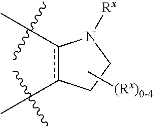

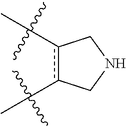

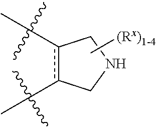

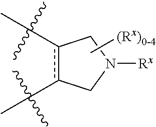

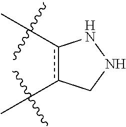

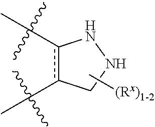

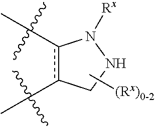

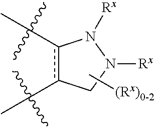

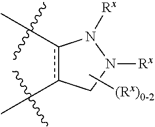

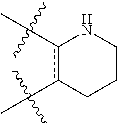

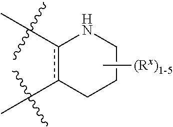

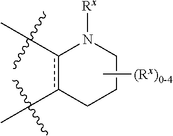

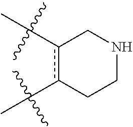

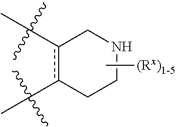

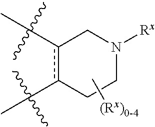

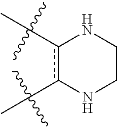

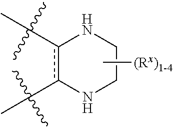

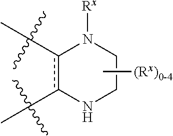

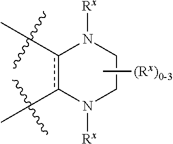

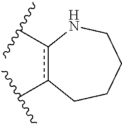

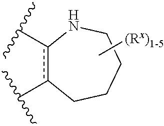

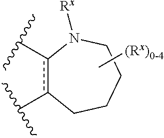

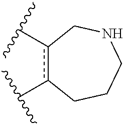

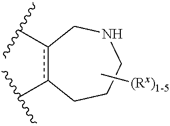

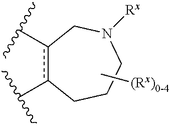

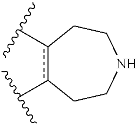

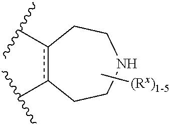

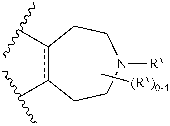

wherein each R^{x} is independently as defined above and described herein.

In certain embodiments, Ring B is selected from

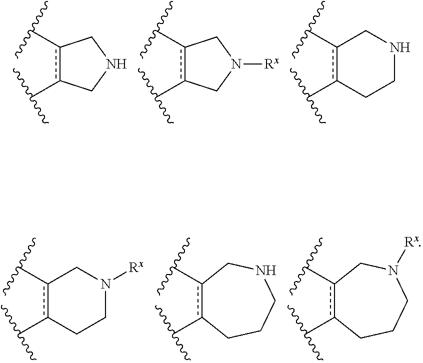

In some embodiments, Ring B is selected from

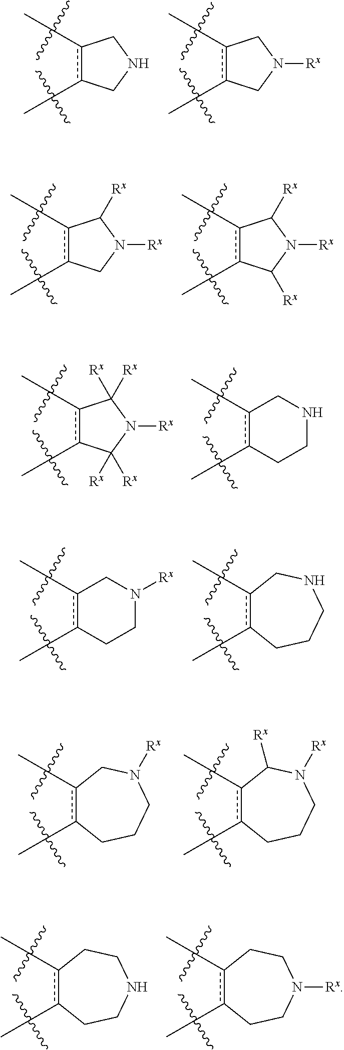

It will be appreciated that the Ring B groups in Table 2A or Table 2B can be fused to the aryl ring of formula I in either orientation, such that compounds comprising the Ring B moiety

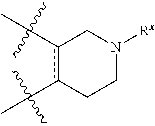

have one of the following structures, referred to, infra, as III-c and III-e:

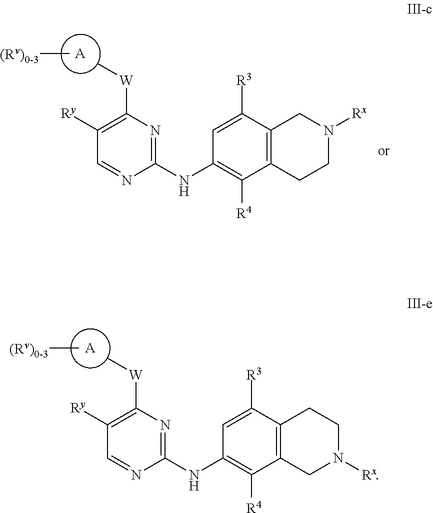

In some embodiments, Ring B is

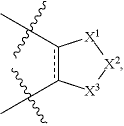

wherein each of X¹, X² and X³ is independently selected from —CH₂—, CH(R^{x})—, —C(R^{x})₂—, —NH—, or —N(R^{x})—, provided that at least one of X¹, X² and X³ is —NH— or —N(R^{x})—. In some embodiments, one of X¹, X² and X³ is selected from —NH— or —N(R^{x})—. In some embodiments, two of X¹, X² and X³ are independently selected from —NH— or —N(R^{x})—.

In some embodiments, Ring B is

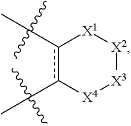

wherein each of X¹, X², X³ and X⁴ is independently selected from —CH₂—, CH(R^{x})—, —C(R^{x})₂—, —NH—, or —N(R^{x})—, provided that at least one X¹, X², X³ and X⁴ is —NH— or —N(R^{x})—. In some embodiments, one of X¹, X², X³ and X⁴ is selected from —NH— or —N(R^{x})—. In some embodiments, two of X¹, X², X³ and X⁴ are independently selected from —NH— or —N(R^{x})—.

In some embodiments, Ring B is

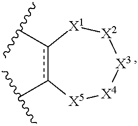

wherein each of X¹, X², X³, X⁴ and X⁵ is independently selected from —CH₂—, CH(R^{x})—, —C(R^{x})₂—, —NH—, or —N(R^{x})—, provided that at least one X¹, X², X³, X⁴ and X⁵ is —NH— or —N(R^{x})—. In some embodiments, one of X¹, X², X³, X⁴ and X⁵ is selected from —NH— or —N(R^{x})—. In some embodiments, two of X¹, X², X³, X⁴ and X⁵ are independently selected from —NH— or —N(R^{x})—.

As described generally above, R¹ is -L-Y, wherein:
L is an optionally substituted bivalent C₂₋₈ unsaturated, straight or branched, hydrocarbon chain, wherein one, two, or three methylene units of L are optionally and independently replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N— or —C(═N₂)—; and
Y is hydrogen, halogen, —CN, C₁₋₆ aliphatic optionally substituted with oxo, halogen, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 groups independently selected from -Q-Z, oxo, —NO₂, halogen, —CN, and C₁₋₆ aliphatic, wherein:
   Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —SO— or —SO₂—; and
   Z is hydrogen or C₁₋₆ aliphatic optionally substituted with oxo, halogen, or CN.

In some embodiments, L is an optionally substituted bivalent C₂₋₈ unsaturated, straight or branched, hydrocarbon chain, wherein one or two methylene units of L are optionally and independently replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N— or —C(═N₂)—.

In some embodiments, L is an optionally substituted bivalent C₂₋₈ unsaturated, straight or branched, hydrocarbon chain, wherein one or two methylene units of L are optionally and independently replaced by —N(R)—, —N(R)C(O)—, —C(O)N(R)—, —N(R)SO₂—, —SO₂N(R)—, —O—, —C(O)—, —OC(O)—, —C(O)O—, —S—, —SO— or —SO₂—.

In some embodiments, L is an optionally substituted bivalent C₂₋₈ unsaturated, straight or branched, hydrocarbon chain, wherein one or two methylene units of L are optionally and independently replaced by —N(R)—, —O—, —C(O)—, —S—, —SO— or —SO₂—.

In some embodiments, L is an optionally substituted bivalent C₂₋₈ unsaturated, straight or branched, hydrocarbon chain, wherein three methylene units of L are optionally and independently replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—.

In some embodiments, L is an optionally substituted bivalent C₂₋₈ unsaturated, straight or branched, hydrocarbon chain, wherein L has at least one double bond and one or two methylene units of L are optionally replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—.

In some embodiments, L is an optionally substituted bivalent C₂₋₈ unsaturated, straight or branched, hydrocarbon chain, wherein L has at least one double bond and one methylene unit of L is replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—.

In some embodiments, L is an optionally substituted bivalent C₂₋₈ unsaturated, straight or branched, hydrocarbon chain, wherein L has at least one double bond and one or two methylene units of L are replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—.

In some embodiments, L is an optionally substituted bivalent C₂₋₈ unsaturated, straight or branched, hydrocarbon chain, wherein L has at least one double bond and one methylene unit of L is replaced by —C(O)—, and one additional methylene unit of L is optionally replaced by cyclopropylene, —O— or —N(R)—.

In some embodiments, L is an optionally substituted bivalent C₂₋₈ unsaturated, straight or branched, hydrocarbon chain, wherein L has at least one double bond and one methylene unit of L is replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—.

In some embodiments, L is an optionally substituted bivalent C₂₋₈ unsaturated, straight or branched, hydrocarbon chain, wherein L has at least one double bond and one methylene unit of L is replaced by —N(R)C(O)—, —C(O)N(R)—, —N(R)SO₂—, —SO₂N(R)—, —C(O)—, —OC(O)—, or —C(O)O—.

In some embodiments, L is an optionally substituted bivalent C₂₋₈ unsaturated, straight or branched, hydrocarbon chain, wherein L has at least one double bond and one or two methylene units of L are optionally and independently replaced by cyclopropylene, —N(R)—, —O—, —C(O)—, —S—, —SO—, or —SO₂—.

In some embodiments, L is an optionally substituted bivalent C₂₋₈ unsaturated, straight or branched, hydrocarbon chain, wherein L has at least one double bond and one or two methylene units of L are replaced by cyclopropylene, —N(R)—, —O—, or —C(O)—.

In some embodiments, L is an optionally substituted bivalent C₂₋₈ unsaturated, straight or branched, hydrocarbon chain, wherein L has at least one double bond and one methylene unit of L is replaced by —C(O)—, and one additional methylene unit of L is replaced by —N(R)—.

In some embodiments, L is an optionally substituted bivalent C₂₋₈ unsaturated, straight or branched, hydrocarbon chain, wherein L has at least one double bond and one methylene unit of L is replaced by —C(O)—, and one additional methylene unit of L is replaced by cyclopropylene.

In some embodiments, L is an optionally substituted bivalent C₂₋₈ unsaturated, straight or branched, hydrocarbon chain, wherein L has at least one double bond and one methylene unit of L is replaced by —N(R)—, —O—, or —C(O)—.

In some embodiments, L is an optionally substituted bivalent C₂₋₈ unsaturated, straight or branched, hydrocarbon chain, wherein L has at least one double bond and one methylene unit of L is replaced by —C(O)—.

In some embodiments, L is an optionally substituted bivalent C₂₋₈ unsaturated, straight or branched, hydrocarbon chain, wherein L has at least one double bond and one methylene unit of L is replaced by —N(R)C(O)—, —C(O)N(R)—, —N(R)SO₂—, —SO₂N(R)—, —C(O)—, —OC(O)—, or —C(O)O—, and one or two additional methylene units of L are optionally and independently replaced by —N(R)—, —O—, or —C(O)—.

In some embodiments, L is an optionally substituted bivalent C₂₋₈ unsaturated, straight or branched, hydrocarbon chain, wherein L has at least one double bond and one methylene unit of L is replaced by —N(R)C(O)—, —C(O)N(R)—, —N(R)SO₂—, —SO₂N(R)—, —C(O)—, —OC(O)—, or —C(O)O—, and one additional methylene unit of L is optionally replaced by —N(R)—, —O—, or —C(O)—.

In some embodiments, L is an optionally substituted bivalent C₂₋₈ unsaturated, straight or branched, hydrocarbon chain, wherein L has at least one double bond and one methylene unit of L is replaced by —C(O)—, and one additional methylene unit of L is optionally replaced by —N(R)—, —O—, or —C(O)—.

In some embodiments, L is an optionally substituted bivalent C₂₋₈ straight or branched, hydrocarbon chain wherein L has at least one double bond and one methylene unit of L is replaced by —SO₂—.

In some embodiments, L is —C(O)CH═CH—, —C(O)CH═CHCH₂N(CH₃)—, —C(O)CH═CHCH₂O—, —CH₂CH₂NRC(O)CH═CH—, —SO₂CH═CH—, —SO₂CH═CHCH₂—, —C(O)C(═CH₂)CH₂—, —CH₂CH₂NRC(O)CH═CH—, —C(O)CH═CH-cyclopropylene- or —C(O)C(CN)═CH-cyclopropylene-, wherein R is H or optionally substituted C₁₋₆ aliphatic; and Y is hydrogen or C₁₋₆ aliphatic optionally substituted with oxo, halogen, NO₂, or CN.

In certain embodiments, L is —C(O)CH═CH—. In some embodiments, L is —C(O)CH═CHCH₂N(CH₃)—. In some embodiments, L is —C(O)C(CN)═CH-cyclopropylene-.

In some embodiments, L is an optionally substituted bivalent C₂₋₈ unsaturated, straight or branched, hydrocarbon chain, wherein L has at least one triple bond and one or two methylene units of L are optionally replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—.

In some embodiments, L is an optionally substituted bivalent C₂₋₈ unsaturated, straight or branched, hydrocarbon chain, wherein L has at least one triple bond and one methylene unit of L is replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—.

In some embodiments, L is an optionally substituted bivalent C₂₋₈ unsaturated, straight or branched, hydrocarbon chain, wherein L has at least one triple bond and one or two methylene units of L are replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—.

In some embodiments, L is an optionally substituted bivalent C₂₋₈ unsaturated, straight or branched, hydrocarbon chain, wherein L has at least one triple bond and one methylene unit of L is replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—.

In some embodiments, L is an optionally substituted bivalent C₂₋₈ unsaturated, straight or branched, hydrocarbon chain, wherein L has at least one triple bond and one methylene unit of L is replaced by —N(R)C(O)—, —C(O)N(R)—, —N(R)SO₂—, —SO₂N(R)—, —C(O)—, —OC(O)—, or —C(O)O—.

In some embodiments, L is an optionally substituted bivalent C₂₋₈ unsaturated, straight or branched, hydrocarbon chain, wherein L has at least one triple bond and one or two methylene units of L are optionally and independently replaced by —N(R)—, —O—, —C(O)—, —S—, —SO—, or —SO₂—.

In some embodiments, L is an optionally substituted bivalent C₂₋₈ unsaturated, straight or branched, hydrocarbon chain, wherein L has at least one triple bond and one methylene unit of L is replaced by —N(R)—, —O—, or —C(O)—.

In some embodiments, L is an optionally substituted bivalent C₂₋₈ unsaturated, straight or branched, hydrocarbon chain, wherein L has at least one triple bond and one methylene unit of L is replaced by —C(O)—.

In some embodiments, L is an optionally substituted bivalent C₂₋₈ unsaturated, straight or branched, hydrocarbon chain, wherein L has at least one triple bond and one methylene unit of L is replaced by —N(R)C(O)—, —C(O)N(R)—, —N(R)SO₂—, —SO₂N(R)—, —C(O)—, —OC(O)—, or —C(O)O—, and one or two additional methylene units of L are optionally and independently replaced by —N(R)—, —O—, or —C(O)—.

In some embodiments, L is an optionally substituted bivalent C₂₋₈ unsaturated, straight or branched, hydrocarbon chain, wherein L has at least one triple bond and one methylene unit of L is replaced by —N(R)C(O)—, —C(O)N(R)—, —N(R)SO₂—, —SO₂N(R)—, —C(O)—, —OC(O)—, or —C(O)O—, and one additional methylene unit of L is optionally replaced by —N(R)—, —O—, or —C(O)—.

In some embodiments, L is an optionally substituted bivalent C₂₋₈ unsaturated, straight or branched, hydrocarbon chain, wherein L has at least one triple bond and one methylene unit of L is replaced by —C(O)—, and one additional methylene unit of L is optionally replaced by —N(R)—, —O—, or —C(O)—.

In some embodiments, L is an optionally substituted bivalent C₂₋₈ straight or branched, hydrocarbon chain wherein L has at least one triple bond and one methylene unit of L is replaced by —SO₂—.

In some embodiments, L is —CH₂—C≡CCH₂N(R)—, —CH₂—C≡C—CH₂—, —CH₂C(O)C≡C—, or —C(O)C≡C—; and Y is hydrogen or C₁₋₆ aliphatic optionally substituted with oxo, halogen, NO₂, or CN. In certain embodiments, L is —C(O)C≡C—.

In some embodiments, L is optionally substituted with —OR^{∘}. In certain embodiments, L is optionally substituted with —OR^{∘}, wherein R^{∘} is hydrogen. In some embodiments, L is optionally substituted with one or more groups selected from —CN, halogen or phenyl. In some embodiments, L is optionally substituted with halogen. In some embodiments, L is optionally substituted with —CN. In some embodiments, L is optionally substituted with phenyl.

In some embodiments, L is —C(O)CH═CH—, —C(O)C(F)═CH—, —C(O)C(F)═CH-cyclopropylene-, —C(O)CH═C(F)—, —C(O)C(CN)═CH—, —C(O)C(CN)═CH-cyclopropylene-, —C(O)CH═C(CN)—, —CH(OH)CH═CH—, —CH(OH)C(F)═CH—, —CH(OH)C(CN)═CH—, —CH(OH)CH═C(F)—, or —CH(OH)CH═C(CN)—.

In some embodiments, Y is selected from hydrogen, halogen, —CN, C₁₋₆ aliphatic optionally substituted with oxo, halogen, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 groups independently selected from -Q-Z, oxo, —NO₂, halogen, —CN, and C₁₋₆ aliphatic. In some embodiments, Y is hydrogen. In some embodiments, Y is hydrogen or C₁₋₆ aliphatic optionally substituted with oxo, halogen, NO₂ or CN. In some embodiments, Y is C₁₋₆ aliphatic optionally substituted with oxo, halogen, NO₂ or CN. In some embodiments, Y is hydrogen or C₁₋₆ aliphatic. In some embodiments, Y is C₁₋₆ aliphatic.

In some embodiments, Y is selected from halogen, —CN, C₁₋₆ aliphatic optionally substituted with oxo, halogen, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 groups independently selected from -Q-Z, oxo, —NO₂, halogen, —CN, and C₁₋₆ aliphatic.

In some embodiments, Y is selected from hydrogen, halogen, —CN, or C₁₋₆ aliphatic optionally substituted with oxo, halogen, or CN. In some embodiments, Y is selected from halogen, —CN, or C₁₋₆ aliphatic optionally substituted with oxo, halogen, or CN. In some embodiments, Y is halogen. In some embodiments, Y is —CN. In some embodiments, Y is C₁₋₆ aliphatic optionally substituted with oxo, halogen, or CN. In some embodiments, Y is C₁₋₅ aliphatic optionally substituted with oxo, halogen, or CN. In some embodiments, Y is C₁₋₄ aliphatic optionally substituted with oxo, halogen, or CN. In some embodiments, Y is C₁₋₃ aliphatic optionally substituted with oxo, halogen, or CN. In some embodiments, Y is C₁₋₂ aliphatic optionally substituted with oxo, halogen, or CN.

In some embodiments, Y is a 3-10 membered monocyclic or bicyclic, saturated, partially unsaturated, or aryl ring having 0-3 heteroatoms independently selected from nitrogen, oxygen, or sulfur, and wherein said ring is substituted with 1-4 groups independently selected from -Q-Z, oxo, —NO₂, halogen, —CN, and C₁₋₆ aliphatic. In some embodiments, Y is a 3-10 membered monocyclic saturated ring having 0-3 heteroatoms independently selected from nitrogen, oxygen, or sulfur. In some embodiments, Y is a 8-10 membered bicyclic saturated ring having 0-3 heteroatoms independently selected from nitrogen, oxygen, or sulfur.

In some embodiments, Y is a 3-6 membered saturated, partially unsaturated, or aryl ring having 0-3 heteroatoms independently selected from nitrogen, oxygen, or sulfur. In some embodiments, Y is a 3-6 membered saturated ring having 0-3 heteroatoms independently selected from nitrogen, oxygen, or sulfur. In some embodiments, Y is a 3-membered saturated carbocyclic ring. In some embodiments, Y is a 4-membered saturated carbocyclic ring. In some embodiments, Y is a 5-membered saturated carbocyclic ring. In some embodiments, Y is a 6-membered saturated carbocyclic ring.

In some embodiments, Y is a 3-membered saturated ring having 1 heteroatom independently selected from nitrogen, oxygen, or sulfur. In some such embodiments, Y is selected from oxiranyl and aziridinyl.

In some embodiments, Y is a 4-membered saturated ring having 1 heteroatom independently selected from nitrogen, oxygen, or sulfur. In some such embodiments, Y is selected from oxetanyl and azetidinyl.

In some embodiments, Y is a 5-membered saturated ring having 1-2 heteroatoms independently selected from nitrogen, oxygen, or sulfur. In some such embodiments, Y is selected from pyrrolidinyl, tetrahydrofuranyl, imidazolidinyl, pyrazolidinyl, oxazolidinyl, isoxazolidinyl, thiazolidinyl, isothiazolidinyl, and dioxolanyl.

In some embodiments, Y is a 6-membered saturated ring having 1-2 heteroatoms independently selected from nitrogen, oxygen, or sulfur. In some such embodiments, Y is selected from piperidinyl, oxanyl, thianyl, piperazinyl, morpholinyl, thiomorpholinyl, dioxanyl, and dithianyl.

In some embodiments, Y is a 6-membered saturated ring having 2 heteroatoms independently selected from nitrogen, oxygen, or sulfur. In some such embodiments, Y is selected from piperazinyl, morpholinyl, and thiomorpholinyl.

In some embodiments, Y is a 3-6 membered partially unsaturated carbocyclic ring. In some embodiments, Y is a 3-membered saturated carbocyclic ring. In some embodiments, Y is a 4-membered saturated carbocyclic ring. In some embodiments, Y is a 5-membered saturated carbocyclic ring. In some embodiments, Y is a 6-membered saturated carbocyclic ring.

In some embodiments, Y is a 3-6 membered partially unsaturated ring having 1-3 heteroatoms independently selected from nitrogen, oxygen, or sulfur. In some embodiments, Y is selected from imidazolinyl, pyrazolinyl, oxazolinyl, thiazolinyl, pyranyl and thiopyranyl.

In some embodiments, Y is a 7-10 membered bicyclic partially unsaturated ring having 0-3 heteroatoms independently selected from nitrogen, oxygen, or sulfur.

In some embodiments, Y is phenyl.

In some embodiments, Y is a 5-6 membered heteroaryl ring having 1-3 heteroatoms independently selected from nitrogen, oxygen, or sulfur. In some embodiments, Y is a 5-membered heteroaryl ring having 1-3 heteroatoms independently selected from nitrogen, oxygen, or sulfur. In some such embodiments, Y is selected from pyrrolyl, furanyl, thiophenyl, imidazolyl, oxazolyl, isoxazolyl, triazolyl, oxadiazolyl, and thiadiaozlyl.

In some embodiments, Y is a 6-membered heteroaryl ring having 1-3 nitrogen atoms. In some such embodiments, Y is selected from pyridinyl and pyrimidinyl.

As described generally above, Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —SO—, or —SO₂—. In some embodiments, Q is a covalent bond. In some embodiments, Q is a bivalent C₁₋₆ saturated or unsaturated, straight or branched, hydrocarbon chain, wherein one or two methylene units of Q are optionally and independently replaced by —N(R)—, —S—, —O—, —C(O)—, —SO—, or —SO₂—. In some embodiments, Q is a bivalent C₁₋₆ saturated straight or branched, hydrocarbon chain, wherein one or two methylene units of Q are optionally and independently replaced by —N(R)—, —S—, —O—, —C(O)—, —SO—, or —SO₂—. In some embodiments, Q is a bivalent C₁₋₅, C₁₋₄, C₁₋₃, or C₁₋₂ saturated straight or branched, hydrocarbon chain, wherein one or two methylene units of Q are optionally and independently replaced by —N(R)—, —S—, —O—, —C(O)—, —SO—, or —SO₂—. In some embodiments, Q is a bivalent C₁₋₆ saturated straight or branched, hydrocarbon chain, wherein one or two methylene units of Q are optionally and independently replaced by —N(R)—, —O—, or —C(O)—. In some embodiments, Q is a bivalent C₁₋₆ unsaturated, straight or branched, hydrocarbon chain, wherein one or two methylene units of Q are optionally and independently replaced by —N(R)—, —S—, —O—, —C(O)—, —SO—, or —SO₂—. In some embodiments, Q is a bivalent C₁₋₅, C₁₋₄, C₁₋₃, or C₁₋₂ unsaturated straight or branched, hydrocarbon chain, wherein one or two methylene units of Q are optionally and independently replaced by —N(R)—, —S—, —O—, —C(O)—, —SO—, or —SO₂—. In some embodiments, Q is a bivalent C₁₋₆ unsaturated, straight or branched, hydrocarbon chain, wherein one or two methylene units of Q are optionally and independently replaced by —N(R)—, —O—, or —C(O)—.

As described generally above, Z is hydrogen or C₁₋₆ aliphatic optionally substituted with oxo, halogen, or CN. In some embodiments, Z is hydrogen. In some embodiments, Z is C₁₋₆ aliphatic optionally substituted with oxo, halogen, or CN. In some embodiments, Z is C₁₋₆ aliphatic substituted with oxo, halogen, or CN. In some embodiments, Z is C₁₋₆, C₁₋₅, C₁₋₄, C₁₋₃, or C₁₋₂ aliphatic substituted with oxo, halogen, or CN.

Without wishing to be bound by any particular theory, it is believed that such -L-Y groups, i.e. warhead groups, are particularly suitable for covalently binding to a key cysteine residue in the binding domain of certain protein kinases. Thus, in some embodiments, the -L-Y moiety is capable of covalently binding to a cysteine residue thereby irreversibly inhibiting the enzyme.

In certain embodiments, the -L-Y moiety is capable of covalently binding to a cysteine residue of TEC, thereby irreversibly inhibiting the enzyme. In some embodiments, the cysteine residue is Cys 449.

In certain embodiments, the -L-Y moiety is capable of covalently binding to a cysteine residue of BTK, thereby irreversibly inhibiting the enzyme. In some embodiments, the cysteine residue is Cys 481.

In certain embodiments, the -L-Y moiety is capable of covalently binding to a cysteine residue of ITK, thereby irreversibly inhibiting the enzyme. In some embodiments, the cysteine residue is Cys 442.

In certain embodiments, the -L-Y moiety is capable of covalently binding to a cysteine residue of BMX, thereby irreversibly inhibiting the enzyme. In some embodiments, the cysteine residue is Cys 496.

In certain embodiments, the -L-Y moiety is capable of covalently binding to a cysteine residue of BLK, thereby irreversibly inhibiting the enzyme. In some embodiments, the cysteine residue is Cys 319.

In certain embodiments, the -L-Y moiety is capable of covalently binding to a cysteine residue of EGFR, thereby irreversibly inhibiting the enzyme. In some embodiments, the cysteine residue is Cys 797

In certain embodiments, the -L-Y moiety is capable of covalently binding to a cysteine residue of ErbB2, thereby irreversibly inhibiting the enzyme. In some embodiments, the cysteine residue is Cys 805.

In certain embodiments, the -L-Y moiety is capable of covalently binding to a cysteine residue of ErbB4, thereby irreversibly inhibiting the enzyme. In some embodiments, the cysteine residue is Cys 803.

In certain embodiments, the -L-Y moiety is capable of covalently binding to a cysteine residue of JAK3, thereby irreversibly inhibiting the enzyme. In some embodiments, the cysteine residue is Cys 909.

In certain embodiments, the -L-Y moiety is capable of covalently binding to a cysteine residue of MAP2K7, thereby irreversibly inhibiting the enzyme. In some embodiments, the cysteine residue is Cys 202.

In certain embodiments, the -L-Y moiety is capable of covalently binding to a cysteine residue of TXK, thereby irreversibly inhibiting the enzyme. In some embodiments, the cysteine residue is Cys 350.

One of ordinary skill in the art will recognize that a variety of warhead groups, as defined herein, are suitable for such covalent bonding.

In certain embodiments, R¹ is selected from those set forth in Table 3, below, wherein each wavy line indicates the point of attachment to the rest of the molecule.

TABLE 3
Exemplary R¹ Groups

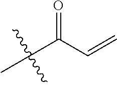 [SMILES: *C(=O)C=C]

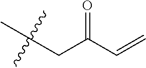 [SMILES: *CC(=O)C=C]

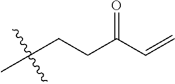 [SMILES: *CCC(=O)C=C]

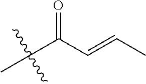 [SMILES: *C(=O)/C=C/C]

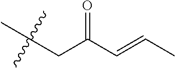 [SMILES: *CC(=O)/C=C/C]

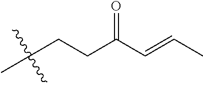 [SMILES: *CCC(=O)/C=C/C]

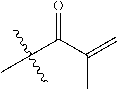 [SMILES: *C(=O)C(=C)C]

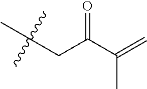 [SMILES: *CC(=O)C(=C)C]

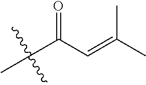 [SMILES: *C(=O)C=C(C)C]

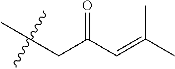 [SMILES: *CC(=O)C=C(C)C]

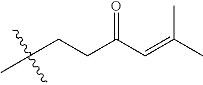 [SMILES: *CCC(=O)C=C(C)C]

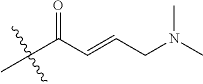 [SMILES: *C(=O)/C=C/CN(C)C]

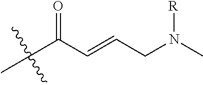

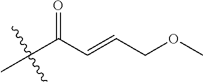 [SMILES: *C(=O)/C=C/COC]

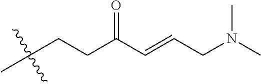 [SMILES: *CCC(=O)/C=C/CN(C)C]

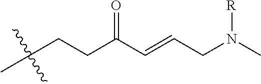

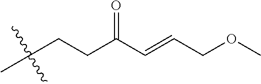 [SMILES: *CCC(=O)/C=C/COC]

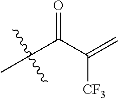 [SMILES: *C(=O)C(=C)C(F)(F)F]

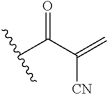 [SMILES: *C(=O)C(=C)C#N]

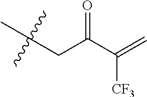 [SMILES: *CC(=O)C(=C)C(F)(F)F]

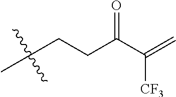 [SMILES: *CCC(=O)C(=C)C(F)(F)F]

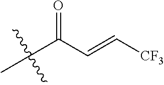 [SMILES: *C(=O)/C=C/C(F)(F)F]

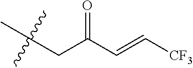 [SMILES: *CC(=O)/C=C/C(F)(F)F]

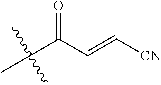 [SMILES: *C(=O)/C=C/C#N]

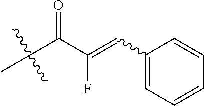 [SMILES: *C(=O)C(F)=Cc1ccccc1]

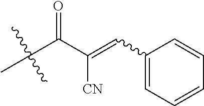 [SMILES: *C(=O)C(C#N)=Cc1ccccc1]

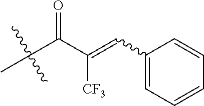 [SMILES: *C(=O)C(=Cc1ccccc1)C(F)(F)F]

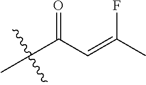 [SMILES: *C(=O)/C=C(/C)F]

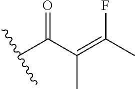 [SMILES: *C(=O)/C(C)=C(/C)F]

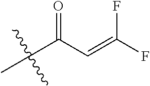 [SMILES: *C(=O)C=C(F)F]

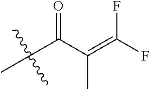 [SMILES: *C(=O)C(C)=C(F)F]

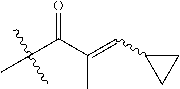 [SMILES: *C(=O)C(C)=CC1CC1]

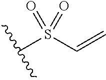 [SMILES: *S(=O)(=O)C=C]

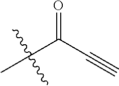 [SMILES: *C(=O)C#C]

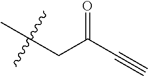 [SMILES: *CC(=O)C#C]

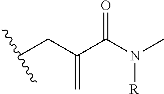

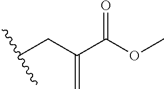 [SMILES: *CC(=C)C(=O)OC]

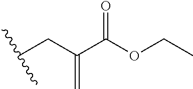 [SMILES: *CC(=C)C(=O)OCC]

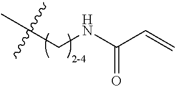

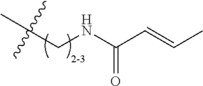

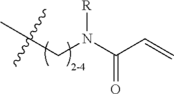

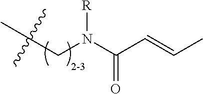

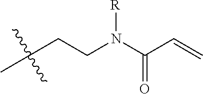

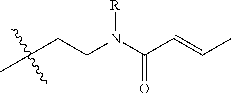

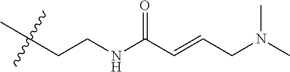 [SMILES: *CCNC(=O)/C=C/CN(C)C]

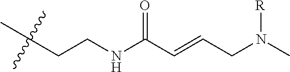

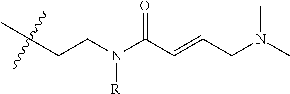

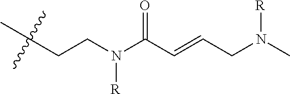

In certain embodiments, R¹ is selected from

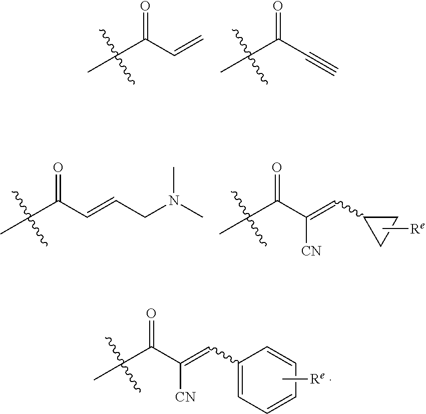

In certain exemplary embodiments, R¹ is

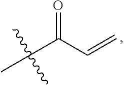 [SMILES: *C(=O)C=C]

As described generally above, R² is selected from hydrogen, C₁₋₆ aliphatic and —C(O)R^{} In some embodiments, R² is hydrogen. In some embodiments, R² is selected from C₁₋₆ aliphatic and —C(O)R^{}. In some embodiments, R² is C₁₋₆ aliphatic. In some embodiments, R² is C₁₋₆, C₁₋₅, C₁₋₄, C₁₋₃, or C₁₋₂ aliphatic. In some embodiments, R² is —CH₃. In some embodiments, R² is —C(O)R^{}. In some embodiments, R² is —C(O)CH₃.

As described generally above, W is —N(R²)CH₂— or —NH—. In some embodiments, W is —N(R²)CH₂—. In some embodiments, W is —NHCH₂—. In some embodiments, W is —N(CH₃)CH₂—. In some embodiments, W is —NH—.

As described generally above, R³ and R⁴ are each independently selected from hydrogen and halogen. In some embodiments, R³ is hydrogen. In some embodiments, R³ is halogen. In some such embodiments, R³ is fluoro. In some embodiments, R⁴ is hydrogen. In some embodiments, R⁴ is halogen. In some such embodiments, R⁴ is fluoro. In some embodiments, each of R³ and R⁴ is hydrogen. In some embodiments, each of R³ and R⁴ is halogen. In some embodiments, R³ is hydrogen and R⁴ is halogen. In some such embodiments, R⁴ is fluoro. In some embodiments, R³ is halogen and R⁴ is hydrogen. In some such embodiments, R³ is fluoro.

As described generally above, each R group is independently hydrogen or an optionally substituted group selected from C₁₋₆ aliphatic, phenyl, a 3-7 membered saturated or partially unsaturated carbocyclic ring, a 4-7 membered heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R is hydrogen. In some embodiments, R is an optionally substituted group selected from C₁₋₆ aliphatic, phenyl, a 3-7 membered saturated or partially unsaturated carbocyclic ring, a 4-7 membered heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R is optionally substituted C₁₋₆ aliphatic. In some embodiments, R is an optionally substituted phenyl. In some embodiments, R is an optionally substituted 3-7 membered saturated or partially unsaturated carbocyclic ring. In some embodiments, R is an optionally substituted a 4-7 membered heterocyclic ring having 1-2 heteroatoms independently selected from nitrogen, oxygen, and sulfur. In some embodiments, R is an optionally substituted 5-6 membered monocyclic heteroaryl ring having 1-4 heteroatoms independently selected from nitrogen, oxygen, and sulfur.

In some embodiments, R is selected from hydrogen and C₁₋₆ aliphatic.

In some embodiments, R is selected from C₁₋₆ aliphatic, phenyl, a 3-7 membered saturated or partially unsaturated carbocyclic ring, a 4-7 membered heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R is C₁₋₆ aliphatic. In some embodiments, R is C₁₋₅, C₁₋₄, C₁₋₃, or C₁₋₂ aliphatic. In some embodiments, R is selected from methyl, ethyl, propyl, isopropyl, butyl, sec-butyl, isobutyl, tert-butyl, pentyl, isopentyl, sec-pentyl, neopentyl, hexyl, 2-methyl-2-pentanyl, 3-methylpentanyl, 2,3-dimethylbutyl, and 2,2-dimethylbutyl.

In some embodiments, R is a 3-7 membered saturated or partially unsaturated carbocyclic ring.

In some embodiments, R is a 4-7 membered heterocyclic ring having 1-2 heteroatoms independently selected from nitrogen, oxygen, and sulfur.

In some embodiments, R is phenyl.

As described generally above, R^{y} is hydrogen, halogen, —CF₃, or C₁₋₄ aliphatic. In some embodiments, R^{y} is halogen. In some such embodiments, R^{y} is fluoro or bromo. In certain embodiments, R^{y} is fluoro. In some embodiments, R^{y} is —CF₃. In some embodiments, R^{y} is C₁₋₄ aliphatic. In some such embodiments, R^{y} is selected from methyl, ethyl, propyl, isopropyl, butyl, sec-butyl, or tert-butyl.

As described generally above, each R^{x} is independently oxo, halogen, —OR, —N(R)₂, —S(O)ₓR, —N(R)(CH₂)_{q}N(R)₂, —N(R)(CH₂)_{q}OR, —O(CH₂)_{q}OR, —O(CH₂)_{q}N(R)₂, an optionally substituted C₂—6 saturated, straight or branched, hydrocarbon chain wherein one or two methylene units are independently replaced by —O—, —N(R)— or —S(O)ₓ, or an optionally substituted group selected from C₁₋₆ aliphatic, phenyl, a 3-7 membered saturated or partially unsaturated carbocyclic ring, a 4-7 membered heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur. In some embodiments, R^{x} is oxo.

In some embodiments, each R^{x} is independently halogen, —OR, —N(R)₂, —S(O)ₓR, —N(R)(CH₂)_{q}N(R)₂, —N(R)(CH₂)_{q}OR, —O(CH₂)_{q}OR, —O(CH₂)_{q}N(R)₂, an optionally substituted C₂₋₆ saturated, straight or branched, hydrocarbon chain wherein one or two methylene units are independently replaced by —O—, —N(R)— or —S(O)ₓ—, or an optionally substituted group selected from C₁₋₆ aliphatic, phenyl, a 3-7 membered saturated or partially unsaturated carbocyclic ring, a 4-7 membered heterocyclic ring having 1-2 heteroatoms independently selected from nitrogen, oxygen, and sulfur, and a 5-6 membered monocyclic heteroaryl ring having 1-4 heteroatoms independently selected from nitrogen, oxygen, and sulfur.

In some embodiments, R^{x} is an optionally substituted C₁₋₆ aliphatic. In some embodiments, R^{x} is an optionally substituted C₁₋₄ aliphatic or C₁₋₂ aliphatic. In some embodiments, R^{x} is propargyl. In some embodiments, R^{x} is C₁₋₄ aliphatic substituted with oxo. In some embodiments, R^{x} is —C(O)CH₃. In some embodiments, R^{x} is C₁₋₄ aliphatic substituted with —C(O)N(R^{∘}₂. In some embodiments, R^{x} is —CH₂C(O)NH₂. In some embodiments, R^{x} is C₁₋₆ aliphatic optionally substituted with halogen. In some such embodiments, R^{x} is —CH₂CF₃. In some embodiments, R^{x} is C₁₋₆ aliphatic optionally substituted with one halogen. In some such embodiments, R^{x} is C₁₋₆ aliphatic optionally substituted with one fluoro. In some embodiments, Rx is C₁₋₆ aliphatic optionally substituted with —OR^{∘}. In some such embodiments, R^{∘} is selected from hydrogen or C₁₋₆ aliphatic. In some embodiments, R^{∘} is hydrogen. In some embodiments, R^{∘} is C₁₋₆ aliphatic. In some embodiments, R^{x} is —CH₂CH₂OH. In some embodiments, R^{x} is —CH₂CH₂OCH₃.

In some embodiments, R^{x} is C₁₋₆ aliphatic optionally substituted with R^{∘}. In some embodiments, R^{x} is —CH₂—R^{∘}. In some embodiments, R^{∘} is C₁₋₆ aliphatic. In some such embodiments, R^{∘} is —CH₃. In some embodiments, R^{∘} is C₁₋₆ aliphatic substituted with a group selected from —(CH₂)₀₋₄R^{}, —(CH₂)₀₋₄OH, —(CH₂)₀₋₄OR^{}, —(CH₂)₀₋₄NH₂, —(CH₂)₀₋₄NHR^{}, —(CH₂)₀₋₄ NR^{}₂, or ═O.

In some embodiments, R^{x} is —CH₂—R^{∘}, wherein R^{∘} is C₁₋₆ aliphatic substituted with —(CH₂)₀₋₄OH. In some embodiments, R^{∘} is C₁ aliphatic substituted with —(CH₂)₀₋₄OH. Thus, in some embodiments, R^{∘} is —CH₂—(CH₂)₀₋₄OH. In some such embodiments, R^{∘} is —CH₂OH.

In some embodiments, R^{x} is —CH₂—R^{∘}, wherein R^{∘} is C₁₋₆ aliphatic substituted with ═O and —(CH₂)₀₋₄NH₂. In some embodiments, R^{∘} is C₁ aliphatic substituted with ═O and —(CH₂)₀₋₄NH₂. Thus, in some embodiments, R^{∘} is —C(O)NH₂.

In some embodiments, R^{x} is —CH₂—R^{∘}, wherein R^{∘} is an optionally substituted 3-6-membered saturated, partially unsaturated, or aryl ring having 0-4 heteroatoms independently selected from nitrogen, oxygen, or sulfur, or two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur. In some embodiments, R^{∘} is an optionally substituted 3-6 membered carbocyclic ring. In some embodiments, R^{x} is —CH₂—R^{∘}, wherein R^{∘} is an optionally substituted 3-membered carbocyclic ring. In certain embodiments, R^{x} is —CH₂—R^{∘}, wherein R^{∘} is cyclopropyl. In some embodiments, R^{∘} is cyclobutyl.

In some embodiments, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur. In some such embodiments, two independent occurrences of R^{∘}, taken together with their intervening atom(s), form a 4-membered carbocyclic ring. Thus, in some embodiments, two independent occurrences of R^{∘}, taken together with their intervening atom(s), form a cyclobutyl ring.

In some embodiments, two independent occurrences of R^{∘}, taken together with their intervening atom(s), form a 4-membered heterocyclic ring having 1 heteroatom selected from nitrogen, oxygen, or sulfur. In some such embodiments, two independent occurrences of R^{∘}, taken together with their intervening atom(s), form a 4-membered heterocyclic ring having 1 oxygen atom. Thus, in some embodiments, two independent occurrences of R^{∘}, taken together with their intervening atom(s), form an oxetanyl ring.

In some embodiments, R^{∘} is a 3-6-membered saturated, partially unsaturated, or aryl ring having 0-4 heteroatoms independently selected from nitrogen, oxygen, or sulfur wherein R^{∘}is substituted with —(CH₂)₀₋₂R^{}. In some such embodiments, R^{} is C₁₋₆ aliphatic. In some embodiments, R^{∘} is an optionally substituted 4-6-membered saturated heterocyclic ring having 1 heteroatom selected from nitrogen, oxygen or sulfur. In some embodiments, R^{∘} is an optionally substituted 4-membered saturated ring having 1 heteroatom selected from nitrogen, oxygen or sulfur. In certain embodiments, R^{∘} is oxetanyl.

In some embodiments, R^{x} is —CH₂—R^{∘}, wherein R^{∘} is an optionally substituted 6-membered saturated heterocyclic ring having 1 heteroatom selected from nitrogen, oxygen or sulfur. In certain embodiments, R^{∘} is an optionally substituted group selected from piperidinyl and tetrahydropyranyl. In some embodiments, R^{∘} is tetrahydropyranyl.

In some embodiments, R^{x} is C₁₋₆ aliphatic. In some embodiments, R^{x} is a C₁₋₄ aliphatic. In certain embodiments, R^{x} is a straight or branched C₁₋₄ aliphatic chain. In some embodiments, R^{x} is lower alkyl. In some such embodiments, R^{x} is selected from —CH₃ and —CD₃. In some such embodiments, R^{x} is —CH₃. In some embodiments, R^{x} is —CD₃. In some embodiments, R^{x} is ethyl. In some embodiments, R^{x} is C₃₋₅ aliphatic. In some embodiments, R^{x} is isopropyl. In some embodiments, R^{x} is tert-butyl. In some embodiments, R^{x} is neopentyl (—CH₂C(CH₃)₃).

In some embodiments, R^{x} is C₁₋₆ aliphatic optionally substituted with —OR^{∘} or —C(O)NR^{∘}₂. In some embodiments, R^{x} is C₁₋₆ aliphatic optionally substituted with R^{∘}, wherein R^{∘} is an optionally substituted 3-6-membered saturated, partially unsaturated, or aryl ring having 0-4 heteroatoms independently selected from nitrogen, oxygen, or sulfur. In some such embodiments, R^{∘} is cyclopropyl. In some embodiments, R^{x} is C₁₋₆ aliphatic optionally substituted with R^{∘}, wherein R^{∘} is a 4-membered heterocyclic ring having 1 heteroatom selected from nitrogen, oxygen, or sulfur.

In some embodiments, R^{x} is an optionally substituted phenyl. In some embodiments, R^{x} is phenyl optionally substituted with halogen. In some such embodiments, R^{x} is phenyl optionally substituted with fluoro.

In some embodiments, R^{x} is an optionally substituted 3-7 membered saturated or partially unsaturated carbocyclic ring. In some embodiments, R^{x} is cyclopropyl. In some embodiments, R^{x} is an optionally substituted 4-7 membered saturated or partially unsaturated carbocyclic ring. In some embodiments, R^{x} is cyclobutyl. In some embodiments, R^{x} is cyclopentyl.

In some embodiments, R^{x} is an optionally substituted 4-7 membered heterocyclic ring having 1-2 heteroatoms independently selected from nitrogen, oxygen, and sulfur. In some embodiments, R^{x} is an optionally substituted 4-membered heterocyclic ring having 1 heteroatom selected from nitrogen, oxygen, and sulfur. In some such embodiments, R^{x} is oxetanyl.

In some embodiments, R^{x} is an optionally substituted 5-membered heterocyclic ring having 1-2 heteroatoms independently selected from nitrogen, oxygen, and sulfur.

In some embodiments, R^{x} is an optionally substituted 6-membered heterocyclic ring having 1-2 heteroatoms independently selected from nitrogen, oxygen, and sulfur. In some such embodiments, R^{x} is an optionally substituted group selected from piperidinyl and tetrahydropyranyl.

In some embodiments, R^{x} is selected from those groups in Table 4A:

TABLE 4A

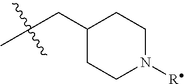

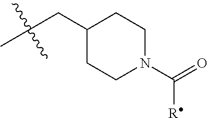

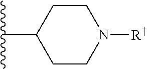

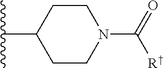

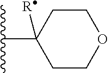

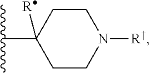

wherein:
each R^{†} is selected from hydrogen and C₁₋₆ aliphatic optionally substituted with halogen; and
each R^{} is selected from hydrogen and C₁₋₆ aliphatic optionally substituted with halogen.

In some embodiments, R^{x} is selected from those groups in Table 4A-i:

TABLE 4A-i

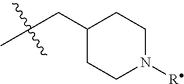

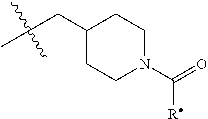

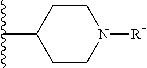

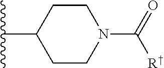

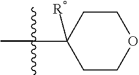

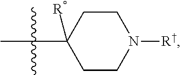

wherein:
each R^{∘} is selected from hydrogen and C₁₋₆ aliphatic optionally substituted with halogen;
each R^{†} is selected from hydrogen and C₁₋₆ aliphatic optionally substituted with halogen; and
each R^{} is selected from hydrogen and C₁₋₆ aliphatic optionally substituted with halogen.

In some embodiments, R^{∘} is hydrogen. In certain embodiments, R^{∘} is C₁₋₆ aliphatic. In some embodiments, R^{∘} is —CH₃. In some embodiments, R^{∘} is —CD₃.

In some embodiments, R^{†} is hydrogen. In certain embodiments, R^{†} is C₁₋₆ aliphatic. In some embodiments, R^{†} is —CH₃. In some embodiments, R^{†} is —CD₃.

In some embodiments, R^{} is hydrogen. In certain embodiments, R^{} is C₁₋₆ aliphatic. In some embodiments, R^{} is —CH₃. In some embodiments, R^{} is —CD₃.

In certain embodiments, R^{x} is selected from —CH₃, —CD₃, —CH(CH₃)₂, —C(CH₃)₃, —CH₂C(CH₃)₃, —C(O)CH₃, —CH₂C(O)NH₂, —CH₂CH₂OCH₃, —CH₂CH₂OH, —CH₂CH₂F, —CH₂CF₃, or those groups in Table 4B:

TABLE 4B
Exemplary R^{x} Groups

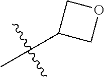 [SMILES: *C1COC1]

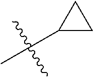 [SMILES: *C1CC1]

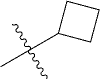 [SMILES: *C1CCC1]

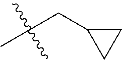 [SMILES: *CC1CC1]

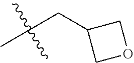 [SMILES: *CC1COC1]

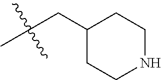 [SMILES: *CC1CCNCC1]

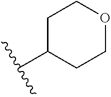 [SMILES: *C1CCOCC1]

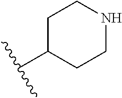 [SMILES: *C1CCNCC1]

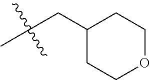 [SMILES: *CC1CCOCC1]

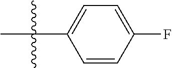 [SMILES: *c1ccc(F)cc1]

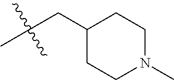 [SMILES: *CC1CCN(C)CC1]

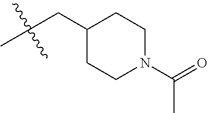 [SMILES: *CC1CCN(C(C)=O)CC1]

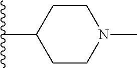 [SMILES: *C1CCN(C)CC1]

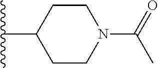 [SMILES: *C1CCN(C(C)=O)CC1]

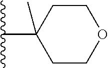 [SMILES: *C1(C)CCOCC1]

In some embodiments, R^{x} is selected from —CH₃, —CD₃, —CH(CH₃)₂, —C(CH₃)₃, —CH₂C(CH₃)₃, —CH₂C≡CH, —C(O)CH₃, —CH₂C(O)NH₂, —CH₂CH₂OCH₃, —CH₂CH₂OH, —CH₂CH₂F, —CH₂ CF₃, oxo

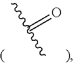 [SMILES: *=O]

or those groups in Table 4B.

In certain embodiments, R^{x} is selected from —CH₃, —CD₃, —CH(CH₃)₂, —C(CH₃)₃, —CH₂C(O)NH₂, —CH₂CH₂OH, or those groups in Table 4C:

TABLE 4C

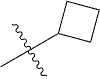 [SMILES: *C1CCC1]

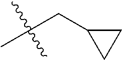 [SMILES: *CC1CC1]

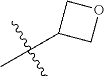 [SMILES: *C1COC1]

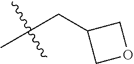 [SMILES: *CC1COC1]

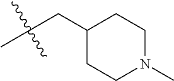 [SMILES: *CC1CCN(C)CC1]

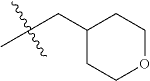 [SMILES: *CC1CCOCC1]

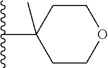 [SMILES: *C1(C)CCOCC1]

In certain embodiments, R^{x} is selected from oxo,

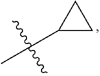 [SMILES: *C1CC1]

—CH₃, —CD₃, —CH(CH₃)₂, —C(CH₃)₃, —CH₂C(O)NH₂, —CH₂CH₂OH, or those groups in Table 4C.

As described generally above, each R^{v} is independently selected from halogen and C₁₋₆ aliphatic. In some embodiments, R^{v} is halogen. In some such embodiments, R^{v} is fluoro. In some embodiments, R^{v} is C₁₋₆ aliphatic. In some embodiments, R^{v} is C₁₋₆, C₁₋₅, C₁₋₄, C₁₋₃, or C₁₋₂ aliphatic. In some such embodiments, R^{v} is selected from methyl, ethyl, propyl or isopropyl.

As described generally above, x is 0, 1 or 2. In some embodiments, x is 0. In some embodiments, x is 1. In some embodiments, x is 2.

In some embodiments, the present invention provides a compound of formula II-a, II-b, II-c, II-d, II-e, II-f, II-g, II-h, II-i, II-j, II-k or II-l:

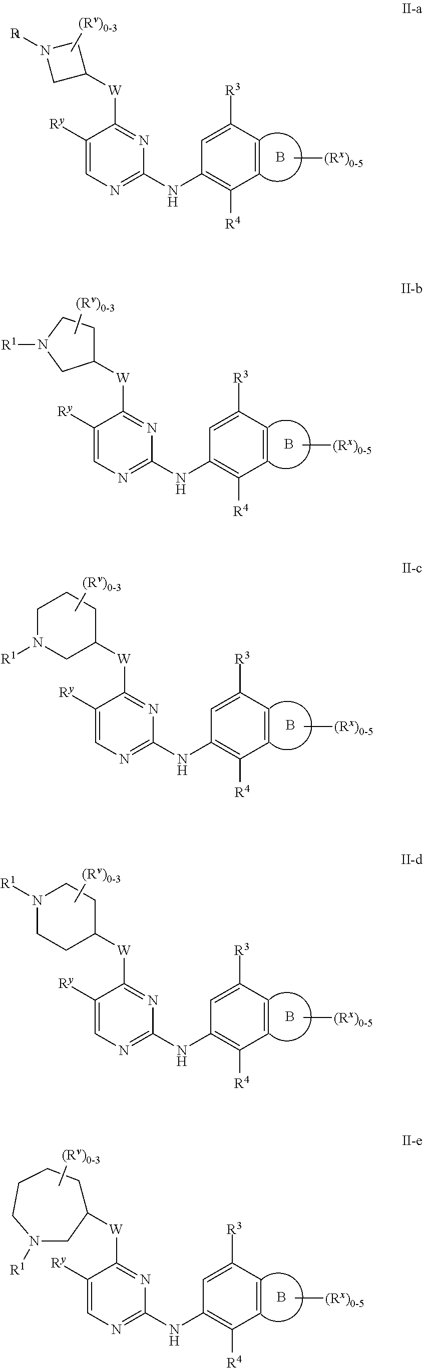

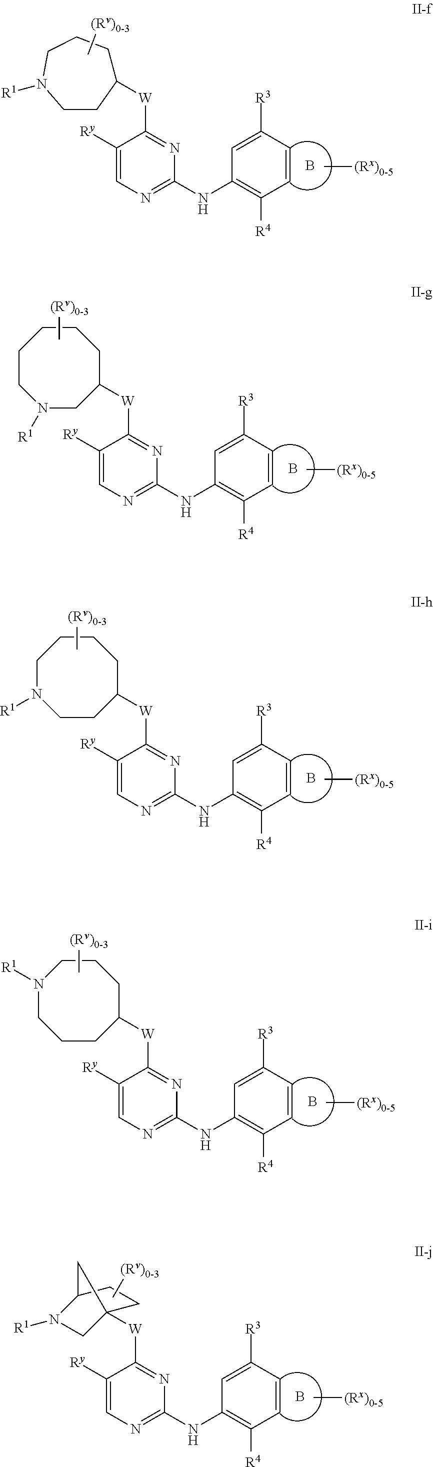

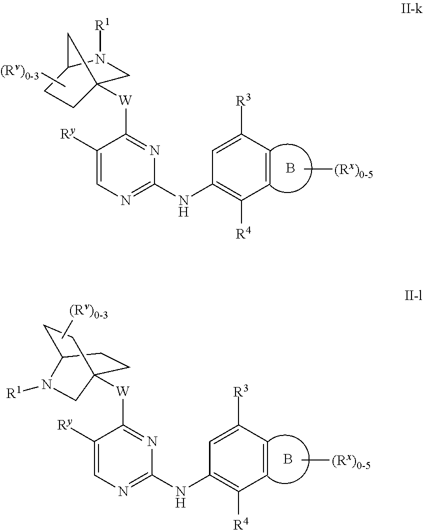

or a pharmaceutically acceptable salt thereof, wherein each of Ring B, W, R¹, R³, R⁴R^{v}, R^{x} and R^{y} are as defined above and described herein.

In some embodiments, the present invention provides a compound of formula II-b-i, II-b-ii, II-c-i, II-c-ii, II-e-i, II-e-ii, II-f-i, II-g-i, II-g-ii, II-h-i or II-h-ii:

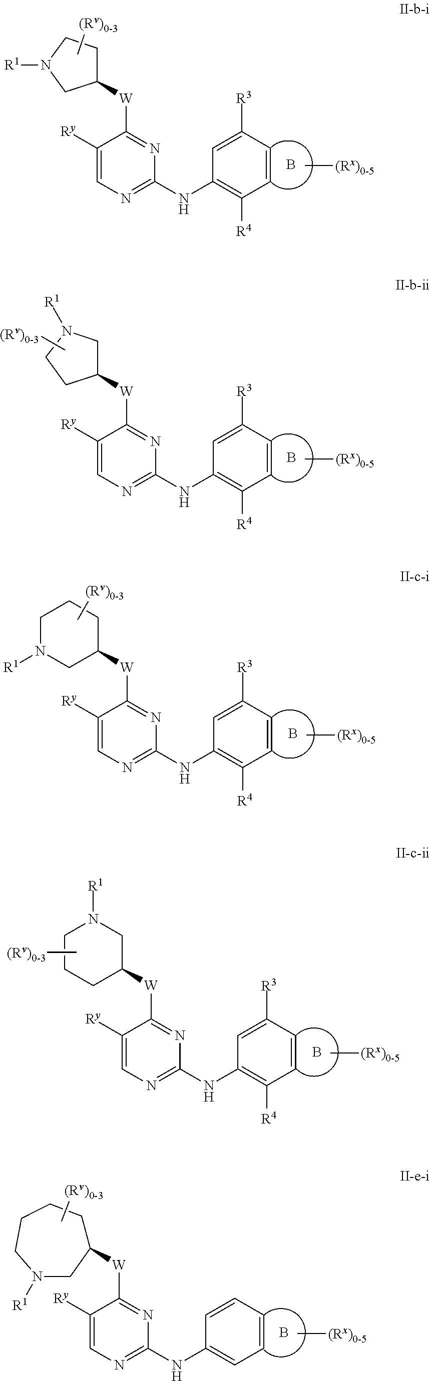

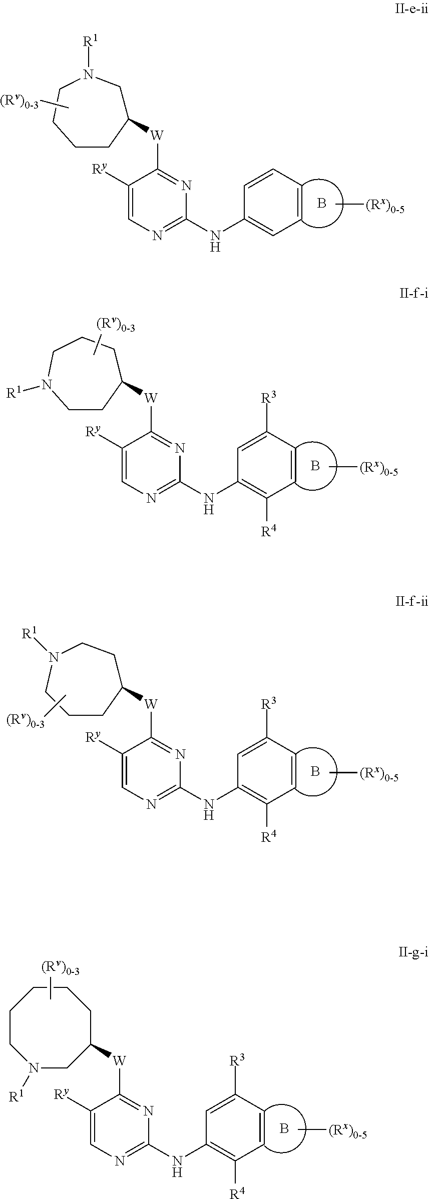

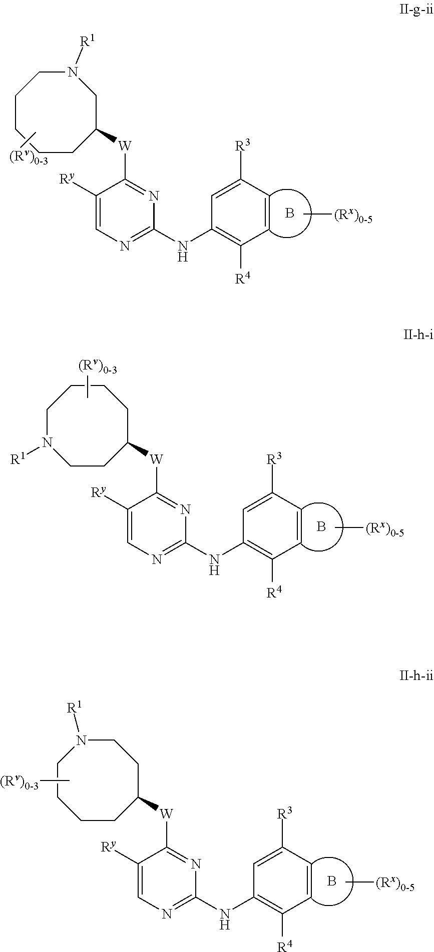

or a pharmaceutically acceptable salt thereof, wherein each of Ring B, W, R¹, R³, R⁴, R^{v}, R^{x} and R^{y} are as defined above and described herein.

In certain embodiments of formulae II-a, II-b, II-b-i, II-b-ii, II-c, II-c-i, II-c-ii, II-d, II-e, II-e-i, II-e-ii, II-f, II-f-i, II-f-ii, II-g, II-g-i, II-g-ii, II-h, II-h-i, II-h-ii, II-i, II-j, II-k or II-l, Ring B is selected from:

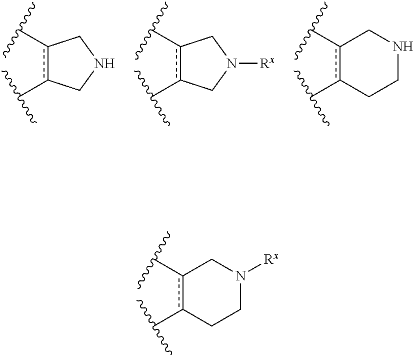

wherein R^{x} is as defined above and described herein.

In some embodiments of formulae II-a, II-b, II-b-i, II-b-ii, II-c, II-c-i, II-c-ii, II-d, II-e, II-e-i, II-e-ii, II-f, II-f-i, II-f-ii, II-g, II-g-i, II-g-ii, II-h, II-h-i, II-h-ii, II-i, II-j, II-k or II-l, Ring B is selected from:

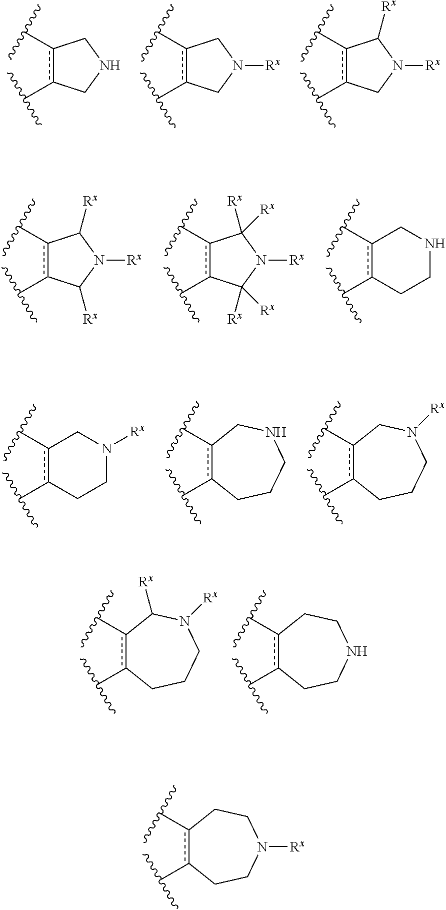

wherein R^{x} is as defined above and described herein.

In some embodiments, the present invention provides a compound of formula III-a, III-b, III-c, III-d, III-e, III-f, III-g or III-h:

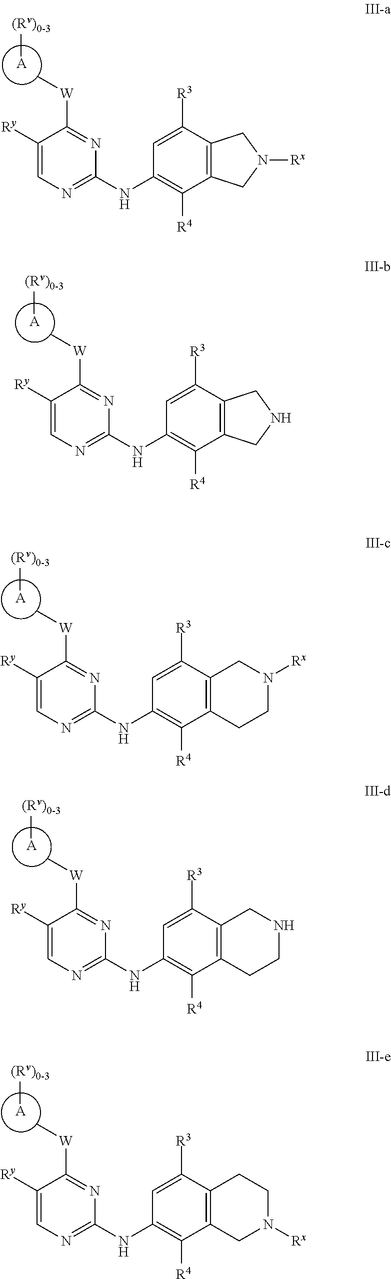

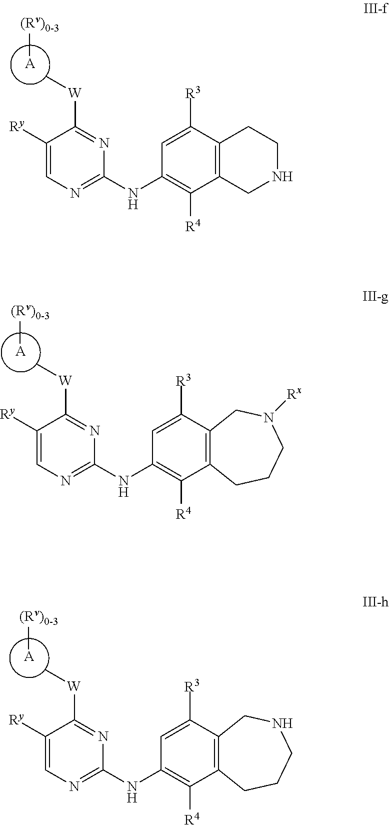

or a pharmaceutically acceptable salt thereof, wherein each of Ring A, W, R³, R⁴, R^{v}, R^{x} and R^{y} are as defined above and described herein.

In some embodiments, the present invention provides a compound of formula III-a, III-b, III-c, III-d, III-e, III-f, III-g or III-h, or a compound of formula III-i, III-k, III-l, III-m, III-n, III-o or III-p:

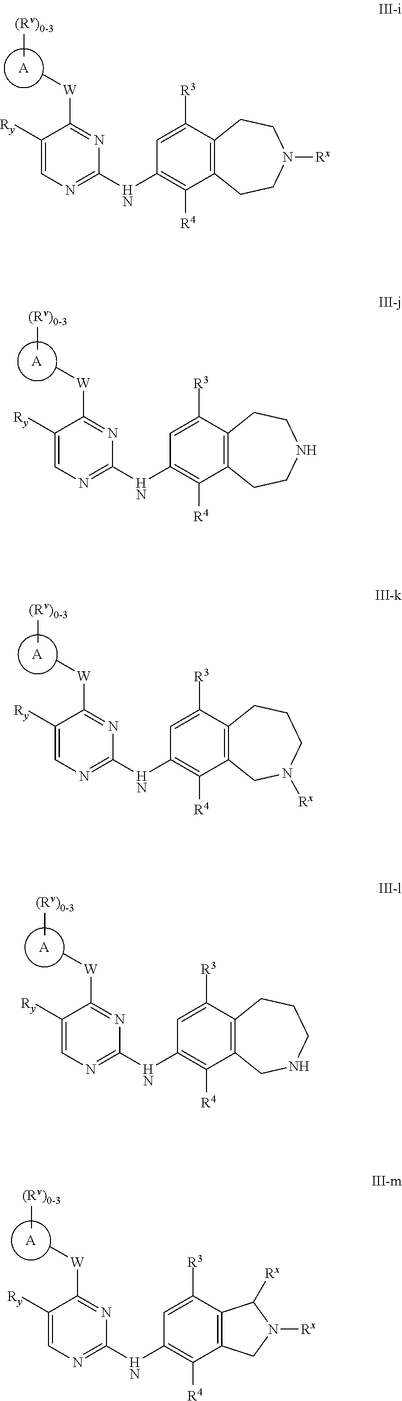

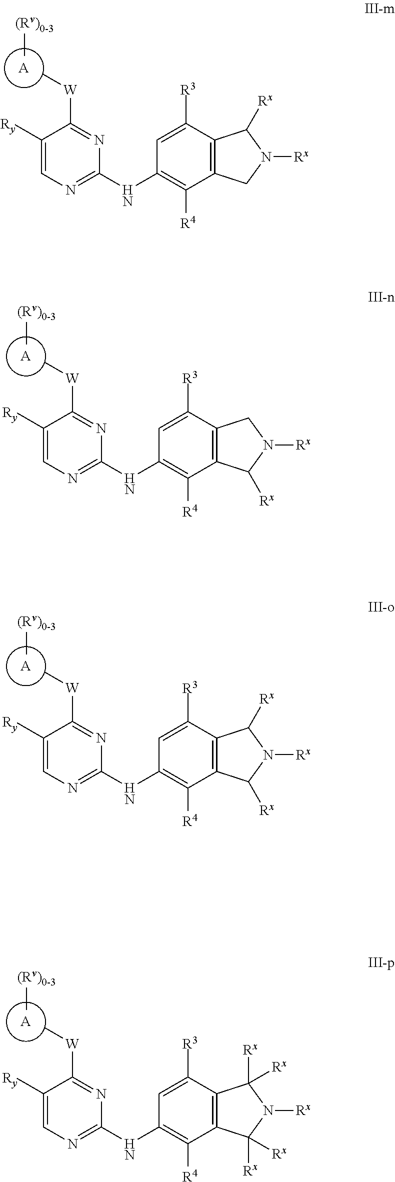

or a pharmaceutically acceptable salt thereof, wherein each of Ring A, W, R³, R⁴, R^{v}, R^{x} and R^{y} are as defined above and described herein.

In some embodiments of formulae III-a, III-f, III-g or III-h, Ring A is selected from:

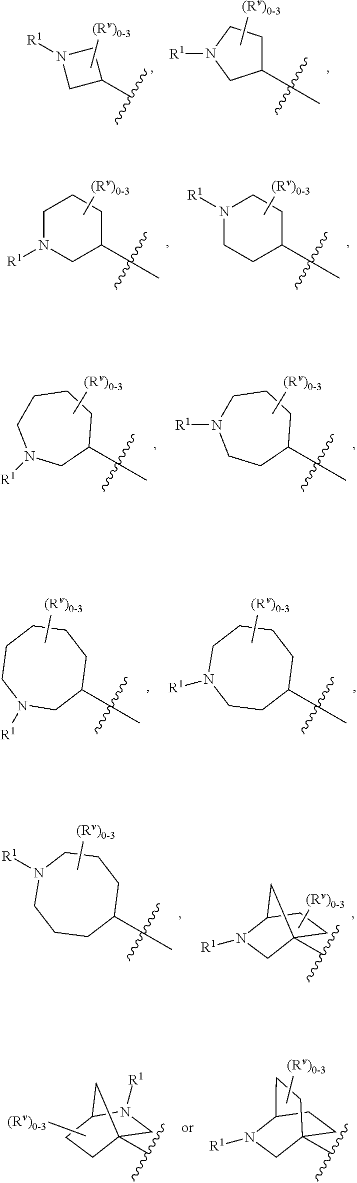

wherein each of R¹ and R^{v} is as defined above and described herein.

In some embodiments of formulae III-a, III-b, III-c, III-d, III-e, III-f, III-g, III-h, III-i , III-j, III-k, III-l, III-m, III-n, III-o, or III-p, Ring A is selected from:

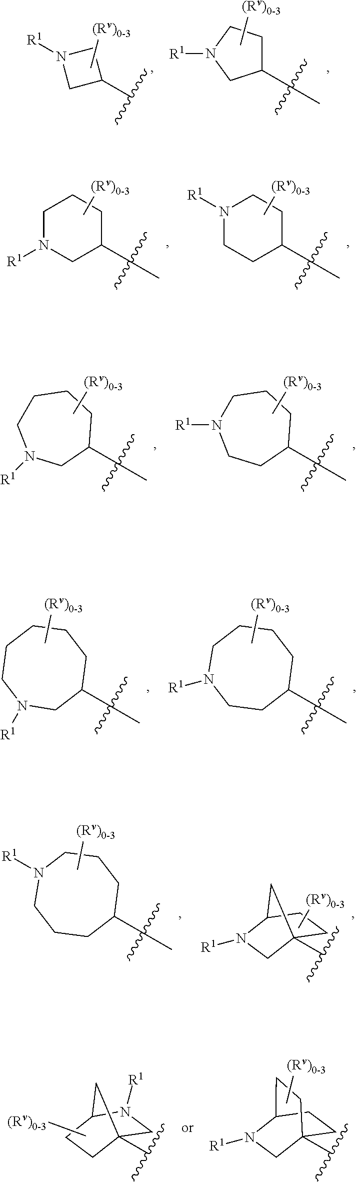

wherein each of R¹ and R^{v} is as defined above and described herein.

In certain embodiments of formulae III-a, III-b, III-c, III-d, III-e, III-f, III-g or III-h, Ring A is selected from:

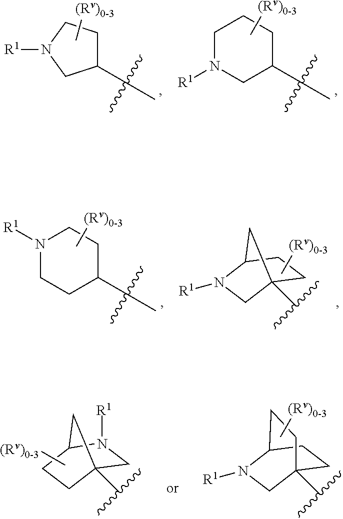

wherein each of R¹ and R^{v} is as defined above and described herein.

In certain embodiments of formulae III-a, III-b, III-c, III-d, III-e, III-f, III-g, III-h, III-i , III-j, III-k, III-l , III-m, III-n, III-o or III-p, Ring A is selected from:

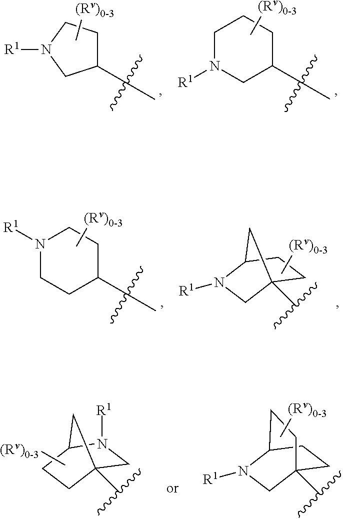

wherein each of R¹ and R^{v} is as defined above and described herein.

In some embodiments of formulae III-a, III-b, III-c, III-d, III-e, III-f, III-g or III-h, Ring A is substituted with 0-3 R^{v} groups and is selected from:

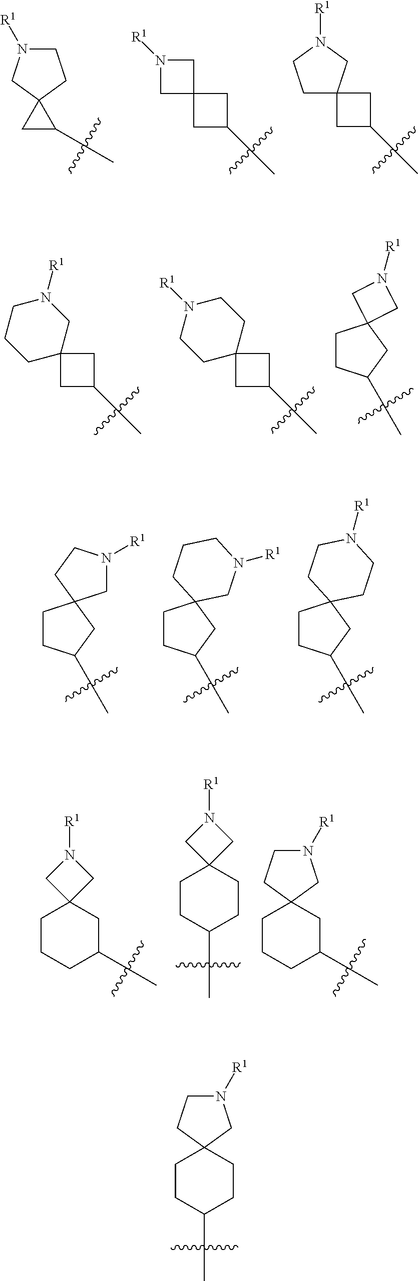

wherein R¹ is as defined above and described herein.

In some embodiments of formulae III-a, III-b, III-c, III-d, III-e, III-f, III-g, III-h, III-i, III-j, III-k, III-l , III-m, III-n, III-o or III-p, Ring A is substituted with 0-3 R^{v} groups and is selected from:

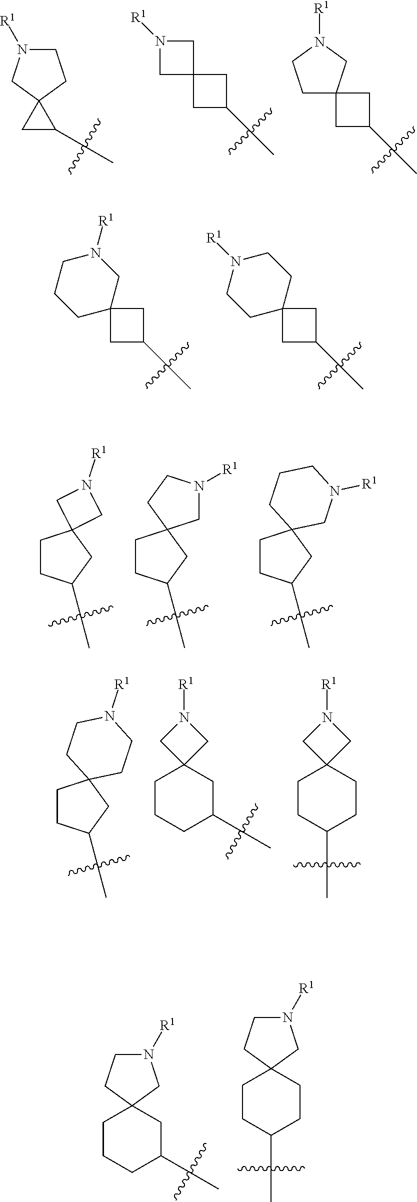

wherein R¹ is as defined above and described herein.

In some embodiments, the present invention provides a compound of formula IV-a or IV-b:

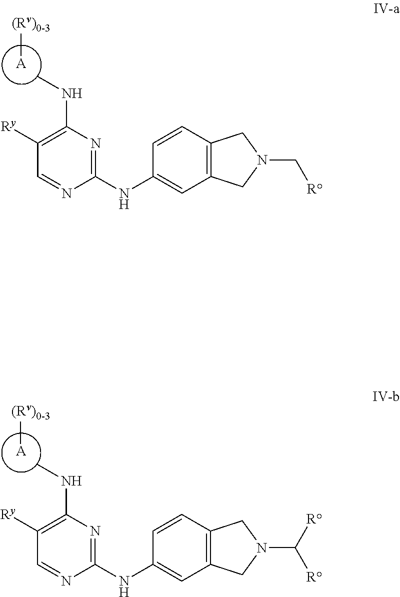

wherein each of Ring A, R^{v}, R^{y} and R^{∘} is as defined above and described herein.

In some embodiments, the present invention provides a compound of formula IV-c:

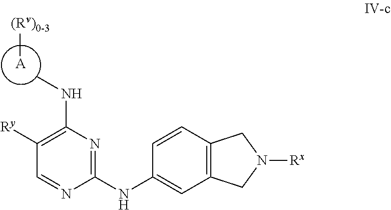

wherein each of Ring A, R^{v}, R^{y} and R^{x} is as defined above and described herein.

In some embodiments of formula IV-c, R^{x} is C₁₋₆ aliphatic. In some embodiments, R^{x} is optionally substituted methyl. In some embodiments, R^{x} is optionally substituted ethyl. In some embodiments, R^{x} is —CH₃. In some embodiments, R^{x} is —CD₃. In some embodiments, R^{x} is ethyl (i.e., —CH₂CH₃). In some embodiments, R^{x} is propyl (for example, cyclopropyl). In some embodiments, R^{x} is isopropyl. In some embodiments, R^{x} is butyl (for example, n-butyl, sec-butyl, tert-butyl or cyclobutyl). In some embodiments, R^{x} is pentyl (for example, n-pentyl, isopentyl, neopentyl, etc.). In some embodiments R^{x} is hexyl.

In some embodiments of formula IV-c, R^{x} is C₁₋₆ aliphatic optionally substituted with a group selected from —OH, —C(O)NH₂, or an optionally substituted 6-membered heterocyclic ring.

In some embodiments of formula IV-c, R^{x} is selected from a 4-6 membered carbocyclic ring or a C₁₋₄ straight or branched chain aliphatic optionally substituted with a 3-6 membered saturated or partially unsaturated carbocyclic ring or a 5-6 membered heterocyclic ring having 1-4 heteroatoms independently selected from —N(R^{})—, oxygen or sulfur; wherein R^{} is C₁₋₆ aliphatic.

In some embodiments, the present invention provides a compound of formula IV-d:

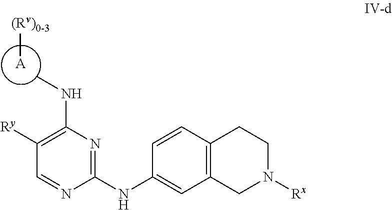

wherein each of Ring A, R^{v}, R^{y} and R^{x} is as defined above and described herein.

In some embodiments, the present invention provides a compound of formula IV-e:

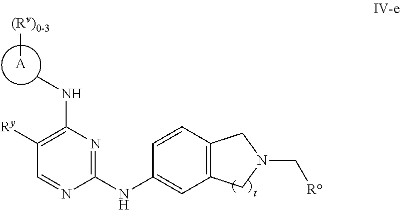

wherein t is 1, 2 or 3 and each of Ring A, R^{v}, R^{y} and R^{∘} is as defined above and described herein.

In some embodiments, the present invention provides a compound of formula IV-f:

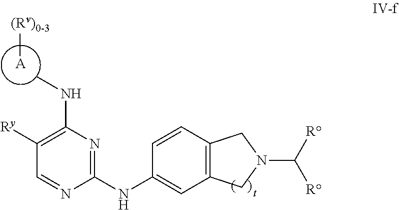

wherein t is 1, 2 or 3 and each of Ring A, R^{v}, R^{y} and R^{∘} is as defined above and described herein.

In some embodiments, the present invention provides a compound of formula IV-g:

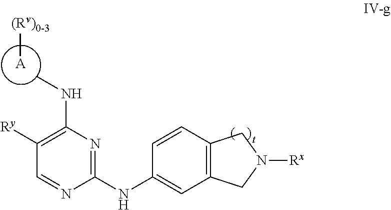

wherein:
t is 1 or 2;
R^{x} is C₁₋₄ straight or branched chain aliphatic; and
each of R^{v} and R^{y} is as defined above and described herein.

In some embodiments, the present invention provides a compound of formula IV-h:

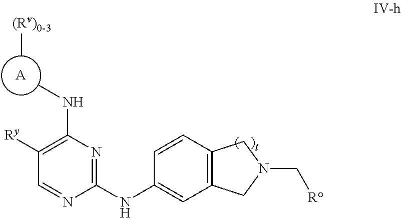

wherein t is 1 or 2 and each of Ring A, R^{v}, R^{y} and R^{∘} is as defined above and described herein.

In some embodiments, the present invention provides a compound of formula IV-i:

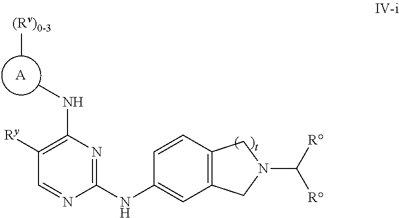

wherein t is 1 or 2 and each of Ring A, R^{v}, R^{y} and R^{∘} is as defined above and described herein.

In some embodiments, the present invention provides a compound of formula IV-j:

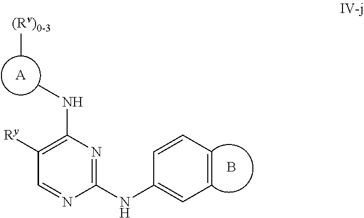

wherein each of Ring A, Ring B, R^{v} and R^{y} is as defined above and described herein.

In some embodiments of formula IV-j, Ring B is a 5-7 membered heterocyclo ring having one —N(CH(R^{∘})—, wherein R^{∘} is as defined above and described herein.

In some embodiments of formula IV-j, Ring B is a 5-7 membered heterocyclo ring having one —N(CH(R^{∘}₂)—, wherein R^{∘} is as defined above and described herein.

In some embodiments of formula IV-j, Ring B is a 5-7 membered heterocyclo ring having one —NH— or one —N(R^{x})—; and R^{x} is selected from a 4-6 membered carbocyclic ring, a 4-5 membered heterocyclic ring having one heteroatom independently selected from nitrogen, oxygen or sulfur, or a C₁₋₄ straight or branched chain aliphatic optionally substituted with —(CH₂)₀₋₄OH, —(CH₂)₀₋₄C(O)NH₂, a 3-6 membered saturated or partially unsaturated carbocyclic ring, or a 4-6 membered heterocyclic ring having 1-4 heteroatoms independently selected from —N(R^{})—, oxygen or sulfur, wherein R^{} is C₁₋₆ aliphatic.

In some embodiments, the present invention provides a compound of formula IV-k, IV-l or IV-m:

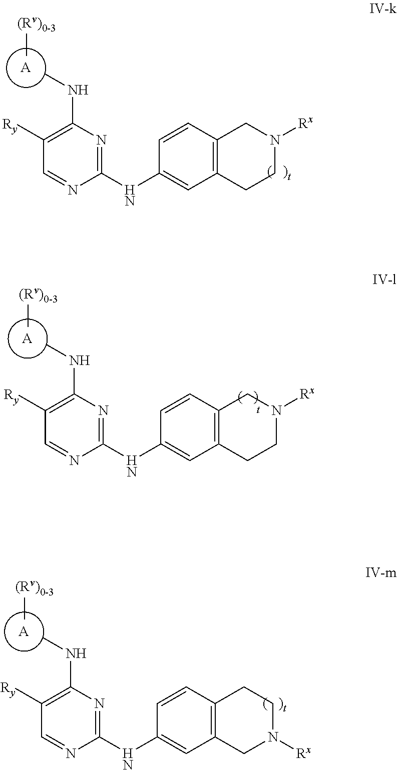

wherein t is 1 or 2 and each of Ring A, Rᵥ, R^{y} and R^{x} is as defined above and described herein.

In some embodiments of formulae IV-k, IV-I and IV-m, R^{x} is selected from —CH₃, —CH(CH₃)₂, —C(CH₃)₃, —C(O)CH₃,

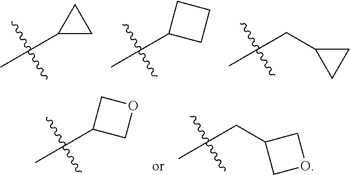

In some embodiments, the present invention provides a compound of formula V-a or V-b:

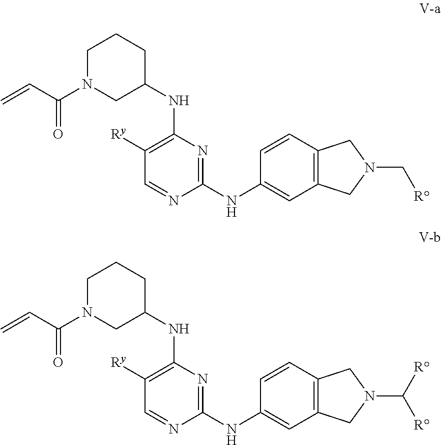

wherein each of R^{y} and R^{∘} is as defined above and described herein.

In some embodiments, the present invention provides a compound of formula V-c:

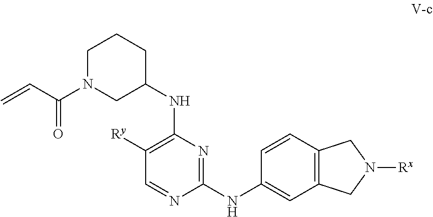

wherein each of R^{y} and R^{x} is as defined above and described herein.

In some embodiments of formula V-c, R^{x} is C₁₋₆ aliphatic optionally substituted with a group selected from —OH, —C(O)NH₂, or a 6-membered heterocyclic ring.

In some embodiments of formula V-c, R^{x} is C₁₋₆ aliphatic.

In some embodiments of formula V-c, R^{x} is selected from a 4-6 membered carbocyclic ring or a C₁₋₄ straight or branched chain aliphatic optionally substituted with a 3-6 membered saturated or partially unsaturated carbocyclic ring or a 5-6 membered heterocyclic ring having 1-4 heteroatoms independently selected from —N(R^{})—, oxygen or sulfur; wherein R^{} is C₁₋₆ aliphatic.

In some embodiments, the present invention provides a compound of formula V-d:

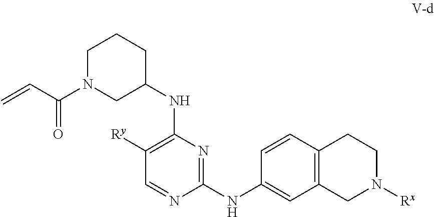

wherein each of R^{y} and R^{x} is as defined above and described herein.

In some embodiments, the present invention provides a compound of formula V-e:

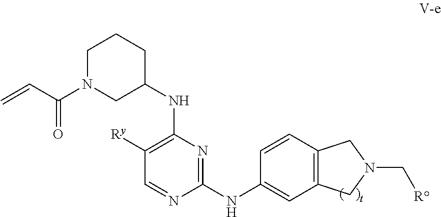

wherein t is 1, 2 or 3 and each of R^{y} and R^{∘} is as defined above and described herein.

In some embodiments, the present invention provides a compound of formula V-f:

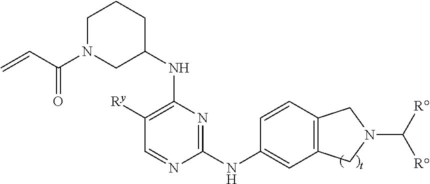

wherein t is 1, 2 or 3 and each of R^{y} and R^{∘} is as defined above and described herein.

In some embodiments, the present invention provides a compound of formula V-g:

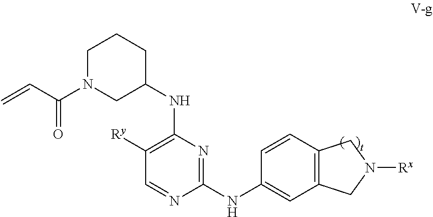

wherein:
t is 1 or 2;
Rₓ is C₁₋₄ straight or branched chain aliphatic; and
R_{y} is as defined above and described herein.

In some embodiments, the present invention provides a compound of formula V-h:

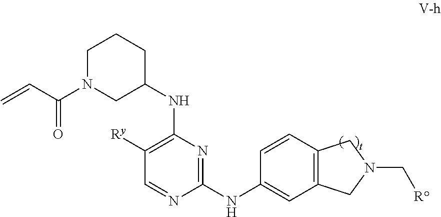

wherein t is 1 or 2 and each of R^{y} and R^{∘} is as defined above and described herein.

In some embodiments, the present invention provides a compound of formula V-i:

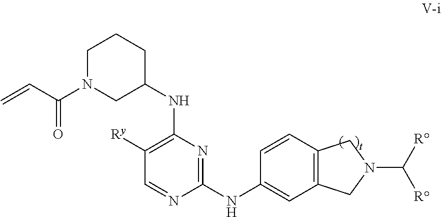

wherein t is 1 or 2 and each of R^{y} and R^{∘} is as defined above and described herein.

In some embodiments, the present invention provides a compound of formula V-j:

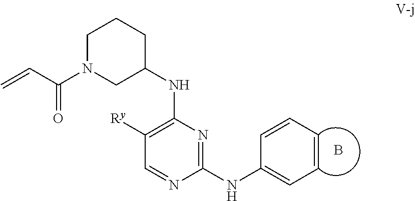

wherein each of R^{y} and Ring B is as defined above and described herein.

In some embodiments of formula V-j, Ring B is a 5-7 membered heterocyclo ring having one —N(CH(R^{∘})—, wherein R^{∘} is as defined above and described herein.

In some embodiments of formula V-j, Ring B is a 5-7 membered heterocyclo ring having one —N(CH(R^{∘}₂)—, wherein R^{∘} is as defined above and described herein.

In some embodiments of formula V-j, Ring B is a 5-7 membered heterocyclo ring having one —NH— or one —N(R^{x})—; and R^{x} is selected from a 4-6 membered carbocyclic ring, a 4-5 membered heterocyclic ring having one heteroatom independently selected from nitrogen, oxygen or sulfur, or a C₁₋₄ straight or branched chain aliphatic optionally substituted with —(CH₂)₀₋₄OH, —(CH₂)₀₋₄C(O)NH₂, a 3-6 membered saturated or partially unsaturated carbocyclic ring, or a 4-6 membered heterocyclic ring having 1-4 heteroatoms independently selected from —N(R^{})—, oxygen or sulfur, wherein R^{} is C₁₋₆ aliphatic.

In some embodiments, the present invention provides a compound of formula V-k, V-l or V-m:

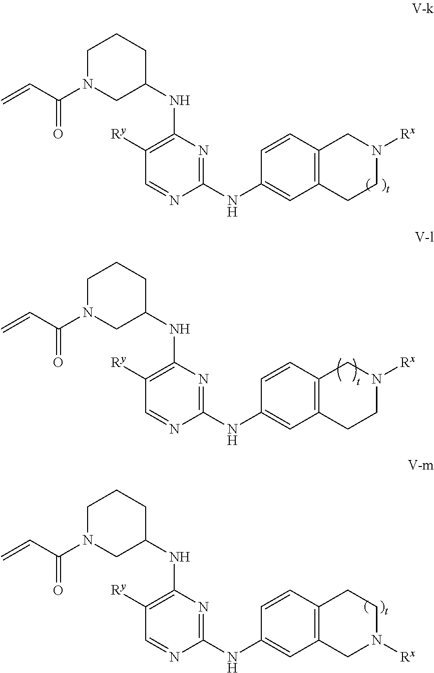

wherein t is 1 or 2 and each of Ring A, R^{y} and R^{x} is as defined above and described herein.

In some embodiments of formulae V-k, V-l and V-m, R^{x} is selected from —CH₃, —CH(CH₃)₂, —C(CH₃)₃, —C(O)CH₃,

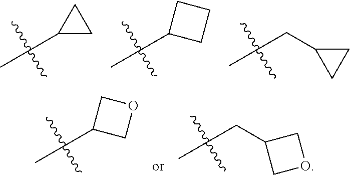

It will be appreciated that, when a formula described herein contains more than one R^{∘} (e.g., IV-b, IV-f, IV-i, IV-j, V-b, V-f, V-i, V-j, etc.), each R^{∘} is, independent of the other, as defined above and described herein. In some embodiments, each R^{∘} is the same. In some embodiments, each R^{∘} is different.

In certain embodiments of formulae IV-a, IV-b, IV-e, IV-f, IV-h, IV-i, IV-j, V-a, V-b, V-e, V-f, V-h, V-i and V-j, R^{∘} is hydrogen.

In certain embodiments, it will be appreciated that one or more nitrogen atoms in Ring B are basic nitrogen atoms. For example, it will be appreciated that the Ring B nitrogen atom in formulae IV-a, IV-b, IV-e, IV-f, IV-h, IV-i, V-a, V-b, V-e, V-f, V-h and V-i is a basic nitrogen atom. In some embodiments, the Ring B nitrogen atom in formulae IV-a, IV-b, IV-e, IV-f, IV-h, IV-i, IV-k, IV-l, IV-m, V-a, V-b, V-e, V-f, V-h, V-i, V-k, V-l and V-m is a basic nitrogen atom. In certain embodiments, Ring B comprises at least one basic nitrogen atom. In some embodiments, Ring B comprises one basic nitrogen atom. In some such embodiments, the basic nitrogen is a secondary nitrogen atom. That is, in some embodiments, the basic nitrogen atom in Ring B is not substituted. In some embodiments, the basic nitrogen is a tertiary nitrogen atom. That is, in some embodiments, Ring B comprises a basic nitrogen that is substituted with a moiety that does not substantially reduce or diminish its basicity. Such moieties include aliphatic groups and carbocyclic or heterocyclic rings.

In certain embodiments of formulae I, II-a, II-b, II-c, II-d, II-e, II-f, II-g, II-h, II-i, II-j, II-k, II-l , III-a, III-c, III-e, III-g, IV-c, IV-d, IV-g, IV-j, V-c, V-d, V-g and V-j, the Ring B nitrogen is a basic nitrogen. In certain embodiments of formulae I, II-a, II-b, II-c, II-d, II-e, II-f, II-g, II-h, II-i, II-j, II-k, II-l , III-a, III-c, III-e, III-g, III-i, III-k, III-m, III-n, III-o, IV-c, IV-d, IV-g, IV-j, IV-k, IV-l, IV-m, V-c, V-d, V-g, V-j, V-k, V-l and V-m, the Ring B nitrogen is a basic nitrogen. That is, in certain embodiments of formulae I, II-a, II-b, II-c, II-d, II-e, II-f, II-g, II-h, II-i, II-j, II-k, II-l , IV-j and V-j, the nitrogen atom in Ring B is either unsubstituted or is substituted with a moiety that does not substantially reduce or diminish its basicity. In certain embodiments of formulae III-a, III-c, III-e, III-g, IV-c, IV-d, IV-g, IV-j, V-c, V-d, V-g and V-j, the Ring B nitrogen is a basic nitrogen such that R^{x} is a moiety which does not substantially reduce or diminish its basicity. In certain embodiments of formulae III-a, III-c, III-e, III-g, III-i, III-k, III-m, III-n, III-o, IV-c, IV-d, IV-g, IV-j, IV-k, IV-l, IV-m, V-c, V-d, V-g, V-j, V-k, V-l and V-m, the Ring B nitrogen is a basic nitrogen such that R^{x} is a moiety which does not substantially reduce or diminish its basicity.

In certain embodiments, the compound of formula I is selected from the compounds in Table 5:

TABLE 5

| | |
|---|---|
| 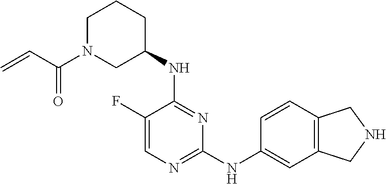 [SMILES: C=CC(=O)N1CCC[C@@H](Nc2nc(Nc3ccc4c(c3)CNC4)ncc2F)C1] | I-1 |
| 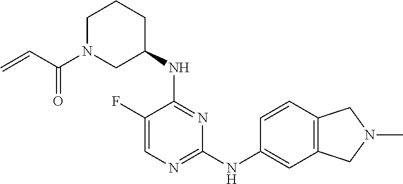 [SMILES: C=CC(=O)N1CCC[C@@H](Nc2nc(Nc3ccc4c(c3)CN(C)C4)ncc2F)C1] | I-2 |
| 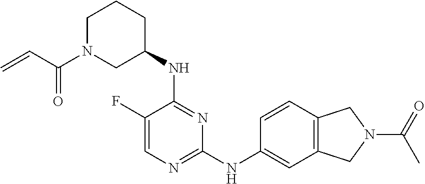 [SMILES: C=CC(=O)N1CCC[C@@H](Nc2nc(Nc3ccc4c(c3)CN(C(C)=O)C4)ncc2F)C1] | I-3 |
| 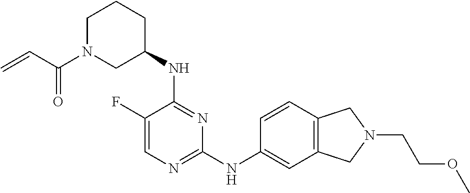 [SMILES: C=CC(=O)N1CCC[C@@H](Nc2nc(Nc3ccc4c(c3)CN(CCOC)C4)ncc2F)C1] | I-4 |
| 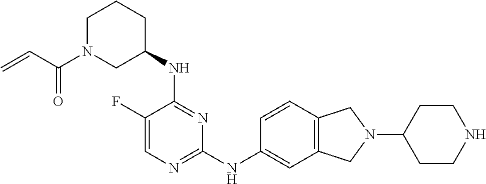 [SMILES: C=CC(=O)N1CCC[C@@H](Nc2nc(Nc3ccc4c(c3)CN(C3CCNCC3)C4)ncc2F)C1] | I-5 |
| 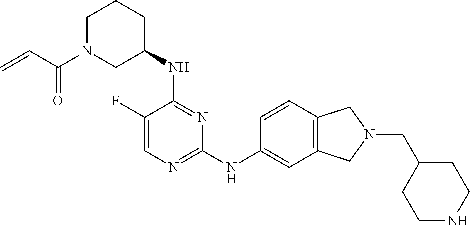 [SMILES: C=CC(=O)N1CCC[C@@H](Nc2nc(Nc3ccc4c(c3)CN(CC3CCNCC3)C4)ncc2F)C1] | I-6 |
| 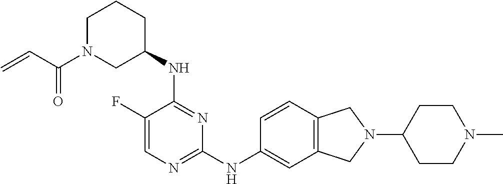 [SMILES: C=CC(=O)N1CCC[C@@H](Nc2nc(Nc3ccc4c(c3)CN(C3CCN(C)CC3)C4)ncc2F)C1] | I-7 |
| 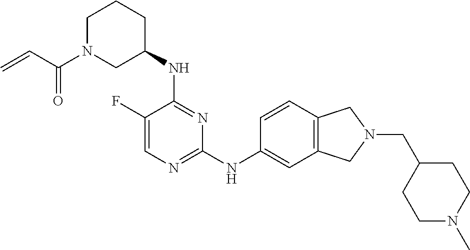 [SMILES: C=CC(=O)N1CCC[C@@H](Nc2nc(Nc3ccc4c(c3)CN(CC3CCN(C)CC3)C4)ncc2F)C1] | I-8 |
| 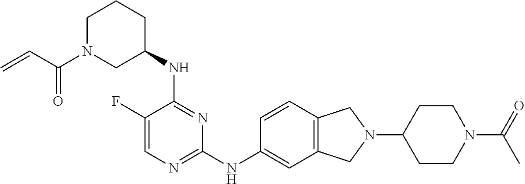 [SMILES: C=CC(=O)N1CCC[C@@H](Nc2nc(Nc3ccc4c(c3)CN(C3CCN(C(C)=O)CC3)C4)ncc2F)C1] | I-9 |
| 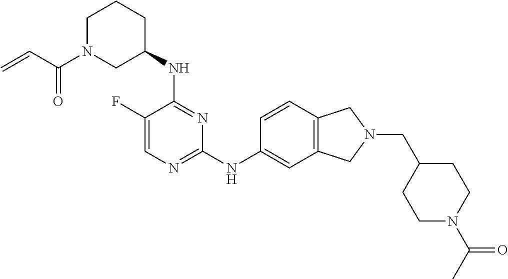 [SMILES: C=CC(=O)N1CCC[C@@H](Nc2nc(Nc3ccc4c(c3)CN(CC3CCN(C(C)=O)CC3)C4)ncc2F)C1] | I-10 |
| 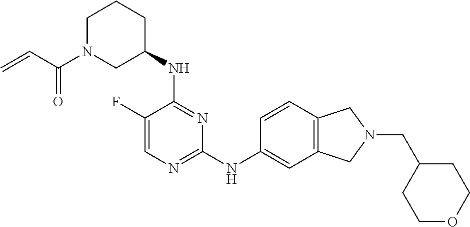 [SMILES: C=CC(=O)N1CCC[C@@H](Nc2nc(Nc3ccc4c(c3)CN(CC3CCOCC3)C4)ncc2F)C1] | I-11 |
| 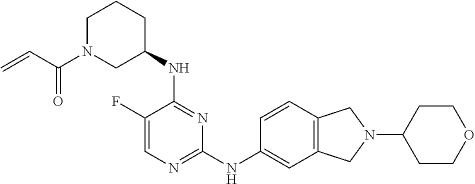 [SMILES: C=CC(=O)N1CCC[C@@H](Nc2nc(Nc3ccc4c(c3)CN(C3CCOCC3)C4)ncc2F)C1] | I-12 |
| 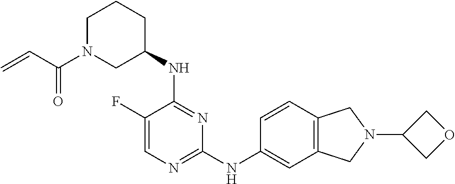 [SMILES: C=CC(=O)N1CCC[C@@H](Nc2nc(Nc3ccc4c(c3)CN(C3COC3)C4)ncc2F)C1] | I-13 |
| 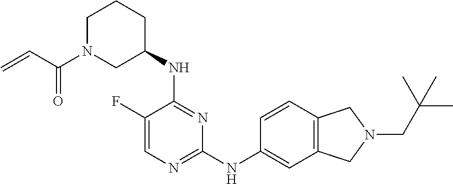 [SMILES: C=CC(=O)N1CCC[C@@H](Nc2nc(Nc3ccc4c(c3)CN(CC(C)(C)C)C4)ncc2F)C1] | I-14 |
| 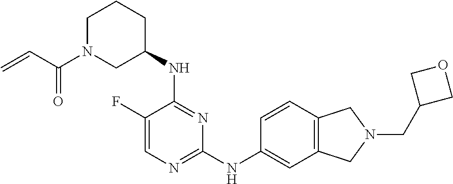 [SMILES: C=CC(=O)N1CCC[C@@H](Nc2nc(Nc3ccc4c(c3)CN(CC3COC3)C4)ncc2F)C1] | I-15 |
| 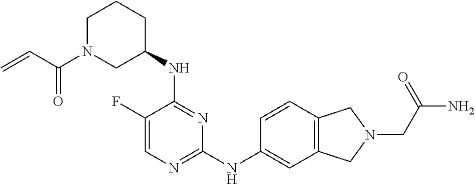 [SMILES: C=CC(=O)N1CCC[C@@H](Nc2nc(Nc3ccc4c(c3)CN(CC(N)=O)C4)ncc2F)C1] | I-16 |
| 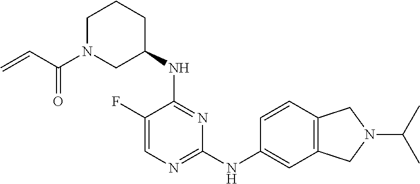 [SMILES: C=CC(=O)N1CCC[C@@H](Nc2nc(Nc3ccc4c(c3)CN(C(C)C)C4)ncc2F)C1] | I-17 |
| 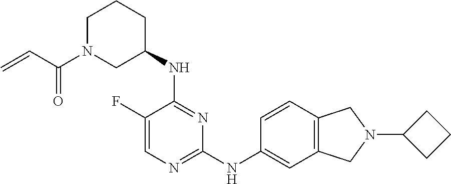 [SMILES: C=CC(=O)N1CCC[C@@H](Nc2nc(Nc3ccc4c(c3)CN(C3CCC3)C4)ncc2F)C1] | I-18 |
| 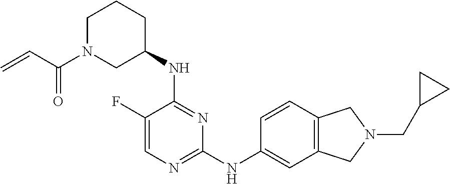 [SMILES: C=CC(=O)N1CCC[C@@H](Nc2nc(Nc3ccc4c(c3)CN(CC3CC3)C4)ncc2F)C1] | I-19 |
| 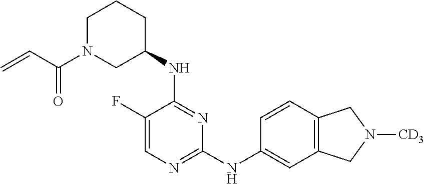 [SMILES: [2H]C([2H])([2H])N1Cc2ccc(Nc3ncc(F)c(N[C@@H]4CCCN(C(=O)C=C)C4)n3)cc2C1] | I-20 |
| 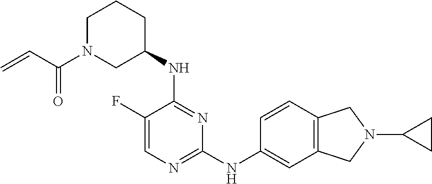 [SMILES: C=CC(=O)N1CCC[C@@H](Nc2nc(Nc3ccc4c(c3)CN(C3CC3)C4)ncc2F)C1] | I-21 |
| 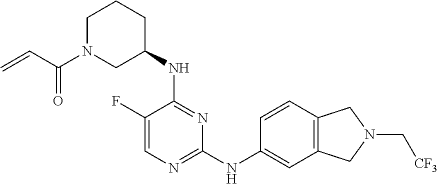 [SMILES: C=CC(=O)N1CCC[C@@H](Nc2nc(Nc3ccc4c(c3)CN(CC(F)(F)F)C4)ncc2F)C1] | I-22 |
| 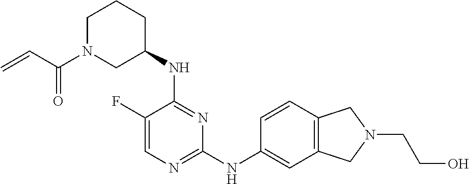 [SMILES: C=CC(=O)N1CCC[C@@H](Nc2nc(Nc3ccc4c(c3)CN(CCO)C4)ncc2F)C1] | I-23 |
| 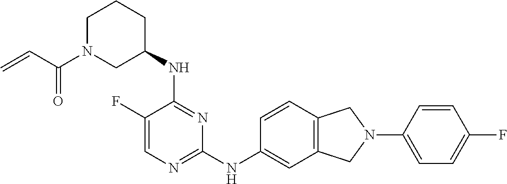 [SMILES: C=CC(=O)N1CCC[C@@H](Nc2nc(Nc3ccc4c(c3)CN(c3ccc(F)cc3)C4)ncc2F)C1] | I-24 |
| 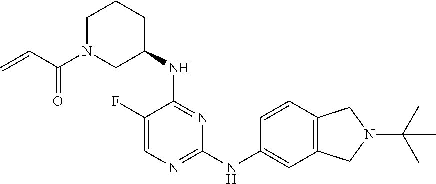 [SMILES: C=CC(=O)N1CCC[C@@H](Nc2nc(Nc3ccc4c(c3)CN(C(C)(C)C)C4)ncc2F)C1] | I-25 |
| 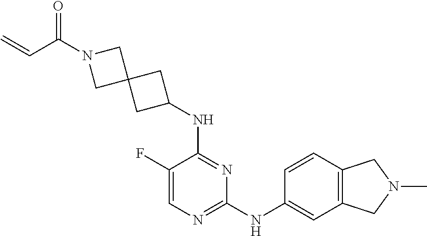 [SMILES: C=CC(=O)N1CC2(CC(Nc3nc(Nc4ccc5c(c4)CN(C)C5)ncc3F)C2)C1] | I-26 |
| 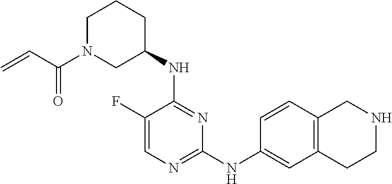 [SMILES: C=CC(=O)N1CCC[C@@H](Nc2nc(Nc3ccc4c(c3)CCNC4)ncc2F)C1] | I-27 |
| 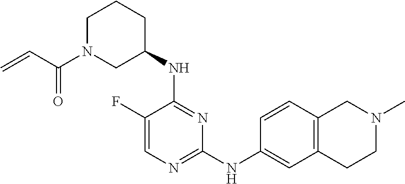 [SMILES: C=CC(=O)N1CCC[C@@H](Nc2nc(Nc3ccc4c(c3)CCN(C)C4)ncc2F)C1] | I-28 |
| 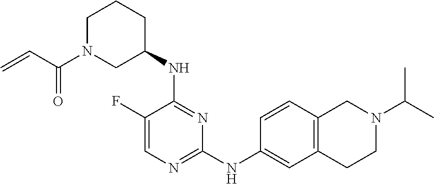 [SMILES: C=CC(=O)N1CCC[C@@H](Nc2nc(Nc3ccc4c(c3)CCN(C(C)C)C4)ncc2F)C1] | I-29 |
| 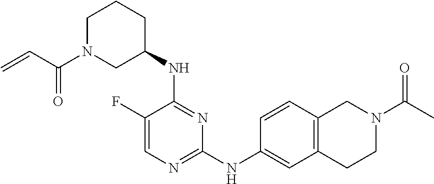 [SMILES: C=CC(=O)N1CCC[C@@H](Nc2nc(Nc3ccc4c(c3)CCN(C(C)=O)C4)ncc2F)C1] | I-30 |
| 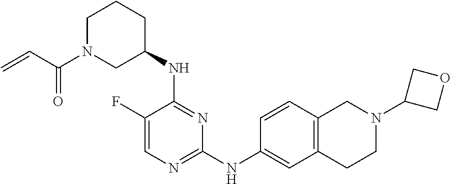 [SMILES: C=CC(=O)N1CCC[C@@H](Nc2nc(Nc3ccc4c(c3)CCN(C3COC3)C4)ncc2F)C1] | I-31 |
| 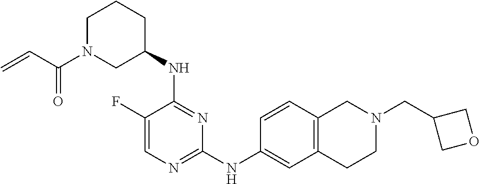 [SMILES: C=CC(=O)N1CCC[C@@H](Nc2nc(Nc3ccc4c(c3)CCN(CC3COC3)C4)ncc2F)C1] | I-32 |
| 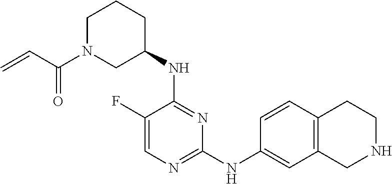 [SMILES: C=CC(=O)N1CCC[C@@H](Nc2nc(Nc3ccc4c(c3)CNCC4)ncc2F)C1] | I-33 |
| 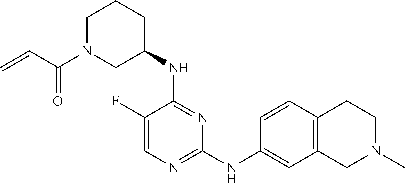 [SMILES: C=CC(=O)N1CCC[C@@H](Nc2nc(Nc3ccc4c(c3)CN(C)CC4)ncc2F)C1] | I-34 |
| 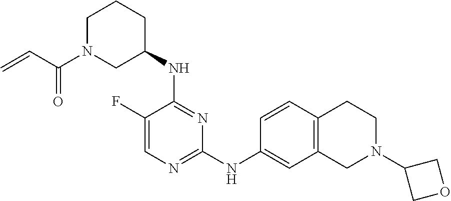 [SMILES: C=CC(=O)N1CCC[C@@H](Nc2nc(Nc3ccc4c(c3)CN(C3COC3)CC4)ncc2F)C1] | I-35 |
| 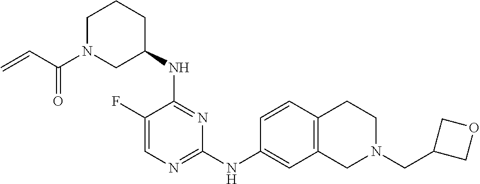 [SMILES: C=CC(=O)N1CCC[C@@H](Nc2nc(Nc3ccc4c(c3)CN(CC3COC3)CC4)ncc2F)C1] | I-36 |
| 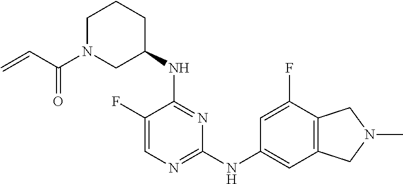 [SMILES: C=CC(=O)N1CCC[C@@H](Nc2nc(Nc3cc(F)c4c(c3)CN(C)C4)ncc2F)C1] | I-37 |
| 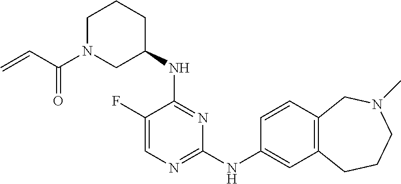 [SMILES: C=CC(=O)N1CCC[C@@H](Nc2nc(Nc3ccc4c(c3)CCCN(C)C4)ncc2F)C1] | I-38 |
| 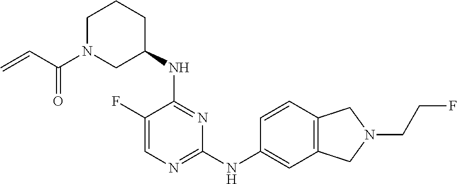 [SMILES: C=CC(=O)N1CCC[C@@H](Nc2nc(Nc3ccc4c(c3)CN(CCF)C4)ncc2F)C1] | I-39 |
| 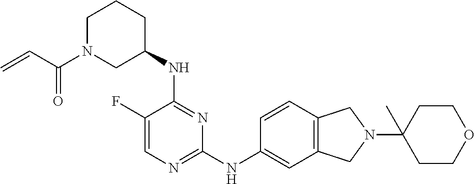 [SMILES: C=CC(=O)N1CCC[C@@H](Nc2nc(Nc3ccc4c(c3)CN(C3(C)CCOCC3)C4)ncc2F)C1] | I-40 |
| 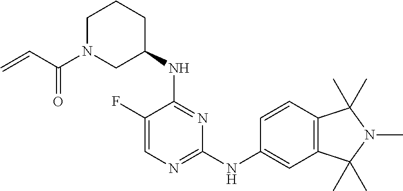 [SMILES: C=CC(=O)N1CCC[C@@H](Nc2nc(Nc3ccc4c(c3)C(C)(C)N(C)C4(C)C)ncc2F)C1] | I-41 |
| 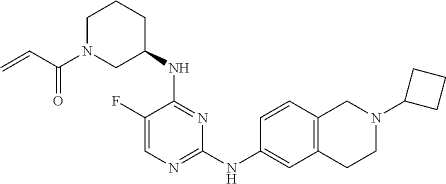 [SMILES: C=CC(=O)N1CCC[C@@H](Nc2nc(Nc3ccc4c(c3)CCN(C3CCC3)C4)ncc2F)C1] | I-42 |
| 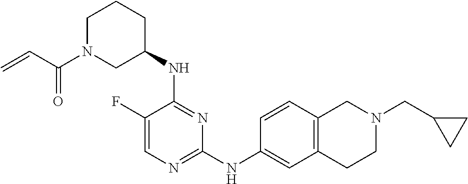 [SMILES: C=CC(=O)N1CCC[C@@H](Nc2nc(Nc3ccc4c(c3)CCN(CC3CC3)C4)ncc2F)C1] | I-43 |
| 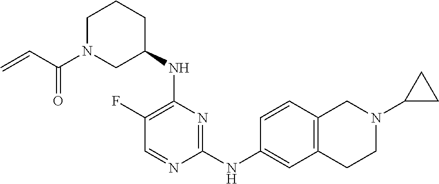 [SMILES: C=CC(=O)N1CCC[C@@H](Nc2nc(Nc3ccc4c(c3)CCN(C3CC3)C4)ncc2F)C1] | I-44 |
| 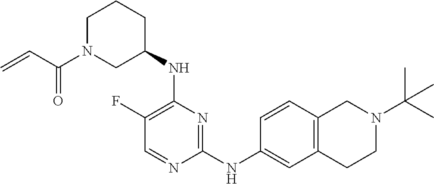 [SMILES: C=CC(=O)N1CCC[C@@H](Nc2nc(Nc3ccc4c(c3)CCN(C(C)(C)C)C4)ncc2F)C1] | I-45 |
| 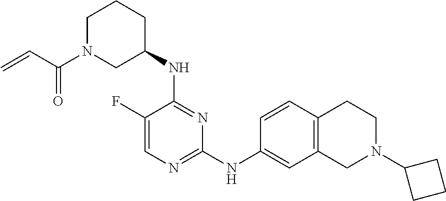 [SMILES: C=CC(=O)N1CCC[C@@H](Nc2nc(Nc3ccc4c(c3)CN(C3CCC3)CC4)ncc2F)C1] | I-46 |
| 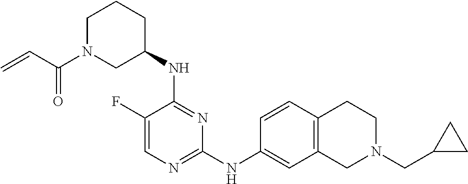 [SMILES: C=CC(=O)N1CCC[C@@H](Nc2nc(Nc3ccc4c(c3)CN(CC3CC3)CC4)ncc2F)C1] | I-47 |
| 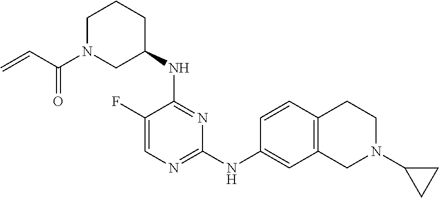 [SMILES: C=CC(=O)N1CCC[C@@H](Nc2nc(Nc3ccc4c(c3)CN(C3CC3)CC4)ncc2F)C1] | I-48 |
| 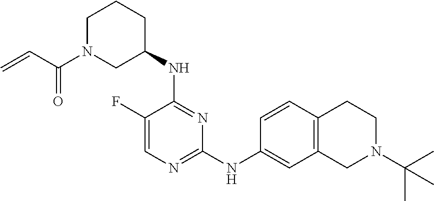 [SMILES: C=CC(=O)N1CCC[C@@H](Nc2nc(Nc3ccc4c(c3)CN(C(C)(C)C)CC4)ncc2F)C1] | I-49 |
| 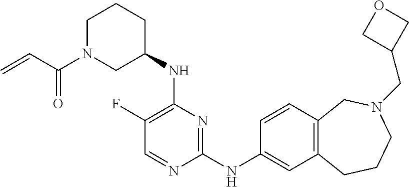 [SMILES: C=CC(=O)N1CCC[C@@H](Nc2nc(Nc3ccc4c(c3)CCCN(CC3COC3)C4)ncc2F)C1] | I-50 |
| 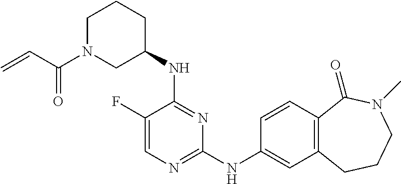 [SMILES: C=CC(=O)N1CCC[C@@H](Nc2nc(Nc3ccc4c(c3)CCCN(C)C4=O)ncc2F)C1] | I-51 |
| 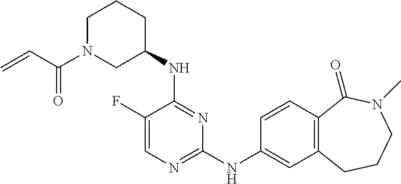 [SMILES: C=CC(=O)N1CCC[C@@H](Nc2nc(Nc3ccc4c(c3)CCCN(C)C4=O)ncc2F)C1] | I-52 |
| 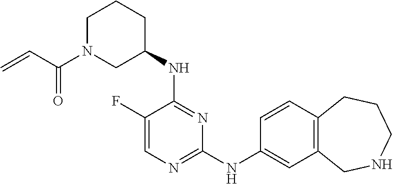 [SMILES: C=CC(=O)N1CCC[C@@H](Nc2nc(Nc3ccc4c(c3)CNCCC4)ncc2F)C1] | I-53 |
| 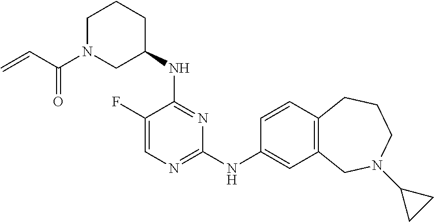 [SMILES: C=CC(=O)N1CCC[C@@H](Nc2nc(Nc3ccc4c(c3)CN(C3CC3)CCC4)ncc2F)C1] | I-54 |
| 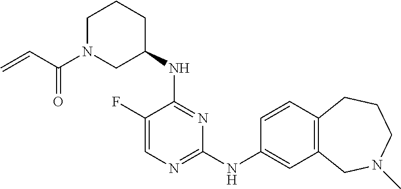 [SMILES: C=CC(=O)N1CCC[C@@H](Nc2nc(Nc3ccc4c(c3)CN(C)CCC4)ncc2F)C1] | I-55 |
| 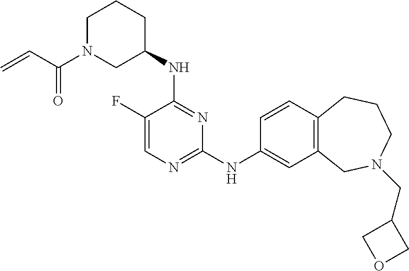 [SMILES: C=CC(=O)N1CCC[C@@H](Nc2nc(Nc3ccc4c(c3)CN(CC3COC3)CCC4)ncc2F)C1] | I-56 |
| 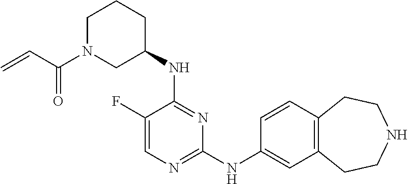 [SMILES: C=CC(=O)N1CCC[C@@H](Nc2nc(Nc3ccc4c(c3)CCNCC4)ncc2F)C1] | I-57 |
| 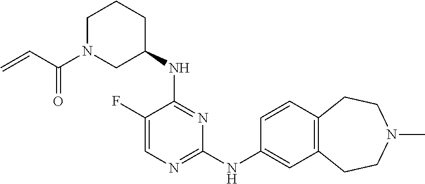 [SMILES: C=CC(=O)N1CCC[C@@H](Nc2nc(Nc3ccc4c(c3)CCN(C)CC4)ncc2F)C1] | I-58 |
| 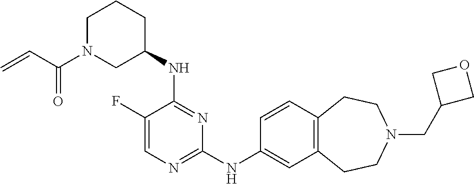 [SMILES: C=CC(=O)N1CCC[C@@H](Nc2nc(Nc3ccc4c(c3)CCN(CC3COC3)CC4)ncc2F)C1] | I-59 |
| 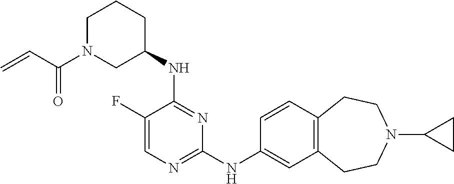 [SMILES: C=CC(=O)N1CCC[C@@H](Nc2nc(Nc3ccc4c(c3)CCN(C3CC3)CC4)ncc2F)C1] | I-60 |
| 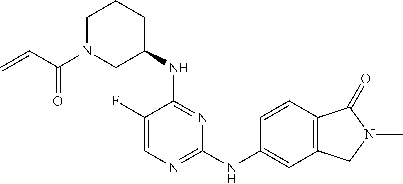 [SMILES: C=CC(=O)N1CCC[C@@H](Nc2nc(Nc3ccc4c(c3)CN(C)C4=O)ncc2F)C1] | I-61 |
| 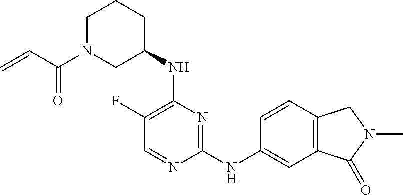 [SMILES: C=CC(=O)N1CCC[C@@H](Nc2nc(Nc3ccc4c(c3)C(=O)N(C)C4)ncc2F)C1] | I-62 |
| 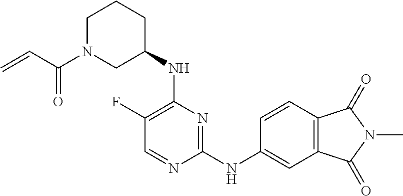 [SMILES: C=CC(=O)N1CCC[C@@H](Nc2nc(Nc3ccc4c(c3)C(=O)N(C)C4=O)ncc2F)C1] | I-63 |
| 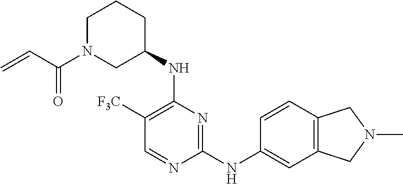 [SMILES: C=CC(=O)N1CCC[C@@H](Nc2nc(Nc3ccc4c(c3)CN(C)C4)ncc2C(F)(F)F)C1] | I-64 |
| 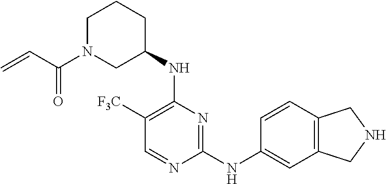 [SMILES: C=CC(=O)N1CCC[C@@H](Nc2nc(Nc3ccc4c(c3)CNC4)ncc2C(F)(F)F)C1] | I-65 |
| 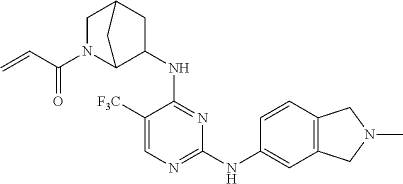 [SMILES: C=CC(=O)N1CC2CC(Nc3nc(Nc4ccc5c(c4)CN(C)C5)ncc3C(F)(F)F)C1C2] | I-66 |
| 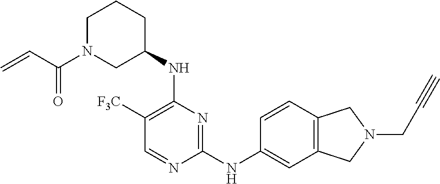 [SMILES: C#CCN1Cc2ccc(Nc3ncc(C(F)(F)F)c(N[C@@H]4CCCN(C(=O)C=C)C4)n3)cc2C1] | I-67 |

In certain embodiments, the present invention provides any compound depicted in Table 5, above, or a pharmaceutically acceptable salt thereof

In certain embodiments, the present invention provides a compound selected from:

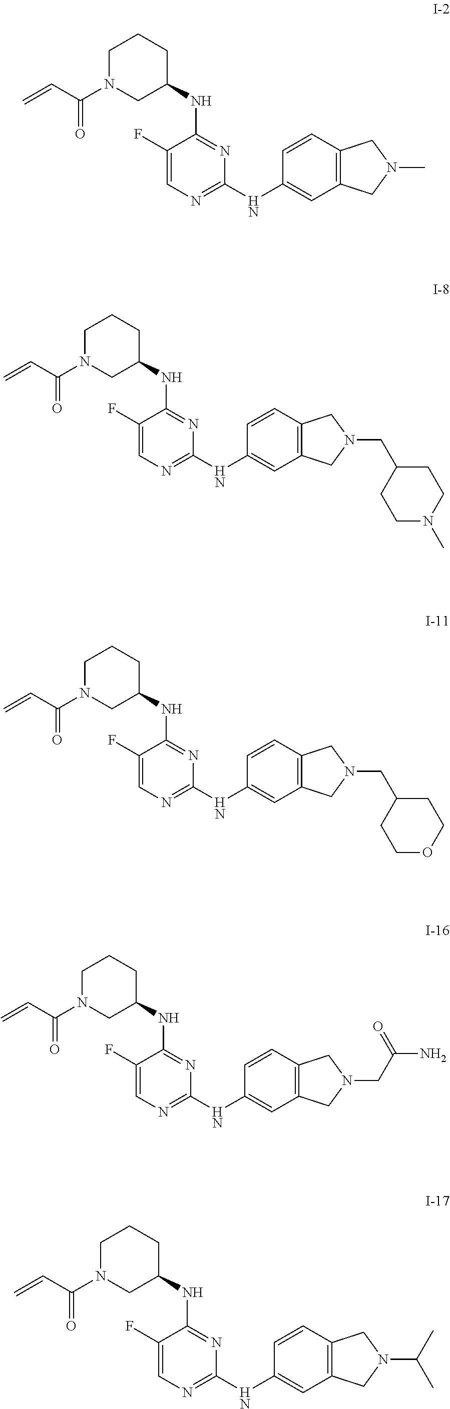

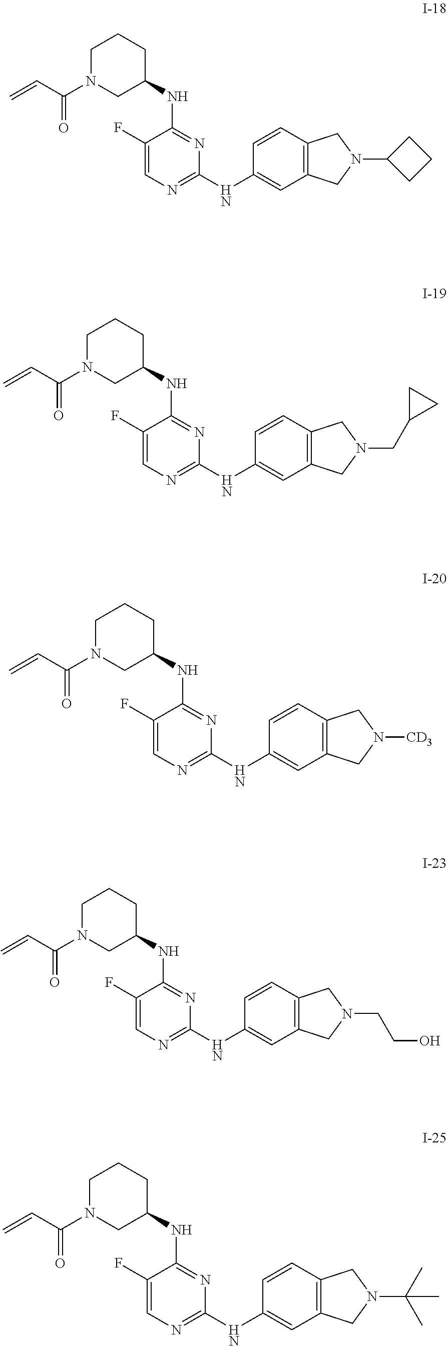 [SMILES: C=CC(=O)N1CCC[C@@H](Nc2nc(Nc3ccc4c(c3)CN(C3CCC3)C4)ncc2F)C1 ; C=CC(=O)N1CCC[C@@H](Nc2nc(Nc3ccc4c(c3)CN(CC3CC3)C4)ncc2F)C1 ; [2H]C([2H])([2H])N1Cc2ccc(Nc3ncc(F)c(N[C@@H]4CCCN(C(=O)C=C)C4)n3)cc2C1 ; C=CC(=O)N1CCC[C@@H](Nc2nc(Nc3ccc4c(c3)CN(CCO)C4)ncc2F)C1 ; C=CC(=O)N1CCC[C@@H](Nc2nc(Nc3ccc4c(c3)CN(C(C)(C)C)C4)ncc2F)C1]

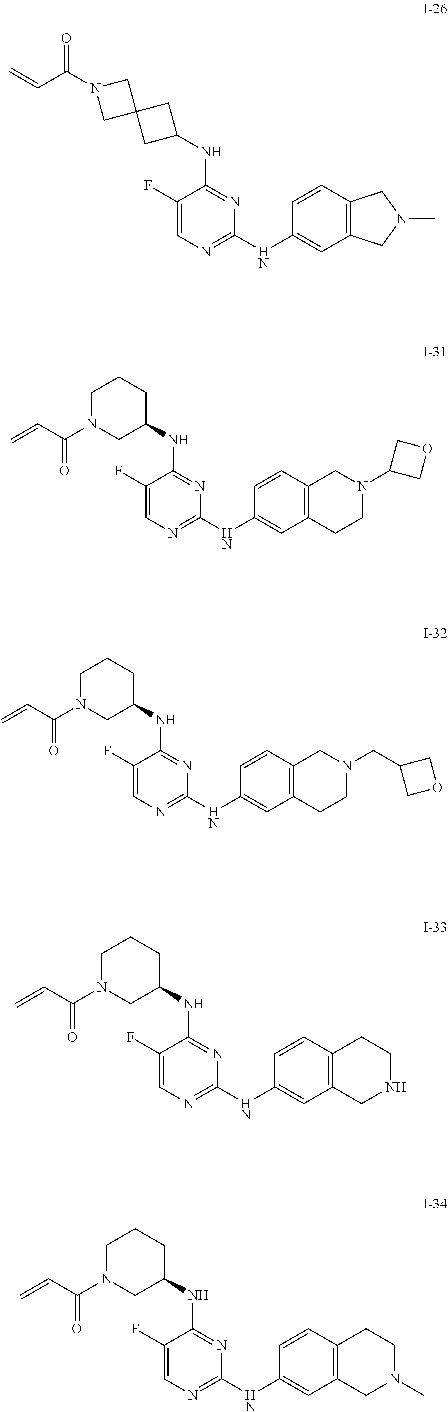

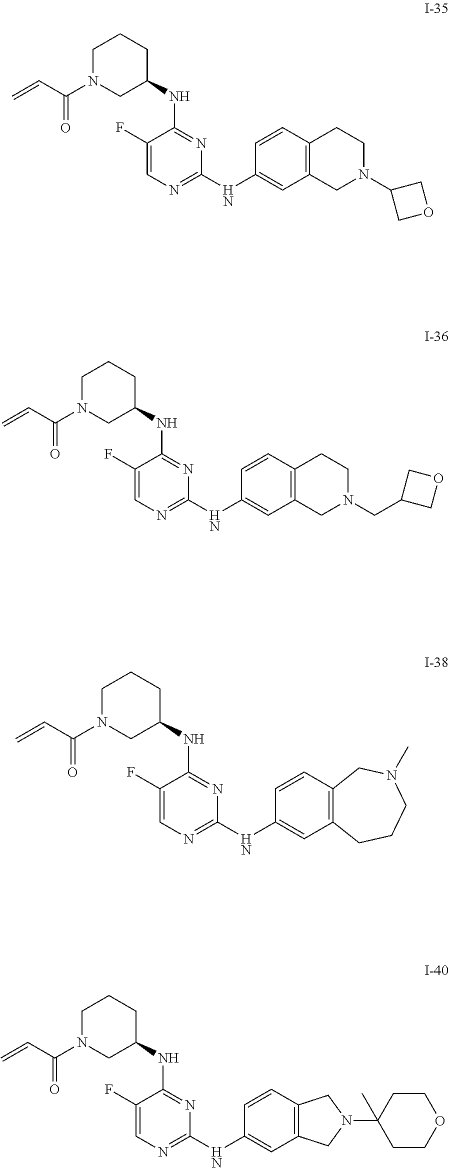

or a pharmaceutically acceptable salt thereof

In certain embodiments, the present invention provides a compound selected from:

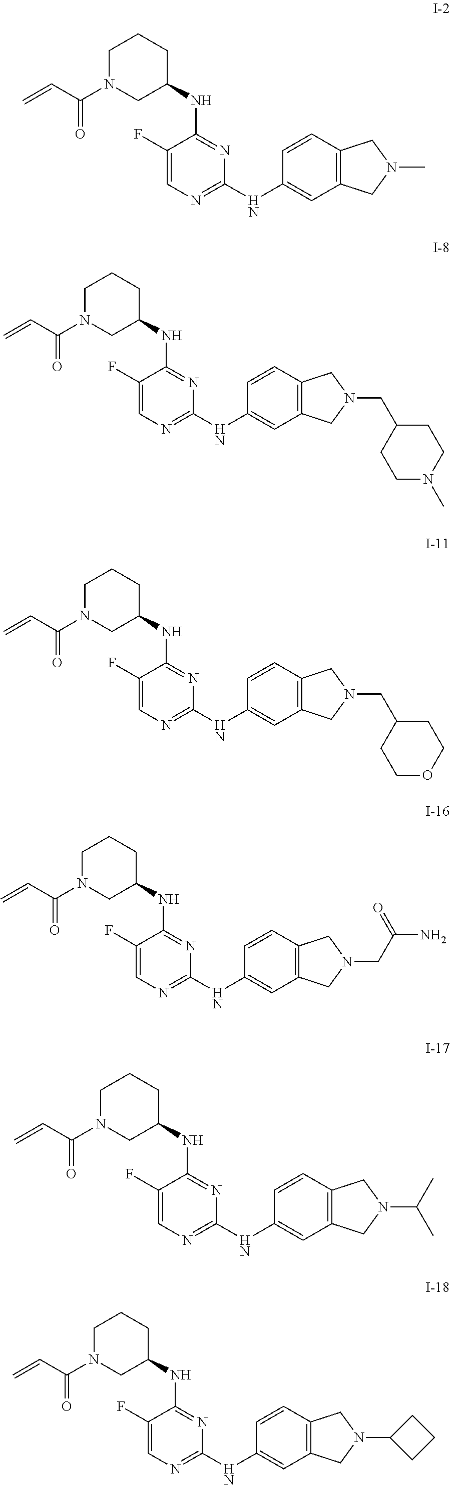

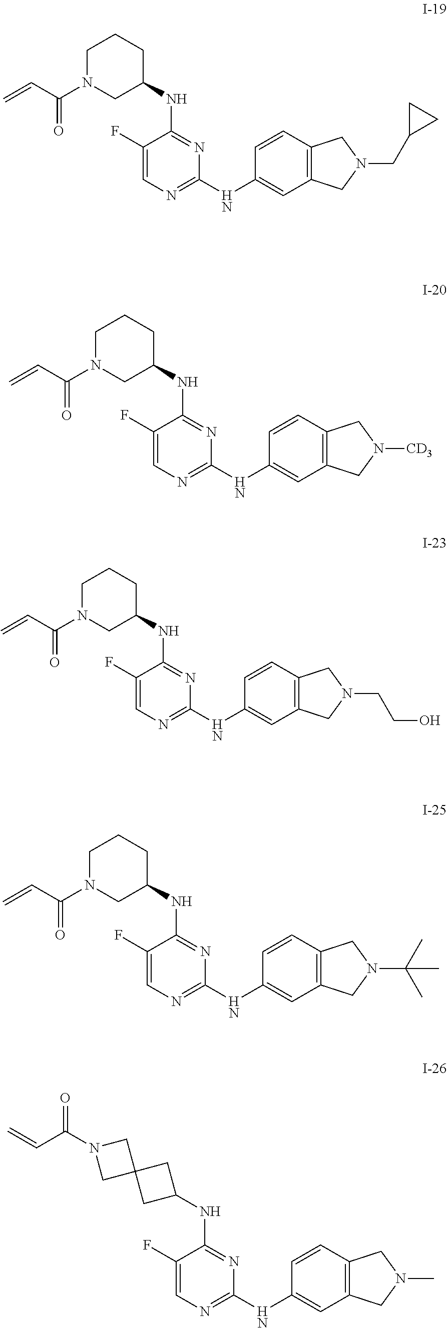

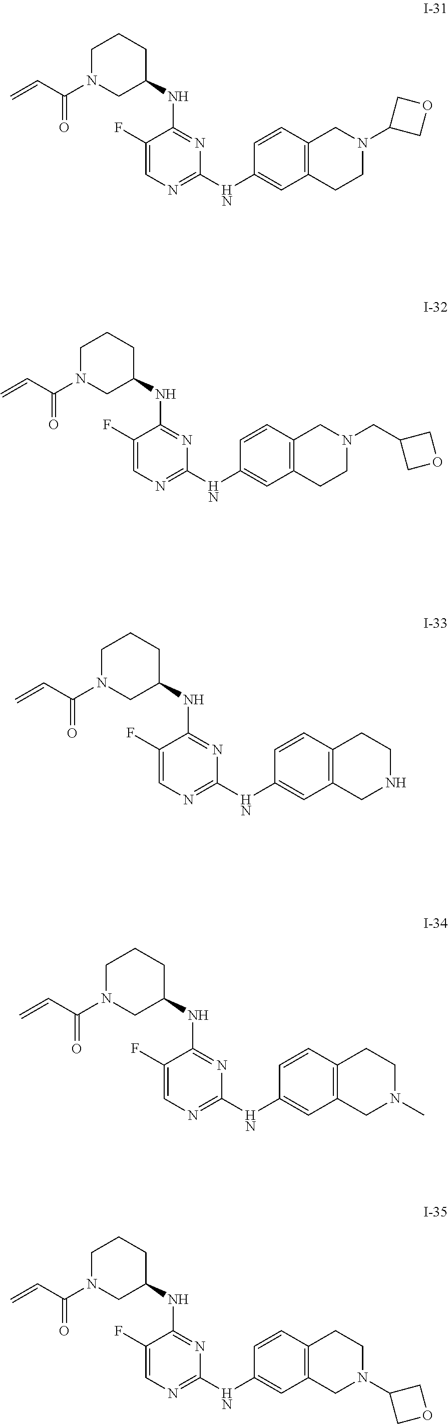

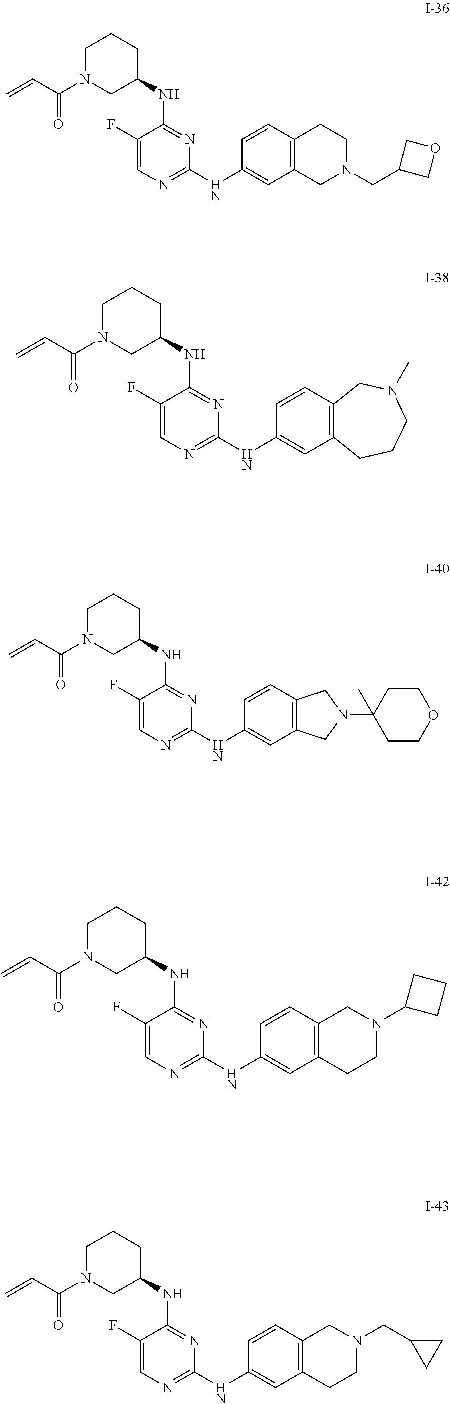

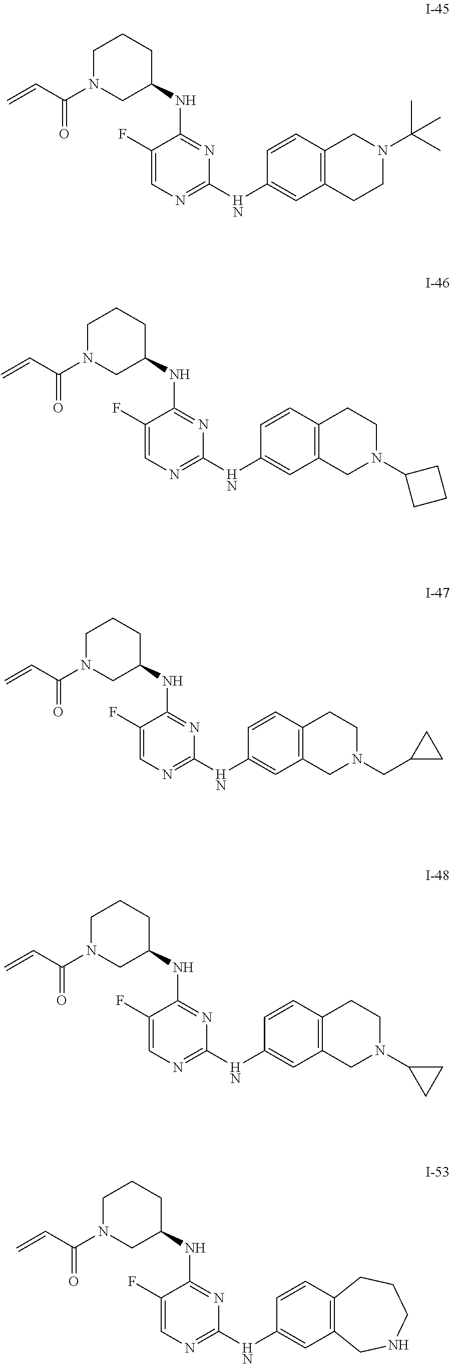

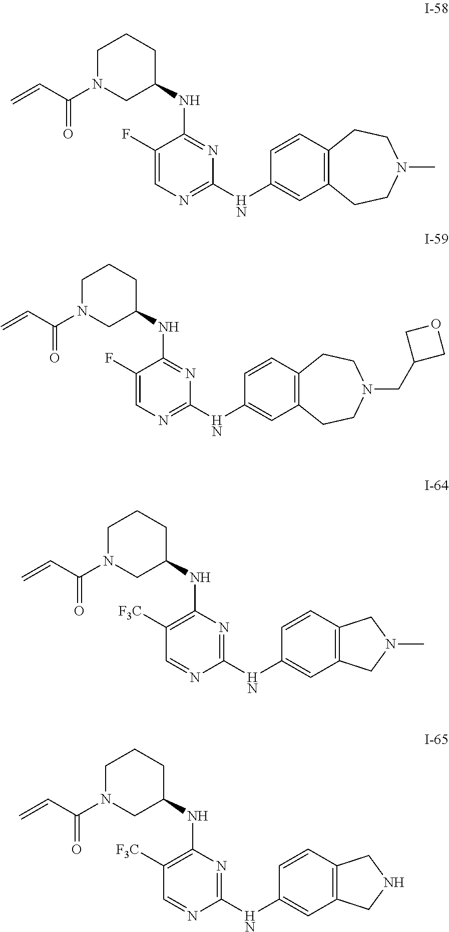

or a pharmaceutically acceptable salt thereof

The activity of a compound of formula I, for example, as an inhibitor of BTK, or a mutant thereof, may be assayed in vitro, in vivo or in a cell line. In vitro assays include assays that determine inhibition of either the phosphorylation activity and/or the subsequent functional consequences, or ATPase activity of activated BTK, or a mutant thereof. Alternate in vitro assays quantitate the ability of the inhibitor to bind to BTK. Inhibitor binding may be measured by radiolabeling the inhibitor prior to binding, isolating the inhibitor/BTK complex and determining the amount of radiolabel bound. Alternatively, inhibitor binding may be determined by running a competition experiment where new inhibitors are incubated with BTK-kinase bound to known radioligands. Detailed conditions for assaying a compound utilized in this invention as an inhibitor of BTK-kinase or a mutant thereof, are set forth in the Examples below.

### 4. Uses, Formulation and Administration

### Pharmaceutically Acceptable Compositions

Among other things, the present invention provides compositions comprising a compound of formula I, or a pharmaceutically acceptable salt or derivative thereof, and a pharmaceutically acceptable carrier, adjuvant, or vehicle. The amount of compound in compositions of this invention is such that is effective to measurably inhibit a protein kinase, particularly BTK, or a mutant thereof, in a biological sample or in a patient. In certain embodiments, a compound or composition of this invention is formulated for administration to a patient in need of such composition. The compounds and compositions, according to the methods of the present invention, may be administered using any amount and any route of administration effective for treating or lessening the severity of any disease or disorder described herein. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will vary from subject to subject, depending on a variety of factors, including the disorder being treated and the severity of the disorder; the activity of the specific compound employed; the specific composition employed and its route of administration; the species, age, body weight, sex and diet of the patient; the general condition of the subject; the time of administration; the rate of excretion of the specific compound employed; the duration of the treatment; drugs used in combination or coincidental with the specific compound employed, and the like.

Compositions of the present invention may be administered orally, parenterally, by inhalation or nasal spray, topically (e.g., as by powders, ointments, or drops), rectally, buccally, intravaginally, intraperitoneally, intracisternally or via an implanted reservoir, depending on the severity of the condition being treated. Preferably, the compositions are administered orally, intraperitoneally or intravenously. In certain embodiments, the compounds of the invention are administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

The term “parenteral” as used herein includes subcutaneous, intravenous, intramuscular, intra-articular, intra-synovial, intrasternal, intrathecal, intrahepatic, intralesional and intracranial injection or infusion techniques. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Pharmaceutically acceptable compositions of this invention may be orally administered in any orally acceptable dosage form including, but not limited to, capsules, tablets, aqueous suspensions or solutions. In such solid dosage forms the active compound may be admixed with at least one inert diluent such as sucrose, lactose or starch. Such dosage forms may also comprise, as is normal practice, additional substances other than inert diluents, e.g., lubricants and other tableting aids such a magnesium stearate and microcrystalline cellulose. When aqueous suspensions are required for oral use, the active ingredient is combined with emulsifying and suspending agents. If desired, certain sweetening, flavoring or coloring agents may also be added.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and/or i) lubricants such as talc, calcium stearate, magnesium stearate, solid polyethylene glycols, sodium lauryl sulfate, and mixtures thereof. In the case of capsules, tablets and pills, the dosage form may also comprise buffering agents. The active compounds can also be in micro-encapsulated form with one or more excipients as noted above.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings (i.e. buffering agents) and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Alternatively, pharmaceutically acceptable compositions of this invention may be administered in the form of suppositories for rectal or vaginal administration. These can be prepared by mixing the compounds of the present invention with suitable non-irritating excipients or carriers that are solid at room temperature but liquid at body (e.g. rectal or vaginal) temperature and therefore will melt in the rectum or vaginal cavity to release the active compound. Such materials include cocoa butter, a suppository wax (e.g., beeswax) and polyethylene glycols.

Pharmaceutically acceptable compositions of this invention may also be administered topically, especially when the target of treatment includes areas or organs readily accessible by topical application, including diseases of the eye, the skin, or the lower intestinal tract. Topical application for the lower intestinal tract can be effected in a rectal suppository formulation (see above) or in a suitable enema formulation.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulations, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration.

### Uses of Compounds and Pharmaceutically Acceptable Compositions

Compounds and compositions described herein are generally useful for the inhibition of protein kinase activity of one or more enzymes, and in particular, BTK.

Protein tyrosine kinases are a class of enzymes that catalyze the transfer of a phosphate group from ATP or GTP to a tyrosine residue located on a protein substrate. A variety of cellular processes are promoted by these signals, including proliferation, carbohydrate utilization, protein synthesis, angiogenesis, cell growth, and cell survival.

### Bruton's Tyrosine Kinase (BTK)

Bruton's tyrosine kinase (“BTK”), a member of TEC-kinases (e.g., TEC, BTK, ITK, RLK or BMX), is a key signaling enzyme expressed in all hematopoietic cell types except T lymphocytes and natural killer cells. BTK plays an essential role in the B-cell signaling pathway linking cell surface B-cell receptor (BCR) stimulation to downstream intracellular responses.

BTK is a key regulator of B-cell development, activation, signaling, and survival (Kurosaki, Curr Op Imm, 2000, 276-281; Schaeffer and Schwartzberg, Curr Op Imm 2000, 282-288). In addition, BTK plays a role in a number of other hematopoietic cell signaling pathways, e.g., Toll like receptor (TLR) and cytokine receptor-mediated TNF-α production in macrophages, IgE receptor (Fc_epsilon_RI) signaling in mast cells, inhibition of Fas/APO-1 apoptotic signaling in B-lineage lymphoid cells, and collagen-stimulated platelet aggregation. See, e.g., C. A. Jeffries, et al., (2003), Journal of Biological Chemistry 278:26258-26264; N. J. Horwood, et al., (2003), The Journal of Experimental Medicine 197: 1603-1611; Iwaki et al. (2005), Journal of Biological Chemistry 280(48):40261-40270; Vassilev et al. (1999), Journal of Biological Chemistry 274(3): 1646-1656, and Quek et al. (1998), Current Biology 8(20): 1137-1140.

Patients with mutations in BTK have a profound block in B cell development, resulting in the almost complete absence of mature B lymphocytes and plasma cells, severely reduced Ig levels and a profound inhibition of humoral response to recall antigens (reviewed in Vihinen et al (2000) Frontiers in Bioscience 5: d917-928). Mice deficient in BTK also have a reduced number of peripheral B cells and greatly decreased serum levels of IgM and IgG3. BTK deletion in mice has a profound effect on B cell proliferation induced by anti-IgM, and inhibits immune responses to thymus-independent type II antigens (Ellmeier et al, J Exp Med 192: 1611-1623 (2000)). BTK also plays a crucial role in mast cell activation through the high-affinity IgE receptor (Fc_epsilon_RI). BTK deficient murine mast cells have reduced degranulation and decreased production of proinflammatory cytokines following Fc_epsilon_RI cross-linking (Kawakami et al. Journal of Leukocyte Biology 65: 286-290).

BTK has been implicated in a number of disorders, including diabetes. BTK deficiency in non-obese diabetic mice dramatically protects against diabetes and improves B cell-related tolerance, as indicated by failure to generate autoantibodies to insulin (Kendall, et al. J. Immunol. 183: 6403-6412 (2009)). Modulation of BTK and improvement of B cell-related tolerance can therefore be used in treatment of diabetes, particularly T cell-mediated autoimmune diabetes, e.g. type I diabetes.

BTK is also implicated in various cancers. For example, BTK is upregulated in pancreatic cancer cells compared with normal pancreas cells, and BTK is also upregulated in chronic pancreatitis cells, which is sometimes a precursor to pancreatic cancer (Crnogorac-Jurcevic, et al. Gastroenterology 129: 1454-1463 (2005)). Due to the key role of BTK in regulation of B-cell development, activation, signaling, and survival, BTK is involved in many B cell-related cancers.

Provided compounds are inhibitors of BTK and are therefore useful for treating one or more disorders associated with activity of BTK. Thus, in some embodiments, the present invention provides a method for treating a BTK-mediated disorder comprising the step of administering to a patient in need thereof a compound of the present invention, or pharmaceutically acceptable composition thereof.

In some embodiments, the present invention provides a method of inhibiting a B cell receptor comprising contacting a cell with a compound of formula I. In some embodiments, the present invention provides a method of inhibiting BTK comprising contacting a cell with a compound of formula I.

As used herein, the term “BTK-mediated” disorders or conditions as used herein means any disease or other deleterious condition in which BTK, or a mutant thereof, is known to play a role. Accordingly, another embodiment of the present invention relates to treating or lessening the severity of one or more diseases in which BTK, or a mutant thereof, is known to play a role. Specifically, the present invention relates to a method of treating or lessening the severity of a disease or condition selected from a proliferative disorder or an autoimmune disorder, wherein said method comprises administering to a patient in need thereof a compound or composition according to the present invention. In some embodiments, the present invention provides a method of treating or lessening the severity of a B cell-mediated disorder, comprising administering to a patient in need thereof a compound of formula I. In some embodiments, the present invention provides a method of treating or lessening the severity of a BTK-mediated disorder, comprising administering to a patient in need thereof a compound of formula I.

In some embodiments, the present invention provides a method of inhibiting a T cell receptor comprising contacting a cell with a compound of formula I.

In some embodiments, the present invention provides a method of treating or lessening the severity of a T cell-mediated disorder, comprising administering to a patient in need thereof a compound of formula I.

In certain embodiments, the present invention provides methods of treating or lessening the severity of one or more of a proliferative disease or disorder (e.g., cancer), an autoimmune disease or disorder, an inflammatory disease or disorder or a fibrotic condition. In particular embodiments, the compounds and compositions, according to the present invention, are useful in treating or lessening the severity of an autoimmune disease or disorder and/or an inflammatory disease or disorder.

In some embodiments, the present invention provides a method for treating or lessening the severity of an autoimmune disease selected from inflammatory bowel disease (IBD), Crohn's disease, ulcerative colitis, arthritis (including inflammatory arthritis), lupus, rheumatoid arthritis, psoriatic arthritis, osteoarthritis, Still's disease, juvenile arthritis, myasthenia gravis, Hashimoto's thyroiditis, Ord's thyroiditis, Graves' disease, Sjogren's syndrome (including anterior scleritis), multiple sclerosis, Guillain-Barre syndrome, mixed connective tissue disease, neuromyelitis optica (Devic's disease), acute disseminated encephalomyelitis, Addison's disease, opsoclonus myoclonus syndrome, ankylosing spondylitis, non-radiographic spondyloarthritis, antiphospholipid antibody syndrome, aplastic anemia, autoimmune hepatitis, Goodpasture's syndrome, Henoch-Schoenlein purpura, membranous optic neuritis, scleroderma, morphea, primary biliary cirrhosis, sclerosing cholangitis, Reiter's syndrome, Takayasu's arteritis, temporal arteritis, warm autoimmune hemolytic anemia, antineutrophil cytoplasmic Ab (ANCA)-associated vasculitis (including Churg-Strauss syndrome, microscopic polyangiitis, mixed cryoglobulinemia and Wegener's granulomatosis), osteoporosis, palmoplantar pustulosis, Parkinson's disease, paroxysmal nocturnal hemoglobinuria, psoriasis, Parsonage-Turner's syndrome, perivasculitis, pemphigus follaceus, alopecia universalis, molluscum contagiosum, Morvan's syndrome, Muckle-Wells syndrome, multifocal motor neuropathy, multiple organ dysfunction syndrome, myeloperoxidase deficiency, nasal polyps, Nezelof syndrome, Behcet's disease, chronic fatigue, POEMS syndrome, Proteus syndrome, polymyalgia rheumatic, post-poliomyelitis syndrome, post-surgical adhesions, post-transplant lymphoproliferation, post-stroke inflammation, premature graft loss (transplant rejection), primary biliary cirrhosis, progesterone dermatitis, Parry-Romberg syndrome, progressive massive fibrosis, NASH, prostatitis, pruritis, Rasmussen's encephalitis, reperfusion syndrome, pyoderma gangrenosum, sarcoidosis, Schnitzler synderome, seborrheic dermatitis, scleritis, episcleritis, rheumatic fever, serum sickness, Sweet's syndrome, Susac's syndrome, synovitis, systemic inflammatory response syndrome, Wiskott-Aldrich syndrome, WHIM syndrome, undifferentiated connective tissue disease, sero-negative arthritis, Tolosa-Hunt syndrome, Wolf-Parkinson White syndrome, thrombosis, tendonitis, toxic epidermal necrolysis, tropical spastic paraparesis, Sprue, transient ischemic attack, dysautonomia, endometriosis, osteodystrophia deformans (Paget's disease of bone), interstitial cystitis, neuromyotonia, vulvodynia, systemic lupus erythematosus (SLE) (including lupus nephritis, neuropsychiatric and childhood-onset SLE), vasculitis, idiopathic thrombocytopenic purpura (ITP) (also known as immune thrombocytopenia or primary immune thrombocytopenia) (including chronic idiopathic thrombocytopenic purpura), cutaneous lupus erythematosus, cytokine storm, cystic fibrosis, dermatitis herpetiformis, diabetic neuropathies, diabetic macular edema, diabetic retinopathy, autoimmune thyroiditis, autoimmune Cushings syndrome, systemic sclerosis, diffuse cerebral sclerosis of Schilder, Lyme neuroborreliosis, DiGeorge syndrome, Viking disease, Dressler syndrome, sicca syndrome, Sjögren's syndrome, autoimmune hyperlipidemia, autoimmune pancreatitis, autoimmune orchitis, autoimmune oophoritis, autoimmune hypophysitis, autoimmune hypoparathyroidism, autoimmune inner ear disease, relapsing polychondritis, autoimmune lymphoproliferative syndrome, autoimmune metaplastic atrophic gastritis, autoimmune neutropenia, autoimmune peripheral neuropathy, autosomal recessive hyper IgM syndrome, autosplenectomy, endocarditis, Fanconi's anemia, Felty syndrome, familial Mediterranean fever, fibrodyspalsis ossificans progressive, progeria, Friedreich's ataxia, clostria difficile gastroenteritis, focal segmental glomerulosclerosis, Evan's syndrome, epidermolysis bullosa acquisita, eosinophilic pneumonia, eosinophilic gastroenteritis, eosinophilic fasciitis, eosinophilic esophagitis, enthesitis related arthritis, enterohepatitis, endometrosis, Duchenne muscular dystrophy, giant cell arteritis, glaucoma, gout, Goodpasture syndrome, Guillian Barre syndrome, heavy chain disease, light chain disease, Heerfordt's syndrome, hepatic fibrosis, hepatitis A, hepatitis B, hepatitis C, Hepatitis D, hereditary angioedema, histiocytosis, HIV associated arteritis, Hughes-Stovin syndrome, hyper IgE syndrome, hypercholesterolemia, hyperhydrosis, hydradenitis suppurativa, autoimmune gastritis, pernicious anemia, celiac disease, membranous glomerulonephropathy, pemphigus vulgaris, bullous pemphigoid, encephalitis, myocarditis, nephritis, osteomyelitis, myositis, hepatitis, gastritis, dermatitis, pancreatitis, cholecystitus, irritable bowel syndrome, Barrett's esophagus, beta thalassemia, Bickerstaff's encephalitis, Blau syndrome, blepharitis, blepharoconjunctivitis, uveitis, retinitis, cataracts, Buerger's disease, bronchiolitis obliterans, bronchiolitis, C3 nephropathy, hypogammaglobulinemia, hypersenstitivity angiitis, Waldenstrom's macroglobulinemia, idiopathic CD4 lymphopenia, Sjogren's disease, autoimmune polyglandular disease (also known as autoimmune polyglandular syndrome), autoimmune alopecia, glomerulonephritis, IgA nephropathy, dermatomyositis (including juvenile dermatomyositis), autoimmune hemolytic and thrombocytopenic states, atherosclerosis, Parkinson's disease, Alzheimer's disease, diabetes (e.g., type I diabetes), Waldenstrom macroglobulinemia, degenerative joint disease, chronic obstructive pulmonary disease, chronic inflammatory demyelinating polyneuropathy, central serous retinopathy, cerebral vasospasm, Chediak Higashi syndrome, chemoprotection, chondrocalcinosis, vitiligo, celiac disease, gluten enteropathy, autoimmune hypopituitarism, autoimmune uveitis, sympathetic ophthalmia, Lambert-Eaton Syndrome, Berger's disease (IgA nephropathy), IgM nephropathy, Immune mediated nephropathy, polyarteritis nodosa, chronic recurrent multifocal osteomyelitis, Schmidt's syndrome, chronic kidney disease, chronic allograft nephropathy, chronic fatigue syndrome, fibromyalgia, granulomatous disease, and antiphospholipid syndrome.

In some embodiments, the autoimmune disease is rheumatoid arthritis. Patients with rheumatoid arthritis can be classified into distinct subsets, including lymphoid, myeloid and fibroid subsets. Dennis et al., “Synovial phenotypes in rheumatoid arthritis correlate with response to biologic therapeutics,” Arthritis Research & Therapy 2014, 16:R90, 1-18; Setiadi, et. al, “Synovial Subset-Derived Baseline Serum Biomarkers Segregate Rheumatoid Arthritis Patients into Subgroups with Distinct Serum Protein and Clinical Characteristics,” Abstract Number 1307, 2013 ACR/ARHP Annual Meeting. In some embodiments, the present invention provides a method of treating one or more of the lymphoid, myeloid and fibroid subsets of rheumatoid arthritis, comprising administering to a patient in one or more subsets a compound of formula I. Such subsets are classified by the presence of certain biomarkers which are detailed in Dennis et al., “Synovial phenotypes in rheumatoid arthritis correlate with response to biologic therapeutics,” Arthritis Research & Therapy 2014, 16:R90, 1-18; Setiadi, et. al, “Synovial Subset-Derived Baseline Serum Biomarkers Segregate Rheumatoid Arthritis Patients into Subgroups with Distinct Serum Protein and Clinical Characteristics,” Abstract Number 1307, 2013 ACR/ARHP Annual Meeting, each of which is hereby incorporated by reference.

In some embodiments, the present invention provides a method for treating or lessening the severity of rheumatoid arthritis in a patient, wherein the patient has one or more biomarkers for the lymphoid subset of rheumatoid arthritis, comprising administering to the patient a compound of formula I. Such biomarkers for the lymphoid subset of rheumatoid arthritis include, for example, high CXCL13 and low soluble ICAM1 expression levels. In some embodiments, the present invention provides a method for treating or lessening the severity of rheumatoid arthritis in a patient, wherein the patient has one or more biomarkers for the myeloid subset of rheumatoid arthritis, comprising administering to the patient a compound of formula I. In some embodiments, the present invention provides a method for treating or lessening the severity of rheumatoid arthritis in a patient, wherein the patient has one or more biomarkers for the fibroid subset of rheumatoid arthritis, comprising administering to the patient a compound of formula I. In some embodiments, the present invention provides a method for treating or lessening the severity of at least one subset of rheumatoid arthritis, comprising administering to the patient a compound of formula I. In some embodiments, the subset of rheumatoid arthritis is lymphoid. In some embodiments, the subset of rheumatoid arthritis is myeloid. In some embodiments, the subset of rheumatoid arthritis is fibroid.

In some embodiments, the present invention provides a method for treating or lessening the severity of a disease or disorder selected from rejection of transplanted organs or tissues, Acquired Immunodeficiency Syndrome (AIDS, also known as HIV), pelvic inflammatory disease, urethritis, skin sunburn, acne, sinusitis, pneumonitis, meningitis, enteritis, gingivitis, appendicitis, cicatricial pemphagoid, Cogan's syndrome, CREST syndrome, condylomata accuminata, common variable immunodeficiency, complex regiona pain syndrome, agammaglobulinemia, allergy, tissue graft rejection, hyperacute rejection of transplanted organs, chronic obstructive pulmonary disease (COPD), septic shock, atopic dermatitis, mycosis fungoides, acute inflammatory responses (such as acute respiratory distress syndrome and ischemia/reperfusion injury).

In some embodiments, the present invention provides a method for treating or lessening the severity of one or more diseases and conditions associated with BTK, wherein the disease or condition is selected from heteroimmune conditions or diseases, which include, but are not limited to graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, contact dermatitis and atopic dermatitis.

In some embodiments, the present invention provides a method for treating or lessening the severity of an inflammatory disease (i.e., diseases with an inflammatory component) selected from asthma, Dego's disease, inflammatory bowel disease (including Crohn's disease and ulcerative colitis), autoimmune enteropathy, appendicitis, blepharitis, bronchiolitis, bronchitis, bursitis, cerebral malaria, cervicitis, cholangitis, cholecystitis, colitis, conjunctivitis, cystitis, dacryoadenitis, dermatitis, dermatomyositis, encephalitis, acute hemorrhagic leukoencephalitis, acute radiation syndrome, age-related macular degeneration, endocarditis, endometritis, enteritis, enterocolitis, epicondylitis, epididymitis, fasciitis, fibrositis, gastritis, gastroenteritis, hepatitis, hidradenitis suppurativa, human immunodeficiency virus (HIV)/acquired immunodeficiency syndrome (AIDS), laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sepsis, sinusitis, stomatitis, allergic rhinoconjunctivitis, alopecia areata, alopecia totalis, synovitis, tendonitis, tonsillitis, uveitis, vaginitis, vasculitis, or vulvitis, dermatitis, contact dermatitis, eczema, atopic syndrome, urticaria, rosacea, scarring, atopic dermatitis, allergy, chronic graft rejection, Henoch-Schonlein purpura, immunoglobulin A nephropathy, interstitial lung disease, interstitial pulmonary fibrosis, alpha 1 antitrypsin deficiency, amyloidosis, amyotrophic lateral sclerosis (ALS), polymyositis, ulcerative colitis and cryoglobulinemia, atherosclerosis, myocardial infarction and thrombosis.

In some embodiments, the present invention provides a method for treating or lessening the severity of one or more diseases and conditions associated with BTK, wherein the disease or condition is selected from a cancer.

In one embodiment, the cancer is a B-cell proliferative disorder, e.g., diffuse large B cell lymphoma, follicular lymphoma, chronic lymphocytic lymphoma, chronic lymphocytic leukemia, small lymphocytic lymphoma, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis. In some embodiments, the cancer is T-cell proliferative disorder, e.g., extranodal T cell lymphoma, cutaneous T cell lymphomas (inclduing Sezary syndrome and Mycosis fungoides, also known as Alibert-Bazen syndrome), anaplastic large cell lymphoma, angioimmunoblastic T cell lymphoma, peripheral T cell lymphoma, peripheral T cell lymphoma not otherwise specified (PTCL-NOS), adult T cell leukemia/lymphoma (ATLL), blastic NK-cell lymphoma, enteropathy-type T cell lymphoma, hematosplenic gamma-delta T-cell lymphoma, lymphoblastic lymphoma, nasal NK/T cell lymphomas, or treatment-related T cell lymphomas. In some embodiments, the cancer is breast cancer, prostate cancer, or cancer of the mast cells (e.g., mastocytoma, mast cell leukemia, mast cell sarcoma, systemic mastocytosis). In one embodiment, the cancer is bone cancer. In another embodiment, the cancer is of other primary origin and metastasizes to the bone. In certain embodiments, the cancer is colorectal cancer or pancreatic cancer.

In some embodiments, the present invention provides a method for treating or lessening the severity of a proliferative disease selected from B-cell proliferative disorder, e.g., diffuse large B cell lymphoma (DLBCL), follicular lymphoma, chronic lymphocytic lymphoma, chronic lymphocytic leukemia, small lymphocytic leukemia, small lymphocytic lymphoma, B-cell prolymphocytic leukemia, lymphoplasmacytic lymphoma/Waldenstrom macroglobulinemia, splenic marginal zone lymphoma, plasma cell myeloma, plasmacytoma, extranodal marginal zone B cell lymphoma, extranodal marginal zone B cell lymphoma of mucosa-associated lymphoid tissue (MALT), nodal marginal zone B cell lymphoma, mantle cell lymphoma, mediastinal (thymic) large B cell lymphoma, intravascular large B cell lymphoma, primary effusion lymphoma, burkitt lymphoma/leukemia, hairy cell leukemia, heavy chain diseases (e.g., alpha heavy chain disease, gamma heavy chain disease, mu heavy chain disease), primary cutaneous B cell lymphoma, ALK+ large cell lymphoma, Castleman's disease, lymphomatoid granulomatosis, breast cancer, prostate cancer, cancer of the mast cells (e.g., mastocytoma, mast cell leukemia, mast cell sarcoma, systemic mastocytosis), multiple myeloma, colorectal cancer, pancreatic cancer, B-cell prolymphocytic leukemia, solitary plasmacytoma of bone, extraosseous plasmacytoma, primary cutaneous follicle center lymphoma, primary mediastinal (thymic) large B-cell lymphoma, intravascular large B-cell lymphoma, primary cutaneous DLBCL and plasmablastic lymphoma.

In some embodiments, the present invention provides a method for treating or lessening the severity of a proliferative disease selected from T-cell proliferative disorder, e.g., extranodal T cell lymphoma, cutaneous T cell lymphomas (inclduing Sezary syndrome and Mycosis fungoides, also known as Alibert-Bazen syndrome), anaplastic large cell lymphoma, angioimmunoblastic T cell lymphoma, peripheral T cell lymphoma, peripheral T cell lymphoma not otherwise specified (PTCL-NOS), adult T cell leukemia/lymphoma (ATLL), blastic NK-cell lymphoma, enteropathy-type T cell lymphoma, hematosplenic gamma-delta T-cell lymphoma, lymphoblastic lymphoma, nasal NK/T cell lymphomas, or treatment-related T cell lymphomas.

In some embodiments, the present invention provides a method for treating or lessening the severity of one or more diseases or conditions associated with BTK including diseases of the bone and joints including, without limitation, rheumatoid arthritis, seronegative spondyloarthropathies (including ankylosing spondylitis, psoriatic arthritis and Reiter's disease), bone resorption disorders (including Paget's disease of bone, bone changes secondary to cancer, such as occur in myeloma and metastases from breast cancer, etc.), Behcet's disease, Sjogren's syndrome, systemic sclerosis, osteoporosis, bone cancer, and bone metastasis.

In some embodiments, the present invention provides a method for treating or lessening the severity of one or more diseases and conditions associated with BTK, wherein the disease or condition is selected from a thromboembolic disorder, e.g., myocardial infarct, angina pectoris (including unstable angina), reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, or deep venous thrombosis.

In some embodiments, the present invention provides a method for treating or lessening the severity of one or more diseases and conditions associated with BTK, including infectious and noninfectious inflammatory events and autoimmune and other inflammatory diseases. These autoimmune and inflammatory diseases, disorders, and syndromes include inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, type I diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, immunodeficiency, centromeric instability, and facial anomalies (ICF syndrome), atopic dermatitis, degenerative joint disease, vitiligo, inclusion body myositis, inflammatory pain, IPEX syndrome, interstitial cystitis, irritable bowel syndrome, Isaac's syndrome, Kawasaki disease, keloids, Kimura disease, Lambert-Eaton syndrome, myasthenia gravis, leukocyte adhesion deficiency, lichen planus, lichen sclerosis, linear IgA disease, mastocytosis, mediastinal fibrosis, Meniere's disease, meningitis, mesangiocapillary glomerulonephritis, microscopic colitis, Miller-Fisher syndrome, pollinosis (also known as hay fever), autoimmune achlorhydria, idiopathic adrenal atrophy, Raynauds, thyrotoxicosis, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease. In certain embodiments, the diabetes is type I diabetes.

In some embodiments, the present invention provides a method for treating or lessening the severity of one or more diseases and conditions associated with BTK, selected from rheumatoid arthritis, multiple sclerosis, B-cell chronic lymphocytic leukemia, acute lymphocytic leukemia, hairy cell leukemia, non-Hodgkin's lymphoma, Hodgkin's lymphoma, multiple myeloma, bone cancer, bone metastasis, osteoporosis, irritable bowel syndrome, Crohn's disease, lupus and renal transplant.

In some embodiments, the present invention provides a method for treating or lessening the severity of a skin disorder selected from bullous skin diseases (e.g., pemphigus vulgaris including childhood/juvenile pemphigus vulgaris, pemphigus foliaceus, paraneoplastic pemphigus, bullous pemphigoid, mucous membrane pemphigoid and epidermolysis bullosa aquisita).

In some embodiments, the present invention provides a method for treating or lessening the severity of a platelet disorder, for example, abberant platelet aggregation. See, for example, Liu et al., Blood 2006, 108: 2596-2603, incorporated by reference in its entirety.

In some embodiments, the present invention provides a method for treating or lessening the severity of a fibrotic condition.

In certain embodiments, the present invention provides a method for the treatment of a disease or disorder selected from an accumulation of excess extracellular matrix; systemic sclerosis/scleroderma, lupus nephritis, connective tissue disease, wound healing, surgical scarring, spinal cord injury, CNS scarring, acute lung injury, pulmonary fibrosis (such as idiopathic pulmonary fibrosis and cystic fibrosis), chronic obstructive pulmonary disease, adult respiratory distress syndrome, acute lung injury, drug-induced lung injury, glomerulonephritis, chronic kidney disease (including diabetic nephropathy), hypertension-induced nephropathy, alimentary track or gastrointestinal fibrosis, renal fibrosis, hepatic or biliary fibrosis, liver fibrosis (nonalcoholic steatohepatitis, Hepatitis C/hepatocellular carcinoma, etc.), cirrhosis (such as primary biliary cirrhosis and cirrhosis due to fatty liver disease (alcoholic and nonalcoholic steatosis)), radiation-induced fibrosis (such as head and neck, gastrointestinal and pulmonary), primary sclerosing cholangitis, restenosis, cardiac fibrosis (such as endomyocardial fibrosis and atrial fibrosis), opthalmic scarring, fibrosclerosis, fibrotic cancers, fibroids, fibroma, fibroadenomas, fibrosarcomas, transplant arteriopathy, keloid, mediastinal fibrosis, myelofibrosis, retroperitoneal fibrosis, progressive massive fibrosis, nephrogenic systemic fibrosis, Crohn's disease, arthrofibrosis, adhesive capsulitis and other conditions such as Dupuytren's Disease, colorectal cancer, tumor metastasis, Myc-mediated solid tumors (such as colon cancer, prostate cancer, myeloma, lymphoma), metabolic disease (such as Type 2 diabetes), metabolic myopathies (such as glycogen and lipid storage disorders), cachexia, hypertension, ankylosing spondylitis, demyelination in multiple sclerosis, cerebral angiopathy and Alzheimer's disease, wherein said method comprises administering to a patient in need thereof a compound of the present invention, or pharmaceutically acceptable composition thereof.

In some embodiments, the disease or disorder being treated is a fibrotic condition selected from systemic sclerosis/scleroderma, lupus nephritis, connective tissue disease, wound healing, surgical scarring, spinal cord injury, CNS scarring, acute lung injury, pulmonary fibrosis, chronic obstructive pulmonary disease, adult respiratory distress syndrome, acute lung injury, drug-induced lung injury, glomerulonephritis, chronic kidney disease, hypertension-induced nephropathy, alimentary track or gastrointestinal fibrosis, renal fibrosis, hepatic or biliary fibrosis, liver fibrosis, cirrhosis, radiation-induced fibrosis, primary sclerosing cholangitis, restenosis, cardiac fibrosis, opthalmic scarring, fibrosclerosis, fibrotic cancers, fibroids, fibroma, fibroadenomas, fibrosarcomas, transplant arteriopathy and keloid.

In some embodiments, the disease or disorder being treated is selected from lupus nephritis, connective tissue disease, wound healing, surgical scarring, spinal cord injury, CNS scarring, acute lung injury, pulmonary fibrosis, drug-induced lung injury, chronic kidney disease, hypertension-induced nephropathy, alimentary track or gastrointestinal fibrosis, renal fibrosis, hepatic or biliary fibrosis, liver fibrosis, cirrhosis, radiation-induced fibrosis, primary sclerosing cholangitis, cardiac fibrosis, ophthalmic scarring, fibrosclerosis, fibrotic cancers, fibroids, fibroma, fibroadenomas, fibrosarcomas, transplant arteriopathy, keloid, mediastinal fibrosis, myelofibrosis, retroperitoneal fibrosis, progressive massive fibrosis, nephrogenic systemic fibrosis, arthrofibrosis, adhesive capsulitis, Dupuytren's Disease, tumor metastasis, Myc-mediated solid tumors, metabolic myopathies, cachexia, hypertension, demyelination in multiple sclerosis, and cerebral angiopathy.

In some embodiments, the disease or disorder being treated is selected from lupus nephritis, connective tissue disease, wound healing, surgical scarring, spinal cord injury, CNS scarring, acute lung injury, pulmonary fibrosis, drug-induced lung injury, chronic kidney disease, hypertension-induced nephropathy, alimentary track or gastrointestinal fibrosis, renal fibrosis, hepatic or biliary fibrosis, liver fibrosis, radiation-induced fibrosis, cardiac fibrosis, ophthalmic scarring, fibrosclerosis, fibrotic cancers, fibroids, fibroma, fibroadenomas, fibrosarcomas, transplant arteriopathy, keloid, mediastinal fibrosis, myelofibrosis, retroperitoneal fibrosis, progressive massive fibrosis, nephrogenic systemic fibrosis, arthrofibrosis, adhesive capsulitis, Dupuytren's Disease, Myc-mediated solid tumors, metabolic myopathies, cachexia, hypertension, demyelination in multiple sclerosis, and cerebral angiopathy.

In some embodiments, the fibrotic condition is pulmonary fibrosis. In certain embodiments, the pulmonary fibrosis is selected from idiopathic pulmonary fibrosis and cystic fibrosis. Idiopathic pulmonary fibrosis (IPF), (also called cryptogenic fibrosing alveolitis) is a fibrotic condition seen most commonly in patients between 40 and 60 years of age. Patients with IPF typically present with progressive shortness of breath and a dry cough. Pulmonary function tests show a restrictive pattern with reduced lung volumes and impairment in gas exchange. Idiopathic pulmonary fibrosis has a poor prognosis, with a mean survival of 4 years from the onset of symptoms.

Pathologically, the large majority of patients with IPF show typical histological findings of usual interstitial pneumonia and/or desquamative interstitial pneumonia. The earliest histological abnormality in IPF is alveolitis with increased cellularity of the alveolar walls. This inflammatory process can lead to progressive fibrosis. Alveolar wall inflammation and intra-alveolar macrophages in IPF indicate disease activity and are potentially reversible. Fibrosis and honeycombing are irreversible.

In some embodiments, chronic kidney disease is diabetic nephropathy.

In some embodiments, liver fibrosis is selected from nonalcoholic steatohepatitis, Hepatitis C/hepatocellular carcinoma.

In some embodiments, cirrhosis is selected from primary biliary cirrhosis and cirrhosis due to fatty liver disease (alcoholic and nonalcoholic steatosis).

In some embodiments, radiation-induced fibrosis is selected from head and neck, gastrointestinal and pulmonary fibrosis.

In some embodiments, cardiac fibrosis is selected from endomyocardial fibrosis and atrial fibrosis.

In some embodiments, Myc-mediated solid tumors selected from colon cancer, prostate cancer, myeloma, lymphoma.

In some embodiments, the metabolic disease is Type 2 diabetes.

In some embodiments, the metabolic myopathy is selected from glycogen and lipid storage disorders.

### EXEMPLIFICATION

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### GENERAL METHODS

### Method A

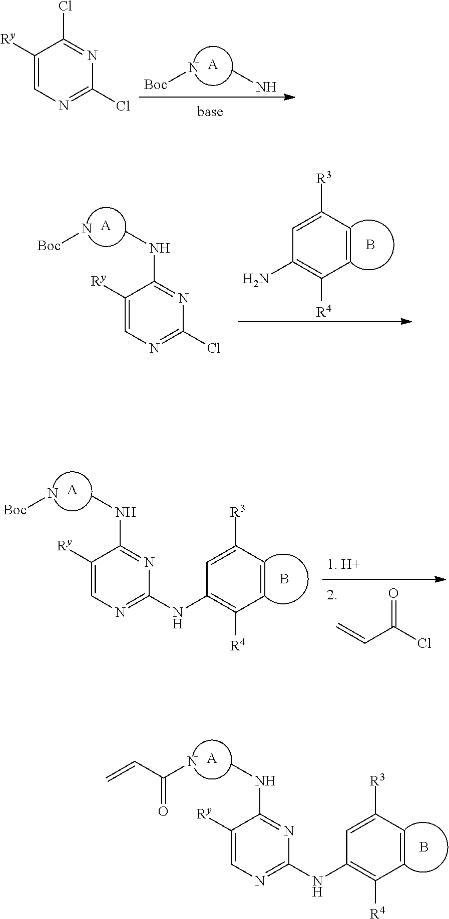

### Method B

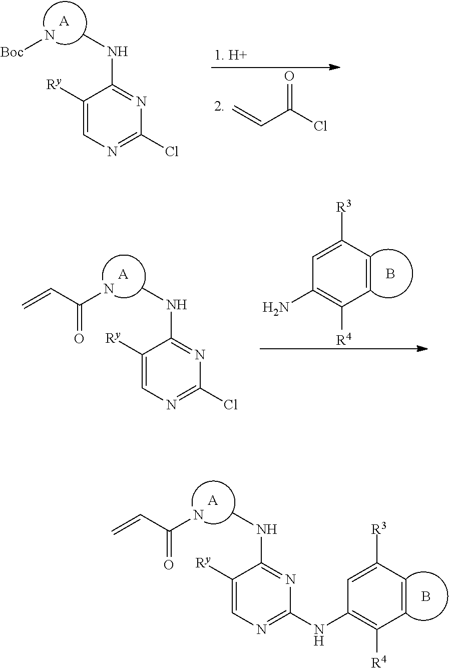

### Preparation of Intermediate A: (R)-tert-Butyl 3-((2-chloro-5-fluoropyrimidin-4-yl)amino)piperidine-1-carboxylate

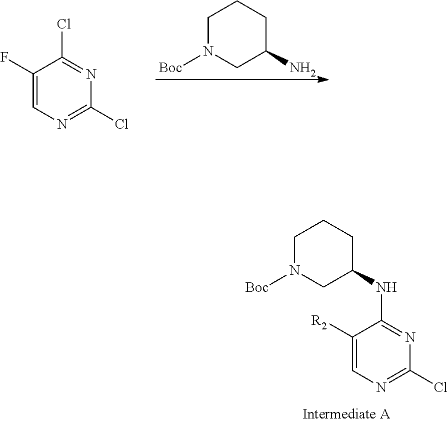

A 4-necked 3 L round bottom flask was charged with NMP (0.85 L). 2,4-dichloro-5-fluoropyrimidine (400 g, 2.4 mol) and (R)-tert-butyl 3-aminopiperidine-1-carboxylate (476 g, 1.0 eq) were added at room temperature. After stirring at room temperature for 10 min, the reaction mixture was cooled to 5-10° C. and DIPEA (483 g, 2.15 eq) was added dropwise over 30 min. The resulting mixture was allowed to stir at room temperature for an additional 30 min. TLC and LCMS showed the reaction was complete. The reaction mixture was poured into ice water (2 L) and extracted with ethyl acetate (2 L). The organic phase was washed with water (1 L×2), brine (500 mL) and dried with Na₂SO₄. The organic solution was concentrated under reduced pressure at 45° C. to afford (R)-tert-butyl 3-((2-chloro-5-fluoropyrimidin-4-yl)amino)piperidine-1-carboxylate (intermediate A) (700 g, 88%) as a yellow solid, which was used in next step without further purification.

### Preparation of Intermediate B

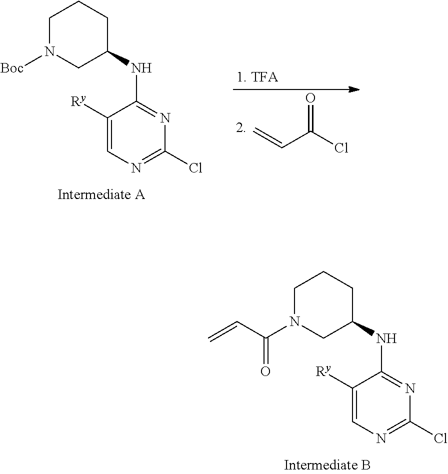

### (R)-2-Chloro-5-fluoro-N-(piperidin-3-yl)pyrimidin-4-amine

A 500 L round bottom flask was charged with intermediate A (50 g, 0.15 mol) and DCM (50 mL). The resulting mixture was cooled to 0° C. TFA (100 mL) was added dropwise and the resulting mixture was allowed to stir at room temperature for 1 h. LCMS showed the reaction was complete. The volatiles were evaporated under reduced pressure at 50° C. The residue was poured into ice water (200 mL) and extracted with DCM (100 mL×2). The aqueous layer was basified with 40% NaOH to pH 4-5. A great amount of solid formed. The resulting suspension was stirred at room temperature for 30 min. The solid was collected by filtration, washed with water and dried to afford the free base (R)-2-chloro-5-fluoro-N-(piperidin-3-yl)pyrimidin-4-amine (31.5 g, 90%) as a yellow solid, which was used in the next step without further purification.

### (R)-1-(3-((2-Chloro-5-fluoropyrimidin-4-yl)amino)piperidin-1-yl)prop-2-en-1-one

A 500 L round bottom flask was charged with (R)-2-chloro-5-fluoro-N-(piperidin-3-yl)pyrimidin-4-amine (31.5 g, 136.6 mmol) and DCM (300 mL). After stirring to a clear solution, an aqueous solution of K₂CO₃ (54.9 g, 397 mmol) in 100 mL water was added, followed by addition of acryloyl chloride (12.67 g, 140 mmol) dropwise at 5-10° C. The resulting mixture was stirred at room temperature for 30 min. The reaction solution was separated and the organic layer was washed with water (100 mL), brine (100 mL) and dried with Na₂SO₄. The organic solution was concentrated under reduced pressure at 50° C. to afford (R)-1-(3-((2-chloro-5-fluoropyrimidin-4-yl)amino)piperidin-1-yl)prop-2-en-1-one (intermediate B) as a yellow oil (35.5 g, 91%), which was used in the next step without further purification.

### Example 1

### Preparation of (R)-1-(3-((5-Fluoro-2-(isoindolin-5-ylamino)pyrimidin-4-yl)amino)piperidin-1-yl)prop-2-en-1-one (I-1)

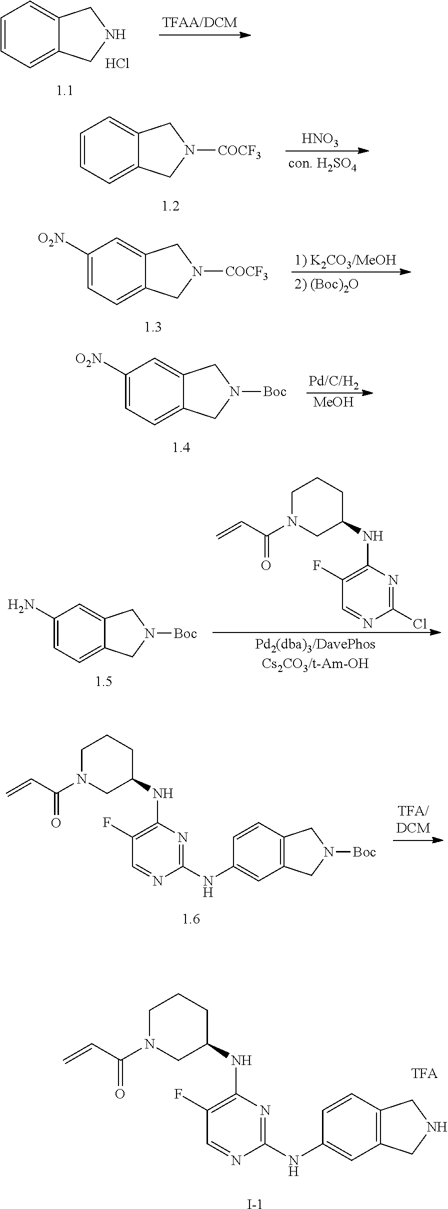

### 2,2,2-Trifluoro-1-(isoindolin-2-yl)ethanone (1.2)

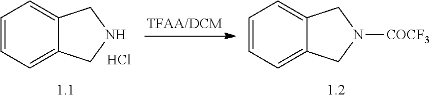

To a solution of isoindoline hydrochloride (1.1, 5.0 g, 32.2 mmol) in DCM (30 ml) was added TFAA (8.1 g, 38.78 mmol) at 0° C. The mixture was allowed to stir at room temperature for 1 h. TLC showed the reaction was complete. The volatiles were evaporated and the residue was partitioned between EtOAc and water. The organic phase was washed with brine, dried over anhydrous Na₂SO₄ and concentrated under reduced pressure to afford crude 2,2,2-trifluoro-1-(isoindolin-2-yl)ethanone (1.2), which was used in next step without further purification.

LCMS: 216.2 [M+1]⁺.

### 2,2,2-Trifluoro-1-(5-nitroisoindolin-2-yl)ethanone (1.3)

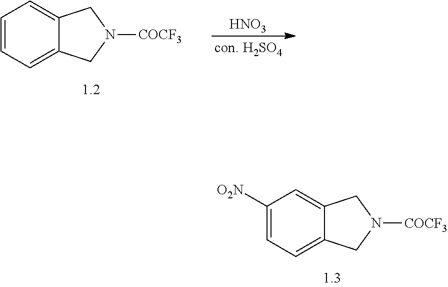

To the solution of crude 2,2,2-trifluoro-1-(isoindolin-2-yl)ethanone (1.2) in con.H₂SO₄ (15 mL) was added fuming nitric acid (35.4 mmol) dropwise at 0° C. The mixture was allowed to stir at room temperature for 10 min. TLC showed the reaction was complete. The reaction mixture was carefully poured into ice water. The resulting off-white solid was collected by filtration, washed with cold water, and dried to afford crude 2,2,2-trifluoro-1-(5-nitroisoindolin-2-yl)ethanone (1.3) (5.8 g), which was used in the next step without further purification.

LCMS: 261.1 [M+1]+.

### tert-Butyl 5-nitroisoindoline-2-carboxylate (1.4)

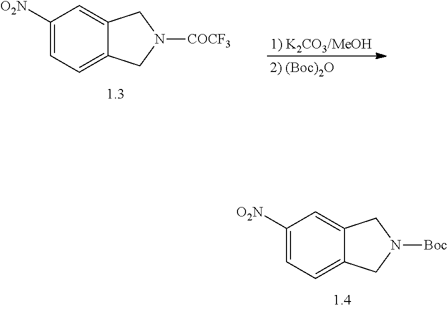

A mixture of 2,2,2-trifluoro-1-(5-nitroisoindolin-2-yl)ethanone (1.3) (5.8 g, 22.3 mmol), K₂CO₃ (6.2 g, 44.6 mmol) in MeOH (32 mL) and H₂O (4 mL) was stirred at room temperature for 30 min. TLC showed the reaction was complete. Boc₂O (4.9 g, 22.3 mmol) was added in portions and the resulting mixture was stirred at room temperature for additional 30 min. TLC showed the reaction was complete. The reaction mixture was partitioned between EtOAc and water. The organic phase was washed with brine, dried over anhydrous Na₂SO₄ and concentrated under reduced pressure. The crude product was purified by column chromatography (eluted with 20% EtOAc in hexane) to afford tert-butyl 5-nitroisoindoline-2-carboxylate (1.4) (3.8 g, 44.7%, 3 steps).

LCMS: 209.2 [M-55]⁺.

### tert-Butyl 5-aminoisoindoline-2-carboxylate (1.5)

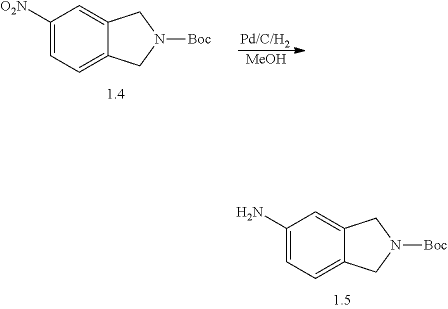 [SMILES: "MeOH" ; CC(C)(C)OC(=O)N1Cc2ccc([N+](=O)[O-])cc2C1 ; "Pd/C/H2" ; CC(C)(C)OC(=O)N1Cc2ccc(N)cc2C1]

To a solution of tert-butyl 5-nitroisoindoline-2-carboxylate (1.4) (3.8 g, 14.4 mmol) in MeOH (50 mL) was added 10% Pd/C (380 mg). The suspension was degassed under vacuum and purged with H₂ several times. The mixture was stirred under H₂ balloon at 50° C. overnight. TLC showed the reaction was complete. The suspension was filtered through celite, which was washed three times with MeOH. The combined filtrate was concentrated and the residue was purified by column chromatography (eluted 50% EtOAc in hexanes) to afford tert-butyl 5-aminoisoindoline-2-carboxylate (1.5) (3.1 g, 92.2%).

LCMS: 235.3[M+1]⁺.

### (R)-tert-Butyl 5-((4-((1-acryloylpiperidin-3-yl)amino)-5-fluoropyrimidin-2-yl)amino)iso-indoline-2-carboxylate (1.6)

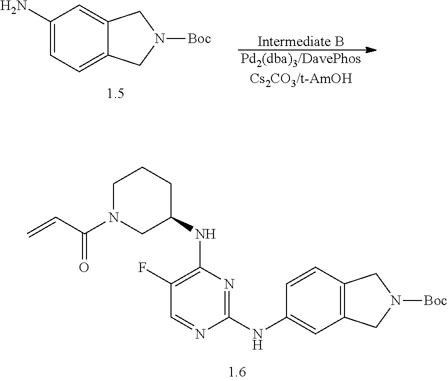 [SMILES: CC(C)(C)OC(=O)N1Cc2ccc(N)cc2C1 ; "Intermediate B" ; "Pd2(dba)3/DavePhosCs2CO3/t-AmOH" ; C=CC(=O)N1CCC[C@@H](Nc2nc(Nc3ccc4c(c3)CN(C(=O)OC(C)(C)C)C4)ncc2F)C1]

A mixture of tert-butyl 5-aminoisoindoline-2-carboxylate (1.5) (400 mg, 1.71 mmol), 1-[(3R)-3-[(2-chloro-5-fluoro-pyrimidin-4-yl)amino]-1-piperidyl]prop-2-en-1-one (intermediate B, 486.1 mg, 1.71 mmol), Cs₂CO₃ (1.11 g, 3.41 mmol), Pd₂(dba)₃ (156 mg, 0.17 mmol) and DavePhos (168 mg, 0.43 mmol) in t-AmOH (10 mL) was heated to reflux for 2 h under nitrogen atmosphere. TLC showed the reaction was complete. The resulting mixture was cooled to room temperature and partitioned between EtOAc and water. The organic phase was washed with brine, dried over anhydrous Na₂SO₄ and concentrated under reduced pressure. The residue was purified by column chromatography (eluted with 50% EtOAc in hexanes) to afford (R)-tert-butyl 5-((4-((1-acryloylpiperidin-3-yl)amino)-5-fluoropyrimidin-2-yl)amino)iso-indoline-2-carboxylate (1.6) (700 mg, 85%).

LCMS: 483.3[M+1]⁺.

### (R)-1-(3-(5-Fluoro-2-(isoindolin-5-ylamino)pyrimidin-4-ylamino)-piperidin-1-yl)prop-2-en-1-one (I-1)

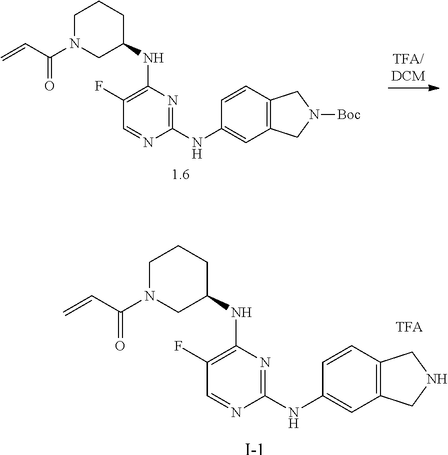 [SMILES: C=CC(=O)N1CCC[C@@H](Nc2nc(Nc3ccc4c(c3)CN(C(=O)OC(C)(C)C)C4)ncc2F)C1 ; C=CC(=O)N1CCC[C@@H](Nc2nc(Nc3ccc4c(c3)CNC4)ncc2F)C1 ; [I-]]

To a solution of (R)-tert-butyl 5-((4-((1-acryloylpiperidin-3-yl)amino)-5-fluoropyrimidin-2-yl)amino)iso-indoline-2-carboxylate (1.6) (600 mg, 1.24 mmol) in DCM (3 mL) was added TFA (6 mL) dropwise at 0° C. The mixture was allowed to stir at room temperature for 30 min. TLC showed the reaction was complete. The volatiles were evaporated and the residue was treated with Et₂O to afford (R)-1-(3-(5-fluoro-2-(isoindolin-5-ylamino)pyrimidin-4-ylamino)-piperidin-1-yl) prop-2-en-1-one (I-1) as the TFA salt (500 mg, 83.9%).

LCMS: 383.3 [M+1]⁺.

¹H NMR (400 MHz, CD₃OD): δ 1.18 (t, 3H), 2.39 (s, 3H), 2.41 (s, 3H), 3.29 (d, 2H), 3.98-4.00 (m, 2H), 4.10-4.14 (m, 4H), 4.60 (br, 1H), 4.75 (s, 1H), 6.33-6.41 (m, 1H), 6.69-6.71 (m, 1H), 6.89-6.95 (m, 1H), 7.24-7.30 (m, 1.5H), 7.35-7.42 (m, 2.5H), 7.49 (s, 1H), 7.71-7.74 (m, 0.5H), 7.78-7.87 (m, 2.5H), 7.92 (d, 0.5H), 8.14-8.17 (m, 1.5H), 8.27-8.33 (m, 1H), 8.77 (d, 1H)

### Example 2

### Preparation of (R)-1-(3-((5-Fluoro-2-((2-methylisoindolin-5-yl)amino)pyrimidin-4-yl)amino)piperidin-1-yl)prop-2-en-1-one (I-2)

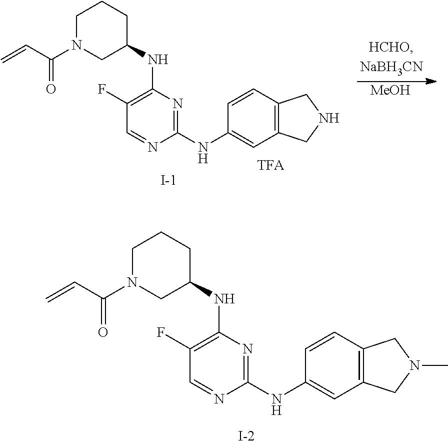

A mixture of (R)-1-(3-(5-fluoro-2-(isoindolin-5-ylamino)pyrimidin-4-ylamino)-piperidin-1-yl) prop-2-en-1-one (I-1) (150 mg, 0.393 mmol), 30% aq HCHO (157 mg) in MeOH (10 mL) was stirred at room temperature for 15 min. NaBH₃CN (50 mg, 0.786 mmol) was added to the mixture. The resulting mixture was stirred at room temperature for 0.5 h. TLC showed the reaction was complete and the mixture was concentrated to dryness. The residue was purified by prep-HPLC to afford (R)-1-(3-((5-fluoro-2-((2-methylisoindolin-5-yl)amino)pyrimidin-4-yl)amino)piperidin-1-yl)prop-2-en-1-one (I-2) as a TFA salt (80.8 mg, 40.3%).

LCMS: 397.3 [M+1]⁺.

¹H NMR (400 MHz, CD₃OD): δ 1.53-1.64 (m, 1H), 1.77-1.87 (m, 1H), 1.93-1.97 (m, 1H), 2.06-2.17 (m, 1H), 2.81-2.89 (m, 1H), 3.14-3.24 (m, 4H), 3.32 (s, 2H), 4.06-4.16 (m, 2H), 4.42-4.71 (m, 3H), 5.47-5.51 (m, 0.5H), 5.85 (dd, 0.5H), 6.08 (d, 0.5H), 6.28 (d, 0.5H), 6.35-6.43 (m, 0.5H), 6.88 (dd, 0.5H), 7.42 (q, 1H), 7.53-7.62 (m, 1H), 7.68 (s, 1H), 7.94-8.01 (m, 1H).

### Example 3

### Preparation of (R)-1-(3-((2-((2-Acetylisoindolin-5-yl)amino)-5-fluoropyrimidin-4-yl)amino)-piperidin-1-yl)prop-2-en-1-one (I-3)

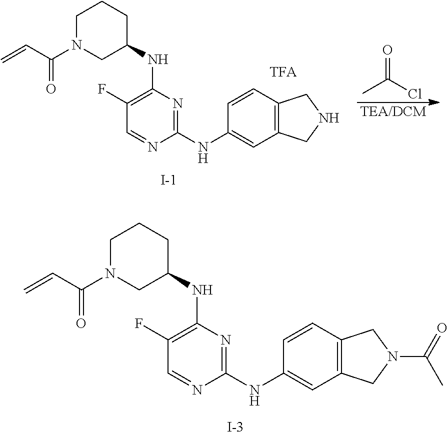

To a solution of (R)-1-(3-(5-fluoro-2-(isoindolin-5-ylamino)pyrimidin-4-ylamino)-piperidin-1-yl) prop-2-en-1-one (I-1) (100 mg, 0.21 mmol) and TEA (42 mg, 0.42 mmol) in DCM (5 mL) was added a solution of acetyl chloride (15 mg, 0.19 mmol) in DCM (5 mL) dropwise at 0° C. The resulting mixture was allowed to stir at room temperature for 0.5 h. TLC showed the reaction was complete. The mixture was quenched with 2N NaHCO₃ and extracted with DCM, dried over anhydrous Na₂SO₄ and concentrated under reduced pressure. The crude product was purified by prep-TLC to afford (R)-1-(3-((2-((2-acetylisoindolin-5-yl)amino)-5-fluoropyrimidin-4-yl)amino)-piperidin-1-yl)prop-2-en-1-one (I-3) (32 mg, 36%) as an off-white solid.

LCMS: 425.3 [M+1]⁺

¹H NMR (400 MHz, CD₃OD): δ 1.45-1.55 (m, 1H), 1.60-1.67 (m, 1H), 1.79-1.80 (m, 1H), 2.00-2.07 (m, 4H), 2.63-2.70 (m, 0.5H), 2.84-2.93 (m, 0.5H), 3.03-3.10 (m, 1H), 3.96-4.22 (m, 2.5H), 4.51-4.71 (m, 4.5H), 5.31-5.36 (d, 0.5H), 5.67-5.71 (m, 0.5H), 5.94-6.17 (m, 1H), 6.27-6.41 (m, 0.5H), 6.69-6.78 (m, 0.5H), 7.07-7.09 (m, 1H), 7.24-7.34 (m, 1H), 7.54-7.67 (m, 2H).

### Example 4

### Preparation of (R)-1-(3-((5-Fluoro-2-((2-(2-methoxyethyl)isoindolin-5-yl)amino)pyrimidin-4-yl)amino)piperidin-1-yl)prop-2-en-1-one (I-4)

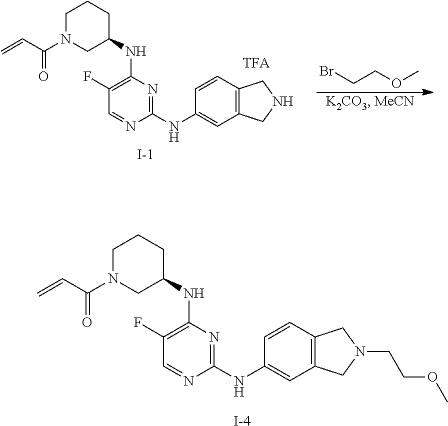

A mixture of (R)-1-(3-(5-fluoro-2-(isoindolin-5-ylamino)pyrimidin-4-ylamino)-piperidin-1-yl) prop-2-en-1-one (I-1) (100 mg, 0.2 mmol), 1-bromo-2-methoxyethane (44 mg, 0.31 mmol), NaI (3.9 mg, 0.026 mmol) and K₂CO₃ (72 mg, 0.52 mmol) in CH₃CN (15 ml) was stirred at 50° C. overnight. TLC showed the reaction was complete. The resulting mixture was cooled to room temperature and partitioned between EtOAc and water. The organic phase was washed with brine, dried over anhydrous Na₂SO₄ and concentrated under reduced pressure The crude product was purified by column chromatography (eluted with 5% MeOH in DCM) to afford (R)-1-(3-((5-fluoro-2-((2-(2-methoxyethyl)isoindolin-5-yl)amino)pyrimidin-4-yl)amino)piperidin-1-yl)prop-2-en-1-one (I-4) (15 mg, 17%) as a yellow solid.

LCMS: 441.3 [M+1]⁺

¹H NMR (400 MHz, CD₃OD): δ 1.47-1.50 (m, 1H), 1.60-1.67 (m, 1H), 1.78-1.83 (m, 1H), 1.99-2.03 (m, 1H), 2.65-2.77 (m, 0.5H), 2.88-2.98 (m, 2.5H), 3.04-3.14 (m, 1H), 3.30 (s, 3H), 3.53 (t, 2H), 3.89-3.98 (m, 6H), 4.14 (d, 0.5H), 4.58 (d, 0.5H), 5.40 (d, 0.5H), 5.70 (d, 0.5H), 5.98 (d, 0.5H), 6.16 (d, 0.5H), 6.36-6.43 (m, 0.5H), 6.71-6.78 (m, 0.5H), 7.02 (d, 1H), 7.25 (t, 1H), 7.52-7.65 (m, 2H).

### Example 5

### Preparation of (R)-1-(3-((5-fluoro-2-((2-(piperidin-4-yl)isoindolin-5-yl)amino)pyrimidin-4-yl)amino)piperidin-1-yl)prop-2-en-1-one (I-5)

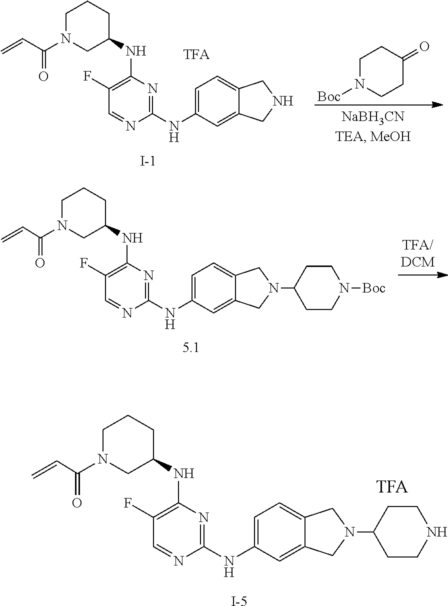

### (R)-tert-Butyl 4-(5-((4-((1-acryloylpiperidin-3-yl)amino)-5-fluoropyrimidin-2-yl)amino)-isoindolin-2-yl)piperidine-1-carboxylate (5.1)

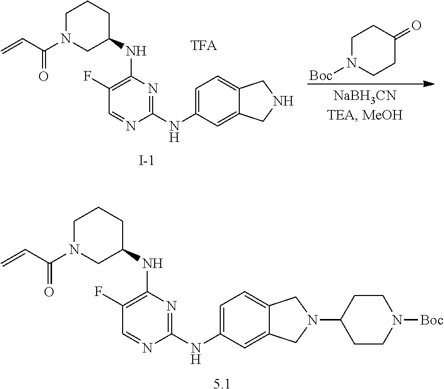 [SMILES: C=CC(=O)N1CCC[C@@H](Nc2nc(Nc3ccc4c(c3)CNC4)ncc2F)C1 ; CC(C)(C)OC(=O)N1CCC(=O)CC1 ; [I-] ; C=CC(=O)N1CCC[C@@H](Nc2nc(Nc3ccc4c(c3)CN(C3CCN(C(=O)OC(C)(C)C)CC3)C4)ncc2F)C1 ; "5.1"]

A mixture of (R)-1-(3-(5-fluoro-2-(isoindolin-5-ylamino)pyrimidin-4-ylamino)-piperidin-1-yl) prop-2-en-1-one (I-1) (600 mg, 1.21 mmol), tert-butyl 4-oxopiperidine-1-carboxylate (450 mg, 2.26 mmol) and TEA (101 mg, 1 mmol) in MeOH (15 mL) was stirred at room temperature for 15 min. NaBH₃CN (400 mg, 6.45 mmol) was added. The resulting mixture was stirred at room temperature for additional 0.5 h. TLC showed the reaction was complete. The reaction mixture was partitioned between EtOAc and water. The organic phase was washed with brine and the mixture was concentrated to dryness. The residue was purified by column chromatography (eluted with 5% MeOH in DCM) to afford (R)-tert-butyl 4-(5-((4-((1-acryloylpiperidin-3-yl)amino)-5-fluoropyrimidin-2-yl)amino)-isoindolin-2-yl)piperidine-1-carboxylate (5.1) (400 mg, 58.4%).

LCMS: 566.3[M+1]⁺.

### (R)-1-(3-((5-fluoro-2-((2-(piperidin-4-yl)isoindolin-5-yl)amino)pyrimidin-4-yl)amino)piperidin-1-yl)prop-2-en-1-one (I-5)

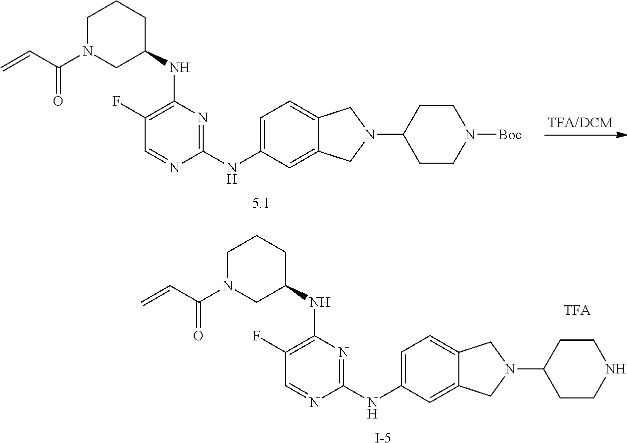

A mixture of (R)-tert-butyl 4-(5-((4-((1-acryloylpiperidin-3-yl)amino)-5-fluoropyrimidin-2-yl)amino)-isoindolin-2-yl)piperidine-1-carboxylate (5.1) (400 mg, 0.71 mmol) and TFA (6 mL) in DCM (3 mL) was stirred at room temperature for 30 min. TLC showed the reaction was complete. The volatiles were removed and the residue was triturated with diethyl ether to afford (R)-1-(3-((5-fluoro-2-((2-(piperidin-4-yl)isoindolin-5-yl)amino)pyrimidin-4-yl) amino)piperidin-1-yl)prop-2-en-1-one (I-5) TFA salt (300 mg, 73.2%) as a yellow solid.

LCMS: 466.3 [M+1]⁺.

¹H NMR (400 MHz, CD₃OD): δ 1.56-1.66 (m, 1H), 1.77-1.87 (m, 1H), 1.93-2.14 (m, 4H), 2.49-2.52 (m, 2H), 2.85-2.91 (m, 1H), 3.10-3.27 (m, 3H), 3.63-3.67 (m, 2H), 3.79 (br, 1H), 4.05-4.17 (m, 2H), 4.41-4.47 (m, 0.5H), 4.67-4.84 (m, 3.5H), 5.51 (d, 0.5H), 5.83 (d, 0.5H), 6.10 (d, 0.5H), 6.26 (d, 0.5H), 6.46 (dd, 0.5H), 6.87 (dd, 0.5H), 7.42 (t, 1H), 7.51-7.62 (m, 1H), 7.67-7.71 (m, 1H), 7.94-7.99 (m, 1H).

### Example 6

### Preparation of (R)-1-(3-((5-Fluoro-2-((2-(piperidin-4-ylmethyl)isoindolin-5-yl)amino)pyrimidin-4-yl)amino)piperidin-1-yl)prop-2-en-1-one (I-6)

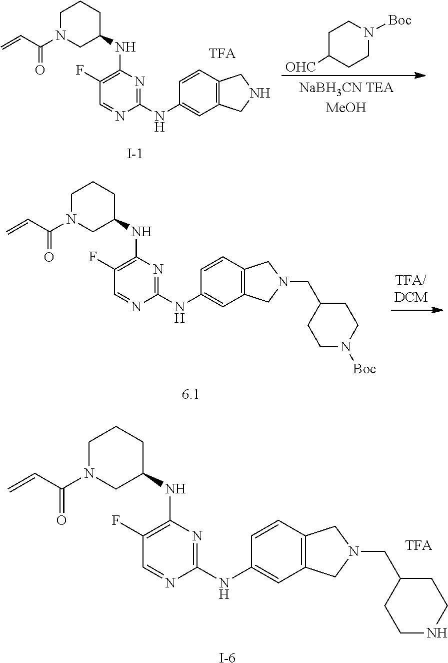

(R)-tert-Butyl 4-((5-((4-((1-acryloylpiperidin-3-yl)amino)-5-fluoropyrimidin-2-yl)amino)-isoindolin-2-yl)methyl)piperidine-1-carboxylate (6.1)

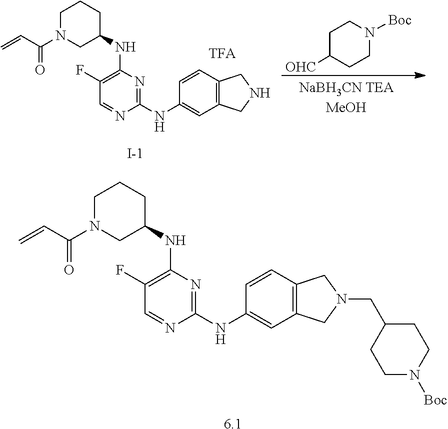 [SMILES: C=CC(=O)N1CCC[C@@H](Nc2nc(Nc3ccc4c(c3)CNC4)ncc2F)C1 ; CC(C)(C)OC(=O)N1CCC(C=O)CC1 ; [I-] ; C=CC(=O)N1CCC[C@@H](Nc2nc(Nc3ccc4c(c3)CN(CC3CCN(C(=O)OC(C)(C)C)CC3)C4)ncc2F)C1 ; "6.1"]

A solution of (R)-1-(3-(5-fluoro-2-(isoindolin-5-ylamino)pyrimidin-4-ylamino)-piperidin-1-yl) prop-2-en-1-one (I-1) (400 mg, 0.81 mmol), tert-butyl 4-formylpiperidine-1-carboxylate (356 mg, 1.67 mmol) and TEA (81 mg, 0.81 mmol) in MeOH (10 mL) was stirred at room temperature for 30 min. NaBH₃CN (105 mg, 1.67 mmol) was added. The resulting mixture was stirred at room temperature for an additional 20 min. TLC showed the reaction was complete. The resulting mixture was partitioned between ethyl acetate and water and the organic phase was washed with brine, dried over anhydrous Na₂SO₄ and concentrated under reduced pressure. The crude product was purified by column chromatography (eluted with 10% EtOAc in hexane) to afford (R)-tert-butyl 4-((5-((4-((1-acryloylpiperidin-3-yl)amino)-5-fluoropyrimidin-2-yl)amino)-isoindolin-2-yl)methyl)piperidine-1-carboxylate (6.1) (350 mg, 72.5%) as a light yellow solid.

LCMS: 580.4[M+1]⁺

### (R)-1-(3-(5-Fluoro-2-(2-(piperidin-4-ylmethyl)isoindolin-5-ylamino) pyrimidin-4-ylamino)piperidin-1-yl)prop-2-en-1-one (I-6)

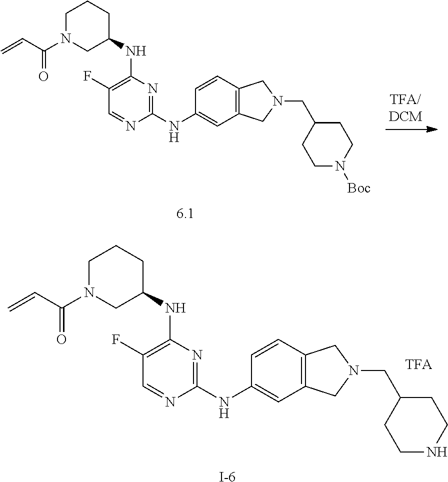

A mixture of (R)-tert-butyl 4-((5-((4-((1-acryloylpiperidin-3-yl)amino)-5-fluoropyrimidin-2-yl)amino)-isoindolin-2-yl)methyl)piperidine-1-carboxylate (6.1) (350 mg, 0.60 mmol) in DCM (3 mL) and TFA (6 mL) was stirred at room temperature for 30 min. TLC showed the reaction was complete. The volatiles were removed and the residue was triturated with diethyl ether to afford (R)-1-(3-(5-fluoro-2-(2-(piperidin-4-ylmethyl)isoindolin-5-ylamino) pyrimidin-4-ylamino)piperidin-1-yl)prop-2-en-1-one (I-6) (330 mg, 94.5%) as a TFA salt.

LCMS: 480.3 [M+1]⁺

¹H NMR (400 MHz, CD₃OD): δ 1.55-1.66 (m, 3H), 1.80-1.97 (m, 2H), 2.12-2.16 (m, 3H), 2.28-2.30 (m, 1H), 2.48-2.72 (m, 1H), 2.85-2.92 (m, 1H), 3.06-3.16 (m, 3H), 3.38-3.52 (m, 4H), 3.88-4.18 (m, 2H), 4.43-4.80 (m, 4H), 5.53 (d, 0.5H), 5.83 (d, 0.5H), 6.09-6.13 (m, 0.5H), 6.24-6.29 (m, 0.5H), 6.43-6.50 (m, 0.5H), 6.83-6.90 (m, 0.5H), 7.40-7.45 (m, 1H), 7.53-7.71 (m, 2H), 7.94-8.00 (m, 1H).

### Example 7

### Preparation of (R)-1-(3-((5-Fluoro-2-((2-(1-methylpiperidin-4-yl)isoindolin-5-yl)amino)-pyrimidin-4-yl)amino)piperidin-1-yl)prop-2-en-1-one (I-7)

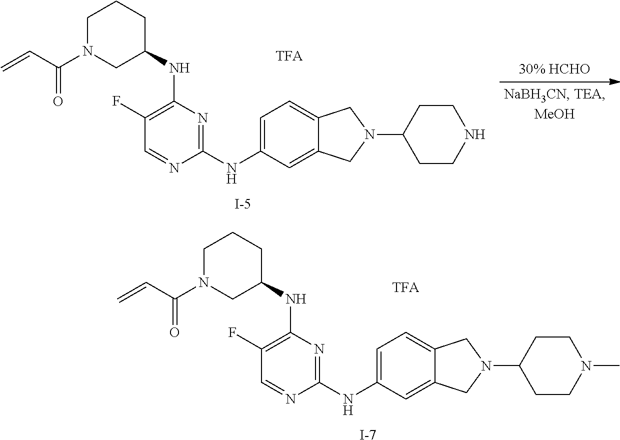

A mixture of (R)-1-(3-((5-fluoro-2-((2-(piperidin-4-yl)isoindolin-5-yl)amino)pyrimidin-4-yl) amino)piperidin-1-yl)prop-2-en-1-one (I-5) (100 mg, 0.173 mmol), TEA (18 mg, 0.18 mmol) and 30% HCHO (80 mg, 0.8 mmol) in MeOH (5 mL) was stirred at room temperature for 15 min. NaBH₃CN (43.6 mg, 0.69 mmol) was added and the resulting mixture was stirred at room temperature for additional 0.5 h. TLC showed the reaction was complete. The reaction mixture was partitioned between DCM and water and the organic phase was washed with brine, dried over anhydrous Na₂SO₄ and concentrated under reduced pressure. The residue was purified by prep-HPLC to afford (R)-1-(3-((5-fluoro-2-((2-(1-methylpiperidin-4-yl)isoindolin-5-yl)amino)-pyrimidin-4-yl)amino)piperidin-1-yl)prop-2-en-1-one (I-7) (35 mg, 34.1%) as TFA salt.

LCMS: 480.3 [M+1]⁺

¹H NMR (400 MHz, CD₃OD): δ 1.47-1.51 (m, 1H), 1.66-1.75 (m, 1H), 1.80-1.84 (m, 1H), 1.99-2.09 (m, 3H), 2.38-2.43 (m, 2H), 2.75-2.83 (m, 4H), 2.98-3.14 (m, 3H), 3.62-3.71 (m, 3H), 3.88-4.06 (m, 2H), 4.30-4.33 (m, 0.5H), 4.41-4.57 (m, 1H), 4.69 (s, 3.5H), 5.40 (d, 0.5H), 5.71 (d, 0.5H), 5.99 (d, 0.5H), 6.14 (d, 0.5H), 6.33 (q, 0.5H), 6.75 (q, 0.5H), 7.33 (q, 1H), 7.42-7.49 (m, 1H), 7.53-7.56 (m, 1H), 7.82-7.89 (m, 1H).

### Example 8

### Preparation of (R)-1-(3-((5-Fluoro-2-((2-(oxetan-3-yl)isoindolin-5-yl)amino)pyrimidin-4-yl)amino)piperidin-1-yl)prop-2-en-1-one (I-8)

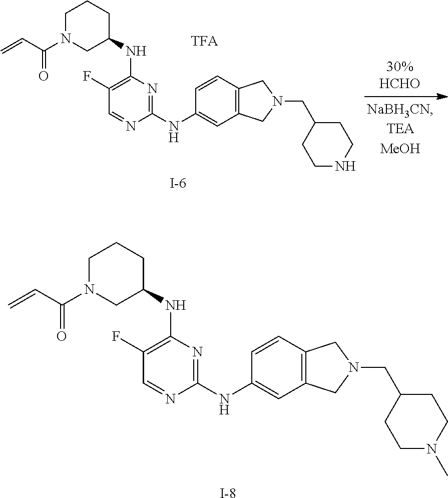

A solution of (R)-1-(3-(5-fluoro-2-(2-(piperidin-4-ylmethyl)isoindolin-5-ylamino) pyrimidin-4-ylamino)piperidin-1-yl)prop-2-en-1-one (I-6) (100 mg, 0.168 mmol), TEA (17 mg, 0.17 mmol) and 30% HCHO (100 mg, 1 mmol) in MeOH (3 mL) was stirred at room temperature for 20 min. NaBH₃CN (21.9 mg, 0.348 mmol) was added and the resulting mixture was stirred at room temperature for additional 1 h. TLC showed the reaction complete. The reaction mixture was partitioned between DCM and water and the organic phase was washed with brine, dried over anhydrous Na₂SO₄ and concentrated under reduced pressure. The residue was purified by prep-TLC to afford (R)-1-(3-((5-fluoro-2-((2-(oxetan-3-yl)isoindolin-5-yl)amino)pyrimidin-4-yl)amino)piperidin-1-yl)prop-2-en-1-one (I-8) (28 mg, 33.7%) as a white solid.

LCMS: 494.4 [M+1]⁺

¹H NMR (400 MHz, CD₃OD): δ 1.36-1.50 (m, 3H), 1.59-1.66 (m, 1H), 1.78-2.01 (m, 5H), 2.54-2.87 (m, 8H), 3.03-3.09 (m, 1H), 3.35-3.38 (m, 2H), 3.76-4.06 (m, 6H), 4.19-4.24 (m, 0.5H), 4.58-4.62 (m, 0.5H), 5.38-5.41 (m, 0.5H), 5.68-5.71 (m, 0.5H), 5.96-6.05 (m, 0.5H), 6.13-6.18 (m, 0.5H), 6.37-6.44 (d, 0.5H), 6.71-6.78 (m, 0.5H), 7.00-7.03 (m, 1H), 7.16-7.29 (m, 1H), 7.54-7.66 (m, 2H).

### Example 9

### Preparation of (R)-1-(3-(2-(2-(1-Acetylpiperidin-4-yl)isoindolin-5-ylamino)-5-fluoropyrimidin-4-ylamino)piperidin-1-yl)prop-2-en-1-one (I-9)

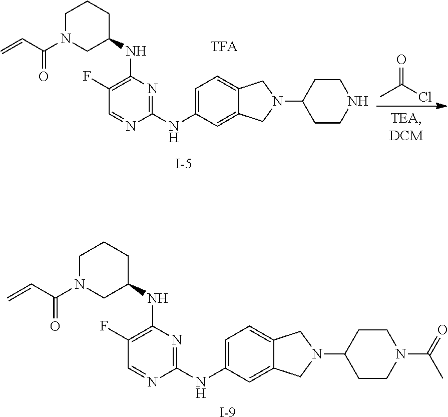

To a solution of (R)-1-(3-((5-fluoro-2-((2-(piperidin-4-yl)isoindolin-5-yl)amino)pyrimidin-4-yl) amino)piperidin-1-yl)prop-2-en-1-one (I-5) (150 mg, 0.259 mmol) and TEA (54 mg, 0.54 mmol) in DCM (10 mL) was added acetyl chloride (188 mg, 0.24 mmol) at 0° C. The resulting mixture was allowed to stirred at room temperature for additional 15 min. The reaction mixture was partitioned between DCM and water and the organic phase was washed with brine, dried over anhydrous Na₂SO₄ and concentrated under reduced pressure. The residue was purified by prep-TLC to afford (R)-1-(3-(2-(2-(1-acetylpiperidin-4-yl)isoindolin-5-ylamino)-5-fluoropyrimidin-4-ylamino)piperidin-1-yl)prop-2-en-1-one (I-9) (50 mg, 38%) as a yellow solid.

LCMS: 508.3 [M+1]⁺

¹H NMR (400 MHz, CD₃OD): δ 1.36-1.81 (m, 4H), 1.90-1.94 (m, 1H), 2.04-2.14 (m, 6H), 2.69-2.83 (m, 2.5H), 2.97-3.04 (m, 0.5H), 3.15-3.24 (m, 2H), 3.89-3.99 (m, 5H), 4.06-4.15 (m, 2H), 4.28 (d, 0.5H), 4.51 (d, 1H), 4.69-4.73 (m, 0.5H), 5.51 (d, 0.5H), 5.81 (d, 0.5H), 6.11 (d, 0.5H), 6.28 (d, 0.5H), 6.52 (q, 0.5H), 6.86 (q, 0.5H), 7.13 (d, 1H), 7.35 (t, 1H), 7.67 (d, 1H), 7.76 (q, 1H).

### Example 10

### Preparation of (R)-1-(3-(2-(2-Acetylisoindolin-5-ylamino)-5-fluoropyrimidin-4-ylamino) piperidin-1-yl)prop-2-en-1-one (I-10)

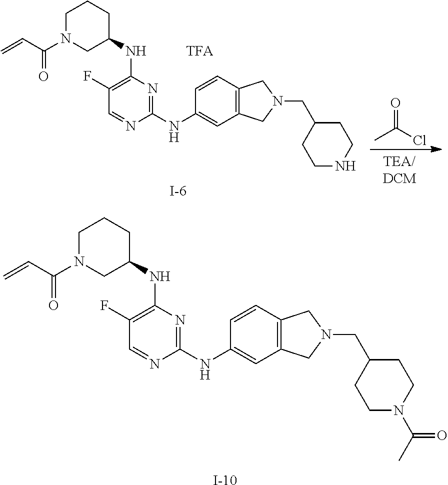

To a solution of (R)-1-(3-(5-fluoro-2-(2-(piperidin-4-ylmethyl)isoindolin-5-ylamino) pyrimidin-4-ylamino)piperidin-1-yl)prop-2-en-1-one (I-6) (100 mg, 0.168 mmol) and TEA (35 mg, 0.348 mmol) in DCM (5 mL) was added a solution of acetyl chloride (13 mg, 0.174 mmol) in DCM (1 mL) dropwise at 0° C. The resulting mixture was allowed to stir at room temperature for additional 30 min. TLC showed the reaction was complete. The reaction mixture was partitioned between DCM and water and the organic phase was washed with brine, dried over anhydrous Na₂SO₄ and concentrated under reduced pressure. The residue was purified by prep-TLC to afford (R)-1-(3-(2-(2-acetylisoindolin-5-ylamino)-5-fluoropyrimidin-4-ylamino) piperidin-1-yl)prop-2-en-1-one (I-10) (30 mg, 34.2%) as a white solid.

LCMS: 522.3 [M+1]⁺.

¹H NMR (400 MHz, CD₃OD): δ 1.04-1.80 (m, 2H), 1.46-1.53 (m, 1H), 1.59-1.66 (m, 1H), 1.75-1.85 (m, 4H), 2.00 (br, 4H), 2.37-2.70 (m, 4H), 2.88-3.10 (m, 3H), 3.83-4.00 (m, 6H), 4.14-4.18 (m, 0.5H), 4.42-4.46 (m, 1H), 4.59-4.62 (m, 0.5H), 5.38-5.41 (m, 0.5H), 5.68-5.71 (m, 0.5H), 5.96-6.01 (m, 0.5H), 6.14-6.18 (m, 0.5H), 6.37-6.44 (m, 0.5H), 6.74-6.78 (m, 0.5H), 7.02-7.04 (m, 1H), 7.21-7.28 (m, 1H), 7.54-7.58 (m, 1H), 7.64-7.66 (m, 1H).

### Example 11

### Preparation of (R)-1-(3-(5-Fluoro-2-(2-((tetrahydro-2H-pyran-4-yl)methyl)isoindolin-5-ylamino)pyrimidin-4-ylamino)piperidin-1-yl)prop-2-en-1-one (I-11)

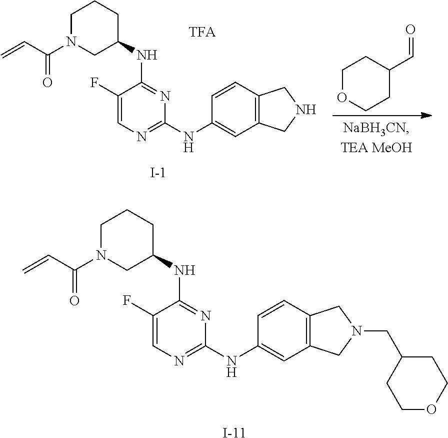

A solution of (R)-1-(3-((5-fluoro-2-(isoindolin-5-ylamino)pyrimidin-4-yl)amino)piperidin-1-yl)prop-2-en-1-one (I-1) (150 mg, 0.302 mmol), TEA (30 mg, 0.3 mmol) and tetrahydro-2H-pyran-4-carbaldehyde (114 mg, 1 mmol) in MeOH (10 mL) was stirred at room temperature for 20 min. NaBH₃CN (44 mg, 0.71 mmol) was added and the resulting mixture was stirred at room temperature for additional 1 h. TLC showed the reaction was complete. The reaction mixture was partitioned between DCM and water and the organic phase was washed with brine, dried over anhydrous Na₂SO₄ and concentrated under reduced pressure. The residue was purified by prep-TLC to afford (R)-1-(3-(5-fluoro-2-(2-((tetrahydro-2H-pyran-4-yl)methyl)isoindolin-5-ylamino)pyrimidin-4-ylamino)piperidin-1-yl)prop-2-en-1-one (I-11) (40 mg, 27.6%) as a yellow solid.

LCMS: 481.3 [M+1]⁺.

¹H NMR (400 MHz, CD₃OD): δ 1.31-1.40 (m, 2H), 1.55-1.65 (m, 1H), 1.72-1.80 (m, 2H), 1.91-1.96 (m, 3H), 2.11-2.14 (m, 1H), 2.76-2.84 (m, 2.5H), 2.98-3.04 (m, 0.5H), 3.15-3.25 (m, 1H), 3.46-3.52 (m, 2H), 3.97-4.30 (m, 8.5H), 4.70-4.74 (m, 0.5H), 5.50 (d, 0.5H), 5.82 (d, 0.5H), 6.10 (d, 0.5H), 6.28 (d, 0.5H), 6.52 (q, 0.5H), 6.86 (q, 0.5H), 7.16 (d, 1H), 7.35-7.41 (m, 1H), 7.67-7.78 (m, 2H).

### Example 12

### Preparation of (R)-1-(3-((5-Fluoro-2-((2-(tetrahydro-2H-pyran-4-yl)isoindolin-5-yl)amino)-pyrimidin-4-yl)amino)piperidin-1-yl)prop-2-en-1-one (I-12)

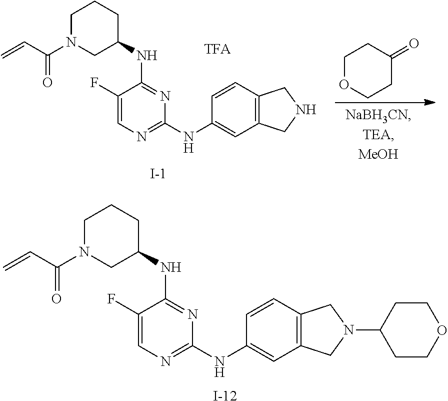

A mixture of (R)-1-(3-((5-fluoro-2-(isoindolin-5-ylamino)pyrimidin-4-yl)amino)piperidin-1-yl)prop-2-en-1-one (I-1) (100 mg, 0.20 mmol), TEA (20 mg, 0.2 mmol) and dihydro-2H-pyran-4(3H)-one (75 mg, 0.75 mmol) in MeOH (10 mL) was stirred at room temperature for 20 min. NaBH₃CN (21.9 mg, 0.35 mmol) was added and the resulting mixture was stirred at room temperature for an additional 1 h. TLC showed the reaction was complete. The reaction mixture was partitioned between DCM and water. The organic phase was washed with brine, dried over anhydrous Na₂SO₄ and concentrated under reduced pressure. The residue was purified by prep-TLC to afford (R)-1-(3-((5-fluoro-2-((2-(tetrahydro-2H-pyran-4-yl)isoindolin-5-yl)amino)-pyrimidin-4-yl)amino)piperidin-1-yl)prop-2-en-1-one (I-12) (15 mg, 16.1%) as a yellow solid.

LCMS: 467.4 [M+1]⁺

¹H NMR (400 MHz, CD₃OD): δ 1.47-1.66 (m, 4H), 1.78-1.84 (m, 1H), 1.89 (d, 2H), 1.99-2.03 (m, 1H), 2.64-2.93 (m, 2H), 3.03-3.13 (m, 1H), 3.33-3.40 (m, 2H), 3.90-4.00 (m, 7H), 4.14-4.18 (m, 0.5H), 4.59-4.63 (m, 0.5H), 5.40 (d, 0.5H), 5.69 (d, 0.5H), 5.97-6.04 (m, 0.5H), 6.14-6.18 (m, 0.5H), 6.41 (q, 0.5H), 6.75 (q, 0.5H), 7.05 (d, 1H), 7.26 (t, 1H), 7.57-7.66 (m, 2H).

### Example 13

### Preparation of (R)-1-(3-((5-Fluoro-2-((2-(oxetan-3-yl)isoindolin-5-yl)amino)pyrimidin-4-yl) amino)piperidin-1-yl)prop-2-en-1-one (I-13)

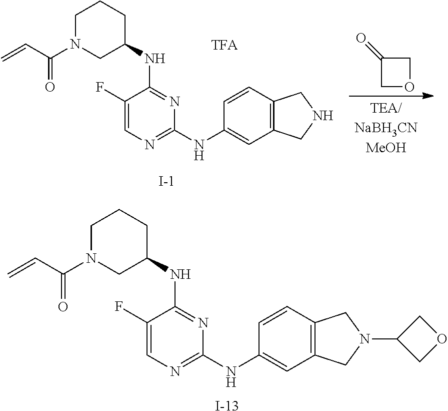

A solution of (R)-1-(3-((5-fluoro-2-(isoindolin-5-ylamino)pyrimidin-4-yl)amino)piperidin-1-yl)prop-2-en-1-one (I-1) (100 mg, 0.2 mmol), TEA (20 mg, 0.2 mmol) and oxetan-3-one (37.6 mg, 0.522 mmol) in MeOH (3 mL) was stirred at room temperature for 20 min. NaBH₃CN (21.9 mg, 0.35 mmol) was added and the resulting mixture was stirred at room temperature for additional 6 h. TLC showed the reaction was complete. The reaction mixture was partitioned between DCM and water. The organic phase was washed with brine, dried over anhydrous Na₂SO₄ and concentrated under reduced pressure. The residue was purified by prep-TLC to afford (R)-1-(3-((5-fluoro-2-((2-(oxetan-3-yl)isoindolin-5-yl)amino)pyrimidin-4-yl) amino)piperidin-1-yl)prop-2-en-1-one (I-13) (32 mg, 36.5%) as a white solid.

LCMS: 439.2 [M+1]⁺

¹H NMR (400 MHz, CDCl₃): δ 1.68-2.09 (m, 4H), 3.28-3.43 (m, 2H), 3.67 (br, 0.5H), 3.94-4.23 (m, 7H), 4.74-4.80 (m, 3.5H), 4.88 (br, 0.5H), 5.11 (br, 0.5H), 5.56 (d, 0.5H), 5.75 (d, 0.5H), 6.24-6.62 (m, 2H), 6.87-7.14 (m, 1H), 7.12-7.14 (m, 1H), 7.26-7.35 (m, 2H), 7.50-7.59 (m, 1H), 7.81 (s, 1H).

### Example 14

### Preparation of (R)-1-(3-((5-Fluoro-2-((2-neopentylisoindolin-5-yl)amino)pyrimidin-4-yl)amino)piperidin-1-yl)prop-2-en-1-one (I-14)

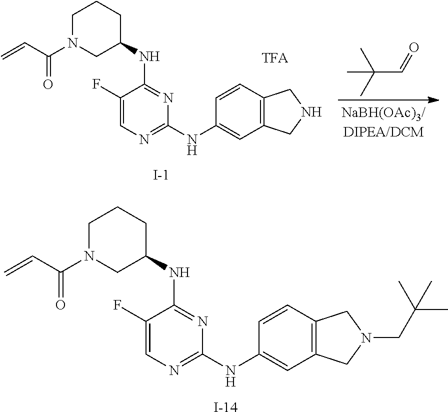

To a solution of (R)-1-(3-((5-fluoro-2-(isoindolin-5-ylamino)pyrimidin-4-yl)amino)piperidin-1-yl)prop-2-en-1-one (I-1) (78 mg, 0.20 mmol) in DCM (10 mL) was added DIPEA, pivaldehyde (72 mg, 0.83 mmol) and sodium triacetoxyborohydride (89 mg, 0.42 mmol). The reaction mixture was stirred at room temperature for 2 h. TLC showed the reaction was complete. The reaction mixture was quenched with water and extracted with DCM. The combined organic layers were washed with water and brine, dried over sodium sulfate and concentrated in vacuo. The crude product was purified by column chromatography (dichloromethane/methanol:20/1) to afford (R)-1-(3-((5-fluoro-2-((2-neopentylisoindolin-5-yl)amino)pyrimidin-4-yl)amino)piperidin-1-yl)prop-2-en-1-one (I-14) as a yellow solid (20.3 mg, 22%).

LCMS: 453.3 [M+1]⁺

¹H NMR (400 MHz, DMSO-d₆): δ 0.913 (s, 9H), 1.37-1.340 (m, 1H), 1.59-1.66 (m, 1H), 1.80-1.83 (m, 1H), 1.97-2.00 (m, 1H), 2.45 (s, 2.5H), 2.66-2.79 (m, 1H), 2.97-3.03 (m, 0.5H), 3.09-3.15 (m, 0.5H), 3.89 (s, 3.5H), 3.99 (br, 2H), 4.21 (d, 0.5H), 4.46 (d, 0.5H), 5.50 (d, 0.5H), 5.69 (d, 0.5H), 6.01-6.15 (m, 1H), 6.59-6.61 (m, 0.5H), 6.81-6.87 (m, 0.5H), 7.00 (d, 1H), 7.32-7.37 (m, 2H), 7.65 (d, 1H), 7.88 (d, 1H).

### Example 15

### Preparation of (R)-1-(3-((5-Fluoro-2-((2-(oxetan-3-ylmethyl)isoindolin-5-yl)amino)pyrimidin-4-yl)amino)piperidin-1-yl)prop-2-en-1-one (I-15)

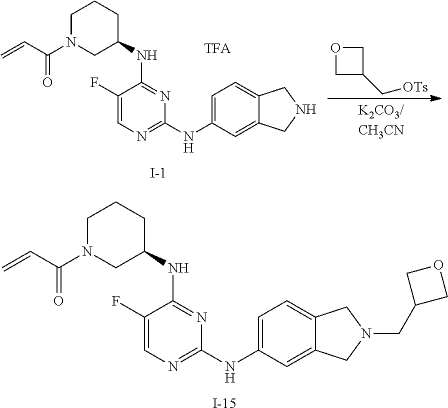

To a stirred solution of (R)-1-(3-((5-fluoro-2-(isoindolin-5-ylamino)pyrimidin-4-yl)amino)piperidin-1-yl)prop-2-en-1-one (I-1) (17 mg, 0.31 mmol) in acetonitrile (15 mL) was added K₂CO₃ (86.5 mg, 0.62 mmol) and oxetan-3-ylmethyl 4-methylbenzenesulfonate. The reaction mixture was stirred at 40° C. overnight under N₂. TLC showed the reaction was complete. The reaction mixture was quenched with water and extracted with EtOAc. The combined organic layers were washed with water and brine, dried over sodium sulfate and concentrated in vacuo. The crude product was purified by column chromatography (dichloromethane:methanol/20:1) to afford (R)-1-(3-((5-fluoro-2-((2-(oxetan-3-ylmethyl)isoindolin-5-yl)amino)pyrimidin-4-yl)amino)piperidin-1-yl)prop-2-en-1-one (I-15) as a yellow solid (26 mg, 18.8%).

LCMS: 453.4 [M+1]⁺

¹H NMR (400 MHz, DMSO-d₆): δ1.35-1.42 (m, 1H), 1.60-1.66 (m, 1H), 1.80-1.83 (m, 1H), 1.96-1.99 (m, 1H), 2.66-2.76 (m, 1H), 2.93-2.97 (m, 2H), 3.00-3.04 (m, 0.5H), 3.12-3.19 (m, 1.5H), 3.69 (d, 4H), 3.99-4.03 (m, 2H), 4.23 (d, 0.5H), 4.30-4.31 (m, 2H), 4.45 (d, 0.5H), 4.67 (t, 2H), 5.48 (d, 0.5H), 5.73 (t, 0.5H), 6.01-6.15 (m, 1H), 6.59-6.66 (m, 0.5H), 6.84-6.91 (m, 0.5H), 7.00 (d, 1H).

### Example 16

### Preparation of (R)-1-(3-(5-Fluoro-2-(2-isopropylisoindolin-5-ylamino)pyrimidin-4-ylamino)piperidin-1-yl)prop-2-en-1-one (I-16)

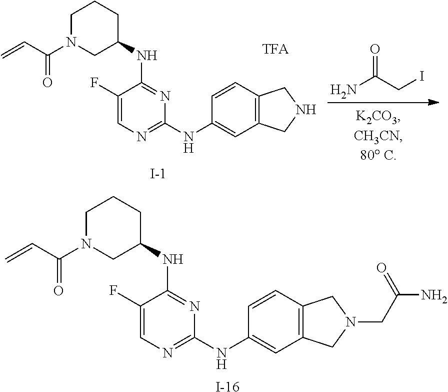

2-Iodoacetamide (78 mg, 0.42 mmol) was added to a mixture of (R)-1-(3-((5-fluoro-2-(isoindolin-5-ylamino)pyrimidin-4-yl)amino)piperidin-1-yl)prop-2-en-1-one (I-1) (160 mg, 0.42 mmol) and K₂CO₃ (232 mg, 1.68 mmol) in CH₃CN (8 mL). The resulting mixture was heated at 80° C. for 1 h. After cooling to room temperature, the reaction mixture was diluted with water and extracted with ethyl acetate. The combined organic layers were washed with water and brine, dried over sodium sulfate and concentrated in vacuo. The crude product was purified by column chromatography (dichloromethane/methanol:15/1) to afford (R)-1-(3-(5-fluoro-2-(2-isopropylisoindolin-5-ylamino)pyrimidin-4-ylamino)piperidin-1-yl)prop-2-en-1-one (I-16) as a yellow solid (46 mg, 26%).

LCMS: 440.2[M+1]⁺.

¹H NMR (400 MHz, DMSO-d₆): δ1.40-1.42 (m, 1H), 1.60-1.67 (m, 1H), 1.79-1.83 (m, 1H), 1.97-2.00 (m, 1H), 2.67-2.79 (m, 1H), 2.98-3.05 (m, 0.5H), 3.13-3.14 (m, 0.5H), 3.24-3.25 (m, 1H), 3.88 (s, 4H), 3.99-4.00 (m, 2H), 4.17-4.20 (m, 0.5H), 4.44-4.47 (m, 0.5H), 5.46-5.49 (m, 0.5H), 5.69-5.72 (m, 0.5H), 6.00-6.15 (m, 1H), 6.56-6.62 (m, 0.5H), 6.81-6.88 (m, 0.5H), 7.03-7.10 (m, 2H), 7.30-7.41 (m, 2H), 7.67 (d, J=32 Hz, 1H), 7.90 (d, J=3.6 Hz, 1H), 9.04 (s, 1H).

### Example 17

### Preparation of (R)-1-(3-((5-Fluoro-2-((2-isopropylisoindolin-5-yl)amino)pyrimidin-4-yl)amino)piperidin-1-yl)prop-2-en-1-one (I-17)

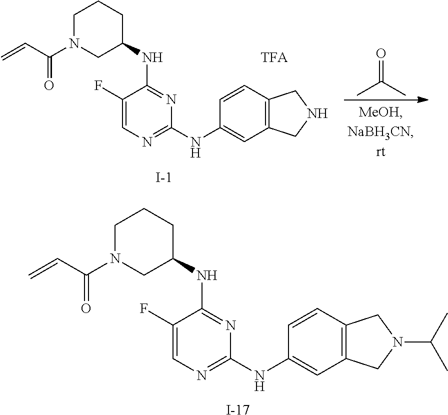

A mixture of acetone (13 mg, 0.22 mmol) and (R)-1-(3-((5-fluoro-2-(isoindolin-5-ylamino)pyrimidin-4-yl)amino)piperidin-1-yl)prop-2-en-1-one (I-1) (79 mg, 0.21 mmol) in MeOH (8 mL) was stirred at 25° C. for 3 h. NaBH₃CN (26 mg, 0.42 mmol) was added. The mixture was stirred for 2 h. The reaction mixture was diluted with water and extracted with ethyl acetate. The combined organic layers were washed with water and brine, dried over sodium sulfate and concentrated in vacuo. The crude product was purified by column chromatography (dichloromethane/methanol:10/1) to afford (R)-1-(3-((5-fluoro-2-((2-isopropylisoindolin-5-yl)amino)pyrimidin-4-yl)amino)piperidin-1-yl)prop-2-en-1-one (I-17) as a yellow solid (48 mg, 55%).

LCMS: 425.2 [M+1]⁺.

¹H NMR (400 MHz, DMSO-d₆): δ1.12 (d, J=5.6 Hz, 6H), 1.40-1.43 (m, 1H), 1.62-1.65 (m, 1H), 1.80-1.84 (m, 1H), 1.97-2.00 (m, 1H), 2.69-2.80 (m, 2H), 3.00-3.04 (m, 0.5H), 3.14-3.17 (m, 0.5H), 3.89-4.03 (m, 6H), 4.20-4.24 (m, 0.5H), 4.46-4.50 (m, 0.5H), 5.52 (d, J=10.4 Hz, 0.5H), 5.69-5.76 (m, 0.5H), 6.07-6.16 (m, 1H), 6.62-6.69 (m, 0.5H), 6.83-6.89 (m, 0.5H), 7.04-7.06 (m, 1H), 7.38-7.39 (m, 1H), 7.68-7.75 (m, 1H), 7.90 (d, J=3.6 Hz, 1H), 9.07-9.08 (m, 1H).

### Example 18

### Preparation of (R)-1-(3-((2-((2-Cyclobutylisoindolin-5-yl)amino)-5-fluoropyrimidin-4-yl)amino)piperidin-1-yl)prop-2-en-1-one (I-18)

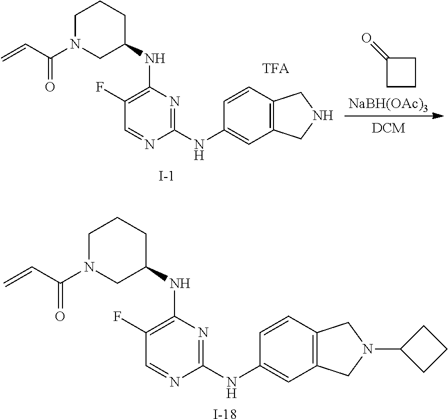

To a solution of (R)-1-(3-((5-fluoro-2-(isoindolin-5-ylamino)pyrimidin-4-yl)amino)piperidin-1-yl)prop-2-en-1-one (I-1) (150.0 mg, 0.39 mmol) in DCM (10 mL) was added cyclobutanone (110 mg, 1.57 mmol) and NaBH(OAc)₃ (83.13 mg, 0.39 mmol). The mixture was stirred at 25° C. for 4 h. TLC showed the reaction was complete. The mixture was quenched with water, extracted with DCM, washed with water and brine and concentrated in vacuo to afford the crude product, which was purified by column chromatography (DCM/MeOH=30/1) to afford (R)-1-(3-((2-((2-cyclobutylisoindolin-5-yl)amino)-5-fluoropyrimidin-4-yl)amino)piperidin-1-yl)prop-2-en-1-one (I-18) as a yellow solid (60 mg, 35%).

LCMS: 437.3 [M+1]+

¹H NMR (400 MHz, DMSO-d₆): δ 1.31 (br, 1H), 1.64-1.73 (m, 3H), 1.80-1.83 (m, 1H), 1.91-1.99 (m, 5H), 2.68-2.78 (m, 1H), 3.01 (t, 0.5H), 3.13 (t, 0.5H), 3.25 (t, 1H), 3.70 (s, 4H), 3.99 (br, 2H), 4.20 (d, 0.5H), 4.47 (d, 0.5H), 5.50 (d, 0.5H), 5.70 (d, 0.5H), 6.03 (d, 0.5H), 6.13 (d, 0.5H), 6.59-6.66 (m, 0.5H), 6.85 (dd, 0.5H), 7.02 (d, 1H), 7.32-7.37 (m, 2H), 7.67 (d, 1H), 7.89 (d, 1H), 9.03 (br, 1H).

### Example 19

### Preparation of (R)-1-(3-((2-((2-(cyclopropylmethyl)isoindolin-5-yl)amino)-5-fluoropyrimidin-4-yl)amino)piperidin-1-yl)prop-2-en-1-one (I-19)

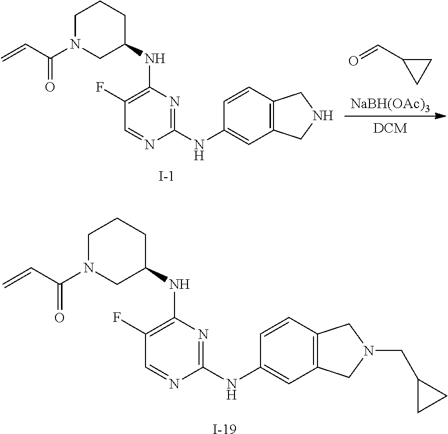

To a solution of (R)-1-(3-((5-fluoro-2-(isoindolin-5-ylamino)pyrimidin-4-yl)amino)piperidin-1-yl)prop-2-en-1-one (I-1) (75 mg, 0.20 mmol) in DCM (20 mL) was added cyclopropane-carbaldehyde (55 mg, 0.78 mmol). The mixture was stirred at 25° C. for 0.5 h. NaBH(OAc)₃ (83.1 mg, 0.39 mmol) was added into the mixture. The reaction was stirred at 25° C. for 4 h. TLC showed the reaction complete. The mixture was quenched with water, extracted with DCM, washed with water and brine and concentrated in vacuo to afford the crude product. The crude compound was purified by HPLC to afford (R)-1-(3-((2-((2-(cyclopropylmethyl)isoindolin-5-yl)amino)-5-fluoropyrimidin-4-yl)amino)piperidin-1-yl)prop-2-en-1-one (I-19) as a white solid (5.7 mg, 6.7%).

LCMS: 437.2 [M+1]+

¹H NMR (400 MHz, CD₃OD): δ 0.51 (br, 2H), 0.82 (br, 2H), 1.22 (br, 1H), 1.57-1.61 (m, 1H), 1.79-1.83 (m, 1H), 1.93-1.98 (m, 1H), 2.14 (br, 1H), 2.87 (dd, 1H), 3.19 (dd, 1H), 3.33-3.37 (m, 2.5H), 4.07-4.16 (m, 2H), 4.44-4.72 (m, 3H), 5.51 (br, 0.5H), 5.82 (d, 0.5H), 6.09 (d, 0.5H), 6.27 (d, 0.5H), 6.43 (br, 0.5H), 6.87 (dd, 0.5H), 7.42 (t, 1H), 7.58 (d, 1H), 7.71 (s, 1H), 7.96 (dd, 1H).

### Example 20

### Preparation of (R)-1-(3-((5-Fluoro-2-((2-deuteromethylisoindolin-5-yl)amino)pyrimidin-4-yl)amino)piperidin-1-yl)prop-2-en-1-one (I-20)

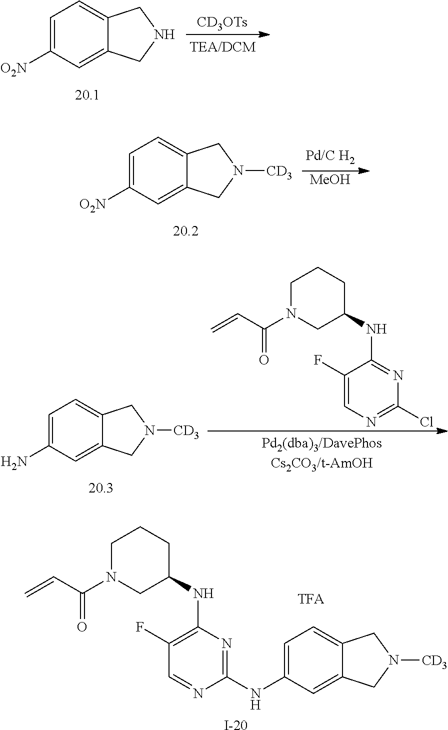

### 2-Deuteromethyl-5-nitroisoindoline (20.2)

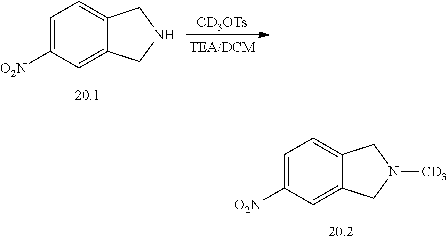 [SMILES: O=[N+]([O-])c1ccc2c(c1)CNC2 ; [2H]C([2H])([2H])N1Cc2ccc([N+](=O)[O-])cc2C1 ; "CD3OTs"]

A mixture of 20.1 (300 mg, 1.83 mmol), TEA (620 mg, 6.10 mmol) and CD₃OTs (692 mg, 3.66 mmol) in DMF (15 mL) was stirred at 100° C. for 2 h. TLC showed the reaction was complete. The reaction mixture was cooled to room temperature and partitioned between EtOAc and water. The organic phase was washed with brine and dried over anhydrous Na₂SO₄. The crude product was purified by column chromatography (eluted with 50% EtOAc in hexanes) to afford 2-deuteromethyl-5-nitroisoindoline (20.2) (140 mg, 42.3%).

LCMS: 182.3[M+1]⁺.

### 2-Deuteromethylisoindolin-5-amine (20.3)

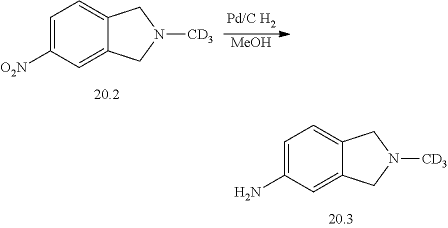 [SMILES: "MeOH" ; [2H]C([2H])([2H])N1Cc2ccc([N+](=O)[O-])cc2C1 ; [2H]C([2H])([2H])N1Cc2ccc(N)cc2C1]

The mixture of 2-deuteromethyl-5-nitroisoindoline (20.2) (140 mg, 0.77 mmol) and Pd/C (10%, 14 mg) in MeOH (10 mL) was stirred at 50° C. for 2 h under H₂ atmosphere. TLC showed the reaction was complete. The reaction solution was filtered through celite, which was washed with MeOH. The combined organic phase was concentrated to afford 2-deuteromethylisoindolin-5-amine (20.3) (80 mg, 69%).

### (R)-1-(3-((5-Fluoro-2-((2-deuteromethylisoindolin-5-yl)amino)pyrimidin-4-yl)amino)-piperidin-1-yl)prop-2-en-1-one (I-20)

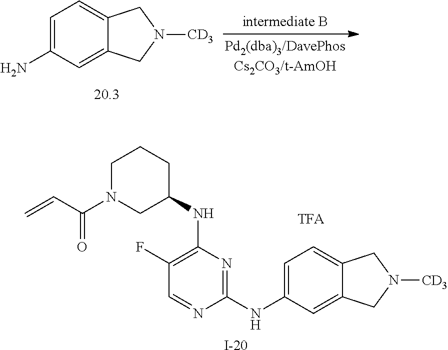

A mixture of 2-deuteromethylisoindolin-5-amine (20.3) (80 mg, 0.53 mmol), (R)-1-(3-(2-chloro-5-fluoropyrimidin-4-ylamino)piperidin-1-yl)prop-2-en-1-one (intermediate B) (180 mg, 0.64 mmol), Pd₂(dba)₃ (73 g, 0.79 mmol), DavePhos (32 mg, 0.79 mmol) and Cs₂CO₃ (345 g, 1.1 mmol) in t-AmOH (10 mL) was stirred at 100° C. for 1.5 h under nitrogen atmosphere. TLC showed the reaction was complete. The reaction mixture was cooled to room temperature and partitioned between EtOAc and H₂O. The organic phase was washed with brine, dried over anhydrous Na₂SO₄ and concentrated under reduced pressure. The crude product was purified by prep-HPLC to afford (R)-1-(3-((5-fluoro-2-((2-deuteromethylisoindolin-5-yl)amino)pyrimidin-4-yl)amino)-piperidin-1-yl)prop-2-en-1-one (I-20) (83.2 mg, 30%) as a TFA salt.

LCMS: 400.3 [M+1]⁺

¹H NMR (400 MHz, CD₃OD): δ 1.55-1.65 (m, 1H), 1.77-1.87 (m, 1H), 1.93-1.98 (m, 1H), 2.11-2.17 (m, 1H), 2.82-2.92 (m, 1H), 3.08-3.24 (m, 1H), 4.07-4.16 (m, 2H), 4.43-4.71 (m, 3H), 5.49 (d, 0.5H), 5.85 (d, 0.5H), 6.08 (d, 0.5H), 6.29 (d, 0.5H), 6.40 (m, 0.5H), 6.87 (dd, 0.5H), 7.40-7.44 (m, 1H), 7.56-7.58 (m, 1H), 7.69 (s, 1H), 7.99 (br, 1H).

### Example 21

### Preparation of (R)-1-(3-((2-((2-cyclopropylisoindolin-5-yl)amino)-5-fluoropyrimidin-4-yl)amino)piperidin-1-yl)prop-2-en-1-one (I-21)

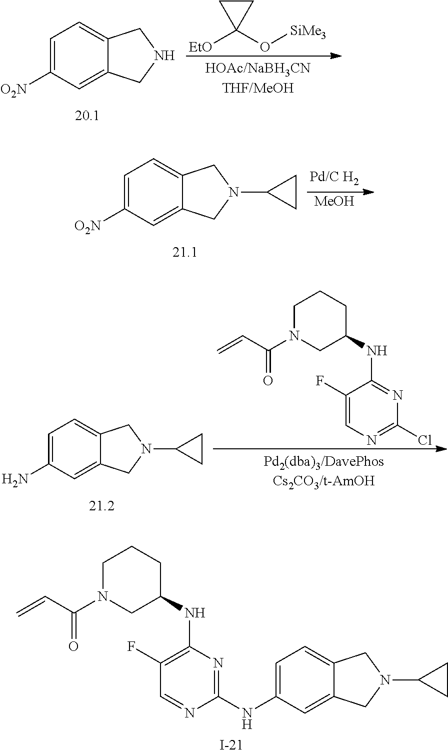

### 2-Cyclopropyl-5-nitroisoindoline (21.1)

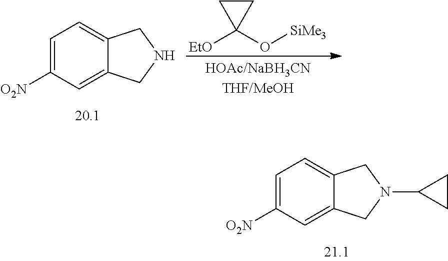 [SMILES: O=[N+]([O-])c1ccc2c(c1)CNC2 ; CCOC1(O[Si](C)(C)C)CC1 ; O=[N+]([O-])c1ccc2c(c1)CN(C1CC1)C2]

To a solution of 5-nitroisoindoline (20.1, 150 mg, 0.91 mmol), HOAc (110 mg, 1.83 mmol), and (1-ethoxycyclopropoxy)-trimethyl-silane (640 mg, 3.64 mmol) in THF (25 mL) and MeOH (2.5 mL) was added NaBH₃CN (86 mg, 1.37 mmol) at 20° C. The mixture was allowed to stir at 60° C. for 18 h. TLC showed the reaction was complete. The reaction mixture was cooled to room temperature and quenched with 1N HCl and extracted with EtOAc. The aqueous layer was basified to pH=10 with solid K₂CO₃ and extracted with DCM. The combined organic phase was washed with water, brine, dried over Na₂SO₄ and concentrated to afford 2-cyclopropyl-5-nitroisoindoline (21.1) (90 mg, 48.2%).

LCMS: 205.2 [M+1]⁺.

### 2-Cyclopropylisoindolin-5-amine (21.2)

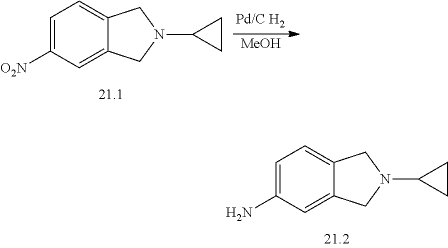 [SMILES: "MeOH" ; O=[N+]([O-])c1ccc2c(c1)CN(C1CC1)C2 ; Nc1ccc2c(c1)CN(C1CC1)C2]

A mixture of 2-cyclopropyl-5-nitroisoindoline (21.1) (90 mg, 0.44 mmol) and Pd/C (10%, 15 mg) in MeOH (10 mL) was stirred at 50° C. for 15 h under H₂ atmosphere. TLC showed the reaction was complete. The reaction solution was filtered through celite, which was washed with MeOH. The combined organic phase was concentrated to afford 2-cyclopropylisoindolin-5-amine (21.2) (50 mg, 65.1%).

### (R)-1-(3-((2-((2-Cyclopropylisoindolin-5-yl)amino)-5-fluoropyrimidin-4-yl)amino)piperidin-1-yl)prop-2-en-1-one (I-21)

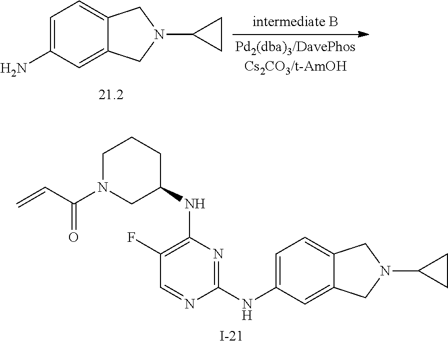

A mixture of 2-cyclopropylisoindolin-5-amine (21.2) (50 mg, 0.18 mmol), 1-[(3R)-3-[(2-chloro-5-fluoro-pyrimidin-4-yl)amino]-1-piperidyl]prop-2-en-1-one (intermediate B, 33 mg, 0.18 mmol), Cs₂CO₃ (110 mg, 0.36 mmol), Pd₂(dba)₃ (16 mg, 0.02 mmol) and DavePhos (16 mg, 0.04 mmol) in t-AmOH (10 mL) was stirred at 100° C. for 2 h under nitrogen atmosphere. TLC showed the reaction was complete. The reaction mixture was cooled to room temperature and partitioned between EtOAc and H₂O. The organic phase was washed with brine, dried over anhydrous Na₂SO₄ and concentrated under reduced pressure. The crude product was purified by prep-HPLC to afford (R)-1-(3-((2-((2-cyclopropylisoindolin-5-yl)amino)-5-fluoropyrimidin-4-yl) amino)piperidin-1-yl)prop-2-en-1-one (I-21) (30 mg, 40.4%).

LCMS: 423.3 [M+1]⁺.

¹H NMR (400 MHz, CD₃OD): δ 0.56-0.61 (m, 4H), 1.58-1.80 (m, 2H), 1.91-1.98 (m, 1H), 2.12-2.18 (m, 2H), 2.77-2.83 (m, 0.5H), 3.01-3.07 (m, 0.5H), 3.16-3.33 (m, 2H), 4.00-4.11 (m, 5H), 4.26 (d, 0.5H), 4.72 (d, 0.5H), 5.51 (d, 0.5H), 5.81 (d, 0.5H), 6.09 (d, 0.5H), 6.29 (d, 0.5H), 6.47-6.55 (m, 0.5H), 6.83-6.89 (m, 0.5H), 7.12 (d, 1H), 7.31-7.36 (m, 1H), 7.60 (s, 0.5H), 7.70-7.76 (m, 1.5H).

### Example 22

### Preparation of (R)-1-(3-((5-fluoro-2-((2-(2,2,2-trifluoroethyl)isoindolin-5-yl)amino)pyrimidin-4-yl)amino)piperidin-1-yl)prop-2-en-1-one (I-22)

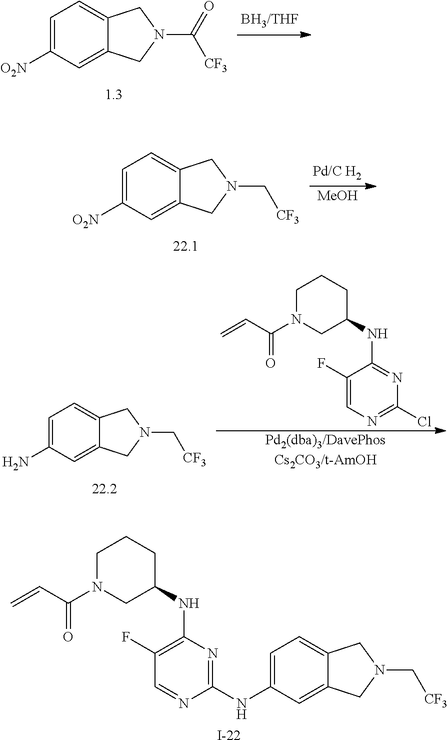

### 5-Nitro-2-(2,2,2-trifluoroethyl)isoindoline (22.1)

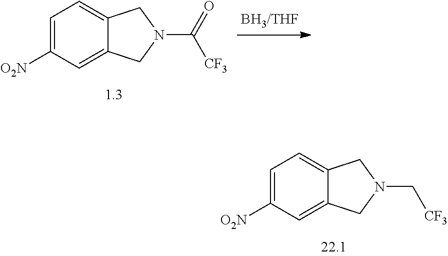 [SMILES: O=C(N1Cc2ccc([N+](=O)[O-])cc2C1)C(F)(F)F ; O=[N+]([O-])c1ccc2c(c1)CN(CC(F)(F)F)C2]

A mixture of 2,2,2-trifluoro-1-(5-nitroisoindolin-2-yl)ethanone (1.3) (500 mg, 1.92 mmol) and BH₃-THF complex (1 M, 15 mL, 15 mmol) in dry THF (20 mL) was stirred at reflux for 3 h. TLC showed the reaction was complete. The reaction mixture was carefully quenched at 0° C. with 2N HCl until gas evolution ceased, then basified with 2N NaOH to pH 10-11. The resulting mixture was extracted with EtOAc and dried over anhydrous Na₂SO₄. The crude product was purified by column chromatography to afford 5-nitro-2-(2,2,2-trifluoroethyl)isoindoline (22.1) (350 mg, 74%).

LCMS: 247.1 [M+1]⁺

### 2-(2,2,2-Trifluoroethyl)isoindolin-5-amine (22.2)

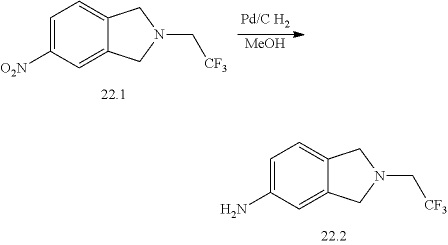 [SMILES: "MeOH" ; O=[N+]([O-])c1ccc2c(c1)CN(CC(F)(F)F)C2 ; Nc1ccc2c(c1)CN(CC(F)(F)F)C2]

A mixture of 5-nitro-2-(2,2,2-trifluoroethyl)isoindoline (22.1) (350 mg, 1.42 mmol) and Pd/C (10%, 50 mg) in MeOH (20 mL) was stirred at 50° C. for 16 h under H₂ atmosphere. TLC showed the reaction was complete. The reaction solution was filtered through celite, which was washed with MeOH. The combined organic phase was concentrated. The crude compound was purified by column chromatography (eluted with 50% EtOAc in hexanes) to afford 2-(2,2,2-trifluoroethyl)isoindolin-5-amine (22.2) (220 mg, 71.7%).

LCMS: 217.2 [M+1]⁺.

### (R)-1-(3-((5-fluoro-2-((2-(2,2,2-trifluoroethyl)isoindolin-5-yl)amino)-pyrimidin-4-yl)amino)piperidin-1-yl)prop-2-en-1-one (I-22)

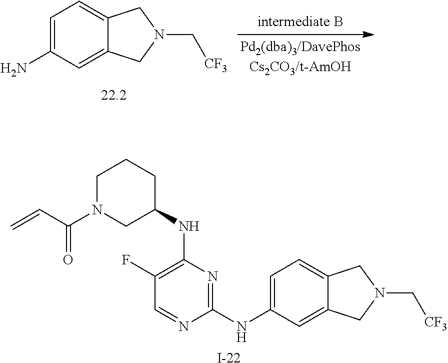

A solution of 2-(2,2,2-trifluoroethyl)isoindolin-5-amine (22.2) (100 mg, 0.46 mmol), 1-[(3R)-3-[(2-chloro-5-fluoro-pyrimidin-4-yl)amino]-1-piperidyl]prop-2-en-1-one (intermediate B) (130 mg, 0.46 mmol), Cs₂CO₃ (300 mg, 0.92 mmol), Pd₂(dba)₃ (42 mg, 0.046 mmol) and DavePhos (42 mg, 0.10 mmol) in t-AmOH (15 mL) was stirred at 100° C. for 2 h under nitrogen atmosphere. TLC showed the reaction was complete. The reaction mixture was cooled to room temperature and partitioned between EtOAc and H₂O. The organic phase was washed with brine, dried over anhydrous Na₂SO₄ and concentrated under reduced pressure. The crude product was purified by prep-HPLC to afford (R)-1-(3-((5-fluoro-2-((2-(2,2,2-trifluoroethyl)isoindolin-5-yl)amino)-pyrimidin-4-yl)amino)piperidin-1-yl)prop-2-en-1-one (I-22) (80 mg, 37.4%).

LCMS: 465.2 [M+1]⁺

¹H NMR (400 MHz, CD₃OD): δ 1.68-2.11 (m, 4H), 3.33-3.48 (m, 4H), 3.65 (br, 0.5H), 3.95-4.12 (m, 6.5H), 4.90 (br, 0.5H), 5.23 (br, 0.5H), 5.54 (d, 0.5H), 5.75 (d, 0.5H), 6.24-6.46 (m, 1.5H), 6.57-6.65 (m, 0.5H), 6.92 (s, 0.5H), 7.12-7.19 (m, 1H), 7.26-7.33 (m, 1.5H), 7.48-7.56 (m, 1H), 7.78-7.81 (m, 1H).

### Example 23

### Preparation of (R)-1-(3-((5-fluoro-2-((2-(2-hydroxyethyl)isoindolin-5-yl)amino)pyrimidin-4-yl)amino)piperidin-1-yl)prop-2-en-1-one (I-23)

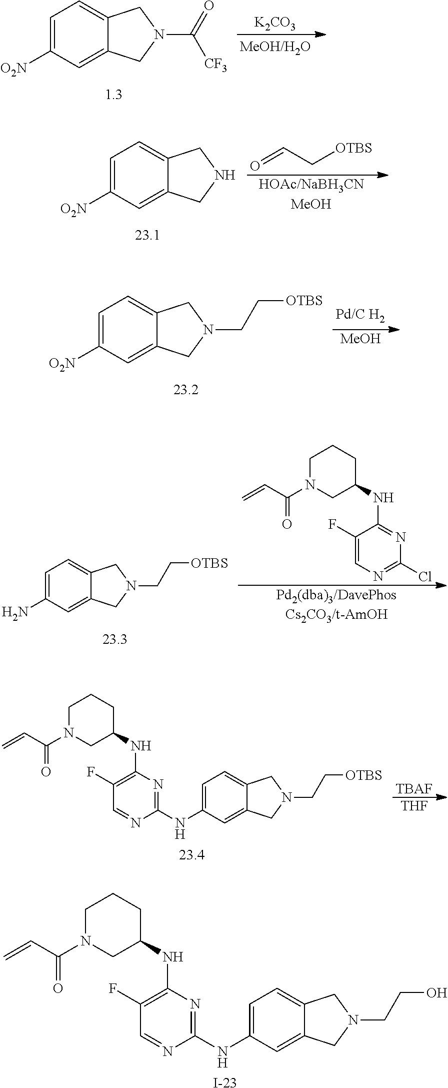

### 5-Nitroisoindoline (23.1)

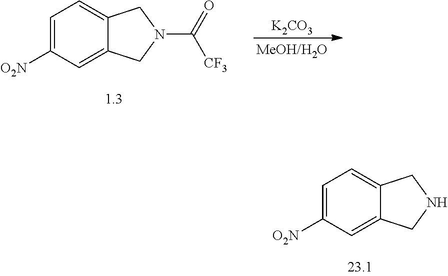

A mixture of 1.3 (500 mg, 1.92 mmol) and K₂CO₃ (530 mg, 3.84 mmol) in MeOH (9 mL), H₂O (1 mL) was stirred at room temperature for 1 h. TLC showed the reaction was complete. The volatiles were removed and the residue was purified by column chromatography to afford 5-nitroisoindoline (23.1) (250 mg, 79.2%).

### 2-(2-((tert-Butyldimethylsilyl)oxy)ethyl)-5-nitroisoindoline (23.2)

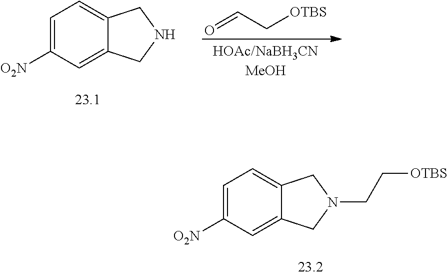 [SMILES: O=[N+]([O-])c1ccc2c(c1)CNC2 ; CC(C)(C)[Si](C)(C)OCC=O ; CC(C)(C)[Si](C)(C)OCCN1Cc2ccc([N+](=O)[O-])cc2C1]

A mixture of 5-nitroisoindoline (23.1) (100 mg, 0.61 mmol), HOAc (0.05 mL, 0.91 mmol) and 2-[tert-butyl (dimethyl)silyl]oxyacetaldehyde (160 mg, 0.91 mmol) in MeOH (10 mL) was stirred at room temperature for 20 min. NaBH₃CN (58 mg, 0.91 mmol) was added and the resulting mixture was stirred at room temperature overnight. TLC showed the reaction was complete. The resulting mixture was partitioned between DCM and 0.5 N NaHCO₃. The organic phase was washed with brine and dried over anhydrous Na₂SO₄. The crude product was purified by column chromatography to afford 2-(2-((tert-butyldimethylsilyl)oxy)ethyl)-5-nitroisoindoline (23.2) (150 mg, 76.4%).

LCMS: 323.3 [M+1]⁺.

### 2-(2-((tert-Butyldimethylsilyl)oxy)ethyl)isoindolin-5-amine (23.3)

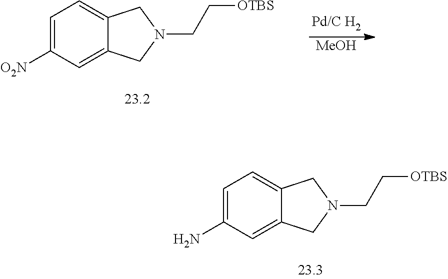 [SMILES: "MeOH" ; CC(C)(C)[Si](C)(C)OCCN1Cc2ccc([N+](=O)[O-])cc2C1 ; CC(C)(C)[Si](C)(C)OCCN1Cc2ccc(N)cc2C1]

The mixture of 2-(2-((tert-butyldimethylsilyl)oxy)ethyl)-5-nitroisoindoline (23.2) (150 mg, 0.46 mmol) and Pd/C (10%, 30 mg) in MeOH (15 mL) was stirred at 50° C. for 16 h under H₂ atmosphere. TLC showed the reaction was complete. The reaction solution was filtered through celite, which was washed with MeOH. The combined organic phase was concentrated to afford 2-(2-((tert-butyldimethylsilyl)oxy)ethyl)isoindolin-5-amine (23.3) (120 mg, 88.2%).

LCMS: 293.3 [M+1]⁺.

### (R)-1-(3-((2-((2-(2-((tert-Butyldimethylsilyl)oxy)ethyl)isoindolin-5-yl)amino)-5-fluoro-pyrimidin-4-yl)amino)piperidin-1-yl)prop-2-en-1-one (23.4)

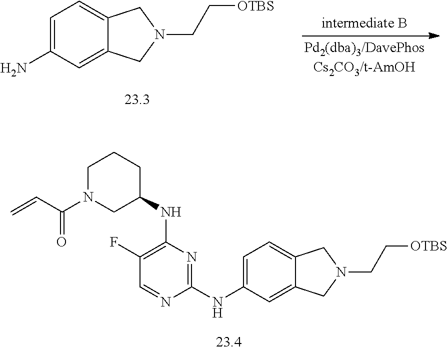 [SMILES: CC(C)(C)[Si](C)(C)OCCN1Cc2ccc(N)cc2C1 ; C=CC(=O)N1CCC[C@@H](Nc2nc(Nc3ccc4c(c3)CN(CCO[Si](C)(C)C(C)(C)C)C4)ncc2F)C1]

A mixture of 2-(2-((tert-butyldimethylsilyl)oxy)ethyl)isoindolin-5-amine (23.3) (120 mg, 0.41 mmol), 1-[(3R)-3-[(2-chloro-5-fluoro-pyrimidin-4-yl)amino]-1-piperidyl]prop-2-en-1-one (intermediate B, 116 mg, 0.41 mmol), Cs₂CO₃ (270 mg, 0.82 mmol), Pd₂(dba)₃ (38 mg, 0.041 mmol) and DavePhos (38 mg, 0.092 mmol) in t-AmOH (10 mL) was stirred at 100° C. for 2 h under nitrogen atmosphere. TLC showed the reaction was complete. The reaction mixture was cooled to room temperature and partitioned between EtOAc and H₂O. The organic phase was washed with brine, dried over anhydrous Na₂SO₄ and concentrated under reduced pressure. The crude product was purified by column chromatography to afford (R)-1-(3-((2-((2-(2-((tert-butyldimethylsilyl)oxy)ethyl)isoindolin-5-yl)amino)-5-fluoro-pyrimidin-4-yl)amino)piperidin-1-yl)prop-2-en-1-one (23.4) (150 mg, 68.1%).

LCMS: 541.4[M+1]⁺

### (R)-1-(3-((5-Fluoro-2-((2-(2-hydroxyethyl)isoindolin-5-yl)amino)-pyrimidin-4-yl)amino)piperidin-1-yl)prop-2-en-1-one (I-23)

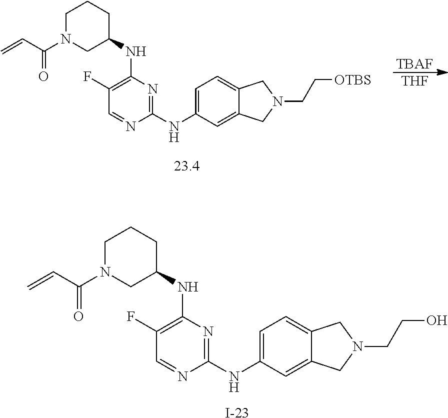

A solution of (R)-1-(3-((2-((2-(2-((tert-butyldimethylsilyl)oxy)ethyl)isoindolin-5-yl)amino)-5-fluoro-pyrimidin-4-yl)amino)piperidin-1-yl)prop-2-en-1-one (23.4) (150 mg, 0.28 mmol) and TBAF (145 mg, 0.56 mmol) in THF (10 mL) was stirred at room temperature for 1 h. TLC showed the reaction was complete. The resulting mixture was partitioned between DCM and water. The organic phase was washed with brine, dried over anhydrous Na₂SO₄ and concentrated under reduced pressure. The crude compound was purified by column chromatography to afford (R)-1-(3-((5-fluoro-2-((2-(2-hydroxyethyl)isoindolin-5-yl)amino)-pyrimidin-4-yl)amino)piperidin-1-yl)prop-2-en-1-one (I-23) (50 mg, 42.4%).

LCMS: 427.3 [M+1]⁺.

¹H NMR (400 MHz, CD₃OD): δ 1.55-1.64 (m, 1H), 1.77-1.82 (m, 1H), 1.93-1.97 (m, 1H), 2.11-2.15 (m, 1H), 2.85 (t, 1H), 3.12-3.24 (m, 1H), 3.53-3.57 (m, 2H), 3.92-4.16 (m, 4H), 4.42-4.86 (m, 5H), 5.51 (d, 0.5H), 5.84 (d, 0.5H), 6.08 (d, 0.5H), 6.29 (d, 0.5H), 6.42 (br, 0.5H), 6.83-6.90 (m, 0.5H), 7.40-7.43 (m, 1H), 7.55-7.58 (m, 1H), 7.70 (s, 1H), 7.98 (s, 1H).

### Example 24

### Preparation of (R)-1-(3-((5-fluoro-2-((2-(4-fluorophenyl)isoindolin-5-yl)amino)pyrimidin-4-yl)amino)piperidin-1-yl)prop-2-en-1-one (I-24)

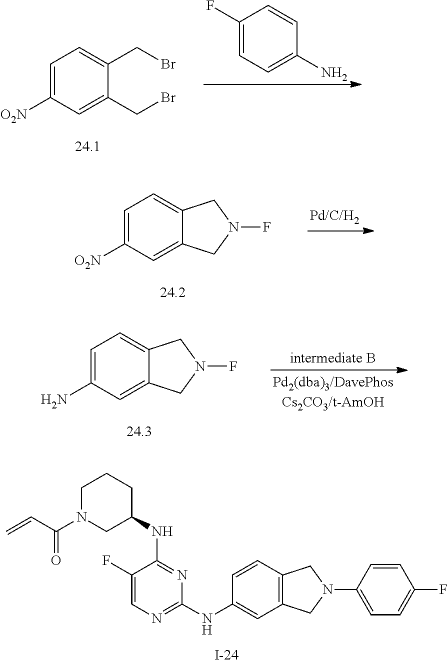

### 2-(4-Fluorophenyl)-5-nitroisoindoline (24.2)

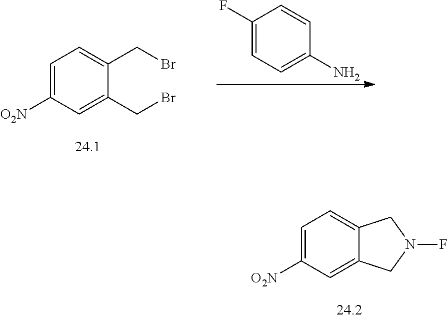 [SMILES: O=[N+]([O-])c1ccc(CBr)c(CBr)c1 ; Nc1ccc(F)cc1 ; O=[N+]([O-])c1ccc2c(c1)CN(F)C2]

4-Fluoroaniline (72 mg, 0.65 mmol) was added to the solution of 1,2-bis(bromomethyl)-4-nitrobenzene (24.1, 200 mg, 0.65 mmol) and DIPEA (167 mg, 1.3 mmol) in toluene (10 mL). The resulting mixture was heated at 110° C. overnight. After cooling to room temperature, the reaction mixture was diluted with water and extracted with ethyl acetate. The combined organic layers were washed with water and brine, dried over sodium sulfate and concentrated in vacuo. The crude product was purified by column chromatography (hexanes/EA:20/1) to afford 2-(4-fluorophenyl)-5-nitroisoindoline (24.2) as a yellow solid (150 mg, 80%).

¹H NMR (400 MHz, DMSO): 4.69 (s, 4H), 6.72-6.64 (m, 2H), 7.17-7.09 (m, 2H), 7.68 (d, J=8.39 Hz, 1H), 8.22 (dd, J=8.30, 2.13 Hz, 1H), 8.29 (d, J=1.84 Hz, 1H).

### 2-(4-Fluorophenyl)-5-nitroisoindoline (24.3)

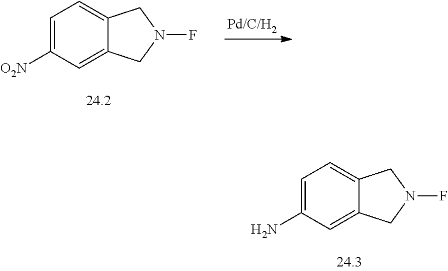 [SMILES: O=[N+]([O-])c1ccc2c(c1)CN(F)C2 ; Nc1ccc2c(c1)CN(F)C2]

Pd/C (100 mg) was added to a solution of 2-(4-fluorophenyl)-5-nitroisoindoline (24.2) (150 mg, 0.58 mmol) in MeOH (15 mL). The reaction mixture was stirred at room temperature overnight under H₂. The reaction mixture was filtered and concentrated to afford 2-(4-fluorophenyl)-5-nitroisoindoline (24.3) as a yellow solid (100 mg, 75%).

LCMS: 229.2[M+1]⁺.

### (R)-1-(3-(5-Fluoro-2-(2-(4-fluorophenyl)isoindolin-5-ylamino)pyrimidin-4-ylamino)-piperidin-1-yl)prop-2-en-1-one (I-24)

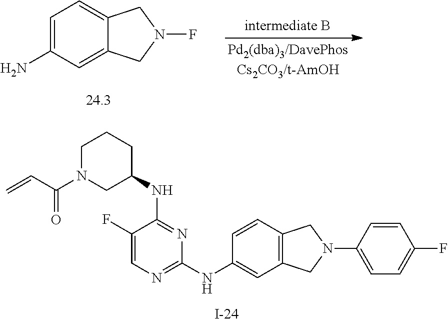

(R)-1-(3-(2-Chloro-5-fluoropyrimidin-4-ylamino)piperidin-1-yl)prop-2-en-1-one (intermediate B, 137 mg, 0.48 mmol) and Cs₂CO₃ (280 mg, 0.86 mmol) were added to a solution of 2-(4-fluorophenyl)-5-nitroisoindoline (24.3) (100 mg, 0.43 mmol) in t-AmOH (10 mL). Pd₂(dba)₃ (40 mg, 0.04 mmol) and DavePhos (25 mg, 0.04 mmol) were added under N₂. The reaction mixture was stirred at 100° C. for 2 h. The reaction mixture was diluted with water and extracted with ethyl acetate. The combined organic layers were washed with water and brine, dried over sodium sulfate and concentrated in vacuo. The crude product was purified by column chromatography (dichloromethane/methanol:10/1) to afford (R)-1-(3-(5-fluoro-2-(2-(4-fluorophenyl)isoindolin-5-ylamino)pyrimidin-4-ylamino)-piperidin-1-yl)prop-2-en-1-one (I-24) as a brown solid (80 mg, 40%).

LCMS: 477.4[M+1]⁺.

¹H NMR (400 MHz, DMSO-d₆): δ 1.52-1.40 (m, 1H), 1.71-1.61 (m, 1H), 1.87-1.79 (m, 1H), 2.02-1.99 (m, 1H), 2.84-2.67 (m, 1H), 3.00 (s, 0.5H), 3.20-3.12 (m, 0.5H), 4.04 (m, 2H), 4.29-4.18 (m, 0.5H), 4.47 (s, 4.5H), 5.55-5.48 (m, 0.5H), 5.77-5.68 (m, 0.5H), 6.18-6.01 (m, 1H), 6.72-6.57 (m, 2.5H), 6.96-6.86 (m, 1H), 7.08 (t, J=8.82 Hz, 2H), 7.19 (d, J=8.27 Hz, 1H), 7.47 (s, 2H), 7.98-7.83 (m, 2H), 9.16 (s, 1H).

### Example 25

### Preparation of (R)-1-(3-((2-((2-(tert-butyl)isoindolin-5-yl)amino)-5-fluoropyrimidin-4-yl)amino)piperidin-1-yl)prop-2-en-1-one (I-25)

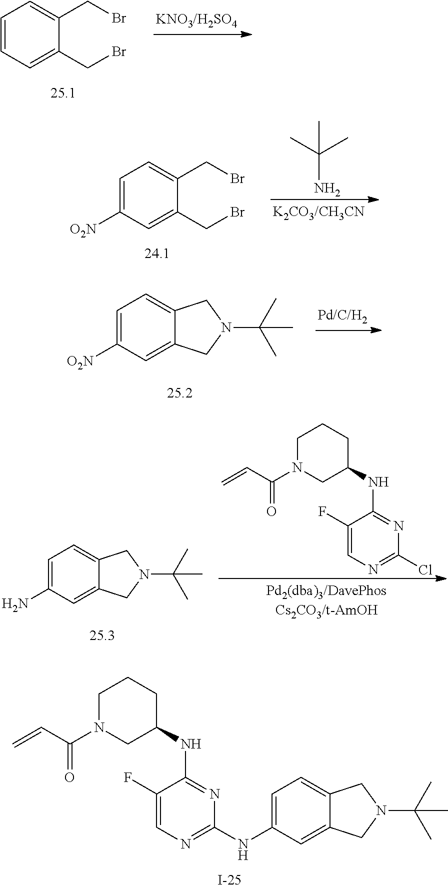

### 1,2-Bis(bromomethyl)-4-nitrobenzene (24.1)

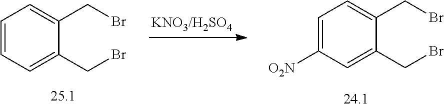 [SMILES: BrCc1ccccc1CBr ; O=[N+]([O-])c1ccc(CBr)c(CBr)c1]

KNO₃ (920 mg, 9.1 mmol) was slowly added to the solution of 1,2-bis(bromomethyl)benzene (25.1) 2.0 g, 7.6 mmol) in H₂SO₄ (10 mL) at 0° C. The mixture was stirred at 0° C. for 2 hand then poured into ice water and filtered to afford 1,2-bis(bromomethyl)-4-nitrobenzene (24.1) as a yellow solid (1.0 g, 43%).

¹H NMR (400 MHz, DMSO-d₆): δ 4.92 (s, 2H), 4.96 (s, 2H), 7.79 (d, J=8.52 Hz, 1H), 8.20 (dd, J=8.49, 2.48 Hz, 1H), 8.41 (d, J=2.45 Hz, 1H).

### 2-tert-Butyl-5-nitroisoindoline (25.2)

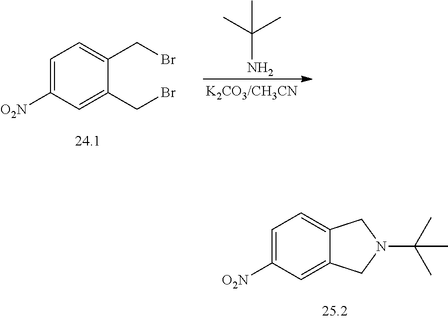

2-Methylpropan-2-amine (71 mg, 0.97 mmol) was added to a solution of 1,2-bis(bromomethyl)-4-nitrobenzene (24.1) (300 mg, 0.97 mmol) and K₂CO₃ (267 mg, 1.94 mmol) in CH₃CN (5 mL). The resulting mixture was heated at 90° C. for 2 h. After cooling to room temperature, the reaction mixture was diluted with water and extracted with ethyl acetate. The combined organic layers were washed with water and brine, dried over sodium sulfate and concentrated in vacuo. The crude product was purified by column chromatography (dichloromethane/methanol:20/1) to afford 2-tert-butyl-5-nitroisoindoline (25.2) as a yellow solid (160 mg, 74%).

LCMS: 221.2 [M+1]⁺.

### 2-tert-Butylisoindolin-5-amine (25.3)

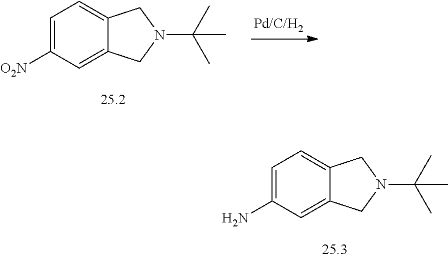 [SMILES: CC(C)(C)N1Cc2ccc([N+](=O)[O-])cc2C1 ; CC(C)(C)N1Cc2ccc(N)cc2C1]

Pd/C (100 mg) was added to a solution of 2-tert-butyl-5-nitroisoindoline (25.2) (160 mg, 0.73 mmol) in MeOH (15 mL). The reaction mixture was stirred at room temperature overnight under H₂. The reaction mixture was filtered and concentrated to afford 2-tert-butylisoindolin-5-amine (25.3) as a yellow solid (130 mg, 94%).

LCMS: 191.3 [M+1]⁺.

### (R)-1-(3-(2-(2-tert-Butylisoindolin-5-ylamino)-5-fluoropyrimidin-4-ylamino)piperidin-1-yl) prop-2-en-1-one (I-25)

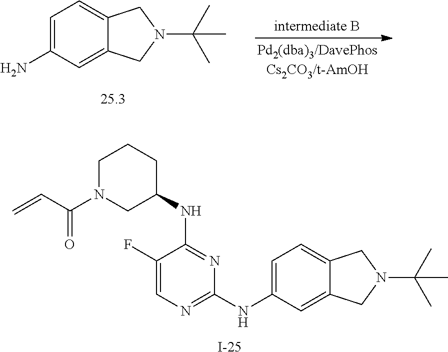

(R)-1-(3-(2-Chloro-5-fluoropyrimidin-4-ylamino)piperidin-1-yl)prop-2-en-1-one (intermediate B) (234 mg, 0.82 mmol) and Cs₂CO₃ (443 mg, 1.36 mmol) were added to a solution of 2-tert-butylisoindolin-5-amine (25.3) (130 mg, 0.68 mmol) in t-AmOH (10 mL). Pd₂(dba)₃ (62 mg, 0.07 mmol) and DavePhos (53 mg, 0.14 mmol) were added under N₂. The reaction mixture was stirred at 100° C. for 2 h. The reaction mixture was diluted with water and extracted with ethyl acetate. The combined organic layers were washed with water and brine, dried over sodium sulfate and concentrated in vacuo. The crude product was purified by column chromatography (dichloromethane/methanol:10/1) to afford (R)-1-(3-(2-(2-tert-Butylisoindolin-5-ylamino)-5-fluoropyrimidin-4-ylamino)piperidin-1-yl) prop-2-en-1-one (I-25) as a yellow solid (75 mg, 25%).

LCMS: 439.2 [M+1]⁺.

¹H NMR (400 MHz, DMSO-d₆): δ 1.23 (s, 9H), 1.52-1.40 (m, 1H), 1.74-1.60 (m, 1H), 1.89-1.78 (m, 1H), 2.05-1.94 (m, 1H), 2.82-2.66 (m, 1H), 3.18-2.99 (m, 1H), 4.55-4.02 (m, 7H), 5.57-5.46 (m, 0.5H), 5.75-5.65 (m, 0.5H), 6.20-6.01 (m, 1H), 6.73-6.61 (m, 0.5H), 6.93-6.82 (m, 0.5H), 7.17-7.05 (m, 1H), 7.56-7.32 (m, 2H), 7.83-7.70 (m, 1H), 7.91 (d, J=3.57 Hz, 1H), 9.27-9.06 (m, 1H).

### Example 26

### Preparation of 1-(6-((5-Fluoro-2-((2-methylisoindolin-5-yl)amino)pyrimidin-4-yl)amino)-2-azaspiro[3.3]heptan-2-yl)prop-2-en-1-one (I-26)

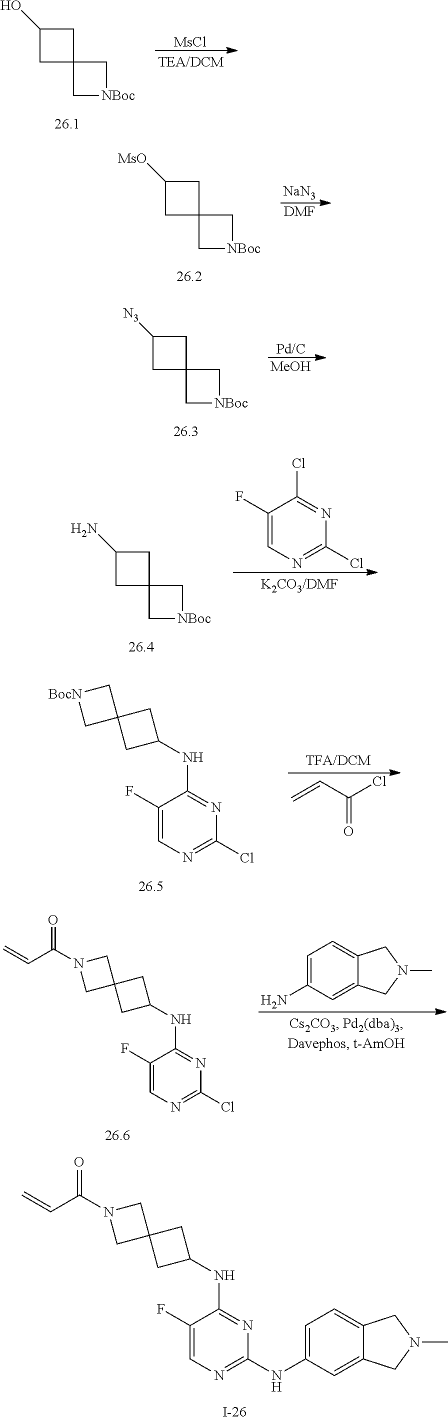

### tert-Butyl 6-((methylsulfonyl)oxy)-2-azaspiro[3.3]heptane-2-carboxylate (26.2)

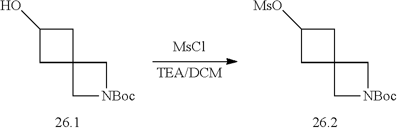 [SMILES: "MsCl" ; CC(C)(C)OC(=O)N1CC2(CC(O)C2)C1 ; "TEA/DCM" ; CC(C)(C)OC(=O)N1CC2(CC(OS(C)(=O)=O)C2)C1]

To a solution of tert-butyl 6-hydroxy-2-azaspiro[3.3]heptane-2-carboxylate (26.1) (500 mg, 2.34 mmol) and TEA (472 mg, 4.68 mmol) in DCM (5 mL) was added MsCl (322 mg, 2.81 mmol) dropwise at 0° C. The resulting mixture was stirred at 0° C. for 2 h. TLC showed complete reaction. The mixture was poured into water and extracted with DCM. The organic phase was washed with water and brine, dried over Na₂SO₄ and concentrated to afford tert-butyl 6-((methylsulfonyl)oxy)-2-azaspiro[3.3]heptane-2-carboxylate (26.2) (700 mg, crude), which was used in next step without further purification.

### tert-Butyl 6-azido-2-azaspiro[3.3]heptane-2-carboxylate (26.3)

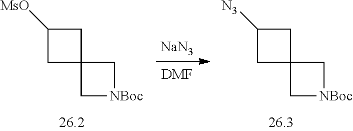

A mixture of tert-butyl 6-((methylsulfonyl)oxy)-2-azaspiro[3.3]heptane-2-carboxylate (26.2) (700 mg, crude), NaN₃ (456 mg, 7.02 mmol) and 15-crown-5 (50 mg) in DMF (10 mL) was stirred at 70° C. overnight. TLC showed the reaction was complete. The reaction mixture was cooled to room temperature and partitioned between EtOAc and water. The organic phase was washed with water and brine, dried over Na₂SO₄ and concentrated under reduced pressure. The crude product was purified by column chromatography to afford tert-butyl 6-azido-2-azaspiro[3.3]heptane-2-carboxylate (26.3) (500 mg, 90%, 2 steps) as a yellow oil.

### tert-Butyl 6-amino-2-azaspiro[3.3]heptane-2-carboxylate (26.4)

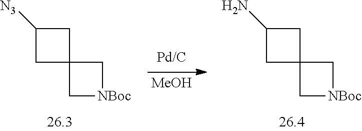 [SMILES: "MeOH" ; CC(C)(C)OC(=O)N1CC2(CC(N=[N+]=[N-])C2)C1 ; CC(C)(C)OC(=O)N1CC2(CC(N)C2)C1]

A mixture of tert-butyl 6-azido-2-azaspiro[3.3]heptane-2-carboxylate (26.3) (500 mg, 2.10 mmol) and 10% Pd/C (50 mg) in MeOH (10 mL) was stirred at room temperature under hydrogen atmosphere overnight. TLC showed complete reaction. Pd/C was removed by filtration and washed with MeOH. The filtrate was concentrated to afford tert-butyl 6-amino-2-azaspiro[3.3]heptane-2-carboxylate (26.4) (400 mg, crude) as a light yellow oil, which was used in the next step without further purification.

### tert-Butyl 6-((2-chloro-5-fluoropyrimidin-4-yl)amino)-2-azaspiro[3.3]heptane-2-carboxylate (26.5)

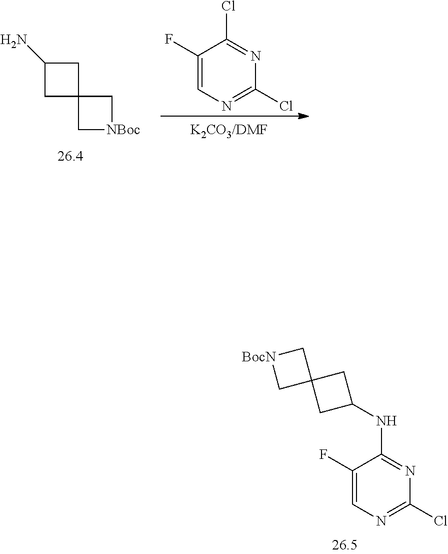 [SMILES: CC(C)(C)OC(=O)N1CC2(CC(N)C2)C1 ; Fc1cnc(Cl)nc1Cl ; CC(C)(C)OC(=O)N1CC2(CC(Nc3nc(Cl)ncc3F)C2)C1]

A mixture of tert-butyl 6-amino-2-azaspiro[3.3]heptane-2-carboxylate (26.4) (400 mg, 1.89 mmol), 2,4-dichloro-5-fluoropyrimidine (315 mg, 1.89 mmol) and K₂CO₃ (525 mg, 3.98 mmol) in DMF (10 mL) was stirred at 50° C. for 2 h. TLC showed the reaction was complete. The reaction mixture was cooled to room temperature and partitioned between EtOAc and water. The organic phase was washed with water and brine, dried over Na₂SO₄ and concentrated under reduced pressure. The crude product was purified by column chromatography to afford tert-butyl 6-((2-chloro-5-fluoropyrimidin-4-yl)amino)-2-azaspiro[3.3]heptane-2-carboxylate (26.5) (600 mg, 92.3%) as a white solid.

¹H NMR (400 MHz, DMSO-d₆): δ 1.44 (s, 9H), 2.13-2.19 (m, 2H), 2.71-2.77 (m, 2H), 3.89 (s, 2H), 4.02 (s, 2H), 4.44-4.53 (m, 1H), 5.33 (br, 1H), 7.89 (d, 1H).

### 1-(6-(2-Chloro-5-fluoropyrimidin-4-ylamino)-2-azaspiro[3.3]heptan-2-yl)prop-2-en-1-one (26.6)

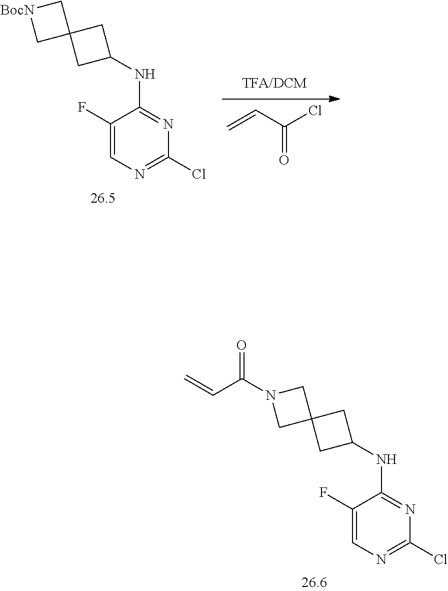

A solution of tert-butyl 6-(2-chloro-5-fluoropyrimidin-4-ylamino)-2-azaspiro[3.3]heptane-2-carboxylate (26.5, 130 mg, 0.38 mmol) and TFA (2 mL) in DCM (2 mL) was stirred at room temperature for 30 min. The mixture was concentrated and the residue was dissolved in DCM. Saturated NaHCO₃ solution (2 mL) was added and the mixture was stirred at 0° C. Acryloyl chloride (31.0 mg 0.34 mmol) was added slowly at 0° C. The reaction solution was stirred at 0° C. for 1 h and TLC showed the reaction was complete. The mixture was poured into water and extracted with DCM. The organic layer was washed with water and brine, dried over Na₂SO₄ and concentrated under reduced pressure. The crude product was purified by column chromatography to afford 1-(6-(2-chloro-5-fluoropyrimidin-4-ylamino)-2-azaspiro[3.3]heptan-2-yl)prop-2-en-1-one (26.6) as a yellow oil (92 mg, 81.6%).

¹H NMR (400 MHz, CDCl₃): δ 2.22-2.76 (m, 2H), 2.75-2.82 (m, 2H), 4.05 (s, 1H), 4.18 (d, 2H), 4.31 (s, 1H), 4.49-4.57 (m, 1H), 5.43 (dd, 1H), 5.68 (t, 1H), 6.12-6.22 (m 1H), 6.33 (d, 1H), 7.90 (d, 1H)

### 1-(6-(5-Fluoro-2-(2-methylisoindolin-5-ylamino)pyrimidin-4-ylamino)-2-azaspiro[3.3]heptan-2-yl)prop-2-en-1-one (I-26)

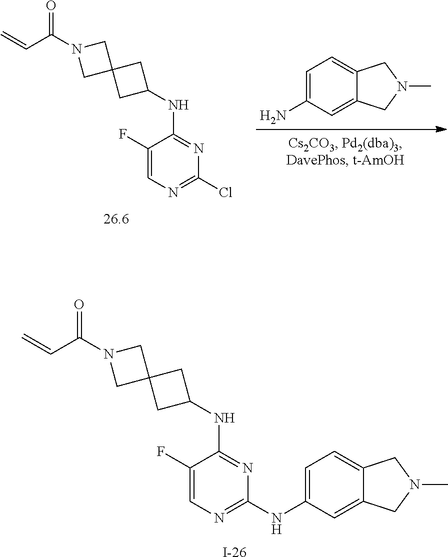

A stirred solution of 1-(6-(2-chloro-5-fluoropyrimidin-4-ylamino)-2-azaspiro[3.3]heptan-2-yl)prop-2-en-1-one (26.6) (100 mg, 0.33 mmol), 2-methylisoindolin-5-amine (50 mg, 0.33 mmol), DavePhos (26.5 mg, 0.06 mmol), Pd₂(dba)₃ (30.8 mg, 0.034 mmol) and Cs₂CO₃ (219 mg, 0.69 mmol) in tert-amyl alcohol (15 mL) was heated at 100° C. overnight under N₂. TLC showed the reaction was complete. The reaction mixture was quenched with water and extracted with ethyl acetate. The combined organic layers were washed with water and brine, dried over sodium sulfate and concentrated in vacuo. The crude product was purified by column chromatography (DCM/MeOH:20/1) to afford 1-(6-(5-fluoro-2-(2-methylisoindolin-5-ylamino)pyrimidin-4-ylamino)-2-azaspiro[3.3]heptan-2-yl)prop-2-en-1-one (I-26) as a white solid (21.2 mg, 15.4%).

LCMS: 409.2 [M+1]⁺

¹H NMR (400 MHz, CD₃OD): 2.38-2.46 (m, 2H), 2.65-2.71 (m, 2H), 3.17 (d, 3H), 4.01 (s, 1H), 4.15 (s, 1H), 4.28 (s, 1H), 4.39 (s, 1H), 4.46-4.61 (m, 3H), 4.94 (s, 2H), 5.76 (d, 1H), 6.23-6.35 (m, 2H), 7.48 (d, 1H), 7.60-7.68 (m, 2H), 7.92 (t, 1H).

### Example 27

### Preparation of (R)-1-(3-((5-Fluoro-2-((1,2,3,4-tetrahydroisoquinolin-6-yl)amino)pyrimidin-4-yl)amino)piperidin-1-yl)prop-2-en-1-one (I-27)

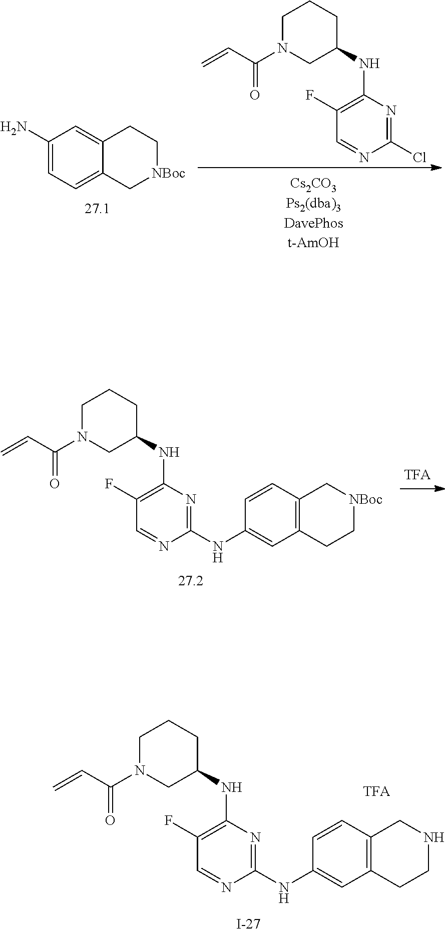

### (R)-tert-Butyl 6-((4-((1-acryloylpiperidin-3-yl)amino)-5-fluoropyrimidin-2-yl)amino)-3,4-dihydroisoquinoline-2(1H)-carboxylate (27.2)

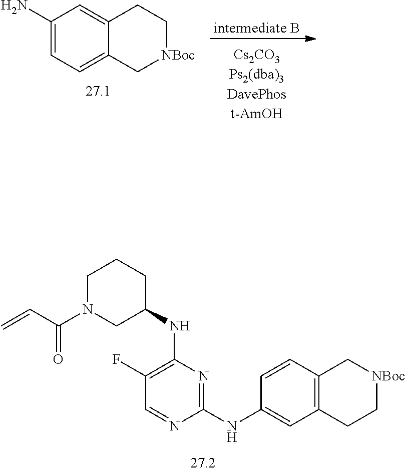

To a solution of tert-butyl 6-amino-3,4-dihydro-1H-isoquinoline-2-carboxylate (27.1) (1.0 g, 4.03 mmol) in toluene (15 mL) was added 1-[(3R)-3-[(2-chloro-5-fluoro-pyrimidin-4-yl)amino]-1-piperidyl]prop-2-en-1-one (intermediate B) (1.38 g, 4.83 mmol), Cs₂CO₃ (2.62 g, 8.05 mmol), Pd₂(dba)₃ (0.37 g, 0.40 mmol) and DavePhos (0.16 g, 0.40 mmol). The mixture was stirred at 100° C. overnight. TLC showed the reaction was complete. The mixture was quenched with water, extracted with EtOAc, washed with brine, dried over Na₂SO₄ and concentrated in vacuo to afford the crude product. The crude compound was purified by column chromatography (DCM/EtOAc=5/1) to afford (R)-tert-butyl 6-((4-((1-acryloylpiperidin-3-yl)amino)-5-fluoropyrimidin-2-yl)amino)-3,4-dihydroisoquinoline-2(1H)-carboxylate (27.2) as a yellow solid (800 mg, 40%).

¹H NMR (400 MHz, DMSO-d₆): δ 1.42 (s, 9H), 1.64-1.66 (m, 1.5H), 1.81-1.84 (m, 1H), 1.97-2.00 (m, 1H), 2.64-2.67 (m, 2.5H), 2.71-2.79 (m, 1H), 2.98-3.05 (m, 1H), 3.12-3.16 (m, 0.5H), 3.49 (s, 2H), 3.99-4.04 (m, 2H), 4.19-4.23 (m, 0.5H), 4.38 (br, 2H), 4.45 (d, 0.5H), 5.45 (d, 0.5H), 5.71 (d, 0.5H), 6.01 (dd, 0.5H), 6.12 (dd, 0.5H), 6.59 (dd, 0.5H), 6.86 (dd, 0.5H), 6.96 (d, 1H), 7.37 (dd, 2H), 7.63 (br, 1H), 7.90 (d, 1H), 9.04 (s, 1H).

### (R)-1-(3-((5-Fluoro-2-((1,2,3,4-tetrahydroisoquinolin-6-yl)amino)pyrimidin-4-yl)amino)-piperidin-1-yl)prop-2-en-1-one (I-27)

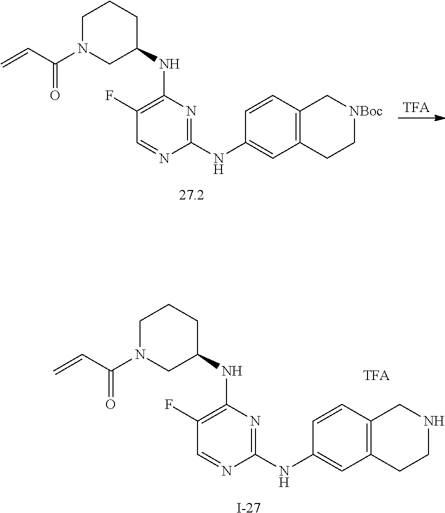

To a solution of (R)-tert-butyl 6-((4-((1-acryloylpiperidin-3-yl)amino)-5-fluoropyrimidin-2-yl)amino)-3,4-dihydroisoquinoline-2(1H)-carboxylate (27.2) (100 mg, 0.20 mmol) in DCM (3 mL) was added 2,2,2-trifluoroacetic acid (3 mL). The mixture was stirred at room temperature for 1 h. TLC showed that the reaction was complete. The mixture was concentrated to remove the excess TFA and washed with Et₂O. The mixture was concentrated in vacuo to afford the TFA salt of (R)-1-(3-((5-fluoro-2-((1,2,3,4-tetrahydroisoquinolin-6-yl)amino)pyrimidin-4-yl)amino)-piperidin-1-yl)prop-2-en-1-one (I-27) as a white solid (100 mg, 97%).

LCMS: 397.3 [M+1]⁺

¹H NMR (400 MHz, CD₃OD): δ 1.56-1.60 (m, 1H), 1.80-1.83 (m, 1H), 1.93-1.98 (m, 1H), 2.15 (t, 1H), 2.81 (t, 0.5H), 2.93 (t, 0.5H), 3.00-3.13 (m, 2.5H), 3.23 (t, 0.5H), 3.50 (t, 2H), 4.06-4.17 (m, 2H), 4.36 (s, 2H), 4.47 (d, 0.5H), 4.64 (d, 0.5H), 5.45 (d, 0.5H), 5.83 (d, 0.5H), 6.15 (d, 0.5H), 6.24-6.32 (m, 1H), 6.85 (dd, 0.5H), 7.25 (t, 1H), 7.39-7.55 (m, 2H), 7.97 (dd, 1H).

### Example 28

### Preparation of (R)-1-(3-((5-Fluoro-2-((2-methyl-1,2,3,4-tetrahydroisoquinolin-6-yl)amino)-pyrimidin-4-yl)amino)piperidin-1-yl)prop-2-en-1-one (I-28)

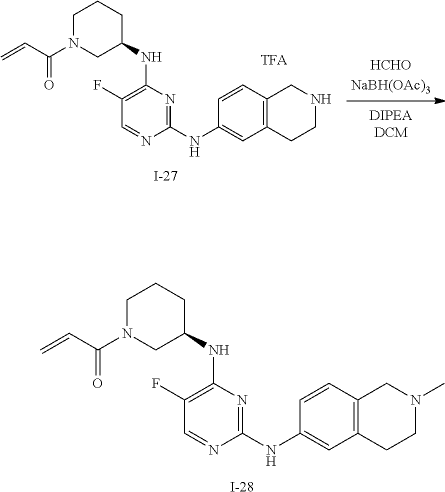

To a solution of (R)-1-(3-((5-fluoro-2-((1,2,3,4-tetrahydroisoquinolin-6-yl)amino)pyrimidin-4-yl)amino)-piperidin-1-yl)prop-2-en-1-one (I-27) (79 mg, 0.20 mmol) in DCM (5 mL) was added DIPEA (51.4 mg, 0.40 mmol) to adjust pH=9 followed by addition of formaldehyde (64.7 mg, 0.80 mmol). The mixture was stirred at room temperature for 30 min. NaBH(OAc)₃ (84.46 mg, 0.40 mmol) was added. The mixture was stirred at room temperature for 3 h. TLC showed that the reaction was complete. The mixture was quenched with water, extracted with DCM, washed with water and concentrated in vacuo to afford the crude product. The crude compound was purified by column chromatography to afford (R)-1-(3-((5-fluoro-2-((2-methyl-1,2,3,4-tetrahydroisoquinolin-6-yl)amino)-pyrimidin-4-yl)amino)piperidin-1-yl)prop-2-en-1-one (I-28) as a yellow solid (30.8 mg, 38%).

LCMS: 411.3 [M+1]⁺

¹H NMR (400 MHz, DMSO-d₆): δ1.41 (br, 1H), 1.63 (br, 1H), 1.79-1.84 (m, 1H), 1.99 (d, 1H), 2.34 (s, 3H), 2.57 (s, 2H), 2.67-2.80 (m, 3H), 3.08-3.13 (m, 1H), 3.42 (s, 2H), 4.01 (d, 2H), 4.19 (d, 0.5H), 4.43 (d, 0.5H), 5.47 (d, 0.5H), 5.71 (d, 0.5H), 6.06 (dd, 1H), 6.60 (dd, 0.5H), 6.85 (d, 1H), 7.33 (dd, 2H), 7.56 (d, 1H), 7.89 (d, 1H), 8.96 (s, 1H).

### Example 29

### Preparation of (R)-1-(3-((5-Fluoro-2-((2-isopropyl-1,2,3,4-tetrahydroisoquinolin-6-yl)amino)-pyrimidin-4-yl)amino)piperidin-1-yl)prop-2-en-1-one (I-29)

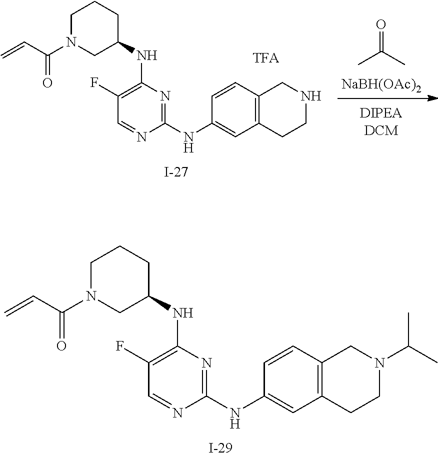

To a solution of (R)-1-(3-((5-fluoro-2-((1,2,3,4-tetrahydroisoquinolin-6-yl)amino)pyrimidin-4-yl)amino)-piperidin-1-yl)prop-2-en-1-one (I-27) (127 mg, 0.32 mmol) was added DIPEA (82.6 mg, 0.64 mmol) to adjust pH=9 followed by addition acetone (74.42 mg, 1.28 mmol). The mixture was stirred at room temperature for 0.5 h. NaBH(OAc)₃ (135.78 mg, 0.64 mmol) was added and the mixture was stirred at room temperature for 3 h. TLC showed the reaction was complete. The mixture was quenched with water, extracted with DCM, washed with brine and concentrated in vacuo to afford the crude product. The crude was purified by column chromatography (DCM/MeOH=20/1) to afford (R)-1-(3-((5-fluoro-2-((2-isopropyl-1,2,3,4-tetrahydroisoquinolin-6-yl)amino)-pyrimidin-4-yl)amino)piperidin-1-yl)prop-2-en-1-one (I-29) as a yellow solid (19.4 mg, 14%).

LCMS: 439.3 [M+1]⁺

¹H NMR (400 MHz, DMSO-d₆): δ 1.14-1.23 (m, 8H), 1.41 (br, 1H), 1.61-1.65 (m, 1H), 1.81 (d, 1H), 1.97 (d, 1H), 2.73-3.17 (m, 7H), 4.02 (d, 2H), 4.17 (d, 0.5H), 4.43 (d, 0.5H), 5.49 (d, 0.5H), 5.71 (d, 0.5H), 6.12 (dd, 1H), 6.63 (t, 0.5H), 6.82-6.93 (m, 1.5H), 7.39-7.66 (m, 3H), 7.90 (s, 1H), 9.05 (s, 1H).

### Example 30

### Preparation of (R)-1-(3-((2-((2-Acetyl-1,2,3,4-tetrahydroisoquinolin-6-yl)amino)-5-fluoro-pyrimidin-4-yl)amino)piperidin-1-yl)prop-2-en-1-one (I-30)

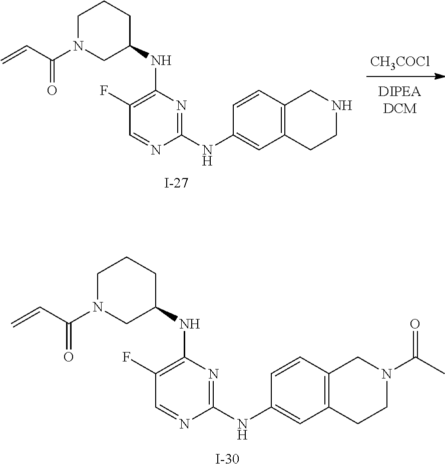

To a solution of (R)-1-(3-((5-fluoro-2-((1,2,3,4-tetrahydroisoquinolin-6-yl)amino)pyrimidin-4-yl)amino)-piperidin-1-yl)prop-2-en-1-one (I-27) (79 mg, 0.20 mmol) in DCM (5 mL) was added DIPEA (77.1 mg, 0.60 mmol) and acetyl chloride (15.6 mg, 0.20 mmol) at 0° C. The mixture was stirred at 0° C. for 0.5 h. TLC showed that the reaction was complete. The mixture was quenched with water, extracted with DCM, washed with water and brine and concentrated in vacuo to afford the crude product. The crude compound was purified by column chromatography (DCM/MeOH=50/1) to afford (R)-1-(3-((2-((2-acetyl-1,2,3,4-tetrahydroisoquinolin-6-yl)amino)-5-fluoro-pyrimidin-4-yl)amino)piperidin-1-yl)prop-2-en-1-one (I-30) as a white solid (26.5 mg, 30%).

LCMS: 439.3 [M+1]+

¹H NMR (400 MHz, DMSO-d₆): δ 1.42 (br, 1H), 1.65 (br, 1H), 1.81-1.84 (m, 1H), 1.97-2.01 (m, 1H), 2.06 (d, 3H), 2.62 (br, 1H), 2.71-2.80 (m, 2H), 3.02-3.12 (m, 1H), 3.58 (d, 2H), 4.00-4.04 (m, 2H), 4.21 (d, 0.5H), 4.47-4.53 (m, 2.5H), 5.45 (t, 0.5H), 5.71 (d, 0.5H), 6.01 (dd, 0.5H), 6.12 (d, 0.5H), 6.56-6.60 (m, 0.5H), 6.86 (dd, 0.5H), 6.97-7.00 (m, 1H), 7.33-7.41 (m, 2H), 7.63 (dd, 1H), 7.91 (d, 1H), 9.04 (d, 1H).

### Example 31

### Preparation of (R)-1-(3-(5-Fluoro-2-(2-(oxetan-3-yl)-1,2,3,4-tetrahydroisoquinolin-6-ylamino)-pyrimidin-4-ylamino)piperidin-1-yl)prop-2-en-1-one (I-31)

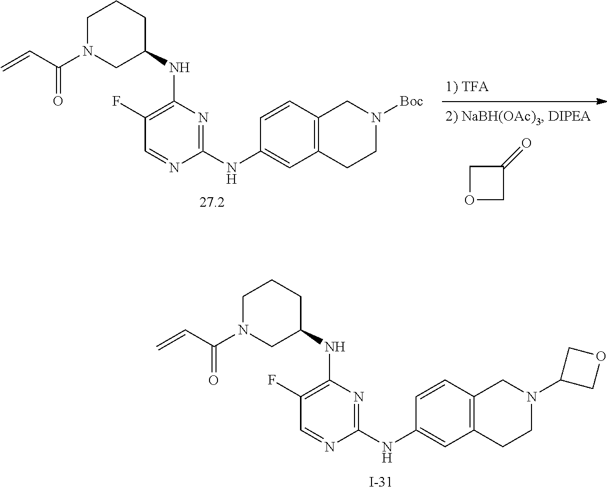

A mixture of (R)-tert-butyl 6-((4-((1-acryloylpiperidin-3-yl)amino)-5-fluoropyrimidin-2-yl)amino)-3,4-dihydroisoquinoline-2(1H)-carboxylate (27.2) (100 mg, 0.2 mmol) in TFA (3 mL) was stirred at room temperature for 30 min. The reaction mixture was concentrated to dryness and diluted with DCM (10 mL). DIPEA (1 mL) and NaBH(OAc)₃ (85 mg, 0.4 mmol) were added. The mixture was stirred at 0° C. for 30 min followed by the addition of oxetan-3-one (58 mg, 0.8 mmol). The suspension was stirred at 0° C. for 3 h. The reaction mixture was quenched with water and extracted with DCM. The combined organic layers were washed with water and brine, dried over sodium sulfate and concentrated in vacuo. The crude product was purified by column chromatography (DCM/methanol:10/1) to afford (R)-1-(3-(5-fluoro-2-(2-(oxetan-3-yl)-1,2,3,4-tetrahydroisoquinolin-6-ylamino)-pyrimidin-4-ylamino)piperidin-1-yl)prop-2-en-1-one (I-31) as a yellow solid (17 mg, 18.9%).

LCMS:453.2[M+1]⁺.

¹H NMR (400 MHz, DMSO˜d₆): δ 1.38˜1.43 (m, 1H), 1.61˜1.64 (m, 1H), 1.80˜1.84 (m, 1H), 1.98˜2.00 (m, 1H), 2.45 (t, 2H), 2.70˜2.79 (m, 3H), 2.99˜3.05 (m, 0.5H), 3.13˜3.18 (m, 0.5H), 3.2˜93.37 (m, 3H), 3.54 (t, 1H), 4.01 (br, 2H), 4.15˜4.20 (m, 0.5H), 4.41˜4.52 (m, 2.5H), 4.60 (t, 2H), 4.70 (dd, 0.5H), 5.69 (dd, 0.5H), 6.06 (dd, 1H), 5.60 (dd, 0.5H), 6.82˜6.88 (m, 1.5H), 7.34 (dd, 2H), 7.57 (d, 1H), 7.89 (d, 1H), 8.97 (s, 1H).

### Example 32

### Preparation of (R)-1-(3-(5-Fluoro-2-(2-(oxetan-3-ylmethyl)-1,2,3,4-tetrahydroisoquinolin-6-ylamino)pyrimidin-4-ylamino)piperidin-1-yl)prop-2-en-1-one (I-32)

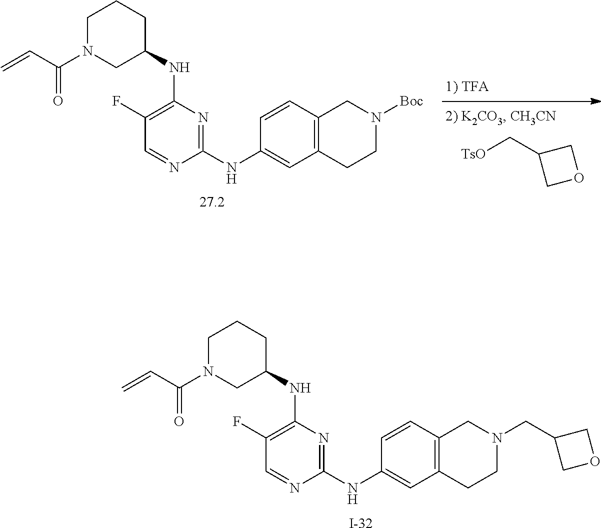

A mixture of (R)-tert-butyl 6-((4-((1-acryloylpiperidin-3-yl)amino)-5-fluoropyrimidin-2-yl)amino)-3,4-dihydroisoquinoline-2(1H)-carboxylate (27.2) (100 mg, 0.2 mmol) in TFA (3 mL) was stirred at room temperature for 30 min. The reaction mixture was concentrated to dryness and diluted with CH₃CN (10 mL). K₂CO₃ (60 mg, 0.4 mmol) and oxetan-3-ylmethyl-4-methylbenzenesulfonate (50 mg, 0.2 mmol) were added. The mixture was stirred at room temperature overnight. The reaction mixture was quenched with water and extracted with ethyl acetate. The combined organic layers were washed with water and brine, dried over sodium sulfate and concentrated in vacuo. The crude product was purified by column chromatography (DCM/methanol:10/1) to afford (R)-1-(3-(5-fluoro-2-(2-(oxetan-3-ylmethyl)-1,2,3,4-tetrahydroisoquinolin-6-ylamino)pyrimidin-4-ylamino)piperidin-1-yl)prop-2-en-1-one (I-32) as a yellow solid (20 mg, 21.5%).

LCMS:467.2 [M+1]⁺.

¹H NMR (400 MHz, DMSO-d₆): δ 1.38-1.43 (m, 1H), 1.61-1.65 (m, 1H), 1.79-1.84 (m, 1H), 1.97-2.01 (m, 1H), 2.56 (br, 2H), 2.66-2.75 (m, 5H), 3.02-3.18 (m, 1H), 3.25 (t, 1H), 3.39 (s, 2H), 4.00 (dd, 2H), 4.19 (dd, 0.5H), 4.29 (t, 2H), 4.41-4.44 (m, 0.5H), 4.66 (dd, 2H), 5.47 (d, 0.5H), 5.71 (dd, 0.5H), 6.02 (dd, 0.5H), 6.10 (dd, 0.5H), 6.60 (dd, 0.5H), 6.82-6.88 (m, 1.5H), 7.28 (t, 1H), 7.35 (d, 1H), 7.55 (d, 1H), 7.90 (d, 1H), 8.95 (s, 1H).

### Example 33

### Preparation of (R)-1-(3-((5-fluoro-2-((1,2,3,4-tetrahydroisoquinolin-7-yl)amino)pyrimidin-4-yl)amino)piperidin-1-yl)prop-2-en-1-one (I-33)

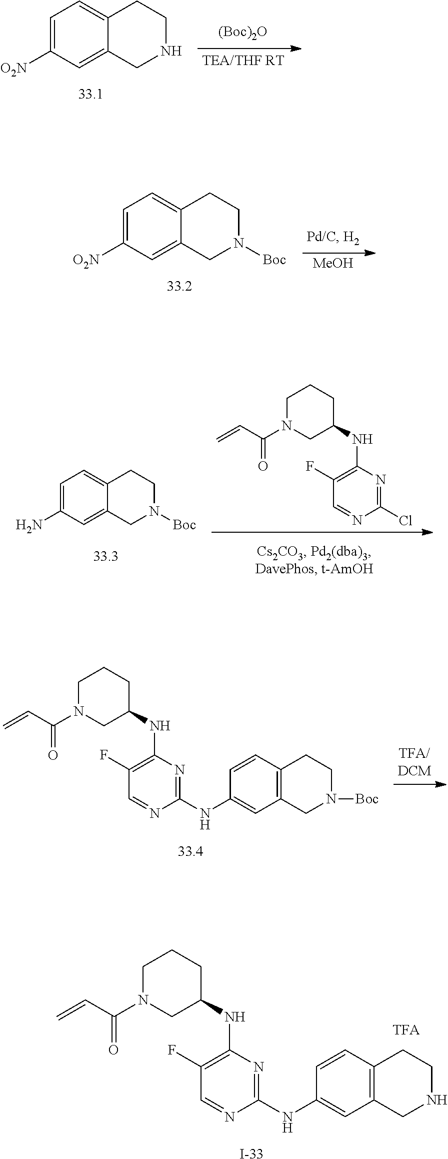

### tert-Butyl 7-nitro-3,4-dihydroisoquinoline-2(1H)-carboxylate (33.2)

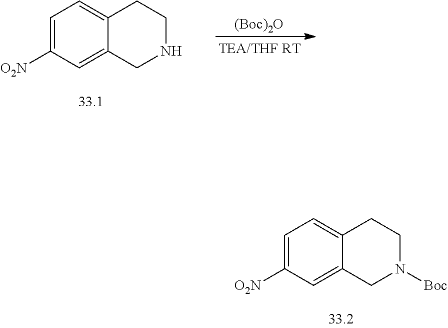 [SMILES: "(Boc)2O" ; O=[N+]([O-])c1ccc2c(c1)CNCC2 ; CC(C)(C)OC(=O)N1CCc2ccc([N+](=O)[O-])cc2C1]

To a solution of 7-nitro-1,2,3,4-tetrahydroisoquinoline (33.1) (4 g, 22.44 mmol) in THF (100 mL) was added TEA (2.27 g, 22.45 mmol) and tert-butoxycarbonyl tert-butyl carbonate (5.4 g, 24.74 mmol). The reaction mixture was stirred at room temperature for 2 h. TLC showed the reaction was complete. The reaction mixture was quenched with water and extracted with ethyl acetate. The combined organic layers were washed with water and brine, dried over sodium sulfate and concentrated in vacuo. The crude product was purified by column chromatography (DCM) to afford tert-butyl 7-nitro-3,4-dihydroisoquinoline-2(1H)-carboxylate (33.2) as a yellow liquid (4.4 g, 70.4%).

LCMS: 279.1 [M+1]⁺.

¹HNMR (400 MHz, CDCl₃): δ 1.50 (s, 9H), 2.93 (t, 2H), 3.69 (t, 2H), 4.66 (s, 2H), 7.29 (t, 1H), 8.03-8.02 (m, 2H).

### tert-Butyl 7-amino-3,4-dihydroisoquinoline-2(1H)-carboxylate (33.3)

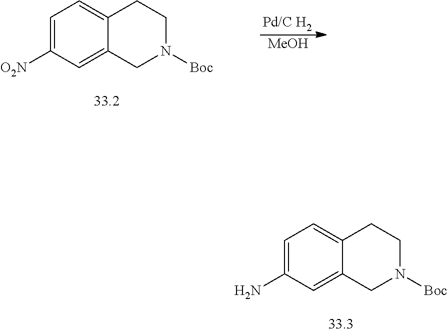 [SMILES: "MeOH" ; CC(C)(C)OC(=O)N1CCc2ccc([N+](=O)[O-])cc2C1 ; "Pd/C H2" ; CC(C)(C)OC(=O)N1CCc2ccc(N)cc2C1]

A solution of tert-butyl 7-nitro-3,4-dihydroisoquinoline-2(1H)-carboxylate (33.2) (4.4 g, 15.81 mmol) and Pd/C (500 mg, 4.69 mmol) in MeOH (100 mL) was stirred at room temperature for 3 h under H₂. The reaction mixture was filtered. The filtrate was concentrated to afford tert-butyl 7-amino-3,4-dihydroisoquinoline-2(1H)-carboxylate (33.3) as a yellow liquid (3.8 g, 96.8%).

¹H NMR (400 MHz, CDCl₃): δ 1.48 (s, 9H), 2.71 (t, 2H), 3.58 (s, 4H), 4.47 (s, 2H), 6.44 (s, 1H), 6.52 (dd, 1H), 6.91 (d, 1H).

### (R)-tert-Butyl 7-(4-(1-acryloylpiperidin-3-ylamino)-5-fluoropyrimidin-2-ylamino)-3,4-dihydroisoquinoline-2(1H)-carboxylate (33.4)

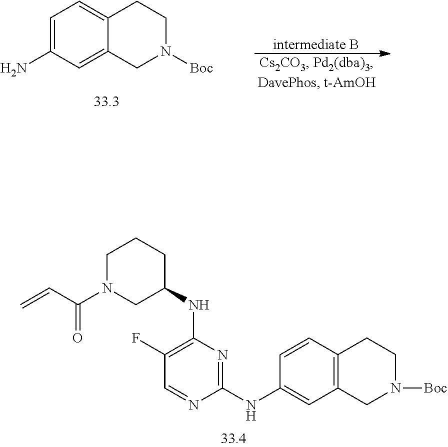 [SMILES: CC(C)(C)OC(=O)N1CCc2ccc(N)cc2C1 ; "intermediate B" ; "Cs2CO3, Pd2(dba)3,DavePhos, t-AmOH" ; C=CC(=O)N1CCC[C@@H](Nc2nc(Nc3ccc4c(c3)CN(C(=O)OC(C)(C)C)CC4)ncc2F)C1]

A stirred solution of tert-butyl 7-amino-3,4-dihydroisoquinoline-2(1H)-carboxylate (33.3) (2.06 g, 8.28 mmol), DavePhos (652 mg, 1.65 mmol), (R)-1-(3-(2-chloro-5-fluoropyrimidin-4-ylamino)piperidin-1-yl)prop-2-en-1-one (intermediate B, 2.36 g, 8.28 mmol), Pd₂(dba)₃ (762 mg, 0.83 mmol) and Cs₂CO₃ (5.41 g, 16.10 mmol) in tert-amyl alcohol (200 mL) was heated at 100° C. overnight under N₂. TLC showed the reaction was complete. The reaction mixture was quenched with water and extracted with ethyl acetate. The combined organic layers were washed with water and brine, dried over sodium sulfate and concentrated in vacuo. The crude product was purified by column chromatography (DCM/MeOH=20/1) to afford (R)-tert-butyl 7-(4-(1-acryloylpiperidin-3-ylamino)-5-fluoropyrimidin-2-ylamino)-3,4-dihydroisoquinoline-2(1H)-carboxylate (33.4) as a yellow solid (3.5 g, 85%).

LCMS: 497.5 [M+1]⁺.

### (R)-1-(3-(5-Fluoro-2-(1,2,3,4-tetrahydroisoquinolin-7-ylamino)pyrimidin-4-ylamino)-piperidin-1-yl)prop-2-en-1-one (I-33)

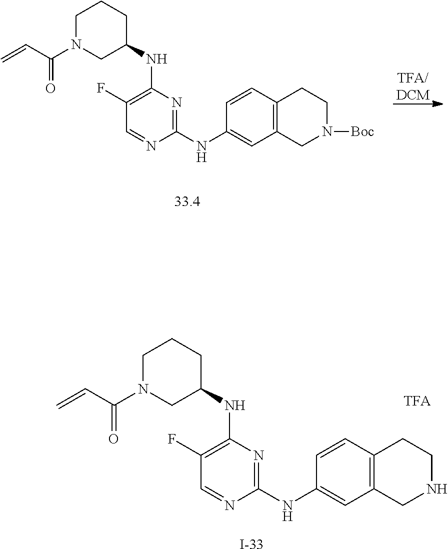

To a solution of (R)-tert-butyl 7-(4-(1-acryloylpiperidin-3-ylamino)-5-fluoropyrimidin-2-ylamino)-3,4-dihydroisoquinoline-2(1H)-carboxylate (33.4) (150 mg, 0.30 mmol) in DCM (20 mL) was added TFA (2.5 mL). The reaction mixture was stirred at room temperature for 1 h. TLC showed the reaction was complete. The reaction solution was concentrated to remove the excess TFA and DCM. The residue was washed with Et₂O three times and dried to afford the TFA salt of (R)-1-(3-(5-fluoro-2-(1,2,3,4-tetrahydroisoquinolin-7-ylamino)pyrimidin-4-ylamino)-piperidin-1-yl)prop-2-en-1-one (I-33) as a yellow solid (100 mg, 83%).

LCMS: 397.2 [M+1]⁺.

¹H NMR (400 MHz, CD₃OD): δ 1.57-1.60 (m, 1H), 1.77-1.83 (m, 1H), 1.93-1.98 (m, 1H), 2.11-2.14 (m, 1H), 2.84-2.94 (m, 1H), 3.04-3.16 (m, 2.5H), 3.19-3.26 (m, 0.5H), 3.48-3.59 (m, 2H), 4.04-4.46 (m, 4.5H), 4.62 (d, 0.5H), 5.46 (d, 0.5H), 5.82 (d, 0.5H), 6.06 (d, 0.5H), 6.24-6.37 (m, 1H), 684 (dd, 0.5H), 7.27 (t, 1H), 7.38-7.53 (m, 2H).

### Example 34

### Preparation of (R)-1-(3-((5-Fluoro-2-((2-methyl-1,2,3,4-tetrahydroisoquinolin-7-yl)amino)-pyrimidin-4-yl)amino)piperidin-1-yl)prop-2-en-1-one (I-34)

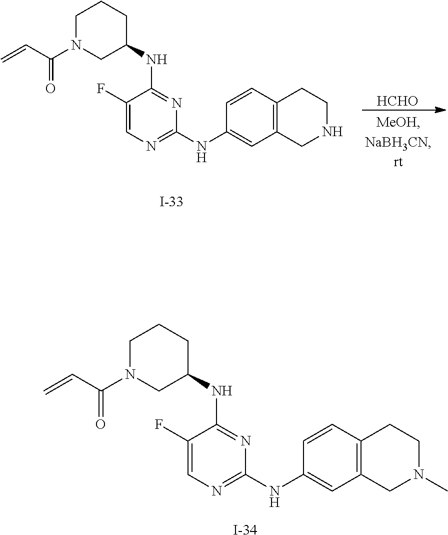

A mixture of formaldehyde (0.3 mL) and (R)-1-(3-(5-fluoro-2-(1,2,3,4-tetrahydroisoquinolin-7-ylamino)pyrimidin-4-ylamino)-piperidin-1-yl)prop-2-en-1-one (I-33) (80 mg, 0.20 mmol) in MeOH (8 mL) was stirred at 25° C. for 1 h. NaBH₃CN (25 mg, 0.40 mmol) was added. The mixture was stirred for 30 min. The reaction mixture was diluted with water and extracted with ethyl acetate. The combined organic layers were washed with water and brine, dried over sodium sulfate and concentrated in vacuo. The crude product was purified by prep-HPLC to afford the TFA salt of (R)-1-(3-((5-fluoro-2-((2-methyl-1,2,3,4-tetrahydroisoquinolin-7-yl)amino)-pyrimidin-4-yl)amino)piperidin-1-yl)prop-2-en-1-one (I-34) as a white solid (70 mg, 66% yield).

LCMS: 411.3[M+1]⁺.

¹H NMR (400 MHz, CD₃OD): δ 1.55-1.65 (m, 1H), 1.78-1.84 (m, 1H), 1.94-1.97 (m, 1H), 2.11-2.15 (m, 1H), 2.85-2.94 (m, 1.5H), 3.07 (d, J=6.4 Hz, 3H), 3.20-3.25 (m, 3H), 4.44-4.46 (m, 0.5H), 4.05-4.17 (m, 2H), 4.36-4.64 (m, 3H), 5.49-5.51 (m, 0.5H), 5.82-5.85 (m, 0.5H), 6.05-6.10 (m, 0.5H), 6.25-6.30 (m, 0.5H), 6.38-6.42 (m, 0.5H), 6.84-6.90 (m, 0.5H), 7.29-7.56 (m, 3H), 7.94-8.00 (m, 1H).

### Example 35

### Preparation of (R)-1-(3-(5-Fluoro-2-(2-(oxetan-3-yl)-1,2,3,4-tetrahydroisoquinolin-7-ylamino)-pyrimidin-4-ylamino)piperidin-1-yl)prop-2-en-1-one (I-35)

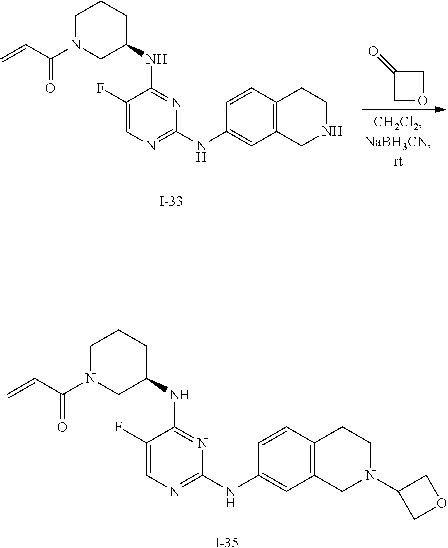

A mixture of (R)-1-(3-(5-fluoro-2-(1,2,3,4-tetrahydroisoquinolin-7-ylamino)pyrimidin-4-ylamino)-piperidin-1-yl)prop-2-en-1-one (I-33) (99 mg, 0.25 mmol) and oxetan-3-one (72 mg, 1 mmol) in DCM (10 mL) was stirred at 25° C. for 1 h. NaBH₃CN (25 mg, 0.40 mmol) was added. The mixture was stirred overnight. The reaction mixture was diluted with water and extracted with ethyl acetate. The combined organic layers were washed with water and brine, dried over sodium sulfate and concentrated in vacuo. The crude product was purified by column chromatography (DCM/methanol:10/1) to afford (R)-1-(3-(5-fluoro-2-(2-(oxetan-3-yl)-1,2,3,4-tetrahydroisoquinolin-7-ylamino)-pyrimidin-4-ylamino)piperidin-1-yl)prop-2-en-1-one (I-35) as a yellow solid (50 mg, 44%).

LCMS: 453 [M+1]⁺.

¹H NMR (400 MHz, DMSO-d₆): 1.37-1.40 (m, 1H), 1.62-1.63 (m, 1H), 1.81-1.84 (m, 1H), 1.97-1.98 (m, 1H), 2.70 (s, 4H), 3.00-3.10 (m, 1H), 3.23-3.30 (m, 1H), 3.59-3.36 (m, 2H), 3.51-3.53 (m, 1H), 4.00-4.04 (m, 2H), 4.23-4.25 (m, 0.5H), 4.44-4.45 (m, 0.5H), 4.50 (s, 2H), 4.60 (s, 2H), 5.47-5.50 (m, 0.5H), 5.70-5.73 (m, 0.5H), 6.00-6.16 (m, 1H), 6.58-6.62 (m, 0.5H), 6.83-6.87 (m, 0.5H), 6.90-6.92 (d, J=8.40 Hz, 1H), 7.23-7.45 (m, 2H), 7.51-7.60 (m, 1H), 7.89 (d, J=3.20 Hz, 1H), 8.98 (s, 1H).

### Example 36

### Preparation of (R)-1-(3-(5-Fluoro-2-(2-(oxetan-3-ylmethyl)-1,2,3,4-tetrahydroisoquinolin-7-ylamino)pyrimidin-4-ylamino)piperidin-1-yl)prop-2-en-1-one (I-36)

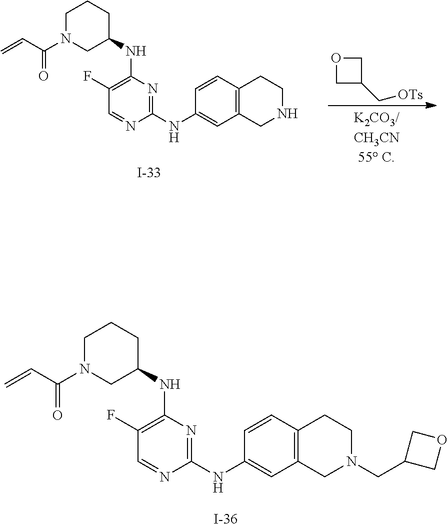

To a solution of (R)-1-(3-(5-fluoro-2-(1,2,3,4-tetrahydroisoquinolin-7-ylamino)pyrimidin-4-ylamino)-piperidin-1-yl)prop-2-en-1-one (I-33) (100 mg, 0.25 mmol) in CH₃CN (150 mL) was added K₂CO₃ (277 mg, 0.50 mmol) and oxetan-3-ylmethyl 4-methylbenzenesulfonate (91.65 mg, 0.37 mmol). The reaction mixture was stirred at 55° C. overnight under N₂. TLC showed the reaction was complete. The reaction mixture was quenched with water and extracted with ethyl acetate. The combined organic layers were washed with water and brine, dried over sodium sulfate and concentrated in vacuo. The crude product was purified by column chromatography (DCM:MeOH=10:1) to afford (R)-1-(3-(5-fluoro-2-(2-(oxetan-3-ylmethyl)-1,2,3,4-tetrahydroisoquinolin-7-ylamino)pyrimidin-4-ylamino)piperidin-1-yl)prop-2-en-1-one (I-36) as a yellow solid (60 mg, 50.9%).

LCMS: 467.1 [M+1]⁺.

¹H NMR (400 MHz, DMSO-d₆): δ 1.40-1.43 (m, 1H), 1.60-1.64 (m, 1H), 1.65-1.86 (m, 1H), 1.98-2.00 (m, 1H), 2.58-2.75 (m, 6H), 2.99-3.14 (m, 1H), 3.23-3.26 (m, 1H), 3.38-3.39 (m, 3H), 4.00-4.03 (m, 2H), 4.22-4.28 (m, 2.5H), 4.43 (d, 0.5H), 4.63-4.68 (m, 2H), 5.48 (d, 0.5H), 5.70 (d, 0.5H), 6.11 (dd, 1H), 6.60 (dd, 0.5H), 6.83-6.89 (m, 1.5H), 7.24-7.39 (m, 2H), 7.49 (s, 1H), 7.88 (d, 1H).

### Example 37

### Preparation of (R)-1-(3-((5-Fluoro-2-((7-fluoro-2-methylisoindolin-5-yl)amino)pyrimidin-4-yl)amino)piperidin-1-yl)prop-2-en-1-one (I-37)

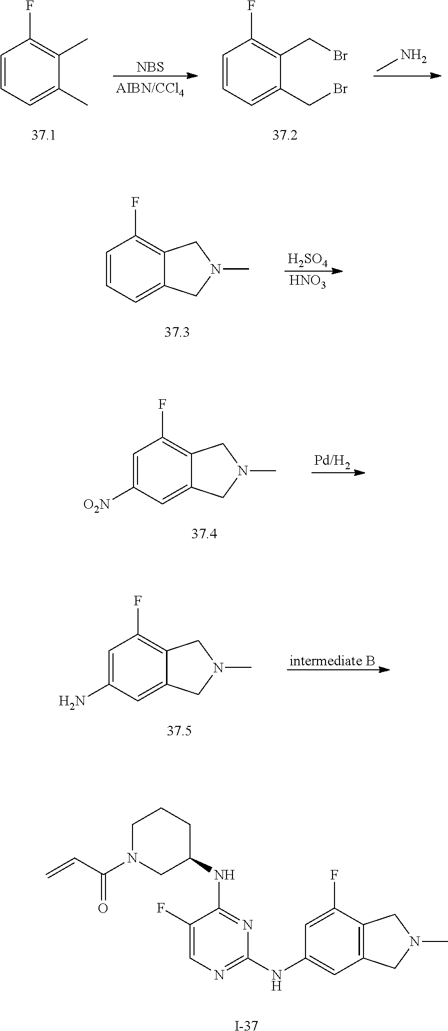

### 1,2-Bis(bromomethyl)-3-fluorobenzene (37.2)

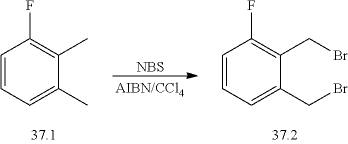

To a solution of 1-fluoro-2,3-dimethylbenzene (37.1, 5 g, 40.3 mmol) in CCl₄ (100 mL) was added NBS (15.3 g, 400 mmol) and AIBN (78 mg, 4 mmol). The mixture was allowed to stir at 80° C. for 3 h. The reaction was cooled to room temperature and filtered. The crude product was purified by column chromatography (hexane) to afford 1,2-bis(bromomethyl)-3-fluorobenzene (37.2) (6 g, 53%).

### 4-Fluoro-2-methylisoindoline (37.3)

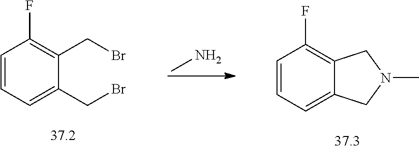

To 1,2-bis(bromomethyl)-3-fluorobenzene (37.2) (3 g, 10.6 mmol) was added methylamine methanolic solution (30% wt/wt, 30 mL). The mixture was allowed to stir at room temperature for 3 h. The reaction was concentrated in vacuo. The crude product was purified by column chromatography (hexane/ethyl acetate:1/1) to afford 4-fluoro-2-methylisoindoline (37.3) as a yellow solid (500 mg, 31%).

### 4-Fluoro-2-methyl-6-nitroisoindoline (37.4)

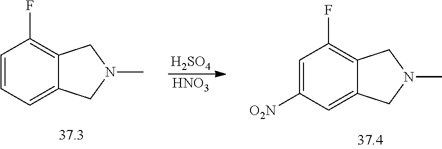

Fuming HNO₃ (210 mg, 3.3 mmol) was added to the solution of 37.3 (500 mg, 3.3 mmol) in H₂SO₄ (15 mL) at 0° C. The reaction mixture was stirred at 0° C. for 1 h. The reaction mixture was diluted with water and extracted with ethyl acetate. The combined organic layers were washed with water and brine, dried over sodium sulfate and concentrated in vacuo. The crude product was purified by column chromatography (hexane/ethyl acetate:1/1) to afford 4-fluoro-2-methyl-6-nitroisoindoline (37.4) as a yellow solid (150 mg, 38.7%).

LCMS: 235.3 [M+1]⁺.

¹H NMR (400 MHz, CDCl₃) δ 2.62 (s, 3H), 3.99 (s, 2H), 4.04 (s, 2H), 7.10 (d, J=8.0 Hz, 1H), 7.93-8.00 (m, 1H).

### 7-Fluoro-2-methylisoindolin-5-amine (37.5)

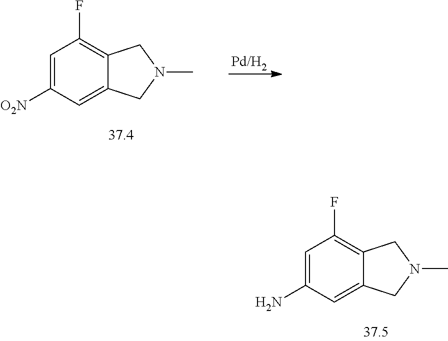

4-Fluoro-2-methyl-6-nitroisoindoline (37.4) (150 mg, 0.77 mmol) was dissolved in MeOH (10 mL). Pd/C (10 mg) was added to the mixture. The reaction was stirred under H₂ overnight. The reaction solution was filtered and the filtrate was concentrated to afford 7-fluoro-2-methylisoindolin-5-amine (37.5) as a yellow solid (100 mg, 78.2%).

### (R)-1-(3-(5-Fluoro-2-(7-fluoro-2-methylisoindolin-5-ylamino)pyrimidin-4-ylamino)piperidin-1-yl)prop-2-en-1-one (I-37)

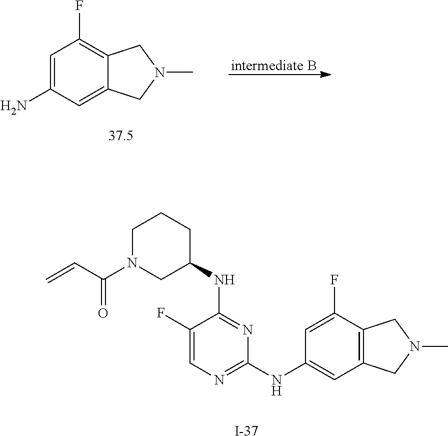

A suspension of 7-fluoro-2-methylisoindolin-5-amine (37.5) (60 mg, 0.36 mmol), Davephos (18 mg, 0.04 mmol), (R)-1-(3-(2-chloro-5-fluoropyrimidin-4-ylamino)piperidin-1-yl)prop-2-en-1-one (intermediate B, 142 mg, 0.5 mmol), Pd₂(dba)₃ (44 mg, 0.04 mmol) and Na₂CO₃ (105 mg, 1.0 mmol) in 2-methyl-2-butanol (20 mL) was heated at 100° C. under nitrogen for 7 h. After cooling to room temperature, the reaction mixture was diluted with water and extracted with ethyl acetate. The combined organic layers were washed with water and brine, dried over sodium sulfate and concentrated in vacuo. The crude product was purified by column chromatography (dichloromethane/methanol:20/1) to afford (R)-1-(3-(5-fluoro-2-(7-fluoro-2-methylisoindolin-5-ylamino)pyrimidin-4-ylamino)piperidin-1-yl)prop-2-en-1-one (I-37) as a yellow solid (60 mg, 40%).

LCMS: 415.3 [M+1]⁺.

¹H NMR (400 MHz, DMSO-d₆) δ 1.30-1.36 (m, 1H), 1.55-1.59 (m, 1H), 1.75-1.79 (m, 1H), 1.90-1.95 (m, 1H), 2.46 (s, 3H), 2.71-2.77 (m, 1H), 2.95-3.11 (m, 1H), 3.78-3.97 (m, 6H), 4.10-4.16 (m, 0.5H), 4.36-4.40 (m, 0.5H), 5.49-5.52 (m, 0.5H), 5.68-5.70 (m, 0.5H), 5.99-6.14 (m, 1H), 6.50-6.57 (m, 0.5H), 6.79-6.83 (m, 0.5H), 6.91 (d, J=8.00 Hz, 1H), 7.33 (d, J=7.66 Hz, 1H), 7.50-7.60 (m, 1H), 7.84 (m, 1H), 8.36 (s, 1H).

### Example 38

### Preparation of (R)-1-(3-((5-Fluoro-2-((2-methyl-2,3,4,5-tetrahydro-1H-benzo[c]azepin-7-yl)amino)pyrimidin-4-yl)amino)piperidin-1-yl)prop-2-en-1-one (I-38)

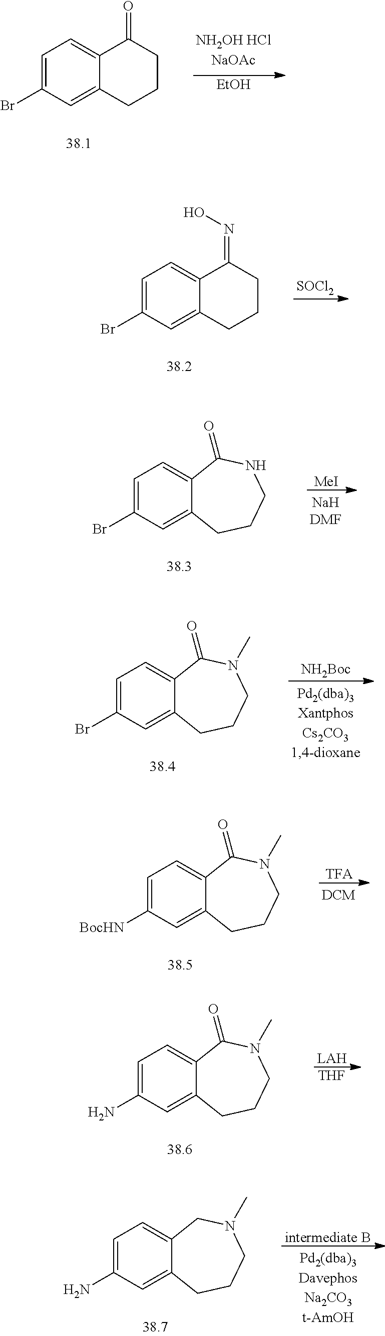

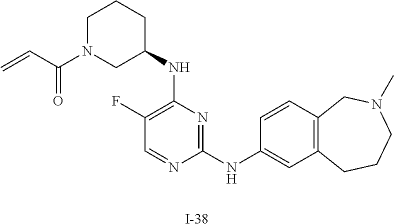

### (Z)-6-Bromo-3,4-dihydronaphthalen-1(2H)-one oxime (38.2)

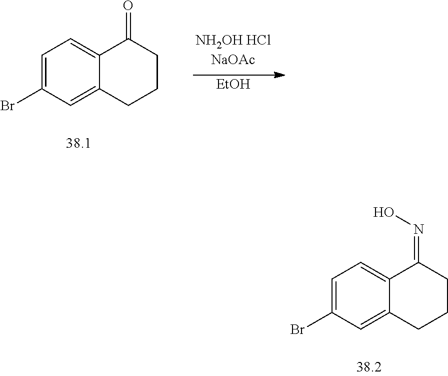 [SMILES: "EtOH" ; O=C1CCCc2cc(Br)ccc21 ; O/N=C1/CCCc2cc(Br)ccc21]

To a solution of 6-bromo-3,4-dihydronaphthalen-1(2H)-one (38.1, 5 g, 0.022 mol) in EtOH (30 mL) was added NH₂OH*HCl (1.86 g, 0.027 mol) and NaOAc (3.66 g, 0.045 mol). The mixture was heated at reflux for 2 h. TLC showed the reaction was complete. The mixture was quenched with water and filtered. The solid was washed with water and dried to afford (Z)-6-bromo-3,4-dihydronaphthalen-1(2H)-one oxime (38.2) as a white solid (4.8 g, 90%).

LCMS: 241.0 [M+1]⁺.

¹H NMR (400 MHz, DMSO-d₆): δ 1.72-1.75 (m, 2H), 2.64 (t, 2H), 2.71 (t, 2H), 7.37 (dd, 1H), 7.42 (d, 1H), 7.76 (d, 1H), 11.22 (s, 1H).

### 7-Bromo-2,3,4,5-tetrahydro-1H-benzo[c]azepin-1-one (38.3)

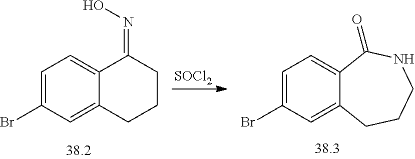

A solution of (Z)-6-bromo-3,4-dihydronaphthalen-1(2H)-one oxime (38.2, 4.8 g, 0.02 mol) in SOCl₂ (15 mL) was heated at 50° C. for 4 h. TLC showed that the reaction was complete. The mixture was concentrated to remove SOCl₂ and adjusted to pH=7 with aqueous sat. NaHCO₃ solution. The resulting mixture was extracted with EtOAc, washed with water and brine and concentrated in vacuo to afford the crude product. The crude product was purified by column chromatography (hexane/EtOAc=1/1) to afford 7-bromo-2,3,4,5-tetrahydro-1H-benzo[c]azepin-1-one (38.3) as a green solid (1.4 g, 29%).

LCMS: 239.9 [M+1]⁺.

¹H NMR (400 MHz, DMSO-d₆): δ 1.86-1.88 (m, 2H), 2.73 (t, 2H), 2.87-2.92 (m, 2H), 7.42-7.44 (m, 1H), 7.52-7.55 (m, 2H), 8.13 (br, 1H).

### 7-Bromo-2-methyl-2,3,4,5-tetrahydro-1H-benzo[c]azepin-1-one (38.4)

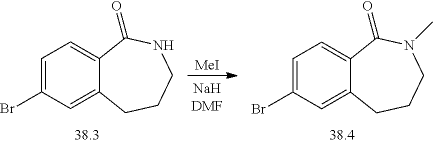

To a solution of 7-bromo-2,3,4,5-tetrahydro-1H-benzo[c]azepin-1-one (38.3, 1 g, 4.18 mmol) in DMF (15 mL) was added NaH (60%, 334 mg, 8.35 mmol) at room temperature. The mixture was stirred for 30 min followed by addition of MeI (1.19 g, 8.39 mmol). The mixture was stirred for 2 h. TLC showed the reaction was complete. The mixture was quenched with water and extracted with EtOAc. The organic solution was washed with water and brine and concentrated in vacuo to afford 7-bromo-2-methyl-2,3,4,5-tetrahydro-1H-benzo[c]azepin-1-one (38.4) as a brown solid (1.06 g, 100%).

LCMS: 254.0[M+1]+

### tert-Butyl (2-methyl-1-oxo-2,3,4,5-tetrahydro-1H-benzo[c]azepin-7-yl)carbamate (38.5)

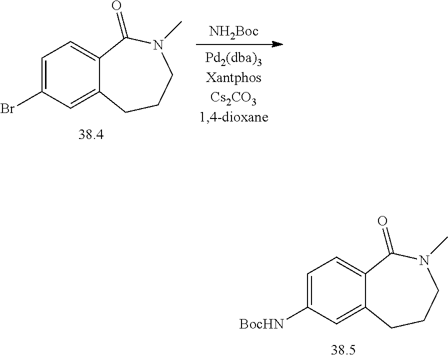 [SMILES: CN1CCCc2cc(Br)ccc2C1=O ; CN1CCCc2cc(NC(=O)OC(C)(C)C)ccc2C1=O ; "NH2Boc"]

To a solution of 7-bromo-2-methyl-2,3,4,5-tetrahydro-1H-benzo[c]azepin-1-one (38.4, 1.06 g, 4.17 mmol) in 1,4-dioxane (15 mL) was added NH₂Boc (1.52 g, 12.99 mmol), Pd₂(dba)₃ (200 mg, 0.219 mmol), Xantphos (125 mg, 0.216 mmol) and Cs₂CO₃ (2.8 g, 8.62 mmol) at room temperature. The mixture was stirred at 100° C. for 4 h. TLC showed the reaction was complete. The mixture was quenched with water and extracted with EtOAc. The organic layer was washed with water and brine and concentrated in vacuo to afford the crude product. The material was purified by column chromatography (DCM/MeOH=50/1) to afford tert-butyl (2-methyl-1-oxo-2,3,4,5-tetrahydro-1H-benzo[c]azepin-7-yl)carbamate (38.5) as a yellow solid (600 mg, 50%).

LCMS: 291.2[M+1]⁺.

### 7-Amino-2-methyl-2,3,4,5-tetrahydro-1H-benzo[c]azepin-1-one (38.6)

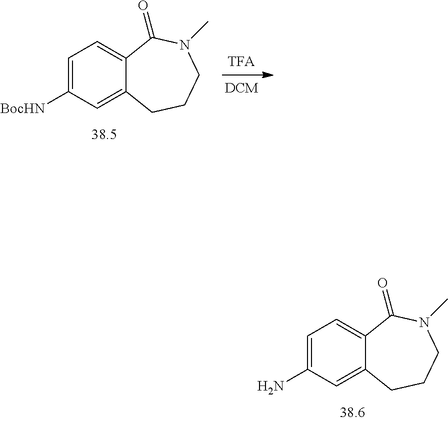 [SMILES: CN1CCCc2cc(NC(=O)OC(C)(C)C)ccc2C1=O ; CN1CCCc2cc(N)ccc2C1=O]

To a solution of tert-butyl 2-methyl-1-oxo-2,3,4,5-tetrahydro-1H-benzo[c]azepin-7-ylcarbamate (38.5, 160 mg, 0.55 mmol) in DCM (4 mL) was added TFA (4 mL) at room temperature. The mixture was stirred for 30 min. TLC showed the reaction was complete. The mixture was concentrated to remove the solvent and adjusted to pH=7 with aqueous NaHCO₃ and extracted with EtOAc. The organic layer was washed with water and brine and concentrated in vacuo to afford 7-amino-2-methyl-2,3,4,5-tetrahydro-1H-benzo[c]azepin-1-one (38.6) as a yellow solid (100 mg, 96%).

¹H NMR (400 MHz, DMSO-d₆): δ 1.90-1.95 (m, 2H), 2.49-2.51 (m, 4H), 2.96 (s, 3H), 3.11-3.15 (m, 2H), 5.44 (d, 2H), 6.32 (d, 1H), 6.42 (dd, 1H), 7.17 (d, 1H).

### 2-Methyl-2,3,4,5-tetrahydro-1H-benzo[c]azepin-7-amine (38.7)

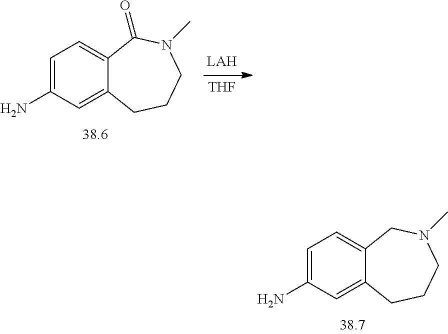

To a solution of 7-amino-2-methyl-2,3,4,5-tetrahydro-1H-benzo[c]azepin-1-one (38.6, 100 mg, 0.526 mmol) in dry THF (10 mL) was added LAH (90 mg, 2.37 mmol) at 0° C. The mixture was stirred for 30 min then refluxed for 4 h. TLC showed the reaction was complete. The mixture was quenched with water and extracted with EtOAc. The organic layer was washed with water and brine and concentrated in vacuo to afford 2-methyl-2,3,4,5-tetrahydro-1H-benzo[c]azepin-7-amine (38.7) as a brown solid (50 mg, 54%).

### (R)-1-(3-((5-Fluoro-2-((2-methyl-2,3,4,5-tetrahydro-1H-benzo[c]azepin-7-yl)amino)pyrimidin-4-yl)amino)piperidin-1-yl)prop-2-en-1-one (I-38)

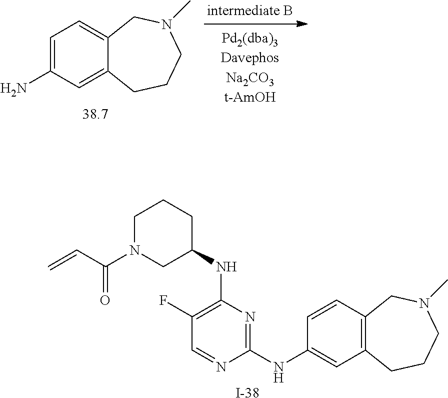

To a solution of 2-methyl-2,3,4,5-tetrahydro-1H-benzo[c]azepin-7-amine (38.7, 50 mg, 0.284 mmol) in t-AmOH (15 mL) was added (R)-1-(3-(2-chloro-5-fluoropyrimidin-4-ylamino) piperidin-1-yl)prop-2-en-1-one (intermediate B, 97 mg, 0.34 mmol), Pd₂(dba)₃ (26 mg, 0.028 mmol, Davephos (11 mg, 0.028 mmol) and Na₂CO₃ (211 mg, 1.99 mmol) at room temperature. The mixture was stirred at 100° C. for 4 h. TLC showed the reaction was complete. The mixture was quenched with water and extracted with EtOAc. The organic layer was washed with water and brine and concentrated in vacuo to get the crude product. The material was purified by prep. HPLC to afford (R)-1-(3-((5-Fluoro-2-((2-methyl-2,3,4,5-tetrahydro-1H-benzo[c]azepin-7-yl)amino)pyrimidin-4-yl)amino)piperidin-1-yl)prop-2-en-1-one (I-38) in the form of TFA salt as a yellow solid (27.6 mg, 23%).

LCMS: 425.2 [M+1]⁺.

¹H NMR (400 MHz, CD₃OD): δ 1.56-1.59 (m, 1H), 2.73 (t, 2H), 1.79-1.82 (m, 1H), 1.94-1.98 (m, 2H), 2.14-2.16 (br, 2H), 2.83-3.26 (m, 8H), 3.49-352 (m, 1H), 3.66-3.69 (m, 1H), 4.06-4.20 (m, 2H), 4.44-4.51 (m, 2.5H), 4.63-4.66 (m, 0.5H), 5.48-5.49 (m, 0.5H), 2.62 (br, 1H), 5.83 (d, 0.5H), 6.08 (d, 0.5H), 6.26 (d, 0.5H), 6.43 (d, 0.5H), 6.86 (dd, 0.5H), 7.39 (d, 1H), 7.50-7.68 (m, 2H), 7.97 (dd, 1H).

### Example 39

### Preparation of (R)-1-(3-((5-Fluoro-2-((2-(2-fluoroethyl)isoindolin-5-yl)amino)pyrimidin-4-yl)amino)piperidin-1-yl)prop-2-en-1-one (I-39)

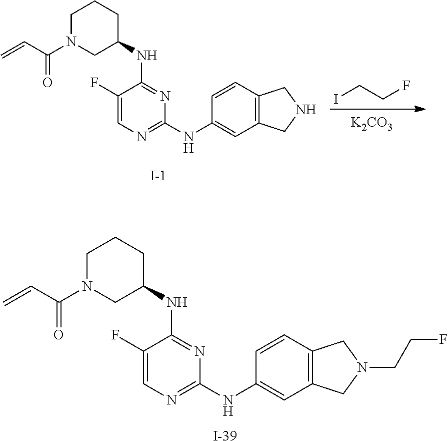

To (R)-1-(3-((5-fluoro-2-(isoindolin-5-ylamino)pyrimidin-4-yl)amino)piperidin-1-yl)prop-2-en-1-one (I-1) 100.mg, 0.26 mmol) in CH₃CN (10 mL) was added 1-fluoro-2-iodo-ethane (136.46 mg, 0.7800 mmol) and K₂ CO₃ (108.26 mg, 0.78 mmol). The mixture was stirred at 90° C. for 3 h. The reaction was concentrated to dryness and the residue redissolved in EtOAc (50 ml), washed with 2×50 ml water then 1×50 ml saturated brine solution. The organic layer was separated, dried over Na₂SO₄ and concentrated to dryness. The crude product was purified by column chromatography (dichloromethane/methanol:85/5) to afford (R)-1-(3-((5-fluoro-2-((2-(2-fluoroethyl)isoindolin-5-yl)amino)pyrimidin-4-yl)amino)piperidin-1-yl)prop-2-en-1-one (I-39) as a yellow solid (45 mg, 40%)

LCMS: 429.2[M+1]⁺.

¹H NMR (400 MHz, DMSO-d₆): δ 1.67-2.01 (m, 4H), 2.67-2.79 (m, 1H), 2.98-3.15 (m, 3H), 3.88 (br, 4H) 4.01 (b, 3H), 4.21 (br, 0.5H), 4.44 (br, 0.5H), 4.53 (br, 1H), 4.65 (br, 1H), 5.48-5.51 (d, 0.5H), 5.69-5.71 (d, 0.5H), 6.01-6.15 (dd, 1H), 6.61-6.68 (m, 0.5H), 6.82-6.89 (m, 0.5H), 7.04 (d, 1H), 7.36-7.41 (m, 2H), 7.88 (d, 1H), 7.90 (d, 1H), 9.05-9.06 (m, 1H).

### Example 40

### Preparation of (R)-1-(3-((5-Fluoro-2-((2-(4-methyltetrahydro-2H-pyran-4-yl)isoindolin-5-yl)amino)pyrimidin-4-yl)amino)piperidin-1-yl)prop-2-en-1-one (I-40)

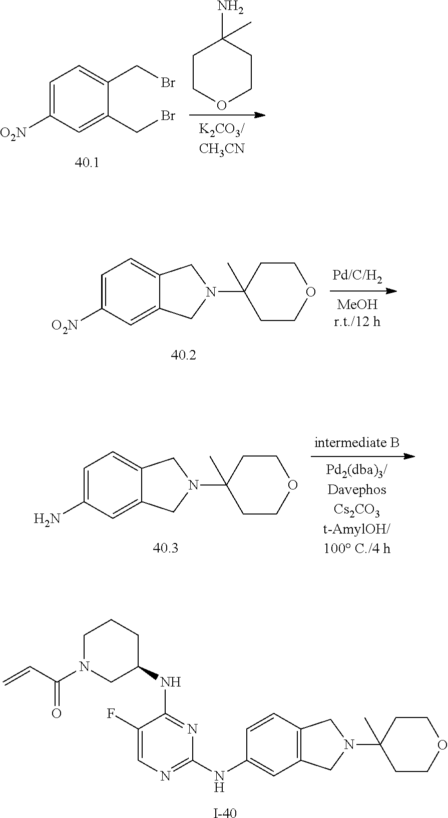

### 2-(4-Methyltetrahydro-2H-pyran-4-yl)-5-nitroisoindoline (40.2)

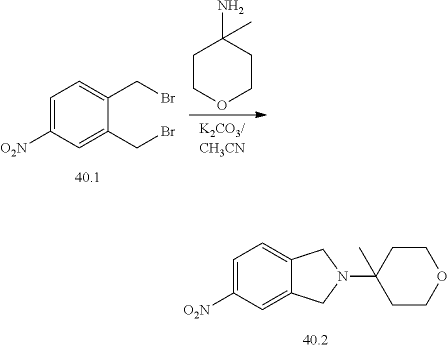

A solution of 1,2-bis(bromomethyl)-4-nitrobenzene (40.1, 200 mg, 0.65 mmol), K₂CO₃ (276 mg, 2.0 mmol) and 4-methyltetrahydro-2H-pyran-4-amine (99 mg, 0.65 mmol) in CH₃CN (15 mL) was stirred at 85° C. overnight. The reaction mixture was concentrated and purified by column chromatography (0 to 50% ethyl acetate in hexanes) to afford 2-(4-methyltetrahydro-2H-pyran-4-yl)-5-nitroisoindoline (40.2) as a yellow solid (150 mg, 88%).

LCMS: 263.1[M+1]⁺.

### 2-(4-Methyltetrahydro-2H-pyran-4-yl)isoindolin-5-amine (40.3)

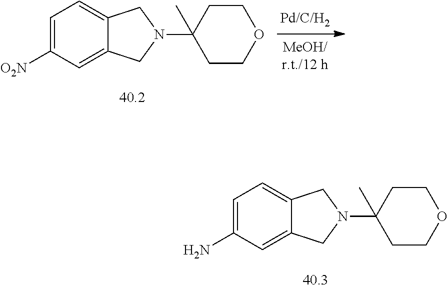 [SMILES: CC1(N2Cc3ccc([N+](=O)[O-])cc3C2)CCOCC1 ; CC1(N2Cc3ccc(N)cc3C2)CCOCC1]

2-(4-Methyltetrahydro-2H-pyran-4-yl)-5-nitroisoindoline (40.2) (150 mg, 0.57 mmol) was dissolved in MeOH (10 mL) and Pd/C (10 mg) was added to the mixture. The reaction was stirred under H₂ overnight. The reaction solution was filtered and the filtrate was concentrated to afford 2-(4-methyltetrahydro-2H-pyran-4-yl)isoindolin-5-amine (40.3) as a yellow oil (100 mg, 75.6%)

LCMS: 233.2[M+1]⁺.

### (R)-1-(3-(5-Fluoro-2-(2-(4-methyltetrahydro-2H-pyran-4-yl)isoindolin-5-ylamino)pyrimidin-4-ylamino)piperidin-1-yl)prop-2-en-1-one (I-40)

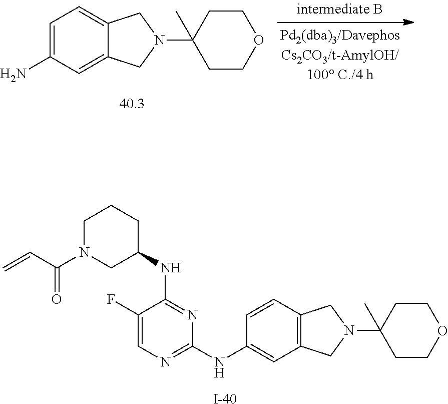

(R)-1-(3-(2-Chloro-5-fluoropyrimidin-4-ylamino)piperidin-1-yl)prop-2-en-1-one (intermediate B, 184 mg, 0.65 mmol), Cs₂CO₃ (278 mg, 0.86 mmol) were added to the solution of 2-(4-methyltetrahydro-2H-pyran-4-yl)isoindolin-5-amine (40.3) (100 mg, 0.43 mmol) in t-amyl alcohol (8 mL). Pd₂(dba)₃ (28 mg, 0.03 mmol) and Davephos (24 mg, 0.06 mmol) were added under N₂. The resulting mixture was heated at 100° C. for 3 h. After cooling to room temperature, the reaction mixture was diluted with water and extracted with ethyl acetate. The combined organic layers were washed with water and brine, dried over sodium sulfate and concentrated in vacuo. The crude product was purified by column chromatography (dichloromethane/methanol:10/1) to afford (R)-1-(3-(5-fluoro-2-(2-(4-methyltetrahydro-2H-pyran-4-yl)isoindolin-5-ylamino)pyrimidin-4-ylamino)piperidin-1-yl)prop-2-en-1-one (I-40) as a yellow solid (50 mg, 24%).

LCMS: 481.2[M+1]⁺.

¹H NMR (400 MHz, DMSO): δ 0.96 (s, 3H), 1.35-1.54 (m, 3H), 1.55-1.75 (m, 3H), 1.78-1.86 (m, 1H), 1.93-2.04 (m, 1H), 2.63-2.79 (m, 1H), 2.98-3.14 (m, 1H), 3.50-3.53 (m, 2H), 3.61-3.73 (m, 2H), 3.83 (s, 4H), 4.01-4.04 (m, 2H), 4.22-4.26 (m, 0.5H), 4.46-4.50 (m, 0.5H), 5.47-5.53 (m, 0.5H), 5.68-5.78 (m, 0.5H), 6.02-6.14 (m, 1H), 6.61-6.68 (m, 0.5H), 6.82-6.86 (m, 0.5H), 7.02-7.04 (m, 1H), 7.34-7.41 (m, 2H), 7.69-7.72 (m, 1H), 7.89 (d, J=3.6 Hz, 1H), 9.03 (s, 1H).

### Example 41

### Preparation of (R)-1-(3-((5-Fluoro-2-((1,1,2,3,3-pentamethylisoindolin-5-yl)amino)pyrimidin-4-yl)amino)piperidin-1-yl)prop-2-en-1-one (I-41)

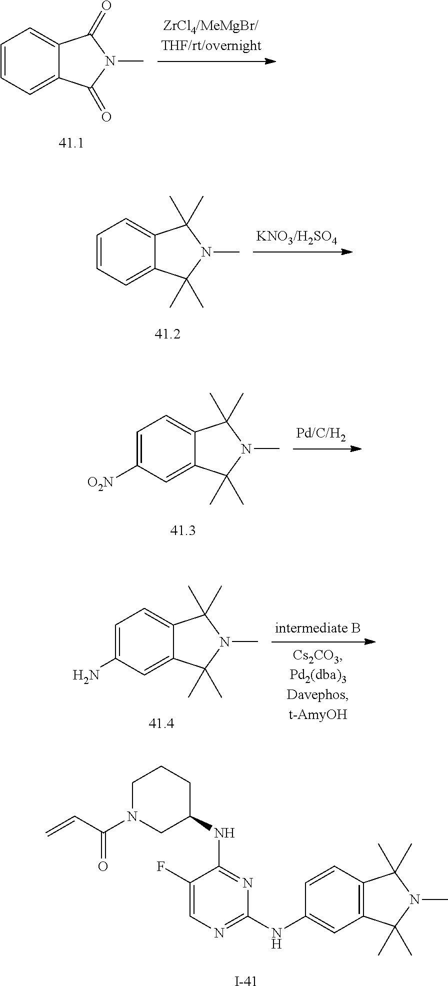

### 1,1,2,3,3-Pentamethylisoindoline (41.2)

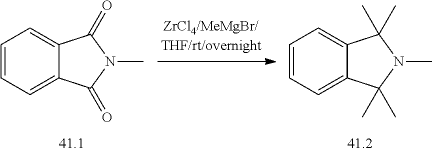

ZrCl₄ (8.7 g, 37.3 mmol) was added slowly to a solution of 2-methylisoindoline-1,3-dione (41.1, 3.0 g, 18.6 mmol) in THF (50 mL) at −20° C. The mixture was stirred at −20° C. for 1 h. MeMgBr (1 M, 111.6 mL, 111.6 mmol) was added slowly to the reaction at −20° C. The mixture was stirred at room temperature overnight. The reaction mixture was poured into 5N NaOH solution (while keeping pH 14) and extracted with DCM. The organic solution was concentrated to afford 1,1,2,3,3-pentamethylisoindoline (41.2) as a brown oil (1.0 g, 28%).

### 1,1,2,3,3-Pentamethyl-5-nitroisoindoline (41.3)

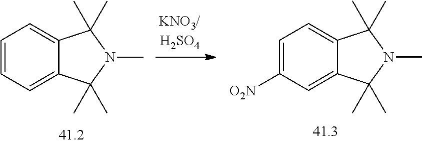 [SMILES: CN1C(C)(C)c2ccccc2C1(C)C ; CN1C(C)(C)c2ccc([N+](=O)[O-])cc2C1(C)C]

KNO₃ (1.1 g, 10.6 mmol) was added slowly to the solution of 1,1,2,3,3-pentamethylisoindoline (41.2) (1.0 g, 5.3 mmol) in H₂SO₄ (20 mL) at 0° C. The mixture was stirred at 0° C. for 2 h. The reaction mixture was poured into cold 5N NaOH solution (keeping pH 14). The aqueous solution was extracted with DCM. The crude product was purified by column chromatography (dichloromethane/methanol:40/1) to afford 1,1,2,3,3-pentamethyl-5-nitroisoindoline (41.3) as a brown solid (200 mg, 16% yield).

LCMS: 235.1 [M+1]⁺.

¹H NMR (400 MHz, CDCl₃): δ 1.37-1.39 (m, 12H), 2.44 (s, 3H), 7.28 (d, J=8.3 Hz, 1H), 8.00 (d, J=2.04 Hz, 1H), 8.14 (dd, J=8.30, 1.98 Hz, 1H).

### 1,1,2,3,3-Pentamethylisoindolin-5-amine (41.4)

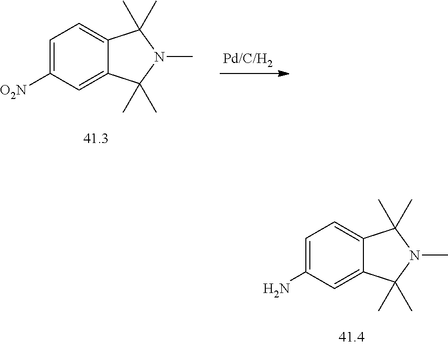 [SMILES: CN1C(C)(C)c2ccc([N+](=O)[O-])cc2C1(C)C ; CN1C(C)(C)c2ccc(N)cc2C1(C)C]

Pd/C (50 mg) was added to the solution of 1,1,2,3,3-pentamethyl-5-nitroisoindoline (41.3) (200 mg, 0.85 mmol) in MeOH (15 mL). The reaction mixture was stirred at 50° C. for 1 h under H₂. The reaction mixture was filtered and the filtrate was concentrated to afford 1,1,2,3,3-pentamethylisoindolin-5-amine (41.4) as a yellow solid (100 mg, 57%).

### (R)-1-(3-(5-Fluoro-2-(1,1,2,3,3-pentamethylisoindolin-5-ylamino)pyrimidin-4-ylamino) piperidin-1-yl)prop-2-en-1-one (I-41)

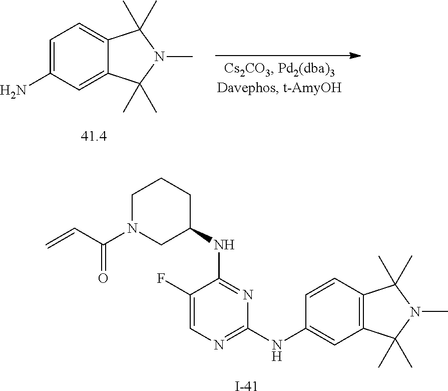

(R)-1-(3-(2-Chloro-5-fluoropyrimidin-4-ylamino)piperidin-1-yl)prop-2-en-1-one (intermediate B, 167 mg, 0.59 mmol) and Cs₂CO₃ (320 mg, 0.98 mmol) were added to a solution of 1,1,2,3,3-pentamethylisoindolin-5-amine (41.4) (100 mg, 0.49 mmol) in t-AmOH (10 mL). Pd₂(dba)₃ (45 mg, 0.05 mmol) and DavePhos (38 mg, 0.10 mmol) were added under N₂. The reaction mixture was stirred at 100° C. for 1 h. The reaction mixture was diluted with water and extracted with ethyl acetate. The combined organic layers were washed with water and brine, dried over sodium sulfate and concentrated in vacuo. The crude product was purified by Prep-HPLC to afford (R)-1-(3-(5-fluoro-2-(1,1,2,3,3-pentamethylisoindolin-5-ylamino)pyrimidin-4-ylamino) piperidin-1-yl)prop-2-en-1-one (I-41) as a white solid (70 mg, 32%).

LCMS: 453.3 [M+1]⁺.

¹H NMR (400 MHz, CD₃OD): δ 1.71 (m, 15H), 2.22-2.09 (m, 1H), 2.80-2.71 (m, 0.5H), 2.91-2.98 (m, 3.5H), 3.19-3.32 (m, 1H), 4.09-4.17 (m, 2H), 4.69-4.40 (m, 1H), 5.43-5.29 (m, 0.5H), 5.87-5.80 (m, 0.5H), 6.00-5.92 (m, 0.5H), 6.32-6.14 (m, 1H), 6.90-6.79 (m, 0.5H), 7.44 (d, J=8.19 Hz, 1H), 7.58-7.49 (m, 1H), 7.75-7.62 (m, 1H), 8.06-7.94 (m, 1H).

### Example 42

### Preparation of (R)-1-(3-((2-((2-cyclobutyl-1,2,3,4-tetrahydroisoquinolin-6-yl)amino)-5-fluoropyrimidin-4-yl)amino)piperidin-1-yl)prop-2-en-1-one (I-42)

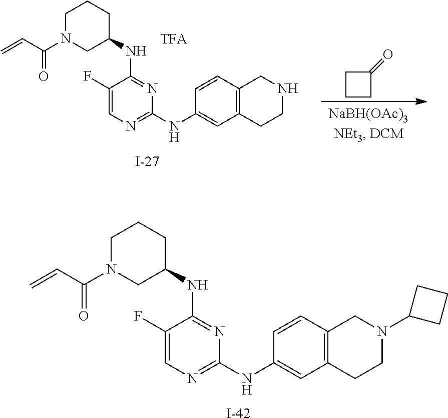

A solution of (R)-1-(3-((5-fluoro-2-((1,2,3,4-tetrahydroisoquinolin-6-yl)amino)pyrimidin-4-yl)amino)piperidin-1-yl)prop-2-en-1-one (I-27) (120 mg, 0.2 mmol) and cyclobutanone (40 mg, 0.6 mmol) in DCM (5 mL) was stirred at ambient temperature for 5 h. The reaction mixture was adjusted to pH 7 with the addition of triethylamine, and the mixture was stirred at ambient temperature for 30 min. Sodium triacetoxyborohydride (128 mg, 0.6 mmol) was added, and the reaction solution was stirred at ambient temperature for 1 h. TLC showed the reaction was complete. The reaction mixture was added to water and extracted with ethyl acetate. The combined organic layers were washed with water and brine, dried over anhydrous sodium sulfate, and concentrated to afford the crude product. The crude product was purified by column chromatography to afford (R)-1-(3-((2-((2-cyclobutyl-1,2,3,4-tetrahydroisoquinolin-6-yl)amino)-5-fluoropyrimidin-4-yl)amino)piperidin-1-yl)prop-2-en-1-one (I-42) as a yellow solid (60 mg, 66%, 2 steps).

LCMS: 451.3[M+1]⁺.

¹H NMR (400 MHz, DMSO-d₆): δ 1.39-1.44 (m, 1H), 1.63-1.85 (m, 4H), 1.98-2.18 (m, 6H), 2.70-3.17 (m, 8H), 4.01 (d, 2H), 4.20 (d, 0.5H), 4.44 (d, 0.5H), 5.49 (d, 0.5H), 5.71 (d, 0.5H), 6.01-6.14 (m, 1H), 6.89-6.66 (m, 0.5H), 6.52-6.89 (m, 0.5H), 6.97 (s, 1H), 7.41 (br, 2H), 7.64 (d, 1H), 7.91 (d, 1H), 9.10 (s, 1H).

### Example 43

### Preparation of (R)-1-(3-((2-((2-(cyclopropylmethyl)-1,2,3,4-tetrahydroisoquinolin-6-yl)amino)-5-fluoropyrimidin-4-yl)amino)piperidin-1-yl)prop-2-en-1-one (I-43)

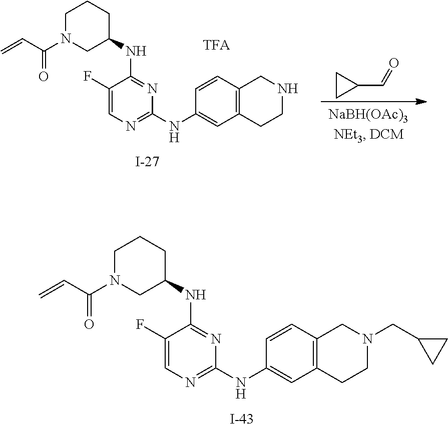

A solution of (R)-1-(3-((5-Fluoro-2-((1,2,3,4-tetrahydroisoquinolin-6-yl)amino)pyrimidin-4-yl)amino)piperidin-1-yl)prop-2-en-1-one (I-27) (120 mg, 0.2 mmol) and cyclopropanecarbaldehyde (28 mg, 0.4 mmol) in DCM (5 mL) was stirred at ambient temperature. The reaction mixture was adjusted to pH 7 with the addition of triethylamine, and the mixture was stirred at ambient temperature for 30 min. Sodium triacetoxyborohydride (128 mg, 0.6 mmol) was added, and the reaction solution was stirred at ambient temperature for 1 h. TLC showed the reaction was complete. The reaction mixture was added to water and extracted with ethyl acetate. The combined organic layers were washed with water and brine, dried over anhydrous sodium sulfate, and concentrated to afford the crude product. The crude product was purified by column chromatography to afford (R)-1-(3-((2-((2-(cyclopropylmethyl)-1,2,3,4-tetrahydroisoquinolin-6-yl)amino)-5-fluoropyrimidin-4-yl)amino)piperidin-1-yl)prop-2-en-1-one (I-43) as a yellow solid. (60 mg, 57%)

LCMS: 451.3[M+1]⁺.

¹H NMR (400 MHz, CD₃OD): δ 0.42-0.49 (m, 2H), 0.76-0.82 (m, 2H), 1.57-1.67 (m, 1H), 1.70-1.79 (m, 1H), 1.93 (dt, 1H), 2.13 (br, 1H), 2.80 (dd, 0.5H), 2.85-3.24 (m, 5.5H), 3.50 (br, 2H), 4.07-4.15 (m, 2H), 4.32 (br, 2H), 4.72 (d, 0.5H), 5.48 (dd, 0.5H), 5.81 (dd, 0.5H), 6.07 (dd, 0.5H), 6.27 (dd, 0.5H), 6.48 (dd, 0.5H), 6.86 (dd, 0.5H), 7.08 (d, 1H), 7.38 (d, 0.5H), 7.45 (d, 0.5H), 7.66 (s, 1H), 7.79 (dd, 1H).

### Example 44

### Preparation of (R)-1-(3-((2-((2-cyclopropyl-1,2,3,4-tetrahydroisoquinolin-6-yl)amino)-5-fluoropyrimidin-4-yl)amino)piperidin-1-yl)prop-2-en-1-one (I-44)

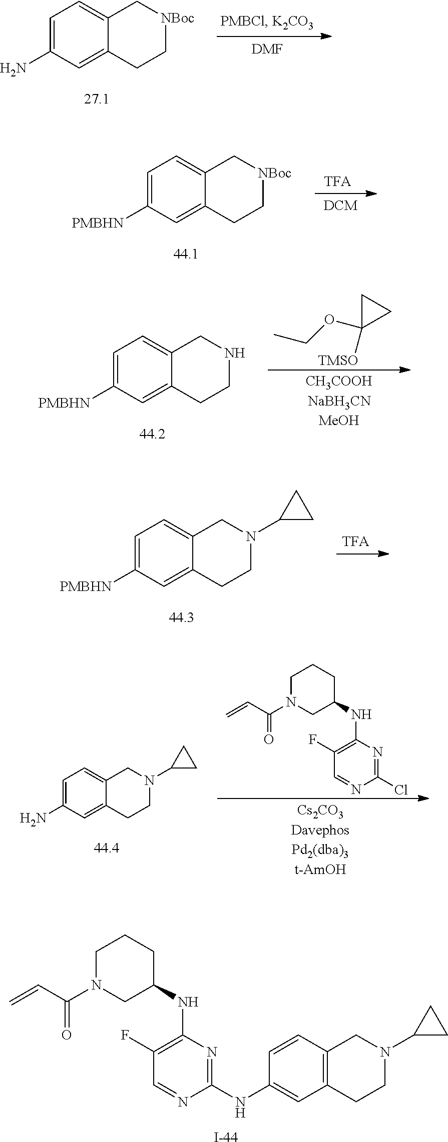

### tert-Butyl 6-((4-methoxybenzyl)amino)-3,4-dihydroisoquinoline-2(1H)-carboxylate (44.1)

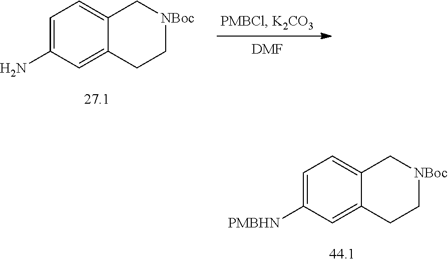

To a solution of tert-butyl 6-amino-3,4-dihydroisoquinoline-2(1H)-carboxylate (27.1) (2.0 g, 8.05 mmol) in DMF (20 mL) was added to 1-(chloromethyl)-4-methoxybenzene (1.5 g, 9.58 mmol) and K₂CO₃ (1.11 g, 8.05 mmol). The reaction mixture was stirred at 100° C. for 3 h under N₂. TLC showed the reaction was complete. The reaction mixture was quenched with the addition of water and extracted with ethyl acetate. The combined organic layers were washed with water and brine, dried over sodium sulfate, and concentrated in vacuo to afford the crude product. The crude product was purified by column chromatography (hexane/ethyl acetate:8/1) to afford tert-butyl 6-((4-methoxybenzyl)amino)-3,4-dihydroisoquinoline-2(1H)-carboxylate (44.1) as a white solid (1.1 g, 37%).

LCMS: 369.2 [M+1]⁺.

### N-(4-Methoxybenzyl)-1,2,3,4-tetrahydroisoquinolin-6-amine (44.2)

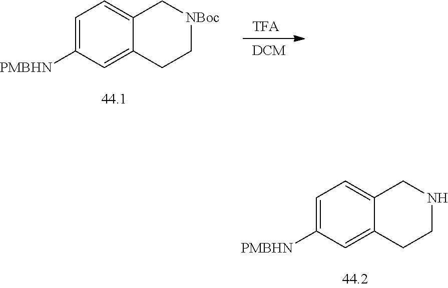

To a solution of tert-butyl 6-((4-methoxybenzyl)amino)-3,4-dihydroisoquinoline-2(1H)-carboxylate (44.1) (565 mg, 1.53 mmol) in DCM (3 mL) was added TFA (3 mL). The reaction mixture was stirred at ambient temperature for 1 h. TLC showed the reaction was complete. The excess DCM and TFA were removed. The reaction mixture was quenched with the addition of a saturated NaHCO₃ aqueous solution and extracted with ethyl acetate. The combined organic layers were washed with water and brine, dried over sodium sulfate, and concentrated to afford the crude product. The crude N-(4-methoxybenzyl)-1,2,3,4-tetrahydroisoquinolin-6-amine (44.2) was used in the next step without further purification (405 mg, 98%).

### 2-Cyclopropyl-N-(4-methoxybenzyl)-1,2,3,4-tetrahydroisoquinolin-6-amine (44.3)

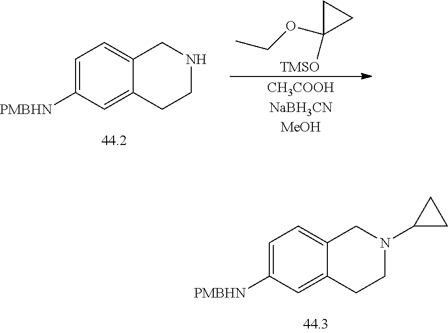

To a solution of N-(4-methoxybenzyl)-1,2,3,4-tetrahydroisoquinolin-6-amine (44.2) (405 mg, 1.51 mmol) in methanol (20 mL) was added (1-ethoxycyclopropoxy)trimethylsilane (316 mg, 1.81 mmol), CH₃COOH (3 mL, 1.51 mmol), and NaBH₃CN (285 mg, 1.51 mmol). The reaction mixture was stirred at 65° C. for 6 h under N₂. TLC showed the reaction was complete. The reaction mixture was filtered, and the filtrate was washed with sodium hydroxide and extracted with DCM. The combined organic layers were washed with water and brine, dried over sodium sulfate, and concentrated to afford to crude product. The crude product was purified by column chromatography (hexane/ethyl acetate:1/3) to afford 2-cyclopropyl-N-(4-methoxybenzyl)-1,2,3,4-tetrahydroisoquinolin-6-amine (44.3) as a colorless oil (200 mg, 43%).

LCMS: 309.1 [M+1]⁺.

¹HNMR (400 MHz, CDCl₃): δ 0.50 (two singlets, 4H), 1.74-1.79 (m, 1H), 2.78 (t, 2H), 2.88 (t, 2H), 3.69 (s, 2H), 3.79 (s, 3H), 4.21 (s, 2H), 6.37 (d, 1H), 6.44 (dd, 1H), 6.82-6.87 (m, 3H), 7.25 (s, 1H), 7.27 (s, 1H).

### 2-Cyclopropyl-1,2,3,4-tetrahydroisoquinolin-6-amine (44.4)

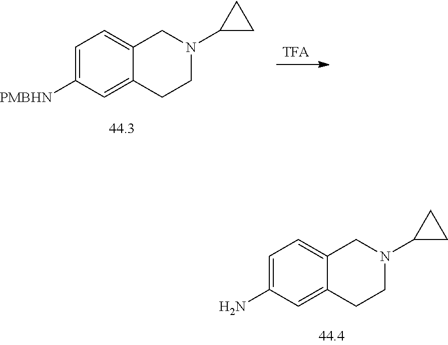

A solution of 2-cyclopropyl-N-(4-methoxybenzyl)-1,2,3,4-tetrahydroisoquinolin-6-amine (44.3) (200 mg, 0.65 mmol) in TFA (6 mL) was stirred at 80° C. overnight. TLC showed the reaction was complete. The excess TFA was removed. The reaction mixture was quenched with the addition of a saturated NaHCO₃ solution and extracted with ethyl acetate. The combined organic layers were washed with water and brine, dried over sodium sulfate and, concentrated to afford the crude product. The crude product was purified by column chromatography (DCM/methanol:40/1) to afford 2-cyclopropyl-1,2,3,4-tetrahydroisoquinolin-6-amine (44.4) as a yellow solid (80 mg, 65%).

¹HNMR (400 MHz, CDCl₃): δ 0.50 (two singlets, 4H), 1.74-1.80 (m, 1H), 2.78 (t, 2H), 2.88 (t, 2H), 3.50 (br, 2H), 3.69 (s, 2H), 6.43 (d, 1H), 6.48 (dd, 1H), 6.82 (d, 1H).

### (R)-1-(3-((2-((2-Cyclopropyl-1,2,3,4-tetrahydroisoquinolin-6-yl)amino)-5-fluoropyrimidin-4-yl)amino)piperidin-1-yl)prop-2-en-1-one (I-44)

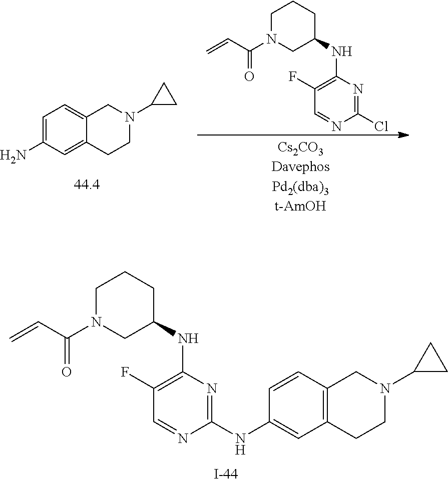

To a solution of 2-cyclopropyl-1,2,3,4-tetrahydroisoquinolin-6-amine (44.4) (79 mg, 0.42 mmol) in tert-amyl alcohol (5 mL) was added (R)-1-(3-((2-chloro-5-fluoropyrimidin-4-yl)amino)piperidin-1-yl)prop-2-en-1-one (intermediate B, 120 mg, 0.42 mmol), Davephos (33 mg, 0.08 mmol), tris(dibenzylideneacetone)dipalladium(O) (39 mg, 0.04 mmol), and Cs₂CO₃ (276 mg, 0.85 mmol). The reaction mixture was stirred at 100° C. for 3 h under N₂. TLC showed the reaction was complete. The reaction mixture was quenched with the addition of water and extracted with ethyl acetate. The combined organic layers were washed with water and brine, dried over sodium sulfate, and concentrated to afford the crude product. The crude product was purified by column chromatography (DCM/methanol:30/1) to afford (R)-1-(3-((2-((2-cyclopropyl-1,2,3,4-tetrahydroisoquinolin-6-yl)amino)-5-fluoropyrimidin-4-yl)amino)piperidin-1-yl)prop-2-en-1-one (I-44) as a yellow solid (70 mg, 38%).

LCMS: 437.3 [M+1]⁺.

¹HNMR (400 MHz, DMSO-d₆): δ 0.36 (s, 2H), 0.44-0.47 (m, 2H), 1.37-1.42 (m, 1H), 1.60-1.67 (m, 1H), 1.72-1.84 (m, 2H), 1.99 (br, 1H), 2.64 (d, 2H), 2.72-2.78 (m, 3H), 3.01 (t, 0.5H), 3.14 (t, 0.5H), 3.59 (t, 2H), 4.00 (br, 2H), 4.18 (d, 0.5H), 4.43 (d, 0.5H), 5.47 (d, 0.5H), 5.71 (d, 0.5H), 6.04 (dd, 1H), 6.60 (dd, 0.5H), 6.86 (dd, 1.5H), 7.28 (t, 1H), 7.37 (d, 1.0H), 7.54 (two singlets, 1H), 7.88 (d, 1H), 8.95 (s, 1H).

### Example 45

### Preparation of (R)-1-(3-((2-((2-(tert-butyl)-1,2,3,4-tetrahydroisoquinolin-6-yl)amino)-5-fluoropyrimidin-4-yl)amino)piperidin-1-yl)prop-2-en-1-one (I-45)

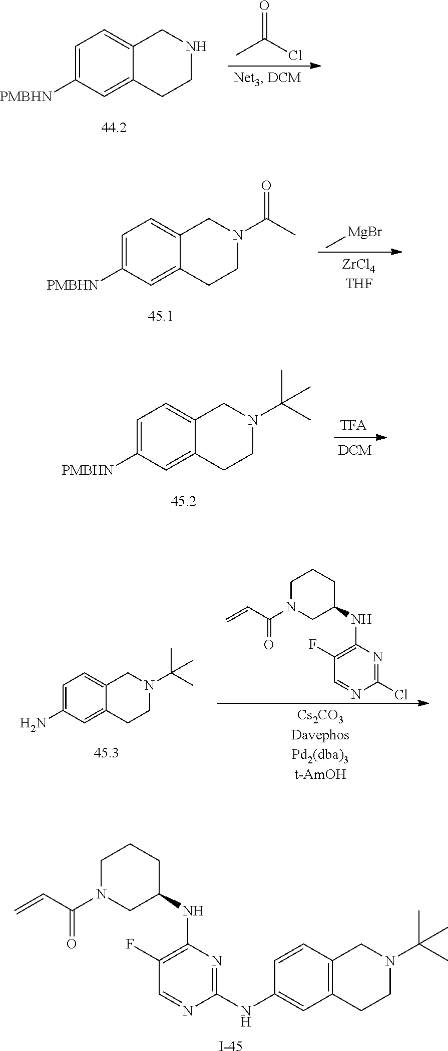

### 1-(6-((4-Methoxybenzyl)amino)-3,4-dihydroisoquinolin-2(1H)-yl)ethanone (45.1)

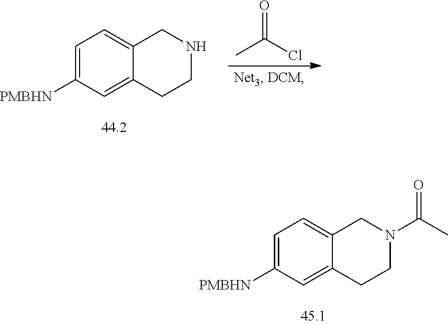

To a solution of N-(4-methoxybenzyl)-1,2,3,4-tetrahydroisoquinolin-6-amine (44.2) (500 mg, 1.86 mmol) in DCM (30 mL) was added acetyl chloride (176 mg, 2.24 mmol) and triethylamine (500 mg, 4.95 mmol). The reaction mixture was stirred at ambient temperature for 0.5 h under N₂. TLC showed the reaction was complete. The reaction mixture was quenched with the addition of water and extracted with ethyl acetate. The combined organic layers were washed with water and brine, dried over sodium sulfate, and concentrated to afford the crude product. The crude product was purified by column chromatography (hexane/ethyl acetate:1/1) to afford 1-(6-((4-methoxybenzyl)amino)-3,4-dihydroisoquinolin-2(1H)-yl)ethanone (45.1) as a yellow oil (570 mg, 99%).

LCMS: 311.1 (M+1)

### 2-(tert-Butyl)-N-(4-methoxybenzyl)-1,2,3,4-tetrahydroisoquinolin-6-amine (45.2)

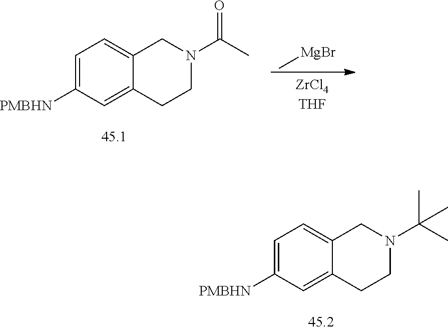

To a solution of 1-(6-((4-methoxybenzyl)amino)-3,4-dihydroisoquinolin-2(1H)-yl)ethanone (45.1) (570 mg, 1.84 mmol) in THF (30 mL) was added ZrCl₄ (714 mg, 3.67 mmol). The reaction mixture was stirred at −10° C. for 1.5 h under N₂. A solution of methylmagnesium bromide in THF (9.2 mL, 11 mmol) was added. The reaction mixture was stirred at ambient temperature for 6 h under N₂. TLC showed the reaction was complete. The reaction mixture was quenched with the addition of a 30% NaOH solution and extracted with DCM. The combined organic layers were washed with water and brine, dried over sodium sulfate, and concentrated to afford the crude product. The crude product was purified by column chromatography (DCM/methanol:20/1) to afford 2-(tert-butyl)-N-(4-methoxybenzyl)-1,2,3,4-tetrahydroisoquinolin-6-amine (45.2) as a yellow solid (70 mg, 12%).

LCMS: 325.2 [M+1]⁺.

### 2-(tert-Butyl)-1,2,3,4-tetrahydroisoquinolin-6-amine (45.3)

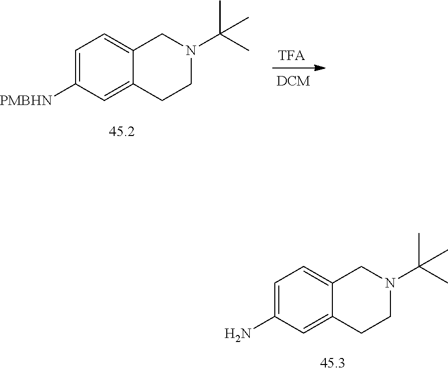

A solution of 2-(tert-butyl)-N-(4-methoxybenzyl)-1,2,3,4-tetrahydroisoquinolin-6-amine (45.2) (70 mg, 0.22 mmol) in TFA (6 mL) was stirred at 80° C. overnight. TLC showed the reaction was complete. The excess TFA was removed. The reaction mixture was quenched with the addition of a saturated NaHCO₃ solution and extracted with ethyl acetate. The combined organic layers were washed with water and brine, dried over sodium sulfate, and concentrated in vacuo to afford the crude product. The crude product, 2-(tert-butyl)-1,2,3,4-tetrahydroisoquinolin-6-amine (45.3) was used in the next step without further purification (40 mg, 90%).

LCMS: 205.1 [M+1]⁺.

### (R)-1-(3-((2-((2-(tert-Butyl)-1,2,3,4-tetrahydroisoquinolin-6-yl)amino)-5-fluoropyrimidin-4-yl)amino)piperidin-1-yl)prop-2-en-1-one (I-45)

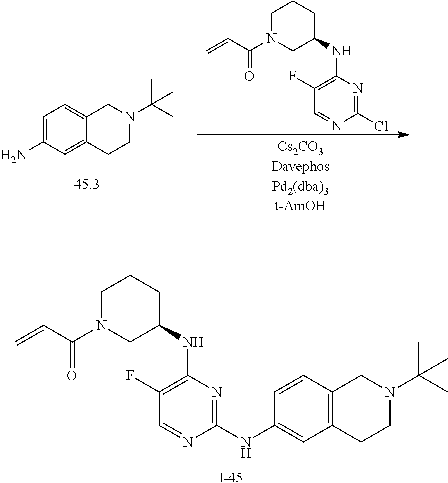

To a solution of 2-(tert-butyl)-1,2,3,4-tetrahydroisoquinolin-6-amine (45.3) (39 mg, 0.19 mmol) in tert-amyl alcohol (5 mL) was added (R)-1-(3-((2-chloro-5-fluoropyrimidin-4-yl)amino)piperidin-1-yl)prop-2-en-1-one (intermediate B, 55 mg, 0.19 mmol), Davephos (15 mg, 0.04 mmol), tris(dibenzylideneacetone)dipalladium(O) (18 mg, 0.02 mmol), and Cs₂CO₃ (128 mg, 0.39 mmol). The reaction mixture was stirred at 100° C. for 3 h under N₂. TLC showed the reaction was complete. The reaction mixture was quenched with the addition of water and extracted with ethyl acetate. The combined organic layers were washed with water and brine, dried over sodium sulfate, and concentrated to afford the crude product. The crude product was purified by column chromatography (DCM/methanol:20/1) to afford (R)-1-(3-((2-((2-(tert-butyl)-1,2,3,4-tetrahydroisoquinolin-6-yl)amino)-5-fluoropyrimidin-4-yl)amino)piperidin-1-yl)prop-2-en-1-one (I-45) as a yellow solid (15 mg, 17.4%).

LCMS: 453.2 [M+1]⁺.

¹HNMR (400 MHz, CD₃OD): δ 1.22 (s, 9H), 1.40-1.70 (m, 2H), 1.75-1.85 (m, 1H), 2.00-2.10 (m, 1H), 2.65-3.15 (m, 6H), 3.75-4.10 (m, 4H), 4.16 (d, 0.5H), 4.57 (d, 0.5H), 5.38 (d, 0.5H), 5.69 (d, 0.5H), 5.97 (d, 0.5H), 6.14 (d, 0.5H), 6.38 (dd, 0.5H), 6.74 (dd, 0.5H), 6.92 (d, 1H), 7.22 (dd, 1H), 7.45 (d, 1H), 7.63-7.66 (m, 1H).

### Example 46

### Preparation of (R)-1-(3-((2-((2-cyclobutyl-1,2,3,4-tetrahydroisoquinolin-7-yl)amino)-5-fluoropyrimidin-4-yl)amino)piperidin-1-yl)prop-2-en-1-one (I-46)

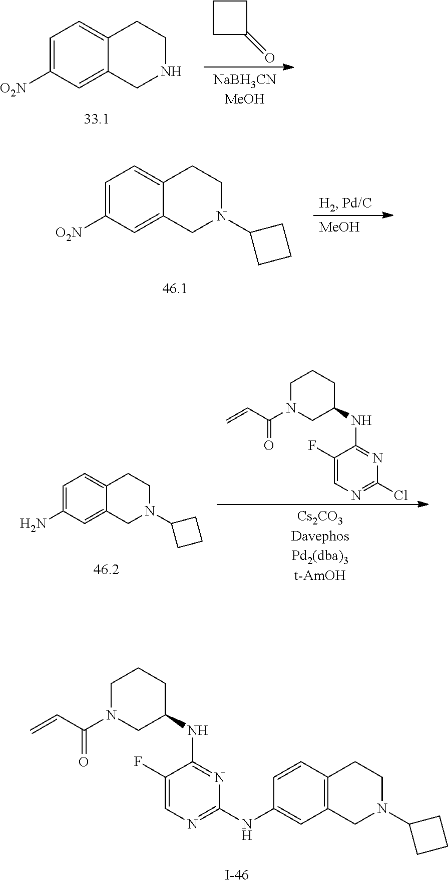

### 2-Cyclobutyl-7-nitro-1,2,3,4-tetrahydroisoquinoline (46.1)

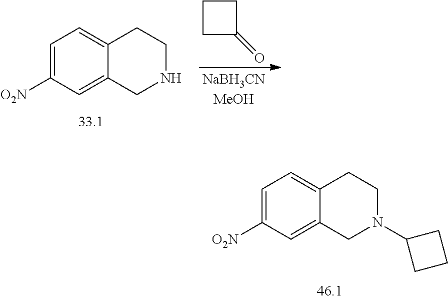

A solution of 7-nitro-1,2,3,4-tetrahydroisoquinoline (33.1) (200 mg, 1.12 mmol) and cyclobutanone (118 mg, 1.68 mmol) in methanol (15 mL) was stirred at ambient temperature for 2 h. NaBH₃CN (141 mg, 2.24 mmol) was added, and the mixture was stirred at ambient temperature for 20 min. The reaction mixture was diluted with water and extracted with ethyl acetate. The combined organic layers were washed with water and brine, dried over sodium sulfate, and concentrated to afford the crude product. The crude product was purified by column chromatography (DCM/methanol:10/1) to afford 2-cyclobutyl-7-nitro-1,2,3,4-tetrahydroisoquinoline (46.1) as a yellow oil (170 mg, 65%).

LCMS: 233.1[M+1]⁺.

### 2-Cyclobutyl-1,2,3,4-tetrahydroisoquinolin-7-amine (46.2)

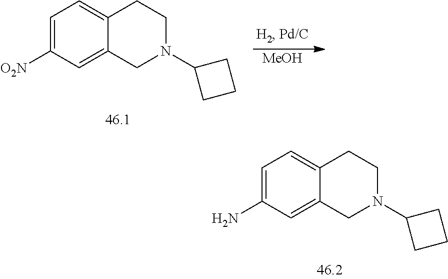 [SMILES: "MeOH" ; O=[N+]([O-])c1ccc2c(c1)CN(C1CCC1)CC2 ; Nc1ccc2c(c1)CN(C1CCC1)CC2]

A mixture of 2-cyclobutyl-7-nitro-1,2,3,4-tetrahydroisoquinoline (46.1) (170 mg, 0.73 mol) and Pd/C (70 mg) in MeOH (10 mL) was stirred at ambient temperature under H₂ overnight. The mixture was filtered, and the filtrate was concentrated to afford 2-cyclobutyl-1,2,3,4-tetrahydroisoquinolin-7-amine (46.2) as a colorless oil (136 mg, 92%), which was used in the next step without further purification.

LCMS: 203.2[M+1]⁺.

### (R)-1-(3-(2-(2-Cyclobutyl-1,2,3,4-tetrahydroisoquinolin-7-ylamino)-5-fluoropyrimidin-4-ylamino)piperidin-1-yl)prop-2-en-1-one (I-46)

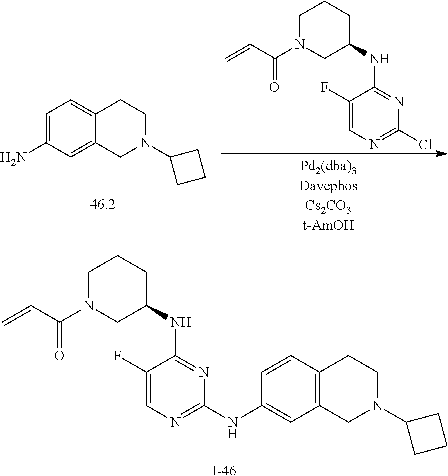

(R)-1-(3-(2-Chloro-5-fluoropyrimidin-4-ylamino)piperidin-1-yl)prop-2-en-1-one (intermediate B, 229 mg, 0.80 mmol) and Cs₂CO₃ (438 mg, 1.34 mmol) were added to the solution of 2-cyclobutyl-1,2,3,4-tetrahydroisoquinolin-7-amine (46.2) (136 mg, 0.67 mmol) in tert-amyl alcohol (10 mL). Tris(dibenzylideneacetone)dipalladium(O) (61 mg, 0.07 mmol) and DavePhos (53 mg, 0.13 mmol) were added under N₂. The reaction mixture was stirred at 100° C. for 3 h. The reaction mixture was diluted with water and extracted with ethyl acetate. The combined organic layers were washed with water and brine, dried over sodium sulfate, and concentrated to afford the crude product. The crude product was purified by column chromatography (DCM/MeOH:10/1) to afford (R)-1-(3-(2-(2-cyclobutyl-1,2,3,4-tetrahydroisoquinolin-7-ylamino)-5-fluoropyrimidin-4-ylamino)piperidin-1-yl)prop-2-en-1-one (I-46) as an off-white solid (60 mg, 20%).

LCMS: 451.2[M+1]⁺.

¹H NMR (400 MHz, DMSO-d₆): δ 1.38-1.42 (m, 1H), 1.60-1.70 (m, 3H), 1.81-1.85 (m, 3H), 1.99-2.03 (m, 3H), 2.40-2.46 (m, 2H), 2.68-2.82 (m, 4H), 3.01-3.09 (m, 1H), 3.27-3.29 (m, 2H), 3.99-4.03 (m, 2H), 4.24-4.27 (m, 0.5H), 4.43-4.46 (m, 0.5H), 5.47-5.50 (m, 0.5H), 5.70-5.72 (m, 0.5H), 6.00-6.15 (m, 1H), 6.58-6.62 (m, 0.5H), 6.82-6.91 (m, 1.5H), 7.24-7.40 (m, 2H), 7.56 (s, 1H), 7.88 (d, 1H), 8.98 (s, 1H).

### Example 47

### Preparation of (R)-1-(3-(2-(2-(Cyclopropylmethyl)-1,2,3,4-tetrahydroisoquinolin-7-ylamino)-5-fluoropyrimidin-4-ylamino)piperidin-1-yl)prop-2-en-1-one (I-47)

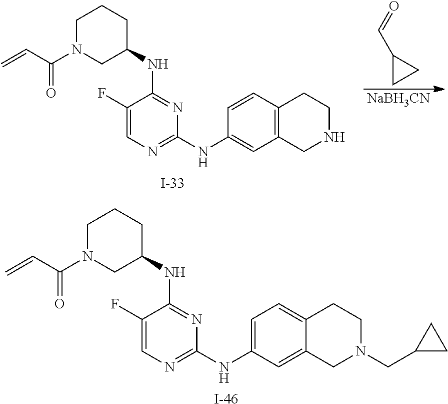

To a solution of (R)-1-(3-(5-fluoro-2-(1,2,3,4-tetrahydroisoquinolin-7-ylamino)pyrimidin-4-ylamino)-piperidin-1-yl)prop-2-en-1-one (I-33) in methanol (10 mL) was added cyclopropanecarbaldehyde (53 mg, 0.76 mmol) and the pH adjusted to 7 with that addition of triethylamine. The mixture was stirred at ambient temperature for 2 h. To the mixture was added NaBH₃CN (71 mg, 1.14 mmol), and the reaction mixture was stirred at ambient temperature for 1 h. The reaction mixture was diluted with water and extracted with ethyl acetate. The combined organic layers were washed with water and brine, dried over sodium sulfate, and concentrated to afford the crude product. The crude product was purified by column chromatography (DCM/methanol:10/1) to afford (R)-1-(3-(2-(2-(cyclopropylmethyl)-1,2,3,4-tetrahydroisoquinolin-7-ylamino)-5-fluoropyrimidin-4-ylamino)piperidin-1-yl)prop-2-en-1-one (I-47) as a white solid (40 mg, 22%, 2 steps).

Mp: 80-82° C.

LCMS: 451.3 [M+1]⁺.

¹H NMR (400 MHz, DMSO-d₆): δ 0.14-0.15 (m, 2H), 0.49-0.51 (m, 2H), 0.90-0.92 (m, 1H), 1.39-1.42 (m, 1H), 1.62-1.65 (m, 1H), 1.80-1.85 (m, 1H), 1.98-2.00 (m, 1H), 2.30-2.35 (m, 2H), 2.67-2.77 (m, 4H), 2.99-3.12 (m, 1H), 3.51-3.54 (m, 2H), 3.99-4.01 (m, 2H), 4.18-4.22 (m, 0.5H), 4.40-4.44 (m, 0.5H), 5.47-5.49 (m, 0.5H), 5.68-5.70 (m, 0.5H), 6.00-6.14 (m, 1H), 6.58-6.64 (m, 0.5H), 6.81-6.92 (m, 1.5H), 7.27-7.39 (m, 2H), 7.52 (s, 1H), 7.89 (d, 1H), 8.96 (s, 1H).

### Example 48

### Preparation of (R)-1-(3-((2-((2-cyclopropyl-1,2,3,4-tetrahydroisoquinolin-7-yl)amino)-5-fluoropyrimidin-4-yl)amino)piperidin-1-yl)prop-2-en-1-one (I-48)

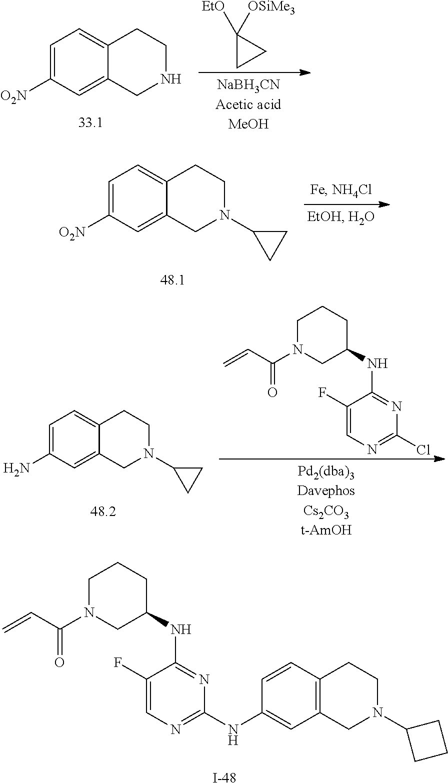

### 2-Cyclopropyl-7-nitro-1,2,3,4-tetrahydroisoquinoline (48.1)

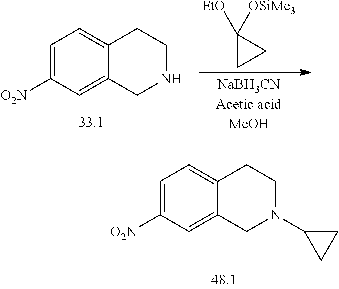

(1-Ethoxycyclopropoxy)trimethylsilane (977 mg, 5.6 mmol) was slowly added to the solution of 7-nitro-1,2,3,4-tetrahydroisoquinoline (33.1) (460 mg, 2.6 mmol), NaBH₃CN (980 mg, 15.6 mmol), and acetic acid (1.6 g, 25.9 mmol) in MeOH (20 mL) at 0° C. The resulting mixture was heated at 65° C. for 4 h. The reaction mixture was diluted with water and extracted with ethyl acetate. The combined organic layers were washed with water and brine, dried over sodium sulfate, and concentrated to afford the crude product. The crude product was purified by column chromatography (ethyl acetate/hexane:1/5) to afford 2-cyclopropyl-7-nitro-1,2,3,4-tetrahydroisoquinoline (48.1) as a yellow solid (500 mg, 88%).

LCMS: 219.1 [M+1]⁺.

### 2-Cyclopropyl-1,2,3,4-tetrahydroisoquinolin-7-amine (48.2)

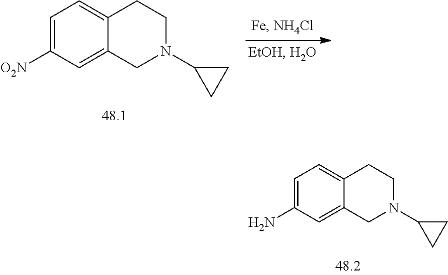 [SMILES: O=[N+]([O-])c1ccc2c(c1)CN(C1CC1)CC2 ; Nc1ccc2c(c1)CN(C1CC1)CC2]

Fe (770 mg, 13.7 mmol) and NH₄Cl (742 mg, 13.7 mmol) were added to the solution of 2-cyclopropyl-7-nitro-1,2,3,4-tetrahydroisoquinoline (48.1) (500 mg, 2.3 mmol) in EtOH (20 mL) and H₂O (2 mL). The mixture was stirred at 90° C. for 1 h. After cooling to room temperature, the reaction mixture was diluted with water and extracted with ethyl acetate. The combined organic layers were washed with water and brine, dried over sodium sulfate, and concentrated to afford the crude product. The crude product was purified by column chromatography (ethyl acetate/hexane:1/1) to afford 2-cyclopropyl-1,2,3,4-tetrahydroisoquinolin-7-amine (48.2) as a yellow solid (300 mg, 69%).

LCMS: 189.1 [M+1]⁺.

### (R)-1-(3-(2-(2-Cyclopropyl-1,2,3,4-tetrahydroisoquinolin-7-ylamino)-5-fluoropyrimidin-4-ylamino)piperidin-1-yl)prop-2-en-1-one (I-48)

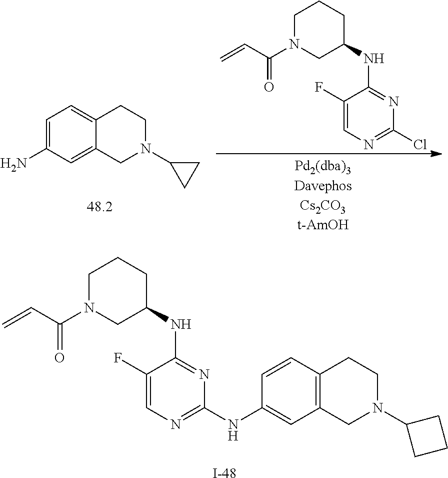

(R)-1-(3-(2-Chloro-5-fluoropyrimidin-4-ylamino)piperidin-1-yl)prop-2-en-1-one (intermediate B, 400 mg, 1.4 mmol) and Cs₂CO₃ (913 mg, 2.8 mmol) were added to the solution of 2-cyclopropyl-1,2,3,4-tetrahydroisoquinolin-7-amine (48.2) (180 mg, 0.95 mmol) in tert-amyl alcohol (10 mL). Tris(dibenzylideneacetone)dipalladium(O) (128 mg, 0.14 mmol) and DavePhos (55 mg, 0.14 mmol) were added under N₂, and the reaction mixture was stirred at 100° C. overnight. The reaction mixture was diluted with water and extracted with ethyl acetate. The combined organic layers were washed with water and brine, dried over sodium sulfate, and concentrated to afford the crude product. The crude product was purified by column chromatography (DCM/MeOH:10/1) to afford (R)-1-(3-(2-(2-cyclopropyl-1,2,3,4-tetrahydroisoquinolin-7-ylamino)-5-fluoropyrimidin-4-ylamino)piperidin-1-yl)prop-2-en-1-one (I-48) as a yellow solid (150 mg, 36%).

Mp: 105-107° C.

LCMS: 437.2 [M+1]⁺.

¹H NMR (400 MHz, DMSO-d₆): δ 0.38-0.49 (m, 4H), 1.41-1.46 (m, 1H), 1.60-1.85 (m, 3H), 1.99-2.01 (m, 1H), 2.60-2.78 (m, 5H), 3.00-3.14 (m, 1H), 3.60 (s, 2H), 4.00-4.02 (m, 2H), 4.22-4.25 (m, 0.5H), 4.44-4.47 (m, 0.5H), 5.46-5.49 (m, 0.5H), 5.68-5.70 (m, 0.5H), 6.00-6.15 (m, 1H), 6.57-6.64 (m, 0.5H), 6.82-6.90 (m, 1.5H), 7.28-7.38 (m, 2H), 7.52 (d, 1H), 7.89 (d, 1H), 8.96 (s, 1H).

### Example 49

### Preparation of (R)-1-(3-((2-((2-(tert-butyl)-1,2,3,4-tetrahydroisoquinolin-7-yl)amino)-5-fluoropyrimidin-4-yl)amino)piperidin-1-yl)prop-2-en-1-one (I-49)

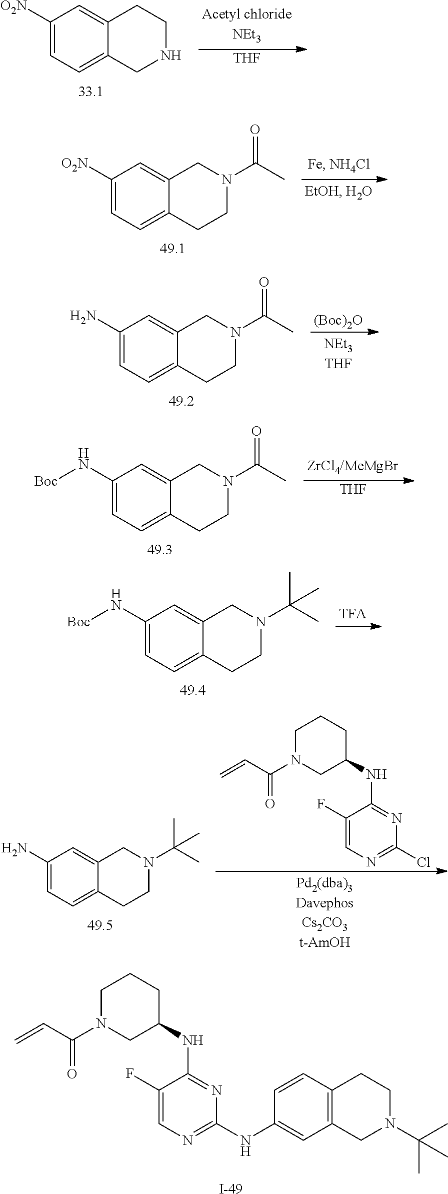

### 1-(7-Nitro-3,4-dihydroisoquinolin-2(1H)-yl)ethanone (49.1)

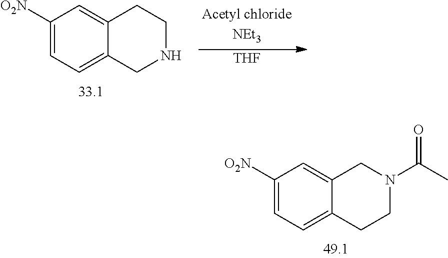 [SMILES: O=[N+]([O-])c1ccc2c(c1)CCNC2 ; CC(=O)N1CCc2ccc([N+](=O)[O-])cc2C1 ; "THF"]

Acetyl chloride (1.3 g, 16.6 mmol) was slowly added to a solution of 7-nitro-1,2,3,4-tetrahydroisoquinoline (33.1) (2.0 g, 11.2 mmol) and triethylamine (2.3 g, 22.4 mmol) in THF (20 mL) at 0° C., and the reaction mixture was stirred at ambient temperature for 1 h. The reaction mixture was diluted with water and extracted with ethyl acetate. The combined organic layers were washed with water and brine, dried over sodium sulfate, and concentrated to afford the crude product. The crude product was purified by column chromatography (DCM/MeOH: 20/1) to afford 1-(7-nitro-3,4-dihydroisoquinolin-2(1H)-yl)ethanone (49.1) as a brown solid (2.4 g, 97%).

LCMS: 221.2 [M+1]⁺.

### 1-(7-Amino-3,4-dihydroisoquinolin-2(1H)-yl)ethanone (49.2)

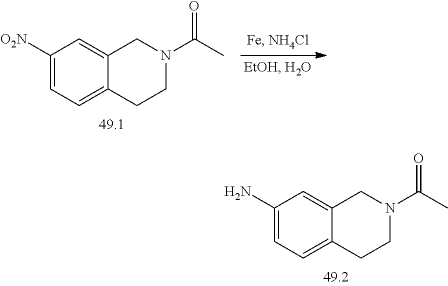 [SMILES: CC(=O)N1CCc2ccc([N+](=O)[O-])cc2C1 ; CC(=O)N1CCc2ccc(N)cc2C1]

Fe (6.1 g, 109 mmol) and NH₄Cl (5.9 g, 109 mmol) were added to a solution of 1-(7-nitro-3,4-dihydroisoquinolin-2(1H)-yl)ethanone (49.1) (2.4 g, 10.9 mmol) in EtOH (50 mL) and H₂O (3 mL). The mixture was stirred at 90° C. for 1 h. After cooling to room temperature, the reaction mixture was diluted with water and extracted with ethyl acetate. The combined organic layers were washed with water and brine, dried over sodium sulfate, and concentrated to afford the crude product. The crude product was purified by column chromatography (DCM/MeOH:20/1) to afford 1-(7-amino-3,4-dihydroisoquinolin-2(1H)-yl)ethanone (49.2) as a brown solid (2.0 g, 96%).

### tert-Butyl 2-acetyl-1,2,3,4-tetrahydroisoquinolin-7-ylcarbamate (49.3)

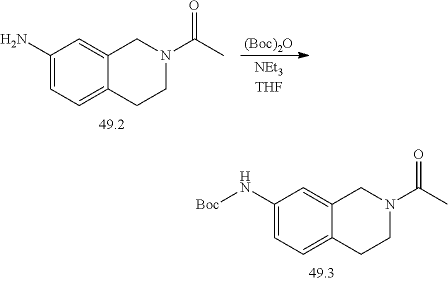 [SMILES: "(Boc)2O" ; CC(=O)N1CCc2ccc(N)cc2C1 ; CC(=O)N1CCc2ccc(NC(=O)OC(C)(C)C)cc2C1]

A solution of 1-(7-amino-3,4-dihydroisoquinolin-2(1H)-yl)ethanone (49.2) (2.0 g, 10.5 mmol), di-tert-butyl dicarbonate (2.7 g, 12.6 mmol), and triethylamine (2.1 g, 21.0 mmol) in THF (10 mL) was stirred at 40° C. for 1 h. After cooling to room temperature, the reaction mixture was diluted with water and extracted with ethyl acetate. The combined organic layers were washed with water and brine, dried over sodium sulfate, and concentrated to afford the crude product. The crude product was purified by column chromatography (DCM/MeOH:20/1) to afford tert-butyl 2-acetyl-1,2,3,4-tetrahydroisoquinolin-7-ylcarbamate (49.3) as a white solid (1.4 g, 46%).

LCMS: 291.0 [M+1]⁺.

### tert-Butyl (2-(tert-butyl)-1,2,3,4-tetrahydroisoquinolin-7-yl)carbamate (49.4)

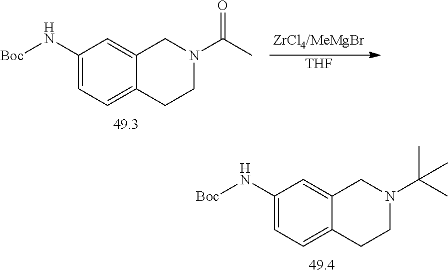 [SMILES: CC(=O)N1CCc2ccc(NC(=O)OC(C)(C)C)cc2C1 ; CC(C)(C)OC(=O)Nc1ccc2c(c1)CN(C(C)(C)C)CC2 ; "THF"]

ZrCl₄ (1.48 g, 2.07 mmol) was slowly added to the solution of tert-butyl 2-acetyl-1,2,3,4-tetrahydroisoquinolin-7-ylcarbamate (49.3) (300 mg, 1.03 mmol) in THF (10 mL) at −20° C. The reaction mixture was stirred at −20° C. for 1 h. Methylmagnesium bromide (6 mL, 1M) was slowly added to the reaction, and the reaction mixture was stirred at ambient temperature overnight. The reaction mixture was diluted with water and extracted with ethyl acetate. The combined organic layers were washed with water and brine, dried over sodium sulfate, and concentrated to afford the crude product. The crude product was purified by column chromatography (DCM/MeOH:10/1) to afford tert-butyl (2-(tert-butyl)-1,2,3,4-tetrahydroisoquinolin-7-yl)carbamate (49.4) as a brown solid (40 mg, 13%).

LCMS: 305.1 [M+1]⁺.

### 2-tert-Butyl-1,2,3,4-tetrahydroisoquinolin-7-amine (49.5)

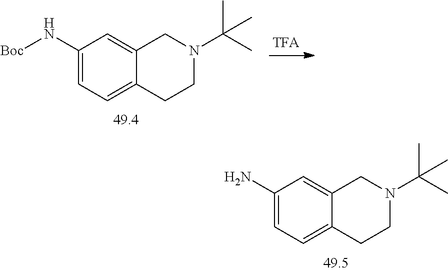 [SMILES: CC(C)(C)OC(=O)Nc1ccc2c(c1)CN(C(C)(C)C)CC2 ; CC(C)(C)N1CCc2ccc(N)cc2C1]

A solution of tert-butyl (2-(tert-butyl)-1,2,3,4-tetrahydroisoquinolin-7-yl)carbamate (49.4) (40 mg, 0.13 mmol) in TFA (2 mL) was stirred at ambient temperature for 30 min. The reaction mixture was evaporated to afford the crude product, 2-tert-butyl-1,2,3,4-tetrahydroisoquinolin-7-amine (49.5), which was used in the next step without further purification.

### (R)-1-(3-(2-(2-tert-Butyl-1,2,3,4-tetrahydroisoquinolin-7-ylamino)-5-fluoropyrimidin-4-ylamino)piperidin-1-yl)prop-2-en-1-one (I-49)

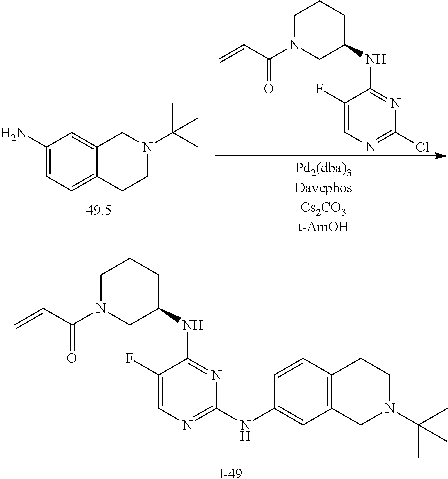

(R)-1-(3-(2-chloro-5-fluoropyrimidin-4-ylamino)piperidin-1-yl)prop-2-en-1-one (44.9 mg, 0.16 mmol) and Cs₂CO₃ (85.7 mg, 0.26 mmol) were added to the solution of 2-tert-butyl-1,2,3,4-tetrahydroisoquinolin-7-amine (49.5) in tert-amyl alcohol (3 mL). Tris(dibenzylideneacetone)dipalladium(O) (12 mg, 0.013 mmol) and DavePhos (10 mg, 0.026 mmol) were added under N₂, and the reaction mixture was stirred at 100° C. for 1 h. The reaction mixture was diluted with water and extracted with ethyl acetate. The combined organic layers were washed with water and brine, dried over sodium sulfate, and concentrated in vacuo to afford the crude product. The crude product was purified by column chromatography (DCM/MeOH:10/1) to afford (R)-1-(3-(2-(2-tert-butyl-1,2,3,4-tetrahydroisoquinolin-7-ylamino)-5-fluoropyrimidin-4-ylamino)piperidin-1-yl)prop-2-en-1-one (I-49) as a yellow solid (25 mg, 42%).

Mp: 118-121° C.

LCMS: 453.4 [M+1]⁺.

¹H NMR (400 MHz, DMSO-d₆): δ 1.07 (s, 9H), 1.40-1.43 (m, 1H), 1.57-1.67 (m, 1H), 1.82-1.85 (m, 1H), 1.99-2.01 (m, 1H), 2.64-2.77 (m, 5H), 2.99-3.09 (m, 1H), 3.54-3.60 (m, 2H), 3.98-4.04 (m, 2H), 4.24-4.44 (m, 1H), 5.45-5.48 (m, 0.5H), 5.68-5.71 (m, 0.5H), 5.99-6.14 (m, 1H), 6.56-6.63 (m, 0.5H), 6.80-6.89 (m, 1.5H), 7.19-7.41 (m, 2H), 7.52-7.58 (m, 1H), 7.88 (d, 1H), 8.95 (s, 1H).

### Example 50

### Preparation of (R)-1-(3-((5-Fluoro-2-((2-(oxetan-3-ylmethyl)-2,3,4,5-tetrahydro-1H-benzo[c]azepin-7-yl)amino)pyrimidin-4-yl)amino)piperidin-1-yl)prop-2-en-1-one (I-50)

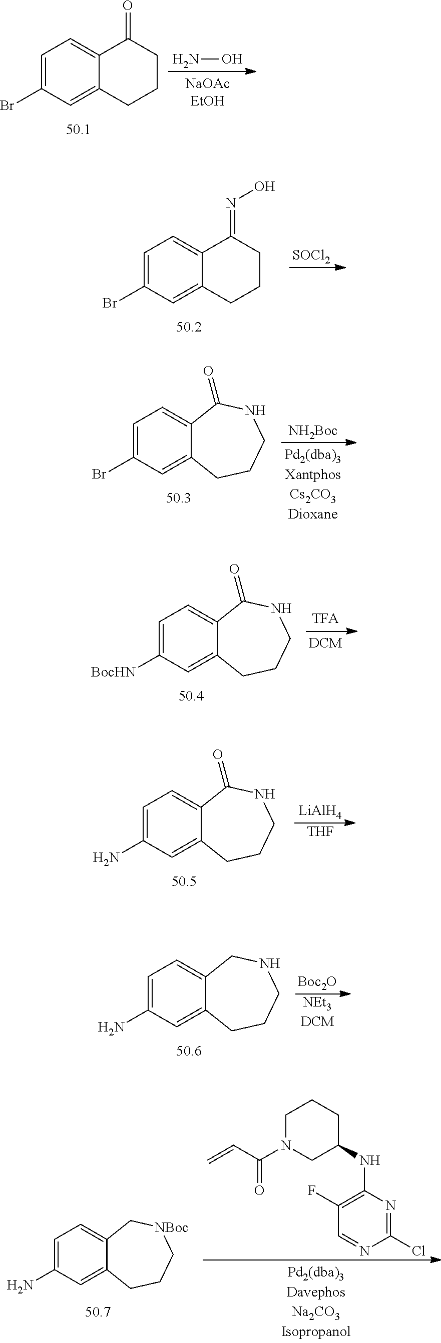

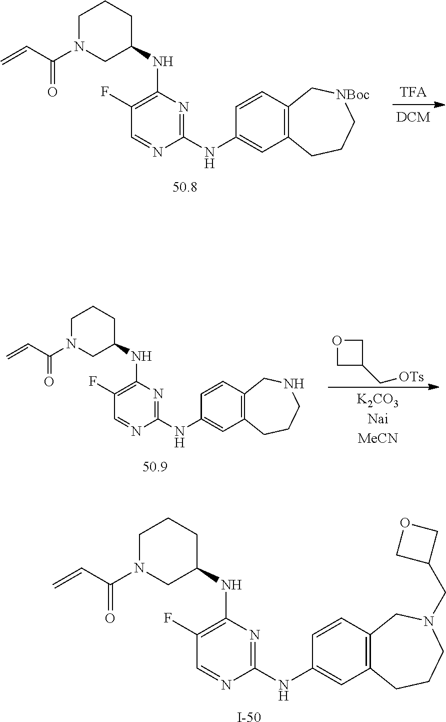

### (E)-6-Bromo-3,4-dihydronaphthalen-1(2H)-one oxime (50.2)

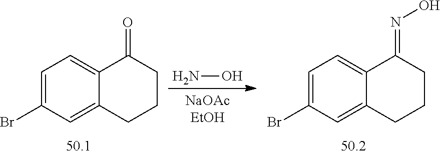

A solution of 6-bromo-3,4-dihydronaphthalen-1(2H)-one (50.1) (5 g, 22.2 mmol), hydroxylamine (0.81 g, 24.4 mmol), and NaOAc (3.64 g, 44.4 mmol) in ethanol (40 ml) was refluxed for 2 h. TLC showed the reaction was complete. The mixture was poured into water and filtered, and the solid was dried to afford (E)-6-bromo-3,4-dihydronaphthalen-1(2H)-one oxime (50.2) as a yellow solid (5.2 g, 97%).

### 7-Bromo-2,3,4,5-tetrahydro-1H-benzo[c]azepin-1-one (50.3)

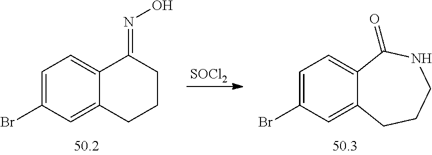

A solution of (E)-6-bromo-3,4-dihydronaphthalen-1(2H)-one oxime (50.2) (5.2 g, 21.7 mmol) in sulfurous dichloride (15 mL) was heated at 50° C. for 2 h. TLC showed the reaction was complete. The reaction mixture was concentrated and poured into a sodium bicarbonate solution and extracted with ethyl acetate. The combined organic layers were washed with water and brine, dried over anhydrous sodium sulfate, and concentrated to afford the crude product. The crude product was purified by column chromatography to afford 7-bromo-2,3,4,5-tetrahydro-1H-benzo[c]azepin-1-one (50.3) as a green solid (2.4 g, 46%).

LCMS: 239.9 [M+1]⁺.

¹HNMR (DMSO-d₆): δ 1.85-1.91 (m, 2H), 2.74 (t, 2H), 2.90 (q, 2H), 7.42-7.44 (m, 1H), 7.53-7.55 (m, 2H), 8.13 (t, 1H).

### tert-Butyl (1-oxo-2,3,4,5-tetrahydro-1H-benzo[c]azepin-7-yl)carbamate (50.4)

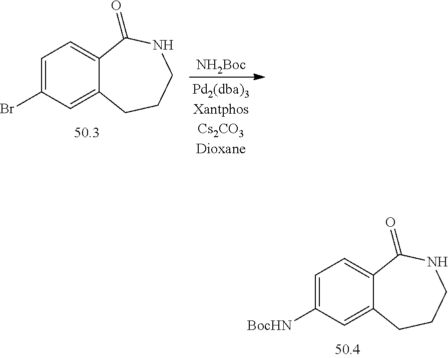 [SMILES: O=C1NCCCc2cc(Br)ccc21 ; CC(C)(C)OC(=O)Nc1ccc2c(c1)CCCNC2=O ; "NH2Boc"]

A solution of 7-bromo-2,3,4,5-tetrahydro-1H-benzo[c]azepin-1-one (50.3) (1 g, 4.16 mmol), tert-butyl carbamate (1.38 g, 11.8 mmol), tris(dibenzylideneacetone)dipalladium(O) (72 mg, 0.08 mmol), Xantphos (45 mg, 0.08 mmol), and Cs₂CO₃ (2.56 g, 7.87 mmol) in dioxane (15 mL) was stirred at 100° C. for 4 h under N₂. TLC showed the reaction was complete. The mixture was poured into water and extracted with ethyl acetate. The combined organic layers were washed with water and brine, dried over anhydrous sodium sulfate, and concentrated to afford the crude product. The crude product was purified by column chromatography to afford tert-butyl (1-oxo-2,3,4,5-tetrahydro-1H-benzo[c]azepin-7-yl)carbamate (50.4) as a brown solid (450 mg, 39%).

LCMS: 277.2 [M+1]⁺.

### 7-Amino-2,3,4,5-tetrahydro-1H-benzo[c]azepin-1-one (50.5)

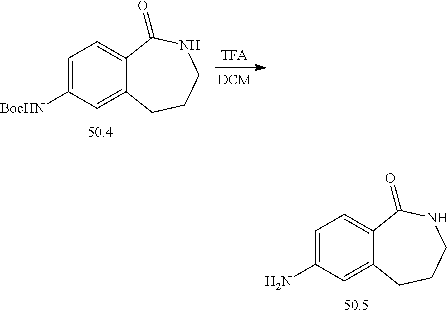 [SMILES: CC(C)(C)OC(=O)Nc1ccc2c(c1)CCCNC2=O ; Nc1ccc2c(c1)CCCNC2=O]

A solution of tert-butyl (1-oxo-2,3,4,5-tetrahydro-1H-benzo[c]azepin-7-yl)carbamate (50.4) (450 mg, 1.63 mmol) and TFA (3 mL) in DCM (3 mL) was stirred at room temperature for 30 min. TLC showed the reaction was complete. The mixture was concentrated and poured into a sodium bicarbonate solution and was extracted with ethyl acetate. The combined organic layers were washed with water and brine, dried over anhydrous sodium sulfate, and concentrated to afford 7-amino-2,3,4,5-tetrahydro-1H-benzo[c]azepin-1-one (50.5) as a brown solid (300 mg, 100%).

LCMS: 177.1 [M+1]⁺.

¹HNMR (DMSO-d₆): δ 1.79-1.85 (m, 2H), 2.57 (t, 2H), 2.90 (t, 3H), 5.48 (s, 2H), 6.34 (s, 1H), 6.43 (dd, 1H), 7.19 (d, 1H), 7.57 (t, 1H).

### 2,3,4,5-Tetrahydro-1H-benzo[c]azepin-7-amine (50.6)

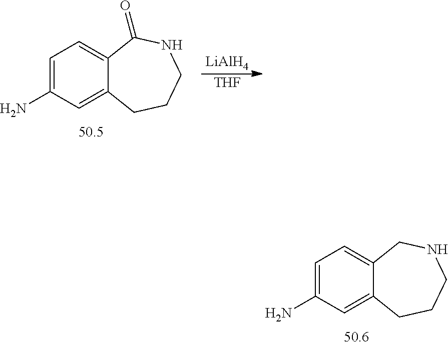

A solution of 7-amino-2,3,4,5-tetrahydro-1H-benzo[c]azepin-1-one (50.5) (300 mg, 1.7 mmol) and LiAlH₄ (258 mg, 6.8 mmol) in THF (10 ml) was heated at reflux overnight. TLC showed the reaction was complete. The mixture was poured into water and extracted with ethyl acetate. The combined organic layers were washed with water and brine, dried over anhydrous sodium sulfate, and concentrated to afford the crude product. The crude product was purified by column chromatography to afford 2,3,4,5-tetrahydro-1H-benzo[c]azepin-7-amine (50.6) as a yellow solid (100 mg, 36%).

### tert-Butyl 7-amino-1,3,4,5-tetrahydro-2H-benzo[c]azepine-2-carboxylate (50.7)

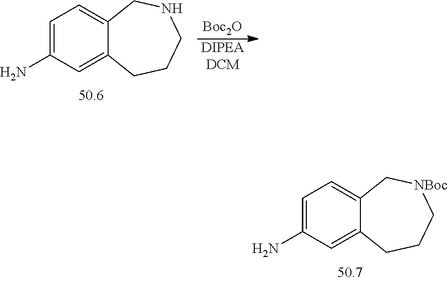

A solution of 2,3,4,5-tetrahydro-1H-benzo[c]azepin-7-amine (50.6) (100 mg, 0.62 mmol), di-tert-butyl dicarbonate (121 mg, 0.55 mmol), and N,N-diisopropylethylamine (DIPEA) (159 mg, 1.23 mmol) in THF (5 mL) was stirred at ambient temperature for 2 h. TLC showed the reaction was complete. The mixture was poured into water and extracted with ethyl acetate. The combined organic layers were washed with water and brine, dried over anhydrous sodium sulfate, and concentrated to afford the crude product. The crude product was purified by column chromatography to afford tert-butyl 7-amino-1,3,4,5-tetrahydro-2H-benzo[c]azepine-2-carboxylate (50.7) as a yellow solid (150 mg, 93%).

LCMS: 263.1 [M+1]⁺.

### (R)-tert-Butyl 7-((4-((1-acryloylpiperidin-3-yl)amino)-5-fluoropyri-midin-2-yl)amino)-4,5-dihydro-1H-benzo[c]azepine-2(3H)-carboxylate (50.8)

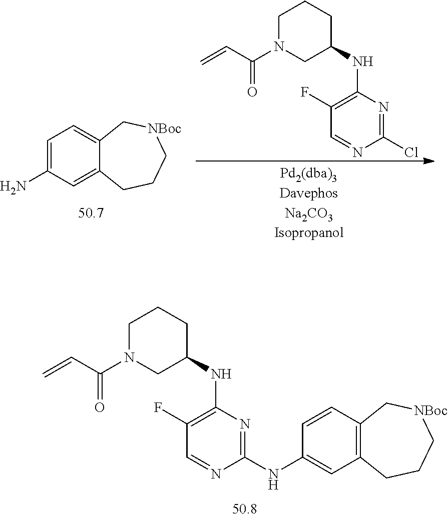 [SMILES: CC(C)(C)OC(=O)N1CCCc2cc(N)ccc2C1 ; C=CC(=O)N1CCC[C@@H](Nc2nc(Cl)ncc2F)C1 ; C=CC(=O)N1CCC[C@@H](Nc2nc(Nc3ccc4c(c3)CCCN(C(=O)OC(C)(C)C)C4)ncc2F)C1]

A solution of tert-butyl 7-amino-4,5-dihydro-1H-benzo[c]azepine-2(3H)-carboxylate (50.7) (150 mg, 0.57 mmol), (R)-1-(3-((2-chloro-5-fluoropyrimidin-4-yl)amino)piperidin-1-yl)prop-2-en-1-one (intermediate B, 195 mg, 0.69 mmol), tris(dibenzylideneacetone)dipalladium(O) (52 mg, 0.06 mmol), Davephos (23 mg, 0.06 mmol), and Cs₂CO₃ (372 mg, 1.14 mmol) in isopropanol (10 mL) was heated at reflux under N₂ for 2 h. TLC showed the reaction was complete. The mixture was poured into water and extracted with ethyl acetate. The combined organic layers were washed with water and brine, dried over anhydrous sodium sulfate, and concentrated to afford the crude product. The crude product was purified by column chromatography to afford (R)-tert-butyl 7-((4-((1-acryloylpiperidin-3-yl)amino)-5-fluoropyri-midin-2-yl)amino)-4,5-dihydro-1H-benzo[c]azepine-2(3H)-carboxylate (50.8) as a yellow solid (200 mg, 69%).

LCMS: 511.7 [M+1]⁺.

### (R)-1-(3-((5-Fluoro-2-((2,3,4,5-tetrahydro-1H-benzo[c]azepin-7-yl)amino)pyrimidin-4-yl)amino)piperidin-1-yl)prop-2-en-1-one (50.9)

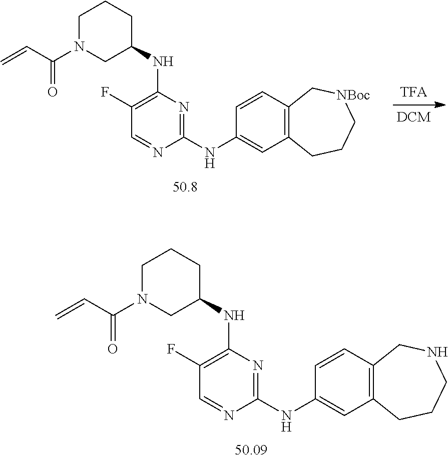 [SMILES: C=CC(=O)N1CCC[C@@H](Nc2nc(Nc3ccc4c(c3)CCCN(C(=O)OC(C)(C)C)C4)ncc2F)C1 ; C=CC(=O)N1CCC[C@@H](Nc2nc(Nc3ccc4c(c3)CCCNC4)ncc2F)C1]

A solution of (R)-tert-butyl 7-((4-(1-acryloylpiperidin-3-yl)amino)-5-fluoropyri-midin-2-yl)amino)-4,5-dihydro-1H-benzo[c]azepine-2(3H)-carboxylate (50.8) (200 mg, 0.39 mmol) and TFA (5 mL) in DCM (5 mL) was stirred at ambient temperature for 30 min. TLC showed the reaction was complete. The mixture was concentrated to afford (R)-1-(3-((5-fluoro-2-((2,3,4,5-tetrahydro-1H-benzo[c]azepin-7-yl)amino)pyrimidin-4-yl)amino)piperidin-1-yl)prop-2-en-1-one (50.9) as a yellow oil, which was used in the next step without further purification.

### (R)-1-(3-((5-Fluoro-2-((2-(oxetan-3-ylmethyl)-2,3,4,5-tetrahydro-1H-benzo[c]azepin-7-yl)amino)pyrimidin-4-yl)amino)piperidin-1-yl)prop-2-en-1-one (I-50)

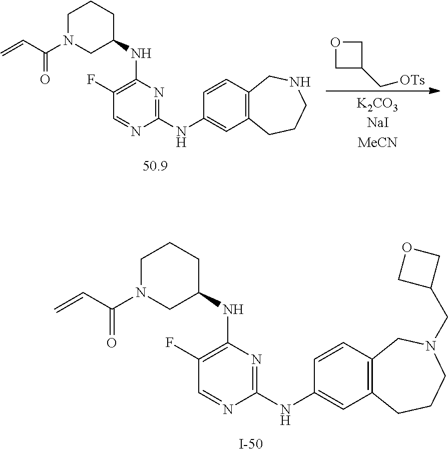

A solution of (R)-1-(3-((5-fluoro-2-((2,3,4,5-tetrahydro-1H-benzo[c]azepin-7-yl)amino)pyrimidin-4-yl)amino)piperidin-1-yl)prop-2-en-1-one (50.9) (0.39 mmol) and oxetan-3-ylmethyl-4-methyl benzenesulfonate (142 mg, 0.58 mmol), K₂CO₃ (83 mg, 0.78 mmol), and NaI (58 mg, 0.39 mmol) in MeCN (5 mL) was heated at reflux overnight. TLC showed the reaction was complete. The mixture was poured into water and extracted with ethyl acetate. The combined organic layers were washed with water and brine, dried over anhydrous sodium sulfate, and concentrated to afford the crude product. The crude product was purified by column chromatography to afford (R)-1-(3-((5-fluoro-2-((2-(oxetan-3-ylmethyl)-2,3,4,5-tetrahydro-1H-benzo[c]azepin-7-yl)amino)pyrimidin-4-yl)amino)piperidin-1-yl)prop-2-en-1-one (I-50) as a yellow solid (50 mg, 27%).

LCMS: 481.4 [M+1]⁺.

¹HNMR (DMSO-d₆): δ 1.39 (br, 1H), 1.55-1.67 (m, 3H), 1.80-1.84 (m, 1H), 1.97-2.01 (m, 1H), 2.54 (s, 2H), 2.69-2.77 (m, 2H), 2.94-3.13 (m, 4H), 3.69 (s, 2H), 4.02 (d, 2H), 4.16-4.24 (m, 2.5H), 4.43 (d, 0.5H), 4.58 (t, 2H), 5.48 (d, 0.5H), 5.71 (d, 0.5H), 6.02 (d, 0.5H), 6.13 (d, 0.5H), 6.63 (dd, 0.5H), 6.86 (dd, 0.5H), 6.95 (d, 1H), 7.30 (t, 1H), 7.40 (t, 1H), 7.53 (s, 1H), 7.89 (s, 1H), 9.00 (s, 1H).

### Example 51

### Preparation of (R)-1-(3-((2-((2-Cyclopropyl-2,3,4,5-tetrahydro-1H-benzo[c]azepin-7-yl)amino)-5-fluoropyrimidin-4-yl)amino)piperidin-1-yl)prop-2-en-1-one (I-51)

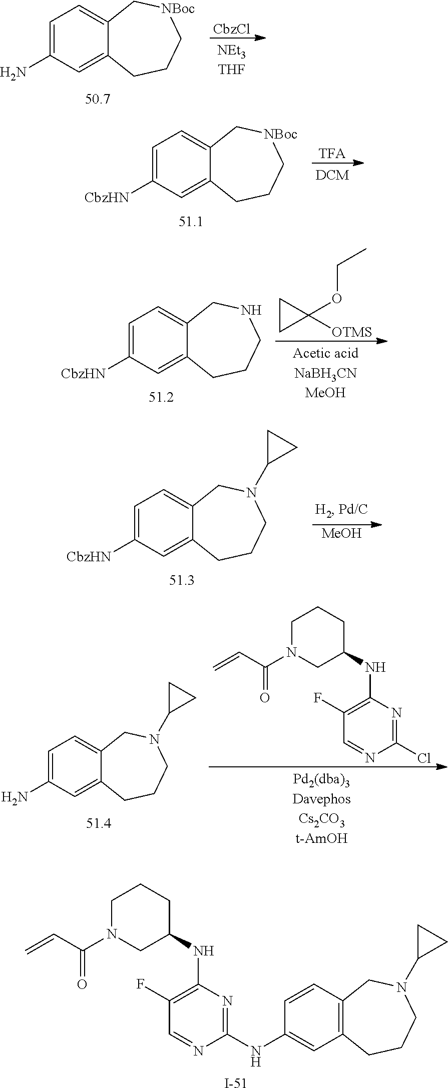

### tert-butyl 7-(((benzyloxy)carbonyl)amino)-1,3,4,5-tetrahydro-2H-benzo[c]azepine-2-carboxylate (51.1)

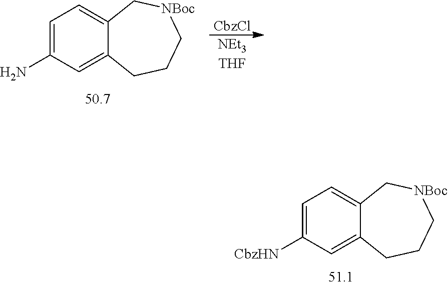 [SMILES: "CbzCl" ; CC(C)(C)OC(=O)N1CCCc2cc(N)ccc2C1 ; "NEt3THF" ; CC(C)(C)OC(=O)N1CCCc2cc(NC(=O)OCc3ccccc3)ccc2C1]

A solution of tert-butyl 7-amino-4,5-dihydro-1H-benzo[c]azepine-2(3H)-carboxylate (50.7) (1 g, 3.81 mmol), CbzCl (975 mg, 5.71 mmol) and triethylamine (770 mg, 7.62 mmol) in THF (15 ml) was stirred at 0° C. for 2 h. TLC showed the reaction was complete. The mixture was poured into water and extracted with ethyl acetate. The combined organic layers were washed with water and brine, dried over anhydrous sodium sulfate, and concentrated to afford the crude product. The crude product was purified by column chromatography to afford tert-butyl 7-(((benzyloxy)carbonyl)amino)-1,3,4,5-tetrahydro-2H-benzo[c]azepine-2-carboxylate (51.1) as a colorless oil (1.1 g, 73%).

LCMS: 419.2 [M+1]⁺.

### Benzyl (2,3,4,5-tetrahydro-1H-benzo[c]azepin-7-yl)carbamate (51.2)

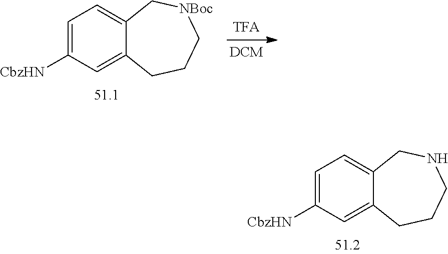 [SMILES: CC(C)(C)OC(=O)N1CCCc2cc(NC(=O)OCc3ccccc3)ccc2C1 ; O=C(Nc1ccc2c(c1)CCCNC2)OCc1ccccc1]

A solution of tert-butyl 7-(((benzyloxy)carbonyl)amino)-1,3,4,5-tetrahydro-2H-benzo[c]azepine-2-carboxylate (51.1) (1.1 g, 2.77 mmol) and TFA (5 mL) in DCM (5 mL) was stirred at ambient temperature for 30 min. TLC showed the reaction was complete. The mixture was concentrated, and the residue was added to a sodium bicarbonate solution. The resulting solution was extracted with ethyl acetate. The combined organic layers were washed with water and brine, dried over anhydrous sodium sulfate, and concentrated to afford benzyl (2,3,4,5-tetrahydro-1H-benzo[c]azepin-7-yl)carbamate (51.2) as a white solid (600 mg, 73%).

### Benzyl (2-cyclopropyl-2,3,4,5-tetrahydro-1H-benzo[c]azepin-7-yl)carbamate (51.3)

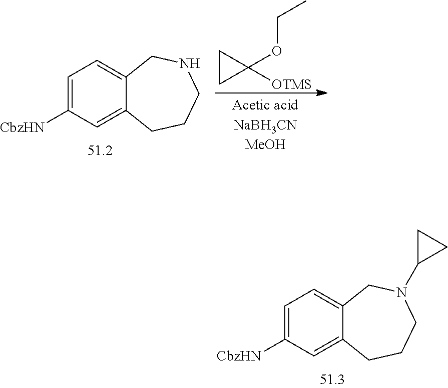

A mixture of benzyl (2,3,4,5-tetrahydro-1H-benzo[c]azepin-7-yl)carbamate (51.2) (200 mg, 0.67 mmol), (1-ethoxycyclopropoxy)trimethylsilane (235 mg, 1.35 mmol), acetic acid (202 mg, 3.37 mmol), sodium cyanoborohydride (128 mg, 2.02 mmol), and molecular sieves in MeOH (10 mL) was stirred at 60° C. for 4 h. TLC showed the reaction was complete. The mixture was filtered and concentrated. The residue was dissolved in DCM and the combined organic layers were washed with a 2 N sodium hydroxide solution, dried over anhydrous sodium sulfate, and concentrated to afford the crude product. The crude product was purified by column chromatography to afford benzyl (2-cyclopropyl-2,3,4,5-tetrahydro-1H-benzo[c]azepin-7-yl)carbamate (51.3) as a white solid (100 mg, 44%).

LCMS: 337.4 [M+1]⁺.

### 2-Cyclopropyl-2,3,4,5-tetrahydro-1H-benzo[c]azepin-7-amine (51.4)

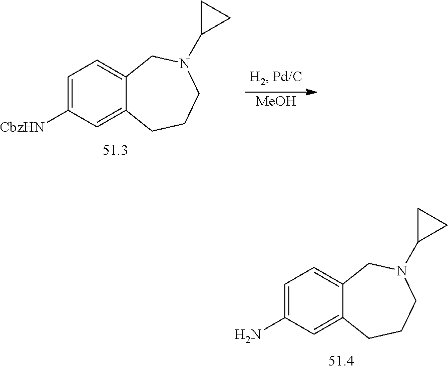 [SMILES: "MeOH" ; O=C(Nc1ccc2c(c1)CCCN(C1CC1)C2)OCc1ccccc1 ; Nc1ccc2c(c1)CCCN(C1CC1)C2]

A solution of benzyl (2-cyclopropyl-2,3,4,5-tetrahydro-1H-benzo[c]azepin-7-yl)carbamate (51.3) (100 mg, 0.3 mmol) and Pd/C (20 mg) in MeOH (10 mL) was stirred at ambient temperature under H₂ overnight. The mixture was filtered, and the filtrate was concentrated to afford 2-cyclopropyl-2,3,4,5-tetrahydro-1H-benzo[c]azepin-7-amine (51.4) as a colorless oil (50 mg, 83%).

### (R)-1-(3-((2-((2-Cyclopropyl-2,3,4,5-tetrahydro-1H-benzo[c]azepin-7-yl)amino)-5-fluoropyrimidin-4-yl)amino)piperidin-1-yl)prop-2-en-1-one (I-51)

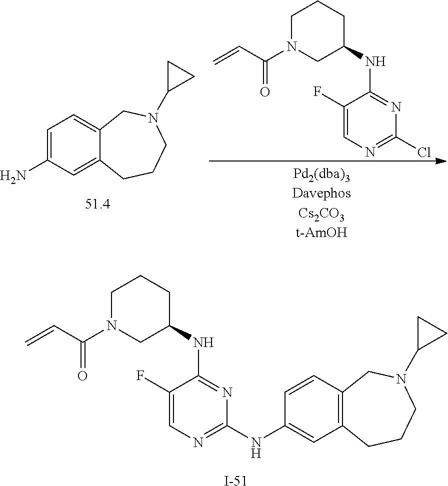

A mixture of 2-cyclopropyl-2,3,4,5-tetrahydro-1H-benzo[c]azepin-7-amine (51.4) (30 mg, 0.15 mmol), (R)-1-(3-((2-chloro-5-fluoropyrimidin-4-yl)amino)piperidin-1-yl)prop-2-en-1-one (intermediate B, 51 mg, 0.18 mmol), tris(dibenzylideneacetone)dipalladium(O) (14 mg, 0.015 mmol), Davephos (6 mg, 0.015 mmol), and Cs₂CO₃ (97 mg, 0.30 mmol) in tert-amyl alcohol (5 mL) was heated at 100° C. for 2 h. TLC showed the reaction was complete. The mixture was poured into water and extracted with ethyl acetate. The combined organic layers were washed with water and brine, dried over anhydrous sodium sulfate, and concentrated to afford the crude product. The crude product was purified by column chromatography to afford (R)-1-(3-((2-((2-Cyclopropyl-2,3,4,5-tetrahydro-1H-benzo[c]azepin-7-yl)amino)-5-fluoropyrimidin-4-yl)amino)piperidin-1-yl)prop-2-en-1-one (I-51) as a yellow solid (17.1 mg, 26%).

LCMS: 451.3 [M+1]⁺.

¹HNMR (DMSO-d₆): δ 0.31 (two singlets, 4H), 1.39 (br, 1H), 1.62 (br, 4H), 1.80-1.83 (m, 1H), 1.97-2.01 (m, 1H), 2.67-2.78 (m, 3H), 3.0-3.17 (m, 3H), 3.75 (s, 2H), 4.02 (d, 2H), 4.23 (d, 0.5H), 4.44 (d, 0.5H), 5.48 (d, 0.5H), 5.71 (d, 0.5H), 6.02 (d, 0.5H), 6.13 (d, 0.5H), 6.63 (dd, 0.5H), 6.86 (dd, 0.5H), 6.93 (d, 1H), 7.31 (t, 1H), 7.40 (br, 1H), 7.52 (d, 1H), 7.89 (s, 1H), 8.99 (s, 1H).

### Example 52

### Preparation of (R)-7-((4-((1-acryloylpiperidin-3-yl)amino)-5-fluoropyrimidin-2-yl)amino)-2-methyl-2,3,4,5-tetrahydro-1H-benzo[c]azepin-1-one (I-52)

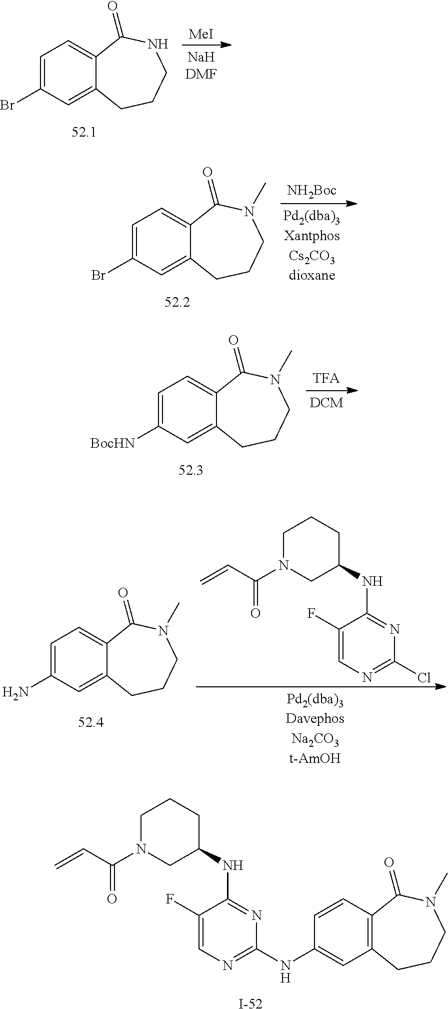

### 7-Bromo-2-methyl-2,3,4,5-tetrahydro-1H-benzo[c]azepin-1-one (52.2)

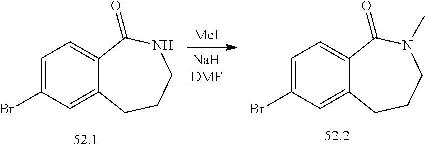

A solution of 7-bromo-2,3,4,5-tetrahydro-1H-benzo[c]azepin-1-one (52.1) (1 g, 4.16 mmol) in DMF (15 ml) was stirred at 0° C. Sodium hydride (333 mg, 8.33 mmol) was added slowly and the mixture was stirred at 0° C. for 30 min. Iodomethane (1.18 g, 8.33 mmol) was added, and the solution was stirred at ambient temperature overnight. TLC showed the reaction was complete. The mixture was added to water and extracted with ethyl acetate. The combined organic layers were washed with water and brine, dried over anhydrous sodium sulfate, and concentrated to afford 7-bromo-2-methyl-2,3,4,5-tetrahydro-1H-benzo[c]azepin-1-one (52.2) as a brown solid (1.1 g, quantitative).

LCMS: 256.0 [M+1]⁺.

### tert-Butyl (2-methyl-1-oxo-2,3,4,5-tetrahydro-1H-benzo[c]azepin-7-yl)carbamate (52.3)

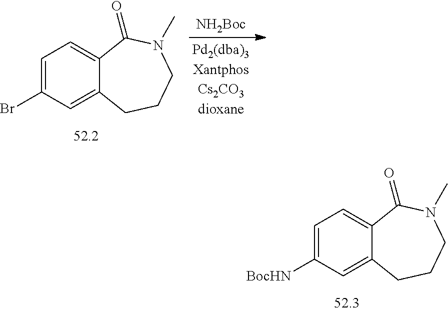 [SMILES: CN1CCCc2cc(Br)ccc2C1=O ; CN1CCCc2cc(NC(=O)OC(C)(C)C)ccc2C1=O ; "NH2Boc"]

A mixture of 7-bromo-2-methyl-2,3,4,5-tetrahydro-1H-benzo[c]azepin-1-one (52.2) (1 g, 3.94 mmol), tert-butyl carbamate (1.38 g, 11.8 mmol), Tris(dibenzylideneacetone)dipalladium(O) (72 mg, 0.08 mmol), Xantphos (45 mg, 0.08 mmol), and Cs₂CO₃ (2.56 g, 7.87 mmol) in dioxane (15 mL) was stirred at 100° C. for 4 h under N₂. TLC showed the reaction was complete. The mixture was quenched with the addition of water and extracted with ethyl acetate. The combined organic layers were washed with water and brine, dried over anhydrous sodium sulfate, and concentrated to afford the crude product. The crude product was purified by column chromatography (DCM/MeOH:60/1) to afford tert-butyl (2-methyl-1-oxo-2,3,4,5-tetrahydro-1H-benzo[c]azepin-7-yl)carbamate (52.3) as a yellow solid (500 mg, 44%).

LCMS: 291.2 [M+1]⁺.

### 7-Amino-2-methyl-2,3,4,5-tetrahydro-1H-benzo[c]azepin-1-one (52.4)

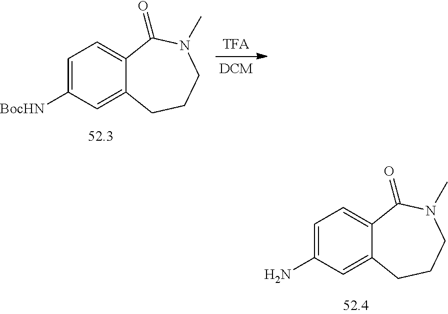 [SMILES: CN1CCCc2cc(NC(=O)OC(C)(C)C)ccc2C1=O ; CN1CCCc2cc(N)ccc2C1=O ; "TFA"]

A solution of tert-butyl (2-methyl-1-oxo-2,3,4,5-tetrahydro-1H-benzo[c]azepin-7-yl)carbamate (52.3) (180 mg, 0.62 mmol) and TEA (4 mL) DCM (4 mL) was stirred at ambient temperature for 30 min. The mixture was concentrated in vacuo, and the residue was poured into a sodium bicarbonate solution and extracted with ethyl acetate. The combined organic layers were washed with water and brine, dried over anhydrous sodium sulfate, and concentrated to afford 7-amino-2-methyl-2,3,4,5-tetrahydro-1H-benzo[c]azepin-1-one (52.4) as a yellow solid (100 mg, 85%).

### (R)-7-((4-((1-Acryloylpiperidin-3-yl)amino)-5-fluoropyrimidin-2-yl)amino)-2-methyl-2,3,4,5-tetrahydro-1H-benzo[c]azepin-1-one (I-52)

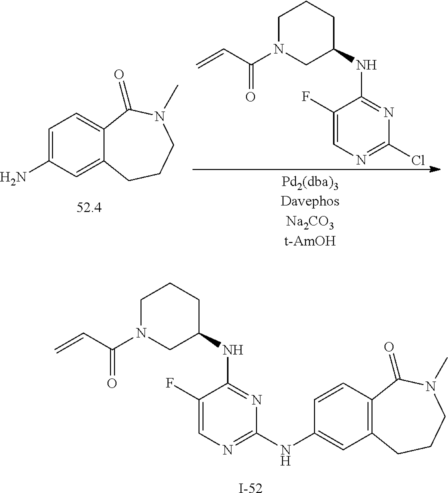

A mixture of 7-amino-2-methyl-2,3,4,5-tetrahydro-1H-benzo[c]azepin-1-one (52.4) (100 mg, 0.53 mmol), 1-[(3R)-3-[(2-chloro-5-fluoro-pyrimdin-4-yl)amino]-1-piperidyl]prop-2-en-1-one (intermediate B, 180 mg, 0.63 mmol), tris(dibenzylideneacetone)dipalladium(O) (48 mg, 0.05 mmol), Davephos (21 mg, 0.05 mmol), and Na₂CO₃ (390 mg, 3.68 mmol) in tert-amyl alcohol (15 mL) was stirred at 100° C. for 4 h under N₂. TLC showed the reaction was complete. The mixture was quenched with the addition of water and extracted with ethyl acetate. The combined organic were washed with water and brine and concentrated in vacuo to afford the crude product. The crude product was purified by column chromatography (DCM/MeOH:50/1) to afford (R)-7-((4-((1-acryloylpiperidin-3-yl)amino)-5-fluoropyrimidin-2-yl)amino)-2-methyl-2,3,4,5-tetrahydro-1H-benzo[c]azepin-1-one (I-52) as a yellow solid (31 mg, 13%),

LCMS: 439.5 [M+1]⁺.

¹HNMR (DMSO-d₆): δ 1.41 (br, 1H), 1.58-1.73 (m, 1H), 1.81-2.01 (m, 4H, 2.56 (t, 2H, 2.67-2.80 (m, 1H), 3.02 (br, 3.5H), 3.12-3.18 (m, 2.5H), 4.03 (br, 2H), 4.21 (d, 0.5H), 4.46 (d, 0.5H), 5.46 (d, 0.5H), 5.70 (d, 0.5H), 5.99 (d, 0.5H), 6.12 (d, 0.5H), 6.62 (dd, 0.5H), 6.84 (dd, 0.5H), 7.32 (d, 1H), 7.45-7.55 (m, 2H), 7.64 (s, 1H), 7.95 (s, 1H), 9.32 (s, 1H).

### Example 53

### Preparation of (R)-1-3((5-fluoro-2-((2,3,4,5-tetrahydro-1H-benzo[c]azepin-8-yl)amino)pyrimidin-4-yl)amino)piperidin-1-yl)prop-2-en-1-one (I-53)

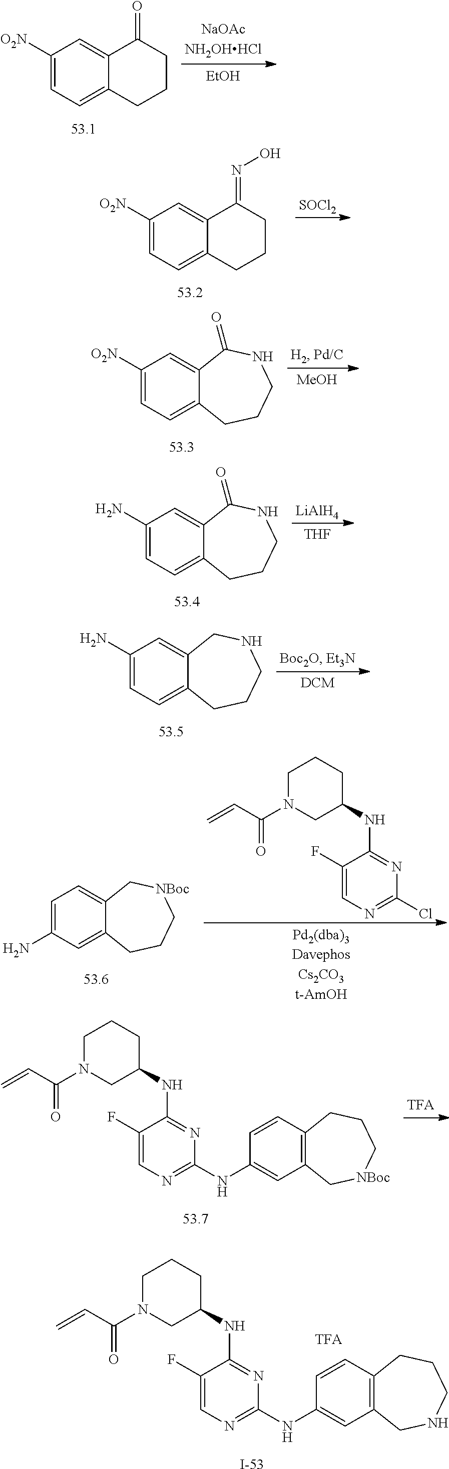

### (E)-7-Nitro-3,4-dihydronaphthalen-1(2H)-one oxime (53.2)

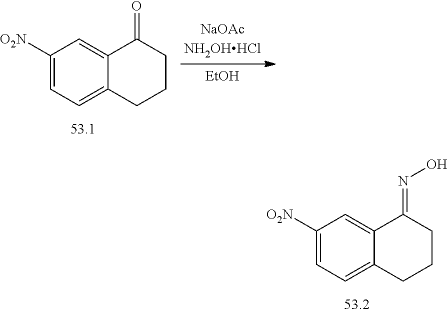 [SMILES: "EtOH" ; O=C1CCCc2ccc([N+](=O)[O-])cc21 ; O=[N+]([O-])c1ccc2c(c1)/C(=N/O)CCC2]

A solution of 7-nitro-3,4-dihydronaphthalen-1(2H)-one (53.1) (1.5 g, 7.85 mmol), NaOAc (1.3 g, 15.8 mmol), and hydroxylamine hydrochloride (6.5 g, 93.6 mmol) in ethanol (25 mL) was stirred at 90° C. for 4 h. The reaction mixture was diluted with water and extracted with ethyl acetate. The combined organic layers were washed with water and brine, dried over sodium sulfate, and concentrated to afford (E)-7-nitro-3,4-dihydronaphthalen-1(2H) one oxime (53.2) as a yellow solid (1.58 g, 97%).

### 8-Nitro-2,3,4,5-tetrahydro-1H-benzo[c]azepin-1-one (53.3)

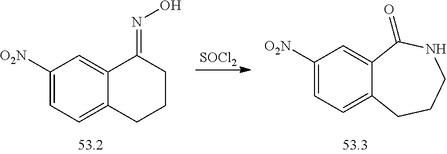

A solution of (E)-7-nitro-3,4-dihydronaphthalen-1(2H)-one oxime (53.2) (7 g, 33.9 mmol) in SOCl₂ (20 mL) was stirred at 50° C. for 6 h. After cooling to ambient temperature, the reaction mixture was concentrated, diluted with water, and extracted with ethyl acetate. The combined organic layers were washed with water and brine, dried over sodium sulfate, and concentrated. The crude product was purified by column chromatography (ethyl acetate/hexane: 1/1) to afford 8-nitro-2,3,4,5-tetrahydro-1H-benzo[c]azepin-1-one (53.3) as a yellow solid (2 g, 28%).

LCMS: 207.0 [M+1]⁺.

### 8-Amino-2,3,4,5-tetrahydro-1H-benzo[c]azepin-1-one (53.4)

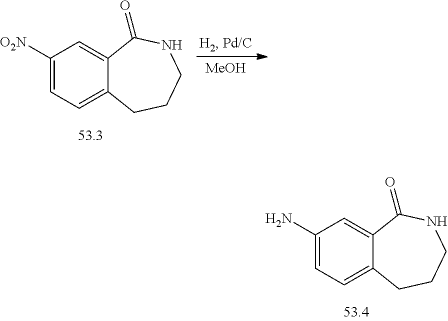 [SMILES: "MeOH" ; O=C1NCCCc2ccc([N+](=O)[O-])cc21 ; "H2, Pd/C" ; Nc1ccc2c(c1)C(=O)NCCC2]

A mixture of 8-nitro-2,3,4,5-tetrahydro-1H-benzo[c]azepin-1-one (53.3) (1.5 g, 7.27 mol) and Pd/C (500 mg) in MeOH (20 mL) was stirred at ambient temperature overnight under H₂. The mixture was filtered and concentrated to afford 8-amino-2,3,4,5-tetrahydro-1H-benzo[c]azepin-1-one (53.4) as a colorless oil (1.15 g, 90%).

LCMS: 177.1 [M+1]⁺.

### tert-Butyl 8-amino-4,5-dihydro-1H-benzo[c]azepine-2(3H)-carboxylate (53.6)

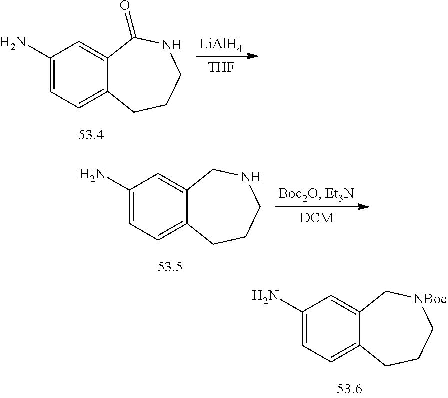

LiAlH₄ (1.6 g, 43.6 mmol) was slowly added into a solution of 8-amino-2,3,4,5-tetrahydro-1H-benzo[c]azepin-1-one (53.4) (1.28 g, 7.27 mmol) in THF (30 mL) at 0° C. The reaction was stirred at 70° C. for 3 h. Sodium sulfate decahydrate was added to the reaction. The resulting mixture was filtered, and the combined organic layers were dried with sodium sulfate before concentrating to dryness. The residue was added into a solution of di-tert-butyl dicarbonate (1.59 g, 7.27 mmol) and triethylamine (734 mg, 7.27 mmol) in DCM (10 mL). The reaction was stirred at ambient temperature for 1 h. The reaction mixture was diluted with water and extracted with DCM. The combined organic layers were washed with water and brine, dried over sodium sulfate, and concentrated to afford the crude product. The crude product was purified by column chromatography (ethyl acetate/hexane:1/1) to afford tert-butyl 8-amino-4,5-dihydro-1H-benzo[c]azepine-2(3H)-carboxylate (53.6) as a yellow solid (500 mg, 26%, 2 steps).

LCMS: 207.2 [M+1-^{t}Bu]⁺.

### (R)-tert-Butyl 8-(4-(1-acryloylpiperidin-3-ylamino)-5-fluoropyrimidin-2-ylamino)-4,5-dihydro-1H-benzo[c]azepine-2(3H)-carboxylate (53.7)

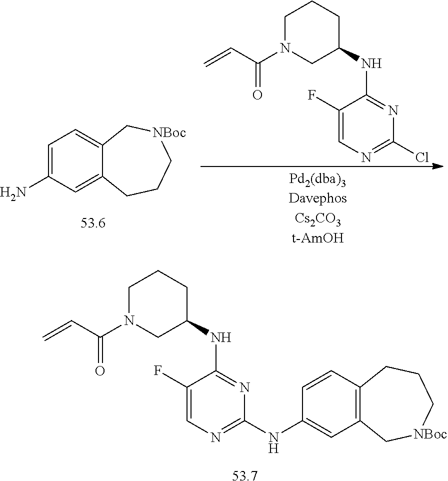

(R)-1-(3-(2-Chloro-5-fluoropyrimidin-4-ylamino)piperidin-1-yl)prop-2-en-1-one (intermediate B, 542 mg, 1.9 mmol) and Cs₂CO₃ (1.2 g, 3.8 mmol) were added to a solution of tert-butyl 8-amino-4,5-dihydro-1H-benzo[c]azepine-2(3H)-carboxylate (53.6) (500 mg, 1.9 mmol) in tert-amyl alcohol (10 mL). Tris(dibenzylideneacetone)dipalladium(O) (174 mg, 0.19 mmol) and DavePhos (150 mg, 0.38 mmol) were added under N₂. The reaction mixture was stirred at 100° C. for 2 h. The reaction mixture was diluted with water and extracted with ethyl acetate. The combined organic layers were washed with water and brine, dried over sodium sulfate, and concentrated to afford the crude product. The crude product was purified by column chromatography (DCM/MeOH:10/1) to afford (R)-tert-butyl 8-(4-(1-acryloylpiperidin-3-ylamino)-5-fluoropyrimidin-2-ylamino)-4,5-dihydro-1H-benzo[c]azepine-2(3H)-carboxylate (53.7) as a yellow solid (850 mg, 87%).

LCMS: 511.6 [M+1]⁺.

### (R)-1-(3-(5-Fluoro-2-(2,3,4,5-tetrahydro-1H-benzo[c]azepin-8-ylamino)pyrimidin-4-ylamino)piperidin-1-yl)prop-2-en-1-one (I-53)

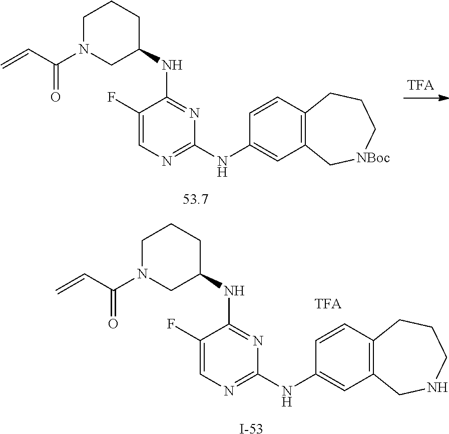

A solution of (R)-tert-butyl 8-(4-(1-acryloylpiperidin-3-ylamino)-5-fluoropyrimidin-2-ylamino)-4,5-dihydro-1H-benzo[c]azepine-2(3H)-carboxylate (53.7) (100 mg, 0.19 mmol) in TFA (2 mL) was stirred at room temperature for 30 min. After evaporation, the crude product was washed with diethyl ether and filtered to afford the TFA salt of (R)-1-(3-(5-fluoro-2-(2,3,4,5-tetrahydro-1H-benzo[c]azepin-8-ylamino)pyrimidin-4-ylamino)piperidin-1-yl)prop-2-en-1-one (I-53) as a red solid (90 mg, 88%).

LCMS: 411.3 [M+1]⁺.

¹H NMR (400 MHz, CD₃OD): δ 1.53-1.62 (m, 1H), 1.75-1.85 (m, 1H), 1.93-2.04 (m, 3H), 2.10-2.18 (m, 1H), 2.84-2.92 (m, 1H), 3.02-3.10 (m, 2H), 3.14-3.25 (m, 1H), 3.48-3.52 (m, 2H), 4.03-4.16 (m, 2H), 4.34-4.43 (m, 2.5H), 4.60-4.64 (m, 0.5H), 5.46-5.49 (m, 0.5H), 5.81-5.84 (m, 0.5H), 6.07-6.11 (m, 0.5H), 6.23-6.28 (m, 0.5H), 6.40-6.46 (m, 0.5H), 6.81-6.88 (m, 0.5H), 7.28-7.33 (m, 1H), 7.46-7.52 (m, 1H), 7.59-7.61 (m, 1H), 7.91-7.95 (m, 1H).

### Example 54

### Preparation of (R)-1-(3-(2-(2-Cyclopropyl-2,3,4,5-tetrahydro-1H-benzo[c]azepin-8-ylamino)-5-fluoropyrimidin-4-ylamino)piperidin-1-yl)prop-2-en-1-one (I-54)

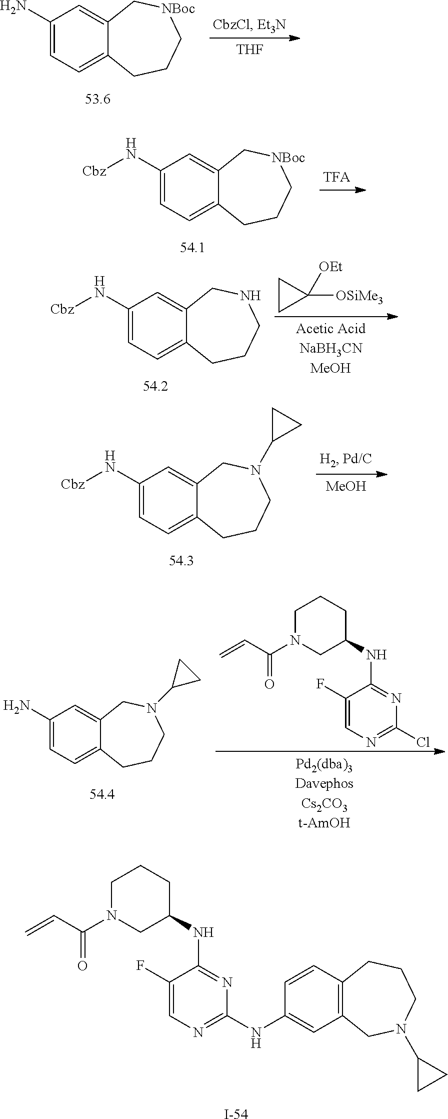

### tert-Butyl 8-(benzyloxycarbonylamino)-4,5-dihydro-1H-benzo[c]azepine-2(3H)-carboxylate (54.1)

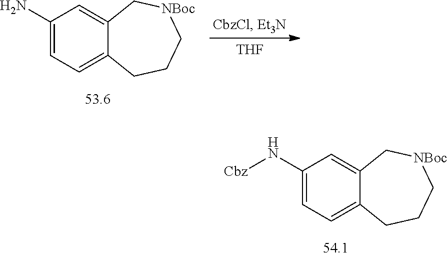

CbzCl (243 mg, 1.4 mmol) was slowly added into a solution of tert-butyl 8-amino-4,5-dihydro-1H-benzo[c]azepine-2(3H)-carboxylate (53.6) (250 mg, 0.95 mmol) and triethylamine (192 mg, 1.9 mmol) in THF (10 mL) at ambient temperature. The reaction was stirred at ambient temperature for 1 h. The reaction mixture was diluted with water and extracted with ethyl acetate. The combined organic layers were washed with water and brine, dried over sodium sulfate, and concentrated to afford the crude product. The crude product was purified by column chromatography (ethyl acetate/hexane:1/5) to afford tert-butyl 8-(benzyloxycarbonylamino)-4,5-dihydro-1H-benzo[c]azepine-2(3H)-carboxylate (54.1) as a yellow oil (290 mg, 77%).

### Benzyl 2,3,4,5-tetrahydro-1H-benzo[c]azepin-8-ylcarbamate (54.2)

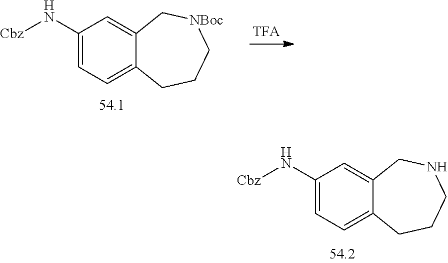 [SMILES: CC(C)(C)OC(=O)N1CCCc2ccc(NC(=O)OCc3ccccc3)cc2C1 ; O=C(Nc1ccc2c(c1)CNCCC2)OCc1ccccc1]

A solution of tert-butyl 8-(benzyloxycarbonylamino)-4,5-dihydro-1H-benzo[c]azepine-2(3H)-carboxylate (54.1) (200 mg, 0.50 mmol) in TFA (2 mL) was stirred at ambient temperature for 30 min. After evaporation, the crude benzyl 2,3,4,5-tetrahydro-1H-benzo[c]azepin-8-ylcarbamate (54.2) was used in the next step without further purification.

### Benzyl 2-cyclopropyl-2,3,4,5-tetrahydro-1H-benzo[c]azepin-8-ylcarbamate (54.3)

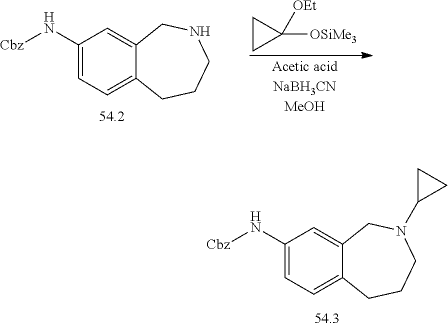

(1-Ethoxycyclopropoxy)trimethylsilane (139 mg, 0.88 mmol) was slowly added to the solution of benzyl 2,3,4,5-tetrahydro-1H-benzo[c]azepin-8-ylcarbamate (54.2), NaBH₃CN (82 mg, 1.3 mmol), and acetic acid (131 mg, 2.2 mmol) in MeOH (10 mL) at 0° C. The resulting mixture was heated at 65° C. for 4 h. The reaction mixture was diluted with water and extracted with ethyl acetate. The combined organic layers were washed with water and brine, dried over sodium sulfate, and concentrated to afford the crude product. The crude product was purified by column chromatography (DCM/MeOH:20/1) to afford benzyl 2-cyclopropyl-2,3,4,5-tetrahydro-1H-benzo[c]azepin-8-ylcarbamate (54.3) as a yellow oil (120 mg, 71%).

LCMS: 337.2 [M+1]⁺.

### 2-Cyclopropyl-2,3,4,5-tetrahydro-1H-benzo[c]azepin-8-amine (54.4)

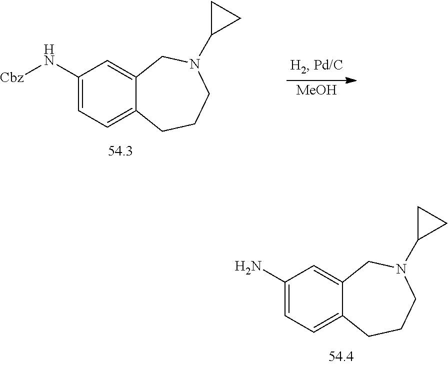 [SMILES: "MeOH" ; O=C(Nc1ccc2c(c1)CN(C1CC1)CCC2)OCc1ccccc1 ; Nc1ccc2c(c1)CN(C1CC1)CCC2]

A mixture of benzyl 2-cyclopropyl-2,3,4,5-tetrahydro-1H-benzo[c]azepin-8-ylcarbamate (54.3) (120 mg, 0.36 mmol) and Pd/C (60 mg) in MeOH (10 mL) was stirred at ambient temperature overnight under H₂. The mixture was filtered and concentrated to afford 2-cyclopropyl-2,3,4,5-tetrahydro-1H-benzo[c]azepin-8-amine (54.4) as a yellow oil (60 mg, 83%).

### (R)-1-(3-(2-(2-Cyclopropyl-2,3,4,5-tetrahydro-1H-benzo[c]azepin-8-ylamino)-5-fluoropyrimidin-4-ylamino)piperidin-1-yl)prop-2-en-1-one (I-54)

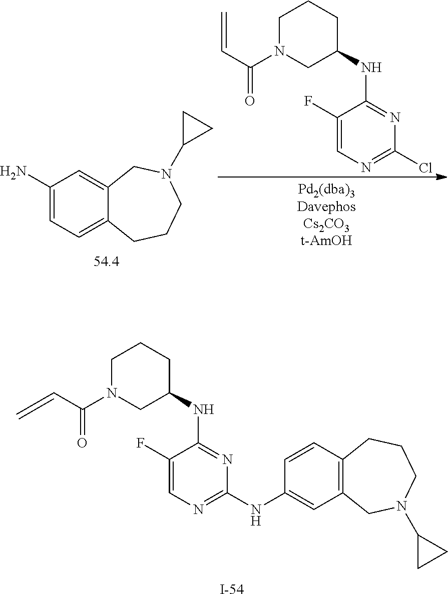

(R)-1-(3-(2-Chloro-5-fluoropyrimidin-4-ylamino)piperidin-1-yl)prop-2-en-1-one (intermediate B, 70 mg, 0.25 mmol) and Cs₂CO₃ (160 mg, 0.49 mmol) were added to a solution of 2-cyclopropyl-2,3,4,5-tetrahydro-1H-benzo[c]azepin-8-amine (54.4) (50 mg, 0.25 mmol) in tert-amyl alcohol (10 mL). Tris(dibenzylideneacetone)dipalladium(O) (23 mg, 0.025 mmol) and DavePhos (19 mg, 0.049 mmol) were added under N₂. The reaction mixture was stirred at 100° C. for 2 h. The reaction mixture was diluted with water and extracted with ethyl acetate. The combined organic layers were washed with water and brine, dried over sodium sulfate, and concentrated to afford the crude product. The crude product was purified by prep-HPLC to afford (R)-1-(3-(2-(2-cyclopropyl-2,3,4,5-tetrahydro-1H-benzo[c]azepin-8-ylamino)-5-fluoropyrimidin-4-ylamino)piperidin-1-yl)prop-2-en-1-one (I-54) (TFA salt) as a yellow solid (50 mg, 44%).

LCMS: 451.2 [M+1]⁺.

¹H NMR (400 MHz, CD₃OD): δ 1.03-1.05 (m, 4H), 1.54-1.61 (m, 1H), 1.76-2.14 (m, 6H), 2.84-3.34 (m, 4H), 3.72-3.77 (m, 2H), 4.03-4.15 (m, 2H), 4.41-4.67 (m, 3H), 5.49-5.52 (m, 0.5H), 5.80-5.84 (m, 0.5H), 6.08-6.13 (m, 0.5H), 6.23-6.27 (m, 0.5H), 6.39-6.45 (m, 0.5H), 6.81-6.87 (m, 0.5H), 7.31-7.36 (m, 1H), 7.46-7.67 (m, 2H), 7.93-7.98 (m, 1H).

### Example 55

### Preparation of (R)-1-(3-((5-fluoro-2-((2-methyl-2,3,4,5-tetrahydro-1H-benzo[c]azepin-8-yl)amino)pyrimidin-4-yl)amino)piperidin-1-yl)prop-2-en-1-one (I-55)

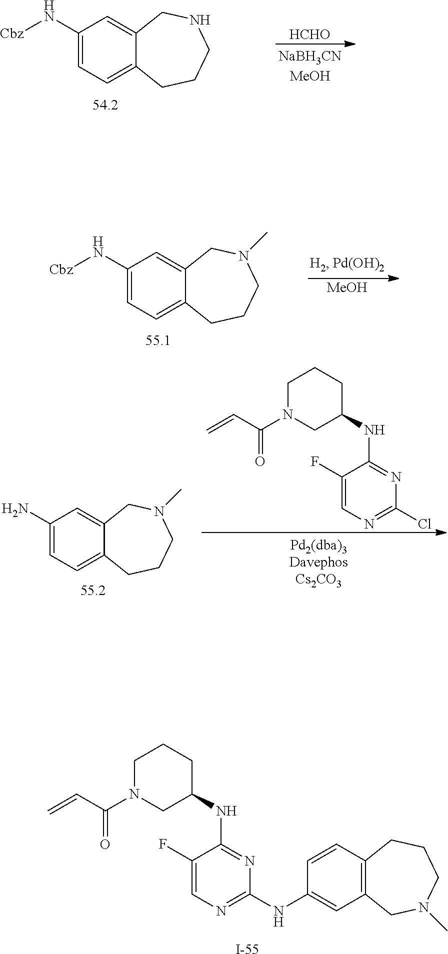

### Benzyl (2-methyl-2,3,4,5-tetrahydro-1H-benzo[c]azepin-8-yl)carbamate (55.1)

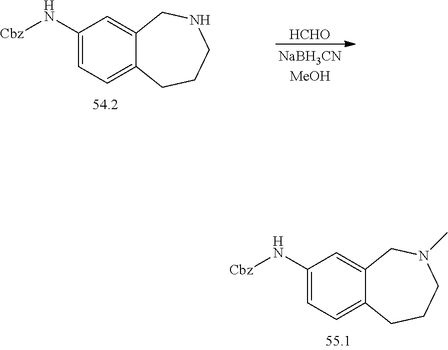 [SMILES: O=C(Nc1ccc2c(c1)CNCCC2)OCc1ccccc1 ; "HCHO" ; CN1CCCc2ccc(NC(=O)OCc3ccccc3)cc2C1]

To a solution of benzyl (2,3,4,5-tetrahydro-1H-benzo[c]azepin-8-yl)carbamate (54.2) in methanol (10 mL) was added HCHO (0.5 mL, 37% in H₂O). The mixture was stirred at room temperature for 3 h. To the mixture was added NaBH₃CN (153 mg, 2.4 mmol), and the reaction mixture was stirred at ambient temperature for 1 h. The reaction mixture was diluted with water and extracted with ethyl acetate. The combined organic layers were washed with water and brine, dried over sodium sulfate, and concentrated to afford the crude product. The crude product was purified by column chromatography (DCM/MeOH:10/1) to afford benzyl (2-methyl-2,3,4,5-tetrahydro-1H-benzo[c]azepin-8-yl)carbamate (55.1) as a yellow oil (160 mg, 68%, 2 steps).

LCMS: 311.3 [M+1]⁺.

### 2-Methyl-2,3,4,5-tetrahydro-1H-benzo[c]azepin-8-amine (55.2)

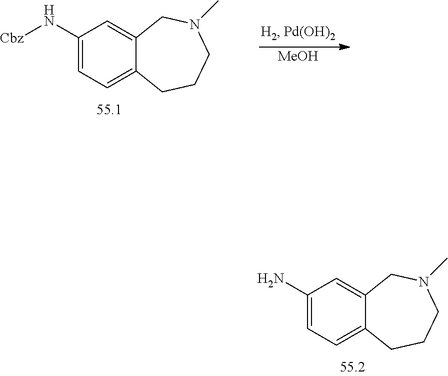 [SMILES: "MeOH" ; CN1CCCc2ccc(NC(=O)OCc3ccccc3)cc2C1 ; CN1CCCc2ccc(N)cc2C1]

A solution of benzyl 2-methyl-2,3,4,5-tetrahydro-1H-benzo[c]azepin-8-ylcarbamate (55.1) (200 mg, 0.64 mol) and Pd(OH)₂ (50 mg) in MeOH (10 mL) was stirred at ambient temperature overnight under H₂. The mixture was filtered and concentrated to afford 2-methyl-2,3,4,5-tetrahydro-1H-benzo[c]azepin-8-amine (55.2) as a colorless oil (108 mg, 95%).

LCMS: 177.1 [M+1]⁺.

### (R)-1-(3-(5-Fluoro-2-(2-methyl-2,3,4,5-tetrahydro-1H-benzo[c]azepin-8-ylamino)pyrimidin-4-ylamino)piperidin-1-yl)prop-2-en-1-one (I-55)

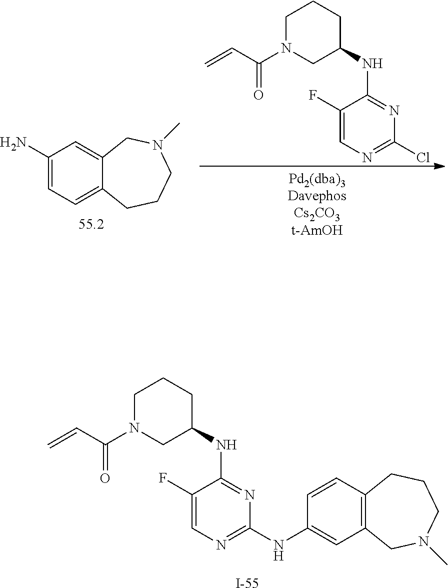

(R)-1-(3-(2-Chloro-5-fluoropyrimidin-4-ylamino)piperidin-1-yl)prop-2-en-1-one (intermediate B, 273 mg, 0.96 mmol) and Cs₂CO₃ (480 mg, 1.48 mmol) were added to the solution of 2-methyl-2,3,4,5-tetrahydro-1H-benzo[c]azepin-8-amine (55.2) (130 mg, 0.74 mmol) in tert-amyl alcohol (10 mL). Tris(dibenzylideneacetone)dipalladium(O) (67 mg, 0.07 mmol) and DavePhos (58 mg, 0.15 mmol) were added under N₂. The reaction mixture was stirred at 100° C. for 2 h. The reaction mixture was diluted with water and extracted with ethyl acetate. The combined organic layers were washed with water and brine, dried over sodium sulfate, and concentrated to afford the crude product. The crude product was purified by column chromatography (DCM/MeOH:10/1) to afford (R)-1-(3-(5-fluoro-2-(2-methyl-2,3,4,5-tetrahydro-1H-benzo[c]azepin-8-ylamino)pyrimidin-4-ylamino)piperidin-1-yl)prop-2-en-1-one (I-55) as a white solid (70 mg, 22%).

LCMS: 425.3 [M+1]⁺.

¹H NMR (400 MHz, CD₃OD): δ 1.52-1.62 (m, 1H), 1.77-1.96 (m, 3H), 2.11-2.17 (m, 2H), 2.83-3.25 (m, 7H), 3.50-3.68 (m, 2H), 4.04-4.16 (m, 2H), 4.47-4.64 (m, 3H), 5.45-5.48 (m, 0.5H), 5.81-5.84 (m, 0.5H), 6.06-6.10 (m, 1H), 6.23-6.40 (m, 1H), 6.81-6.88 (m, 0.5H), 7.30-7.68 (m, 3H), 7.94-7.99 (m, 1H).

### Example 56

### Preparation of (R)-1-(3-(5-Fluoro-2-(2-(oxetan-3-ylmethyl)-2,3,4,5-tetrahydro-1H-benzo[c]azepin-8-ylamino)pyrimidin-4-ylamino)piperidin-1-yl)prop-2-en-1-one (I-56)

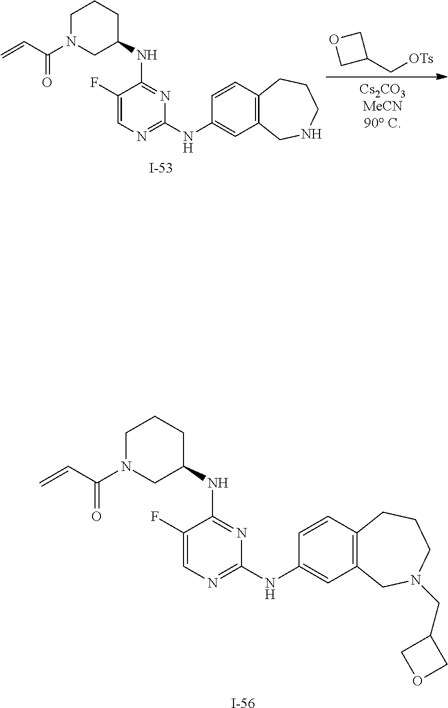

A mixture of (R)-1-(3-(5-fluoro-2-(2,3,4,5-tetrahydro-1H-benzo[c]azepin-8-ylamino)pyrimidin-4-ylamino)piperidin-1-yl)prop-2-en-1-one (I-53) (200 mg, 0.49 mmol), oxetan-3-ylmethyl 4-methylbenzenesulfonate (177 mg, 0.73 mmol), and Cs₂CO₃ (318 mg, 0.97 mmol) in acetonitrile (MeCN) (8 mL) was stirred at 90° C. for 5 h. After cooling to room temperature, the reaction mixture was diluted with water and extracted with ethyl acetate. The combined organic layers were washed with water and brine, dried over sodium sulfate, and concentrated. The crude product was purified by column chromatography (DCM/MeOH:10/1) to afford (R)-1-(3-(5-fluoro-2-(2-(oxetan-3-ylmethyl)-2,3,4,5-tetrahydro-1H-benzo[c]azepin-8-ylamino)pyrimidin-4-ylamino)piperidin-1-yl)prop-2-en-1-one (I-56) as a white solid (30 mg, 13%).

Mp: 103-105° C.

LCMS: 481.2 [M+1]⁺.

¹H NMR (400 MHz, DMSO-d₆): δ 1.38-1.44 (m, 1H), 1.52-1.68 (m, 3H), 1.80-1.85 (m, 1H), 1.98-2.02 (m, 1H), 2.40-2.46 (m, 1H), 2.54-2.56 (m, 1H), 2.67-3.15 (m, 7H), 3.68-3.69 (m, 2H), 3.99-4.01 (m, 2H), 4.18-4.19 (m, 2.5H), 4.38-4.42 (m, 0.5H), 4.58 (t, 2H), 5.47-5.49 (m, 0.5H), 5.69-5.72 (m, 0.5H), 6.00-6.14 (m, 1H), 6.58-6.65 (m, 0.5H), 6.82-6.88 (m, 0.5H), 6.94 (d, 1H), 7.34-7.45 (m, 3H), 7.90 (s, 1H), 8.94 (s, 1H).

### Example 57

### Preparation of (R)-1-(3-((5-fluoro-2-((2,3,4,5-tetrahydro-1H-benzo[d]azepin-7-yl)amino)pyrimidin-4-yl)amino)piperidin-1-yl)prop-2-en-1-one (I-57)

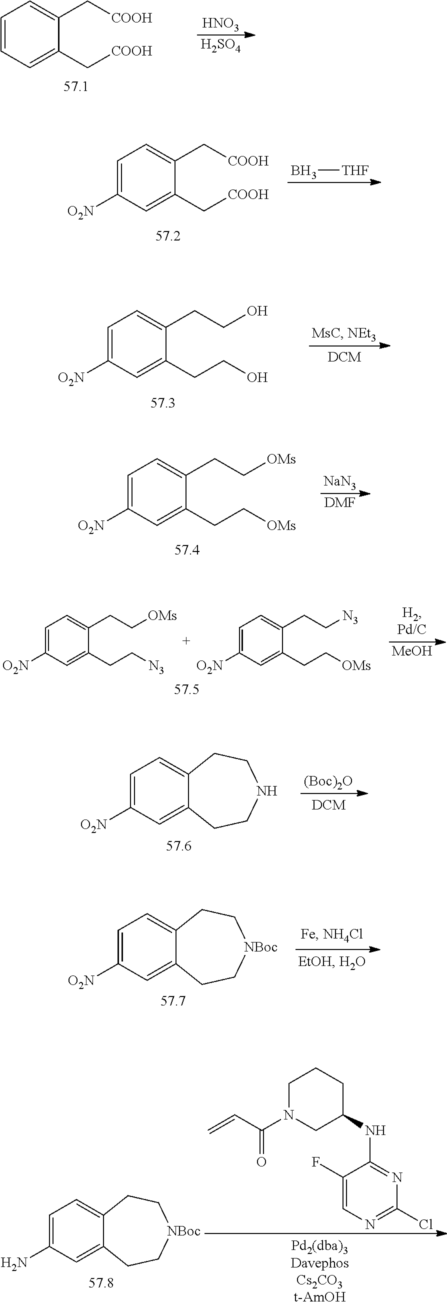

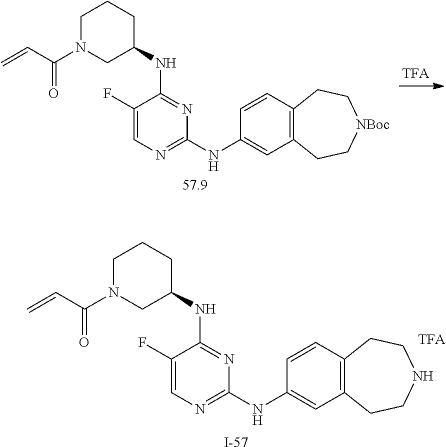

### 2,2′-(4-Nitro-1,2-phenylene)diacetic acid (57.2)

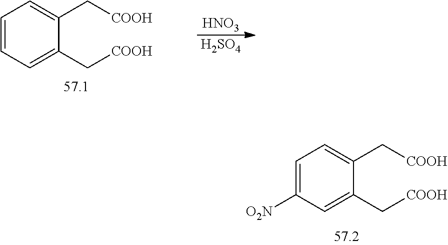 [SMILES: O=C(O)Cc1ccccc1CC(=O)O ; "H2SO4" ; "HNO3" ; O=C(O)Cc1ccc([N+](=O)[O-])cc1CC(=O)O]

HNO₃ (3.2 g, 51.5 mmol) was slowly added to a solution of 2,2′-(1,2-phenylene)diacetic acid (57.1) (10 g, 51.5 mmol) in H₂SO₄ (40 mL) at 0° C. The reaction was stirred at 0° C. for 4 h. The reaction mixture was carefully added to ice water and extracted with ethyl acetate. The combined organic layers were washed with water and brine, dried over sodium sulfate, and concentrated to afford 2,2′-(4-nitro-1,2-phenylene)diacetic acid (57.2) as a yellow solid (10.9 g, 88%).

### 2,2′-(4-Nitro-1,2-phenylene)diethanol (57.3)

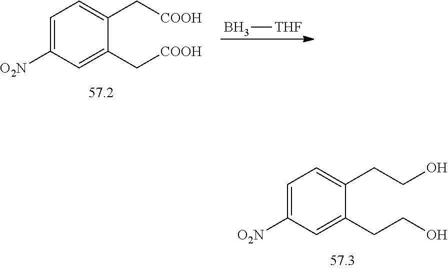

BH₃-THF (91.2 mL, 91.2 mmol) was slowly added to a solution of 2,2′-(4-nitro-1,2-phenylene)diacetic acid (57.2) (10.9 g, 45.6 mmol) in THF (100 mL) at 0° C. The reaction was stirred at 0° C. for 2 h. The reaction mixture was diluted with water and extracted with ethyl acetate. The combined organic layers were washed with water and brine, dried over sodium sulfate, and concentrated to afford the crude product. The crude product was purified by column chromatography (ethyl acetate/hexane:1/10) to afford 2,2′-(4-nitro-1,2-phenylene)diethanol (57.3) as a yellow oil (7.5 g, 78%).

LCMS: 212.0 [M+1]⁺.

### 2,2′-(4-Nitro-1,2-phenylene)bis(ethane-2,1-diyl) dimethanesulfonate (57.4)

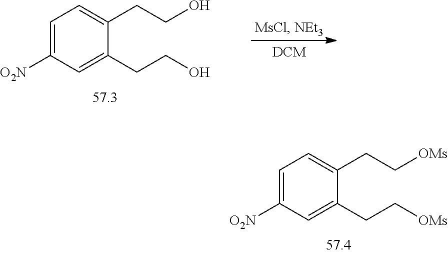 [SMILES: O=[N+]([O-])c1ccc(CCO)c(CCO)c1 ; CS(=O)(=O)OCCc1ccc([N+](=O)[O-])cc1CCOS(C)(=O)=O]

Methanesulfonyl chloride (12.2 g, 106.5 mmol) was slowly added to a solution of 2,2′-(4-nitro-1,2-phenylene)diethanol (57.3) (7.5 g, 35.5 mmol) and triethylamine (10.7 g, 106.5 mmol) in DCM (100 mL) at 0° C. The reaction was stirred at 0° C. for 3 h. The reaction mixture was diluted with water and extracted with DCM. The combined organic layers were washed with water and brine, dried over sodium sulfate, and concentrated to afford the crude product. The crude product was purified by column chromatography (ethyl acetate/hexane:1/20) to afford 2,2′-(4-nitro-1,2-phenylene)bis(ethane-2,1-diyl) dimethanesulfonate (57.4) as a yellow oil (11 g, 85%).

LCMS: 367.9 [M+1]⁺.

### 2-(2-Azidoethyl)-4-nitrophenethyl methanesulfonate and 2-(2-azidoethyl)-5-nitrophenethyl methanesulfonate (57.5)

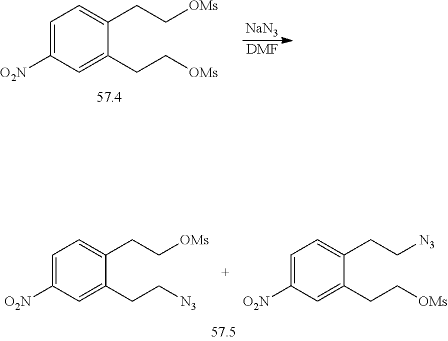

A solution of 2,2′-(4-nitro-1,2-phenylene)bis(ethane-2,1-diyl) dimethanesulfonate (57.4) (11 g, 29.9 mmol) and NaN₃ (1.2 g, 18.5 mmol) in DMF (100 mL) was stirred at ambient temperature overnight. The reaction mixture was diluted with water and extracted with ethyl acetate. The combined organic layers were washed with water and brine, dried over sodium sulfate, and concentrated to afford the crude products. The crude products were purified by column chromatography (ethyl acetate/hexane:1/1) to afford a mixture of 2-(2-azidoethyl)-4-nitrophenethyl methanesulfonate and 2-(2-azidoethyl)-5-nitrophenethyl methanesulfonate (57.5) as a yellow oil (3.6 g, 38%).

### 7-Nitro-2,3,4,5-tetrahydro-1H-benzo[d]azepine (57.6)

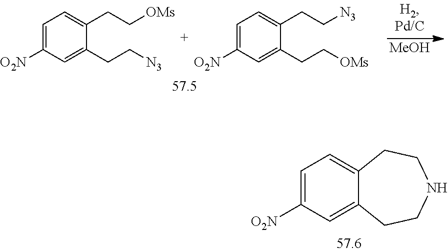 [SMILES: "MeOH" ; CS(=O)(=O)OCCc1ccc([N+](=O)[O-])cc1CCN=[N+]=[N-] ; CS(=O)(=O)OCCc1cc([N+](=O)[O-])ccc1CCN=[N+]=[N-] ; O=[N+]([O-])c1ccc2c(c1)CCNCC2]

A mixture of 2-(2-azidoethyl)-4-nitrophenethyl methanesulfonate and 2-(2-azidoethyl)-5-nitrophenethyl methanesulfonate (57.5) (2 g, 6.4 mol) and Pd/C (70 mg) in MeOH (50 mL) was stirred at ambient temperature overnight under H₂. The mixture was filtered and concentrated to afford 7-nitro-2,3,4,5-tetrahydro-1H-benzo[d]azepine (57.6) as a yellow oil (800 mg, 65%).

LCMS: 193.1 [M+1]⁺.

### tert-Butyl 7-nitro-4,5-dihydro-1H-benzo[d]azepine-3(2H)-carboxylate (57.7)

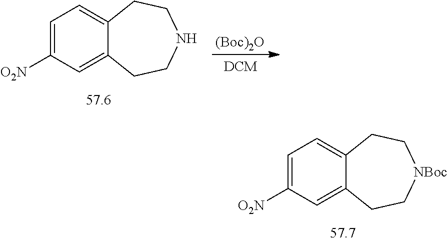 [SMILES: "(Boc)2O" ; O=[N+]([O-])c1ccc2c(c1)CCNCC2 ; CC(C)(C)OC(=O)N1CCc2ccc([N+](=O)[O-])cc2CC1]

A solution of 7-nitro-2,3,4,5-tetrahydro-1H-benzo[d]azepine (57.6) (800 mg, 4.2 mmol) and di-tert-butyl dicarbonate (1.4 g, 6.2 mmol) in DCM (20 mL) was stirred at ambient temperature for 2 h. The reaction mixture was diluted with water and extracted with ethyl acetate. The combined organic layers were washed with water and brine, dried over sodium sulfate, and concentrated to afford the crude product. The crude product was purified by column chromatography (ethyl acetate/hexane:1/5) to afford tert-butyl 7-nitro-4,5-dihydro-1H-benzo[d]azepine-3(2H)-carboxylate (57.7) as a yellow solid (1 g, 82%).

LCMS: 237.1 [M+1-′Bu]⁺.

### tert-Butyl 7-amino-4,5-dihydro-1H-benzo[d]azepine-3(2H)-carboxylate (57.8)

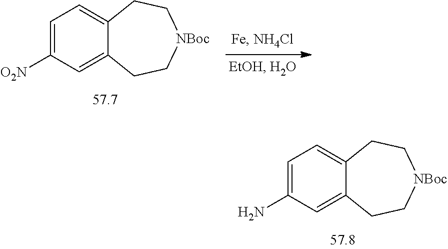 [SMILES: CC(C)(C)OC(=O)N1CCc2ccc([N+](=O)[O-])cc2CC1 ; CC(C)(C)OC(=O)N1CCc2ccc(N)cc2CC1]

Fe (862 mg, 15.4 mmol) and NH₄Cl (831 mg, 15.4 mmol) were added to the solution of tert-butyl 7-nitro-4,5-dihydro-1H-benzo[d]azepine-3(2H)-carboxylate (57.7) (750 mg, 2.6 mmol) in EtOH (20 mL) and H₂O (2 mL). The mixture was stirred at 90° C. for 1 h. After cooling to ambient temperature, the reaction mixture was diluted with water and extracted with ethyl acetate. The combined organic layers were washed with water and brine, dried over sodium sulfate, and concentrated to afford tert-butyl 7-amino-4,5-dihydro-1H-benzo[d]azepine-3(2H)-carboxylate (57.8) as a yellow solid (650 mg, 96%).

### (R)-tert-Butyl 7-(4-(1-acryloylpiperidin-3-ylamino)-5-fluoro pyrimi-din-2-ylamino)-4,5-dihydro-1H-benzo[d]azepine-3(2H)-carboxylate (57.9)

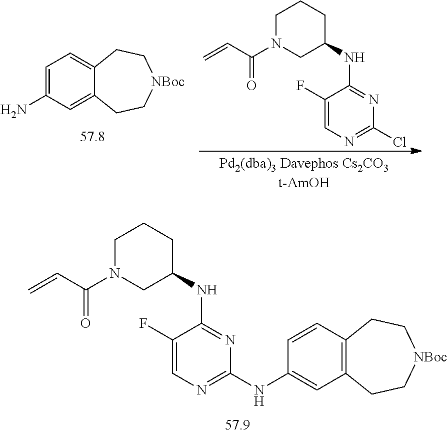 [SMILES: CC(C)(C)OC(=O)N1CCc2ccc(N)cc2CC1 ; C=CC(=O)N1CCC[C@@H](Nc2nc(Cl)ncc2F)C1 ; C=CC(=O)N1CCC[C@@H](Nc2nc(Nc3ccc4c(c3)CCN(C(=O)OC(C)(C)C)CC4)ncc2F)C1]

(R)-1-(3-(2-Chloro-5-fluoropyrimidin-4-ylamino)piperidin-1-yl)prop-2-en-1-one (intermediate B, 1.1 g, 3.5 mmol) and Cs₂CO₃ (1.6 g, 5.0 mmol) were added to the solution of tert-butyl 7-amino-4,5-dihydro-1H-benzo[d]azepine-3 (2H)-carboxylate (57.8) (650 mg, 2.5 mmol) in tert-amyl alcohol (10 mL). Tris(dibenzylideneacetone)dipalladium(O) (226 mg, 0.25 mmol) and DavePhos (194 mg, 0.50 mmol) were added under N₂. The reaction mixture was stirred at 100° C. for 2 h. The reaction mixture was diluted with water and extracted with ethyl acetate. The combined organic layers were washed with water and brine, dried over sodium sulfate, and concentrated. The crude product was purified by column chromatography (DCM/MeOH:20/1) to afford (R)-tert-butyl 7-(4-(1-acryloylpiperidin-3-ylamino)-5-fluoro pyrimi-din-2-ylamino)-4,5-dihydro-1H-benzo[d]azepine-3 (2H)-carboxylate (57.9) as a yellow solid (1.1 g, 87%).

LCMS: 511.5 [M+1]⁺.

### (R)-1-(3-(5-Fluoro-2-(2,3,4,5-tetrahydro-1H-benzo[d]azepin-7-ylamino)pyrimidin-4-ylamino)piperidin-1-yl)prop-2-en-1-one (I-57)

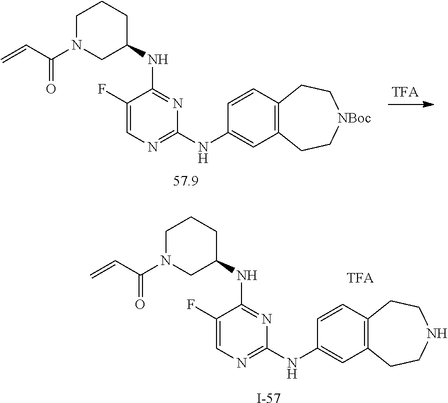

A solution of (R)-tert-butyl 7-(4-(1-acryloylpiperidin-3-ylamino)-5-fluoro pyrimi-din-2-ylamino)-4,5-dihydro-1H-benzo[d]azepine-3(2H)-carboxylate (57.9) (100 mg, 0.19 mmol) in TFA (5 mL) was stirred at ambient temperature for 30 min. After evaporation, the crude product was washed with diethyl ether and filtered to afford the TFA salt of (R)-1-(3-(5-fluoro-2-(2,3,4,5-tetrahydro-1H-benzo[c]azepin-7-ylamino)pyrimidin-4-ylamino)piperidin-1-yl)prop-2-en-1-one (I-57) as a red solid (60 mg, 58%).

LCMS: 411.2 [M+1]⁺:

¹H NMR (400 MHz, CD₃OD): δ 1.53-1.59 (m, 1H), 1.76-1.82 (m, 1H), 1.93-1.97 (m, 1H), 2.11-2.18 (m, 1H), 2.90-3.30 (m, 10H), 4.05-4.17 (m, 2H), 4.45-4.48 (m, 0.5H), 4.61-4.65 (m, 0.5H), 5.44-5.46 (m, 0.5H), 5.82-5.84 (m, 0.5H), 6.01-6.06 (m, 0.5H), 6.25-6.30 (m, 1H), 6.82-6.88 (m, 0.5H), 7.23-7.32 (m, 1.5H), 7.39-7.43 (m, 1H), 7.50-7.51 (m, 0.5H), 7.91-7.98 (m, 1H).

### Example 58

### Preparation of (R)-1-(3-(5-Fluoro-2-(3-methyl-2,3,4,5-tetrahydro-1H-benzo[d]azepin-7-ylamino)pyrimidin-4-ylamino)piperidin-1-yl)prop-2-en-1-one (I-58)

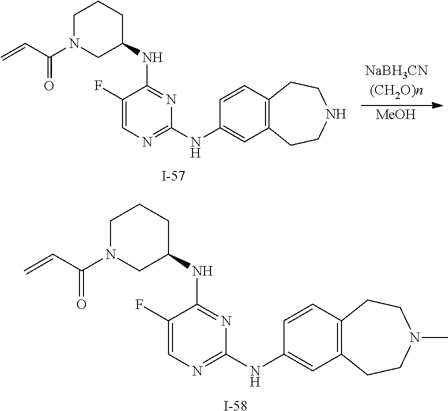

To a solution of (R)-1-(3-(5-fluoro-2-(2,3,4,5-tetrahydro-1H-benzo[d]azepin-7-ylamino)pyrimidin-4-ylamino)piperidin-1-yl)prop-2-en-1-one (I-57) (200 mg, 0.49 mmol) in methanol (20 mL) was added paraformaldehyde ((CH₂O)n) (73 mg, 2.4 mmol). The mixture was stirred at ambient temperature for 1 h. To the mixture was added NaBH₃CN (122 mg, 1.9 mmol), and the mixture was stirred at ambient temperature for 1 h. The reaction mixture was diluted with water and extracted with ethyl acetate. The combined organic layers were washed with water and brine, dried over sodium sulfate, and concentrated to afford the crude product. The crude product was purified by column chromatography (DCM/MeOH:10/1) to afford (R)-1-(3-(5-fluoro-2-(3-methyl-2,3,4,5-tetrahydro-1H-benzo[c/]azepin-7-ylamino)pyrimidin-4-ylamino)piperidin-1-yl)prop-2-en-1-one (I-58) as an off-white solid (50 mg, 24%).

Mp: 102-104° C.

LCMS: 425.2 [M+1]⁺.

¹H NMR (400 MHz, DMSO-d₆): δ 1.37-1.40 (m, 1H), 1.59-1.64 (m, 1H), 1.80-1.84 (m, 1H), 1.97-2.00 (m, 1H), 2.28 (s, 3H), 2.45-2.47 (m, 4H), 2.72-2.75 (m, 5H), 2.98-3.12 (m, 1H), 4.01 (d, 2H), 4.21-4.24 (m, 0.5H), 4.41-4.45 (m, 0.5H), 5.47 (dd, 0.5H), 5.71 (dd, 0.5H), 6.02 (d, 0.5H), 6.12 (d, 0.5 H), 6.58 (dd, 0.5H), 6.83-6.92 (m, 1.5H), 7.27-7.30 (m, 1H), 7.36-7.40 (m, 1H), 7.51 (d, 1H), 7.88 (d, 1H), 8.96 (s, 1H).

### Example 59

### Preparation of (R)-1-(3-(5-Fluoro-2-(3-(oxetan-3-ylmethyl)-2,3,4,5-tetrahydro-1H-benzo[d]azepin-7-ylamino)pyrimidin-4-ylamino)piperidin-1-yl)prop-2-en-1-one (I-59)

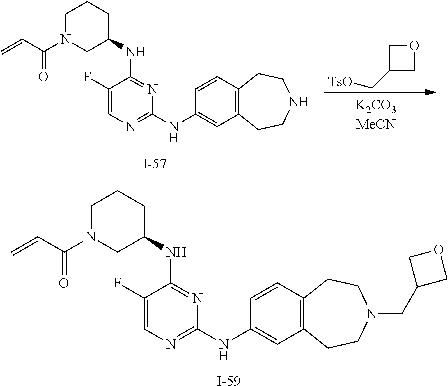

To a solution of 1-[(3R)-3-[[5-fluoro-2-(2,3,4,5-tetrahydro-1H-3-benzazepin-7-ylamino)pyrimidin-4-yl]amino]-1-piperidyl]prop-2-en-1-one (I-57) (200 mg, 0.49 mmol) in MeCN (20 mL) was added oxetan-3-ylmethyl 4-methylbenzene sulfonate (118 mg, 0.49 mmol) and K₂CO₃ (103 mg, 0.97 mmol). The mixture was stirred at 80° C. overnight. The reaction mixture was diluted with water and extracted with ethyl acetate. The combined organic layers were washed with water and brine, dried over sodium sulfate, and concentrated to afford the crude product. The crude product was purified by column chromatography (DCM/MeOH:10/1) to afford (R)-1-(3-(5-fluoro-2-(3-(oxetan-3-ylmethyl)-2,3,4,5-tetrahydro-1H-benzo[d]azepin-7-ylamino)pyrimidin-4-ylamino)piperidin-1-yl)prop-2-en-1-one (I-59) as an off-white solid (55 mg, 24%).

Mp: 104-106° C.

LCMS: 481.3 [M+1]⁺.

¹H NMR (400 MHz, DMSO-d₆): δ 1.36-1.40 (m, 1H), 1.61-1.64 (m, 1H), 1.80-1.83 (m, 1H), 1.91-2.00 (m, 1H), 2.44-2.49 (m, 4H), 2.67-2.74 (m, 6H), 2.97-3.20 (m, 2H), 4.00-4.03 (m, 2H), 4.21-4.27 (m, 2.5H), 4.41-4.45 (m, 0.5H), 4.62-4.65 (m, 2H), 5.46-5.49 (m, 0.5H), 5.69-5.72 (m, 0.5H), 5.99-6.15 (m, 1H), 6.56-6.62 (m, 0.5H), 6.83-6.92 (m, 1.5H), 7.27-7.30 (m, 1H), 7.36-7.40 (m, 1H), 7.46-7.50 (m, 1H), 7.88 (d, 1H), 8.94 (s, 1H).

### Example 60

### Preparation of (R)-1-(3-((2-((3-cyclopropyl-2,3,4,5-tetrahydro-1H-benzo[d]azepin-7-yl)amino)-5-fluoropyrimidin-4-yl)amino)piperidin-1-yl)prop-2-en-1-one (I-60)

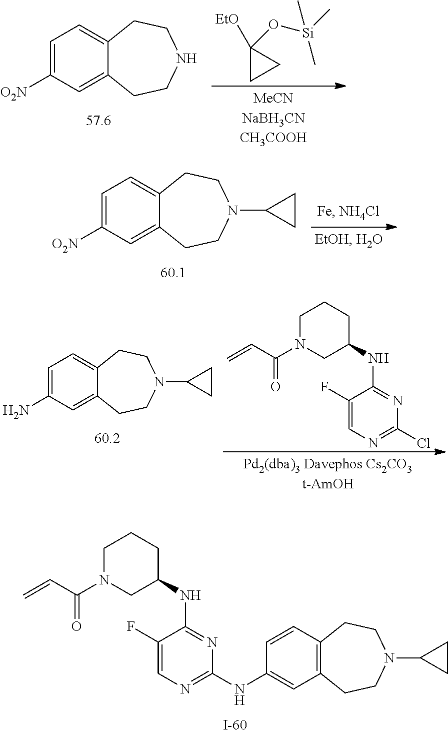

### 3-Cyclopropyl-7-nitro-2,3,4,5-tetrahydro-1H-benzo[d]azepine (60.1)

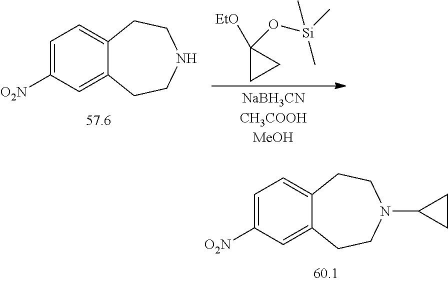 [SMILES: O=[N+]([O-])c1ccc2c(c1)CCNCC2 ; CCOC1(O[Si](C)(C)C)CC1 ; O=[N+]([O-])c1ccc2c(c1)CCN(C1CC1)CC2]

(1-Ethoxycyclopropoxy)trimethylsilane (400 mg, 2.3 mmol) was slowly added to the solution of 7-nitro-2,3,4,5-tetrahydro-1H-benzo[d]azepine (57.6) (200 mg, 1.0 mmol), NaBH₃CN (400 mg, 6.4 mmol), and acetic acid (637 mg, 10.6 mmol) in MeOH (20 mL) at 0° C. The resulting mixture was heated at 65° C. for 4 h. The reaction mixture was diluted with water and extracted with ethyl acetate. The combined organic layers were washed with water and brine, dried over sodium sulfate, and concentrated to afford the crude product. The crude product was purified by column chromatography (ethyl acetate/hexane:1/5) to afford 3-cyclopropyl-7-nitro-2,3,4,5-tetrahydro-1H-benzo[d]azepine (60.1) as a yellow solid (200 mg, 83%).

LCMS: 233.1 [M+1]⁺:

### 3-Cyclopropyl-2,3,4,5-tetrahydro-1H-benzo[d]azepin-7-amine (60.2)

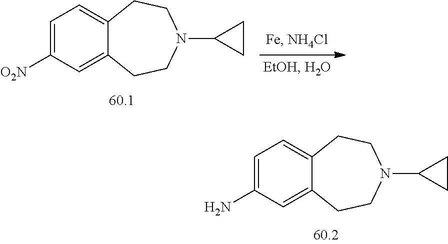 [SMILES: O=[N+]([O-])c1ccc2c(c1)CCN(C1CC1)CC2 ; Nc1ccc2c(c1)CCN(C1CC1)CC2]

Fe (289 mg, 5.2 mmol) and NH₄Cl (279 mg, 13.7 mmol) were added to the solution of 3-cyclopropyl-7-nitro-2,3,4,5-tetrahydro-1H-benzo[d]azepine (60.1) (200 mg, 0.86 mmol) in EtOH (20 mL) and H₂O (2 mL). The mixture was stirred at 90° C. for 1 h. After cooling to room temperature, the reaction mixture was diluted with water and extracted with ethyl acetate. The combined organic layers were washed with water and brine, dried over sodium sulfate, and concentrated to afford the crude product. The crude product was purified by column chromatography (ethyl acetate/hexane:1/1) to afford the title compound 3-cyclopropyl-2,3,4,5-tetrahydro-1H-benzo[c/]azepin-7-amine (60.2) as a yellow solid (80 mg, 46%).

LCMS: 203.1 [M+1]⁺.

### (R)-1-(3-(2-(3-Cyclopropyl-2,3,4,5-tetrahydro-1H-benzo[d]azepin-7-ylamino)-5-fluoropyrimidin-4-ylamino)piperidin-1-yl)prop-2-en-1-one (I-60)

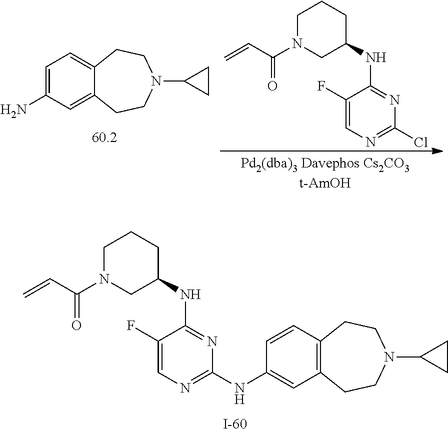

(R)-1-(3-(2-chloro-5-fluoropyrimidin-4-ylamino)piperidin-1-yl)prop-2-en-1-one (intermediate B, 169 mg, 0.95 mmol) and Cs₂CO₃ (258 mg, 0.79 mmol) were added to the solution of (60.2) (80 mg, 0.40 mmol) in tert-amyl alcohol (10 mL). Tris(dibenzylideneacetone)dipalladium(O) (36 mg, 0.04 mmol) and DavePhos (31 mg, 0.08 mmol) were added under N₂. The reaction mixture was stirred at 100° C. for 4 h. The reaction mixture was diluted with water and extracted with ethyl acetate. The combined organic layers were washed with water and brine, dried over sodium sulfate, and concentrated to afford the crude product. The crude product was purified by column chromatography (DCM/MeOH: 10/1) to afford (R)-1-(3-(2-(3-cyclopropyl-2,3,4,5-tetrahydro-1H-benzo[d]azepin-7-ylamino)-5-fluoropyrimidin-4-ylamino)piperidin-1-yl)prop-2-en-1-one (I-60) as an off-white solid (80 mg, 45%).

LCMS: 451.3 [M+1]⁺.

¹H NMR (400 MHz, CD₃OD): δ 1.05-1.13 (m, 4H), 1.54-1.61 (m, 1H), 1.77-1.84 (m, 3H), 1.94-1.98 (m, 1H), 2.11-2.19 (m, 1H), 2.75-3.22 (m, 9H), 3.87-3.92 (m, 2H), 4.05-4.16 (m, 2H), 4.46-4.50 (m, 0.5H), 4.61-4.65 (m, 0.5H), 5.42-5.45 (m, 0.5H), 5.81-5.85 (m, 0.5H), 5.98-6.20 (m, 1H), 6.25-6.30 (m, 0.5H), 6.83-6.90 (m, 0.5H), 7.25-7.55 (m, 3H), 7.91-7.99 (m, 1H).

### Example 61

### Preparation of (R)-5-((4-((1-acryloylpiperidin-3-yl)amino)-5-fluoropyrimidin-2-yl)amino)-2-methylisoindolin-1-one (I-61)

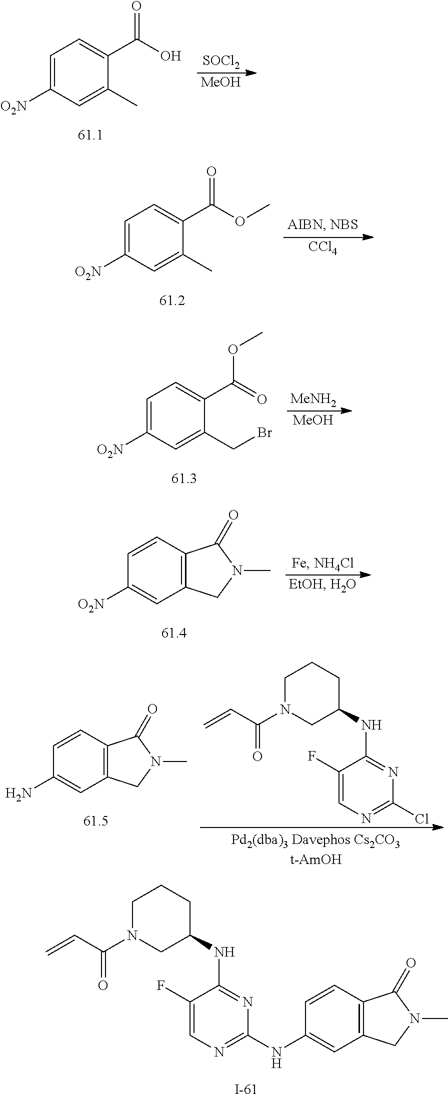

### Methyl 2-methyl-4-nitrobenzoate (61.2)

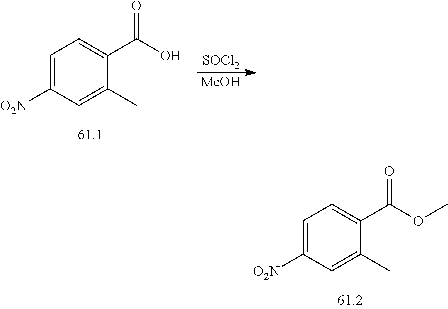

SOCl₂ (2.6 g, 22.0 mmol) was slowly added to the solution of 2-methyl-4-nitrobenzoic acid (61.1) (2.0 g, 11.0 mmol) in MeOH (20 mL) at 0° C. The resulting mixture was heated at 80° C. for 2 h. After cooling to room temperature, the reaction mixture was concentrated to afford methyl 2-methyl-4-nitrobenzoate (61.2) as a yellow oil (2.0 g, 93%).

### Methyl 2-(bromomethyl)-4-nitrobenzoate (61.3)

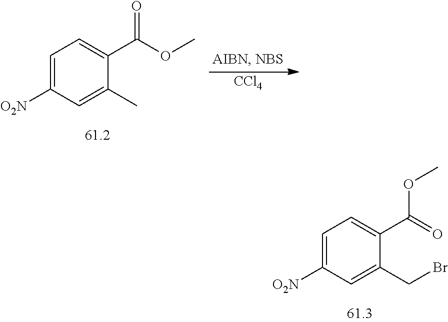

Azobisisobutyronitrile (AIBN) (0.17 g, 1.02 mmol) was added to the solution of methyl 2-methyl-4-nitrobenzoate (61.2) (2.0 g, 10.2 mmol) and N-bromosuccinimide (NBS) (2.7 g, 15.2 mmol) in CCl₄ (50 mL). The mixture was stirred at 80° C. overnight. After cooling to room temperature, the reaction mixture was filtered and, the filtrate was concentrated to afford methyl 2-(bromomethyl)-4-nitrobenzoate (61.3) as a white solid (1.1 g, 39%).

LCMS: 314.0 [M+CH₃CN]⁺.

### 2-Methyl-5-nitroisoindolin-1-one (61.4)

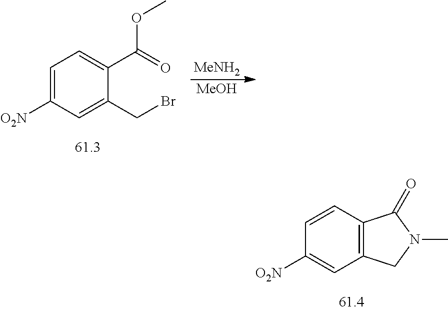

A solution of methyl 2-(bromomethyl)-4-nitrobenzoate (61.3) (1.0 g, 3.6 mmol) and MeNH₂/MeOH (2M; 2 mL) in MeOH (5 mL) was stirred at ambient temperature for 2 h. The reaction mixture was diluted with water and extracted with ethyl acetate. The combined organic layers were washed with water and brine, dried over sodium sulfate, and concentrated to afford the crude produce. The crude product was purified by column chromatography (ethyl acetate/hexane:1/10) to afford 2-methyl-5-nitroisoindolin-1-one (61.4) as a white solid (400 mg, 57%).

LCMS: 193.1 [M+1]⁺.

### 5-Amino-2-methylisoindolin-1-one (61.5)

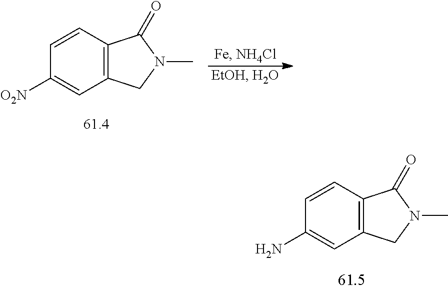

Fe (350 mg, 6.2 mmol) and NH₄Cl (337 mg, 6.2 mmol) were added to the solution of 2-methyl-5-nitroisoindolin-1-one (61.4) (200 mg, 1.0 mmol) in EtOH (10 mL) and H₂O (1 mL). The mixture was stirred at 90° C. for 1 h. After cooling to room temperature, the reaction mixture was diluted with water and extracted with ethyl acetate. The combined organic layers were washed with water and brine, dried over sodium sulfate, and concentrated to afford the crude product. The crude product was purified by column chromatography (ethyl acetate/hexane:1/10) to afford 5-amino-2-methylisoindolin-1-one (61.5) as a brown solid (50 mg, 30%).

LCMS: 163.1 [M+1]⁺.

### (R)-5-(4-(1-Acryloylpiperidin-3-ylamino)-5-fluoropyrimidin-2-ylamino)-2-methylisoindolin-1-one (I-61)

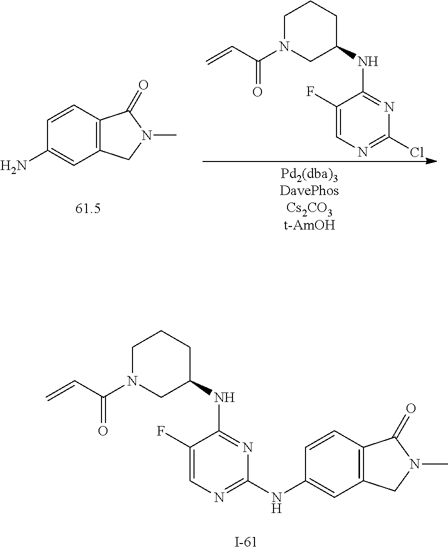

(R)-1-(3-(2-Chloro-5-fluoropyrimidin-4-ylamino)piperidin-1-yl)prop-2-en-1-one (intermediate B, 130 mg, 0.46 mmol) and Cs₂CO₃ (295 mg, 0.91 mmol) were added to the solution of 5-amino-2-methylisoindolin-1-one (61.5) (44 mg, 0.27 mmol) in tert-amyl alcohol (4 mL). Tris(dibenzylideneacetone)dipalladium(O) (41 mg, 0.046 mmol) and DavePhos (18 mg, 0.046 mmol) were added under N₂. The reaction mixture was stirred at 100° C. for 1 h. The reaction mixture was diluted with water and extracted with ethyl acetate. The combined organic layers were washed with water and brine, dried over sodium sulfate, and concentrated to afford the crude product. The crude product was purified by column chromatography (DCM/MeOH:10/1) to afford (R)-5-(4-(1-acryloylpiperidin-3-ylamino)-5-fluoropyrimidin-2-ylamino)-2-methylisoindolin-1-one (I-61) as a yellow solid (50 mg, 45%).

Mp: 120-122° C.

LCMS: 411.3 [M+1]⁺.

¹H NMR (400 MHz, DMSO-d₆): δ 1.42-1.48 (m, 1H), 1.65-1.70 (m, 1H), 1.81-1.85 (m, 1H), 1.99-2.01 (m, 1H), 2.65-2.84 (m, 1H), 2.99-3.05 (m, 3.5H), 3.12-3.18 (m, 0.5H), 4.02-4.05 (m, 2H), 4.18-4.38 (m, 2.5H), 4.50-4.54 (m, 0.5H), 5.45 (d, 0.5H), 5.72-5.75 (m, 0.5H), 6.00 (d, 0.5H), 6.15 (dd, 1H), 6.60-6.66 (m, 0.5H), 6.86-6.93 (m, 0.5H), 7.45-7.64 (m, 3H), 7.96-8.10 (m, 2H), 9.51 (d, J=8 Hz, 1H).

### Example 62

### Preparation of (R)-6-((4-((1-acryloylpiperidin-3-yl)amino)-5-fluoropyrimidin-2-yl)amino)-2-methylisoindolin-1-one (I-62)

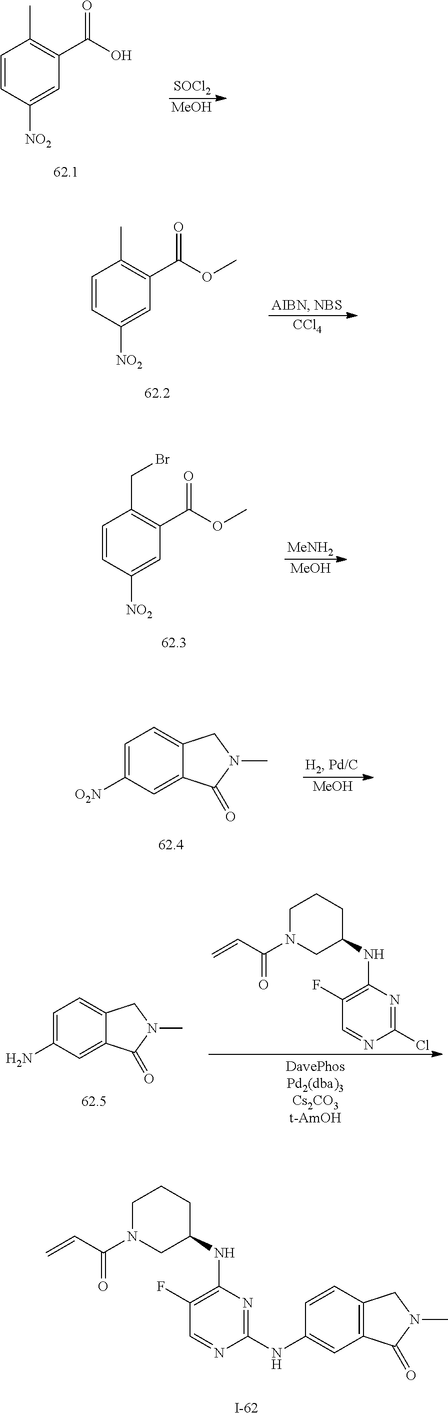

### Methyl 2-methyl-5-nitrobenzoate (62.2)

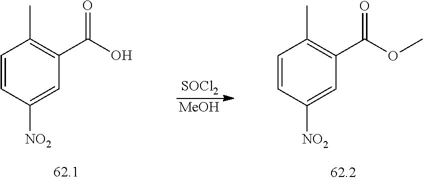

To a solution of 2-methyl-5-nitrobenzoic acid (62.1) (3.0 g, 16.6 mmol) in methanol (30 mL) was added thionyl chloride (10 mL). The reaction mixture was stirred at 70° C. for 3 h. TLC showed the reaction was complete. The excess thionyl chloride was removed. The reaction mixture was quenched with the addition of water and extracted with ethyl acetate. The combined organic layers were washed with water and brine, dried over sodium sulfate, and concentrated to afford the crude product. The crude methyl 2-methyl-5-nitrobenzoate (62.2) was used in the next step without further purification (3.18 g, 98%).

¹HNMR (400 MHz, CDCl₃): δ 2.72 (s, 3H), 3.95 (s, 3H), 7.43 (d, 1H), 8.23 (dd, 1H), 8.78 (d, 1H).

### Methyl 2-(bromomethyl)-5-nitrobenzoate (62.3)

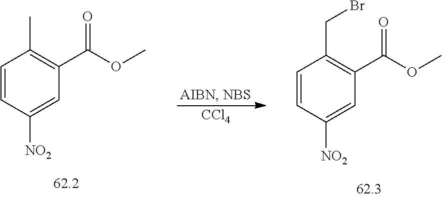

To a solution of methyl 2-methyl-5-nitrobenzoate (62.2) (1.5 g, 7.7 mmol) in carbon tetrachloride (30 mL) was added N-bromosuccinimide (NBS) (1.5 g, 8.42 mmol) and azobisisobutyronitrile (AIBN) (124 mg, 0.76 mmol). The reaction mixture was stirred at 90° C. for 5 h. TLC showed the reaction was complete. The reaction mixture was filtered, and the filtrate was quenched with the addition of water and extracted with ethyl acetate. The combined organic layers were washed with water and brine, dried over sodium sulfate, and concentrated to afford the crude product. The crude product was purified by column chromatography (DCM) to afford methyl 2-(bromomethyl)-5-nitrobenzoate (62.3) as a yellow solid (1.9 g, 90%).

LCMS: 314.0 [M+1]⁺.

### 2-Methyl-6-nitroisoindolin-1-one (62.4)

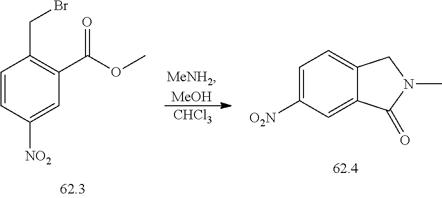

To a solution of methyl 2-(bromomethyl)-5-nitrobenzoate (62.3) (500 mg, 1.82 mmol) in chloroform (15 mL) was added methylamine in methanol solution (2M; 2 mL). The reaction mixture was stirred at 70° C. for 6 h under N₂. TLC showed the reaction was complete. The reaction mixture was quenched with the addition of water and extracted with ethyl acetate. The combined organic layers were washed with water and brine, dried over sodium sulfate, and concentrated to afford the crude product. The crude product was purified by column chromatography (hexane/ethyl acetate:2/1) to afford 2-methyl-6-nitroisoindolin-1-one (62.4) as a red solid (146 mg, 42%).

¹HNMR (400 MHz, CDCl₃): δ 3.24 (s, 3H), 4.50 (s, 2H), 7.62 (d, 1H), 8.41 (dd, 1H), 8.67 (d, 1H).

### 6-Amino-2-methylisoindolin-1-one (62.5)

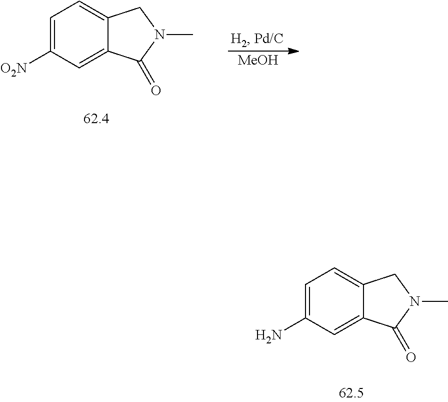

To a solution of 2-methyl-6-nitroisoindolin-1-one (62.4) (146 mg, 0.76 mmol) in methanol (14 mL) was added 15 mg Pd/C. The reaction mixture was stirred at ambient temperature for 3 h under H₂. TLC showed the reaction was complete. The reaction mixture was filtered, and the filtrate was concentrated to afford 6-amino-2-methylisoindolin-1-one (62.5) as a yellow solid (106 mg, 86%).

¹HNMR (400 MHz, CDCl₃): δ 3.16 (s, 3H), 3.82 (br, 2H), 4.25 (s, 2H), 6.83 (dd, 1H), 7.11 (d, 1H), 7.18 (d, 1H).

### ((R)-6-((4-((1-Acryloylpiperidin-3-yl)amino)-5-fluoropyrimidin-2-yl)amino)-2-methylisoindolin-1-one (I-62)

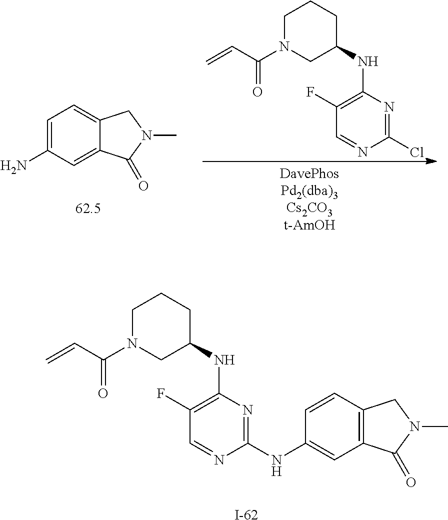

To a solution of 6-amino-2-methylisoindolin-1-one (62.5) (106 mg, 0.65 mmol) in tert-amyl alcohol (15 mL) was added (R)-1-(3-((2-chloro-5-fluoropyrimidin-4-yl)amino)piperidin-1-yl)prop-2-en-1-one (intermediate B, 186 mg, 0.62 mmol), Davephos (51 mg, 0.13 mmol), tris(dibenzylideneacetone)dipalladium(O) (59 mg, 0.07 mmol), and Cs₂CO₃ (426 mg, 1.31 mmol). The reaction mixture was stirred at 100° C. for 3 h under N₂. TLC showed the reaction was complete. The reaction mixture was quenched with the addition of water and extracted with ethyl acetate. The combined organic layers were washed with water and brine, dried over sodium sulfate, and concentrated to afford the crude product. The crude product was purified by column chromatography (DCM/MeOH:20/1) to afford ((R)-6-((4-((1-acryloylpiperidin-3-yl)amino)-5-fluoropyrimidin-2-yl)amino)-2-methylisoindolin-1-one (I-62) as a yellow solid (140 mg, 52%).

LCMS: 411.4 [M+1]⁺.

¹HNMR (400 MHz, DMSO-d₆): δ1.49-1.63 (m, 2H), 1.80 (d, 1H), 1.98-2.03 (m, 1H), 2.77-2.88 (m, 1H), 3.04 (s, 3.5H), 3.18-3.24 (m, 0.5H), 3.94-4.13 (m, 2.5H), 4.34 (d, 2.5H), 5.47 (d, 0.5H), 5.68 (d, 0.5H), 6.04 (dd, 1H), 6.58-6.64 (m, 0.5H), 6.79-6.86 (m, 0.5H), 7.37 (t, 2H), 7.78-7.82 (m, 1H), 7.95 (d, 1H), 8.13 (s, 1H), 9.30 (s, 1H).

### Example 63

### Preparation of (R)-5-((4-((1-Acryloylpiperidin-3-yl)amino)-5-fluoropyrimidin-2-yl)amino)-2-methylisoindoline-1,3-dione (I-63)

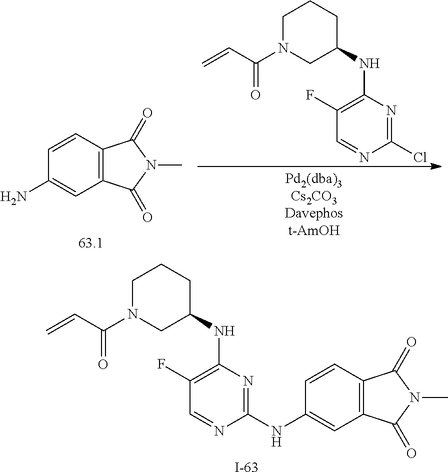

A mixture of (R)-1-(3-((2-chloro-5-fluoropyrimidin-4-yl)amino)piperidin-1-yl)prop-2-en-1-one (intermediate B, 100 mg, 0.35 mmol), 5-amino-2-methylisoindoline-1,3-dione (63.1) (62 mg, 0.35 mmol), Cs₂CO₃ (229 mg, 0.7 mmol), tris(dibenzylideneacetone)dipalladium(O) (32 mg, 0.035 mmol), and Davephos (14 mg, 0.035 mmol) in tert-amyl alcohol (10 mL) was stirred at 100° C. for 3 h. The mixture was quenched with the addition of water and extracted with ethyl acetate. The combined organic layers were washed with water and brine, dried over sodium sulfate and concentrated to dryness to afford the crude product. The crude product was purified by column chromatography (hexanes/ethyl acetate:1/1) to afford (R)-5-((4-((1-acryloylpiperidin-3-yl)amino)-5-fluoropyrimidin-2-yl) amino)-2-methylisoindoline-1,3-dione (I-63) as a yellow solid (50 mg, 33%).

LCMS: 425.5 [M+1]⁺.

¹HNMR (400 MHz, DMSO-d₆): δ 1.52-1.70 (m, 2H), 1.80-1.84 (m, 1H), 2.01-2.05 (m, 1H), 2.73-2.83 (m, 1H), 2.99 (s, 3H), 3.01-3.07 (m, 0.5H), 3.16-3.22 (m, 0.5H), 3.98-4.07 (m, 2H), 4.15-4.19 (m, 0.5H), 4.42-4.47 (m, 0.5H), 5.46 (d, 0.5H), 5.68 (d, 0.5H), 6.03 (dd, 1H), 6.59-6.66 (m, 0.5H), 6.83 (dd, 0.5H), 7.59 (d, 1H), 7.68 (d, 1H), 7.96 (dd, 1H), 8.02 (d, 1H), 8.35 (d, 1H), 9.90 (s, 1H).

### Example 64

### Preparation of (R)-1-(3-((2-((2-methylisoindolin-5-yl)amino)-5-(trifluoromethyl)pyrimidin-4-yl)amino)piperidin-1-yl)prop-2-en-1-one (I-64)

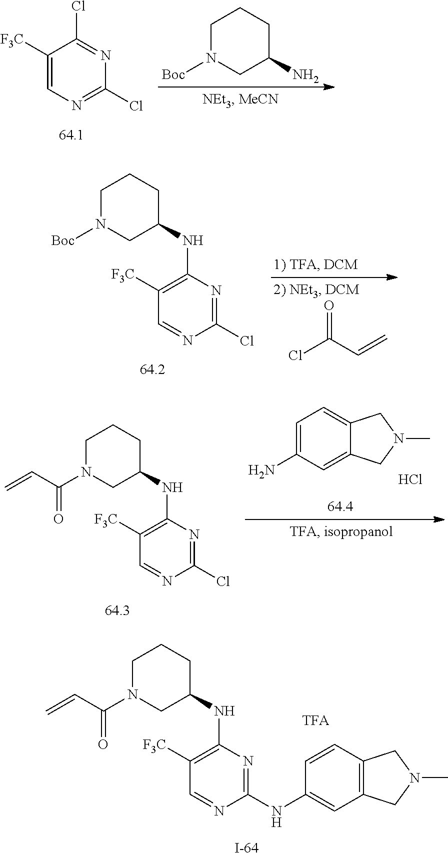

### (R)-tert-Butyl 3-((2-chloro-5-(trifluoromethyl)pyrimidin-4-yl)amino)piperidine-1-carboxylate (64.2)

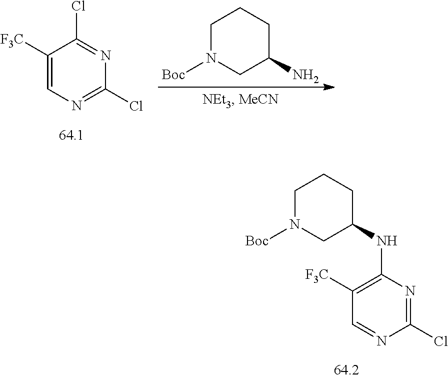 [SMILES: FC(F)(F)c1cnc(Cl)nc1Cl ; CC(C)(C)OC(=O)N1CCC[C@@H](N)C1 ; CC(C)(C)OC(=O)N1CCC[C@@H](Nc2nc(Cl)ncc2C(F)(F)F)C1]

A mixture of 2,4-dichloro-5-(trifluoromethyl)pyrimidine (64.1) (5 g, 23 mmol), (R)-tert-butyl 3-aminopiperidine-1-carboxylate (5.1 g, 25.5 mmol) and triethylamine (4.65 g, 46 mmol) in MeCN (35 mL) was stirred at ambient temperature for 2 h. The mixture was quenched with the addition of water and extracted with ethyl acetate. The combined organics were washed with water and brine, dried over sodium sulfate, and concentrated to dryness to afford the crude product. The crude product was purified by column chromatography (hexanes/ethyl acetate:5/1) to afford (R)-tert-butyl 3-((2-chloro-5-(trifluoromethyl)pyrimidin-4-yl)amino)piperidine-1-carboxylate (64.2) (1.93 g, 22%).

¹HNMR (400 MHz, CDCl₃): δ 1.45 (s, 9H), 1.63 (br, 2H), 1.86 (s, 2H), 3.21-3.22 (m, 1H), 3.50-4.00 (m, 3H), 4.29 (s, 1H), 8.27 (s, 1H).

### (R)-1-(3-((2-Chloro-5-(trifluoromethyl)pyrimidin-4-yl)amino)piperidin-1-yl)prop-2-en-1-one (64.3)

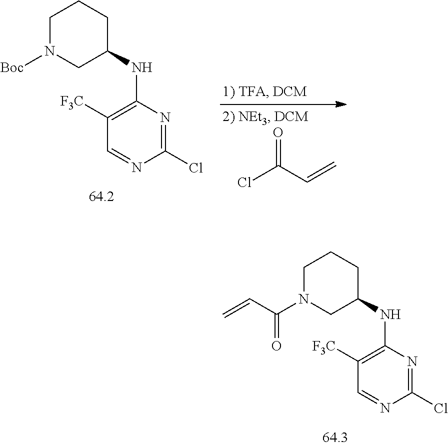

A suspension of (R)-tert-butyl 3-((2-chloro-5-(trifluoromethyl)pyrimidin-4-yl)amino)piperidine-1-carboxylate (64.2) (1.9 g, 5 mmol) in DCM/TFA (10 mL/5 mL) was stirred at ambient temperature for 30 min. The reaction mixture was concentrated, and a saturated NCHO₃ aqueous solution was added to the residue. The resulting mixture was extracted with DCM. The combined organic layers were washed with water and brine, dried over sodium sulfate, and concentrated to dryness to afford the crude intermediate. The crude intermediate was diluted with DCM (10 mL). Acryloyl chloride (406 mg, 4.4 mmol) and triethylamine (907 mg, 10 mmol) were added to the mixture, and the reaction mixture was stirred at ambient temperature for 10 min. The mixture was quenched with the addition of water and extracted with DCM. The combined organic layers were washed with water and brine, dried over sodium sulfate, and concentrated to dryness to afford the crude product. The crude product was purified by column chromatography (hexanes/ethyl acetate:1/1) to afford (R)-1-(3-((2-chloro-5-(trifluoromethyl)pyrimidin-4-yl)amino)piperidin-1-yl)prop-2-en-1-one (64.3) (1.03 g, 62%) as a white solid.

¹HNMR (400 MHz, CDCl₃): δ 1.69 (s, 2H), 2.09 (br, 1H), 2.54-2.76 (m, 0.7H), 3.22-4.34 (m, 5.3H), 5.32-5.34 (m, 0.3H), 5.73 (br, 1H), 6.07 (br, 0.2H), 6.30-6.36 (m, 0.8H), 6.58-6.65 (m, 0.8H), 8.21-8.29 (m, 1H).

### (R)-1-(3-((2-((2-Methylisoindolin-5-yl)amino)-5-(trifluoromethyl)pyrimidin-4-yl)amino)piperidin-1-yl)prop-2-en-1-one (I-64)

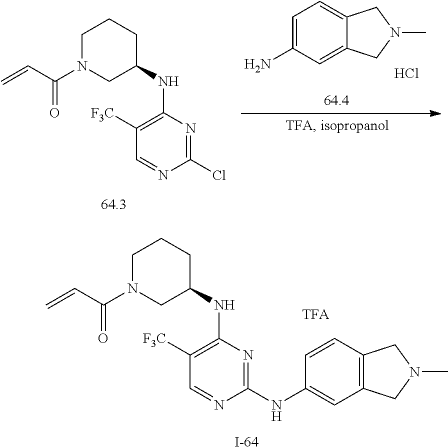

A mixture of (R)-1-(3-((2-chloro-5-(trifluoromethyl)pyrimidin-4-yl)amino)piperidin-1-yl)prop-2-en-1-one (64.3) (100 mg, 0.3 mmol), 2-methylisoindolin-5-amine HCl salt (64.4) (50 mg, 0.27 mmol), and TFA (0.05 mL) in isopropanol (10 mL) was stirred at 90° C. for 1 h. The mixture was quenched with the addition of water and extracted with ethyl acetate. The combined organic layers were washed with water and brine, dried over sodium sulfate, and concentrated to dryness to afford the crude product. The crude product was purified by HPLC to afford the TFA salt of (R)-1-(3-((2-((2-methylisoindolin-5-yl)amino)-5-(trifluoromethyl)pyrimidin-4-yl)amino)piperidin-1-yl)prop-2-en-1-one (I-64) as a white solid (50 mg, 33%).

LCMS: 447.2 [M+1]⁺.

¹HNMR (400 MHz, DMSO-d₆): δ 1.56-1.61 (m, 1H), 1.90 (t, 2H), 2.03-2.12 (m, 1H), 2.85˜2.91 (m, 0.5H), 3.05-3.19 (m, 4H), 3.25-3.28 (m, 0.5H), 4.00-4.10 (m, 1H), 4.28-4.53 (m, 4H), 5.47-5.50 (m, 0.5H), 5.85 (d, 0.5H), 6.05 (dd, 0.5H), 6.29 (d, 0.5H), 6.36-6.41 (m, 0.5H), 6.86 (dd, 0.5H), 6.37-6.40 (m, 1H), 7.55-7.64 (m, 1H), 7.75 (br, 1H), 8.29 (d, 1H).

### Example 65

### Preparation of (R)-1-(3-((2-(Isoindolin-5-ylamino)-5-(trifluoromethyl)pyrimidin-4-yl)amino)piperidin-1-yl)prop-2-en-1-one (I-65)

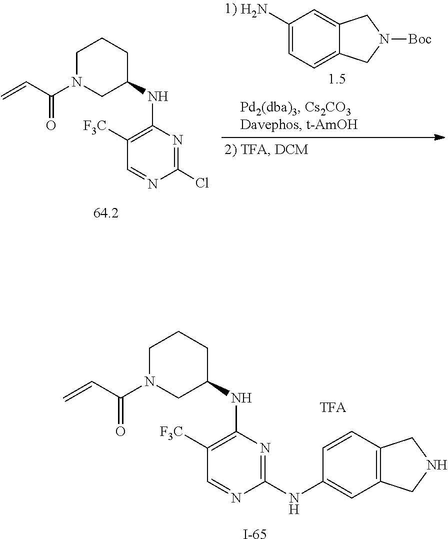

A mixture of (R)-1-(3-((2-chloro-5-(trifluoromethyl)pyrimidin-4-yl) amino)piperidin-1-yl)prop-2-en-1-one (64.2) (100 mg, 0.3 mmol), tert-butyl 5-aminoisoindoline-2-carboxylate (1.5) (64 mg, 0.27 mmol), Cs₂CO₃ (177 mg, 0.54 mmol), tris(dibenzylideneacetone)dipalladium(O) (25 mg, 0.03 mmol), and Davephos (10 mg, 0.03 mmol) in tert-amyl alcohol (10 mL) was stirred at 100° C. for 3 h. The mixture was quenched with the addition of water and extracted with ethyl acetate. The combined organic layers were washed with water and brine, dried over sodium sulfate, and concentrated to dryness to afford the crude product. The crude product was purified by column chromatography (hexanes/ethyl acetate:1/1) to afford (R)-1-(3-((2-(isoindolin-5-ylamino)-5-(trifluoromethyl)pyrimidin-4-yl)amino)piperidin-1-yl)prop-2-en-1-one (I-65).

The product was taken up in DCM/TFA (5 mL/3 mL) and stirred at room temperature for 30 min. The mixture was concentrated to dryness to afford the TFA salt of (R)-1-(3-((2-(isoindolin-5-ylamino)-5-(trifluoromethyl)pyrimidin-4-yl)amino)piperidin-1-yl)prop-2-en-1-one (I-65) as a white solid (35 mg, 21%).

LCMS: 433.1 [M+1]⁺.

¹HNMR (400 MHz, CD₃OD): δ 1.58-1.61 (m, 1H), 1.86-1.91 (m, 2H), 2.03-2.09 (m, 1H), 2.88-2.96 (m, 0.5H), 3.04-3.10 (m, 0.5H), 3.15-3.22 (m, 0.5H), 3.26-3.27 (m, 0.5H), 3.98-4.10 (m, 1H), 4.29-4.39 (m, 1.5H), 4.49-4.63 (m, 4.5H), 5.47 (d, 0.5H), 5.84 (d, 0.5H), 6.05 (d, 0.5H), 6.28 (d, 0.5H), 6.40 (dd, 0.5H), 6.86 (dd, 0.5H), 7.36-7.39 (m, 1H), 7.58 (dd, 1H), 7.81 (s, 1H), 8.24 (d, 1H).

### Example 66

### Preparation of 1-(6-((5-fluoro-2-((2-methylisoindolin-5-yl)amino)pyrimidin-4-yl)amino)-2-azabicyclo[2.2.1]heptan-2-yl)prop-2-en-1-one (I-66)

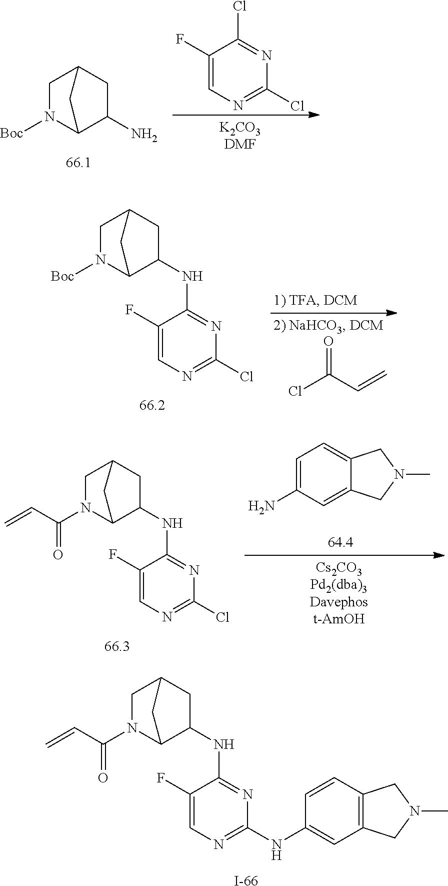

### tert-Butyl 6-((2-chloro-6-fluoropyrimidin-4-yl)amino)-2-azabicyclo[2.2.1]heptane-2-carboxylate (66.2)

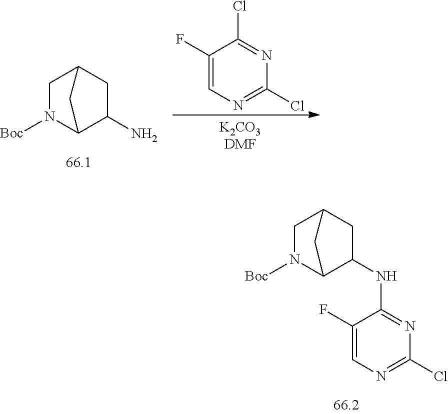 [SMILES: CC(C)(C)OC(=O)N1CC2CC(N)C1C2 ; Fc1cnc(Cl)nc1Cl ; CC(C)(C)OC(=O)N1CC2CC(Nc3nc(Cl)ncc3F)C1C2]

A mixture of tert-butyl 6-amino-2-azabicyclo[2.2.1]heptane-2-carboxylate (66.1) (500 mg, 2.35 mmol), 2,4-dichloro-6-fluoropyrimidine (393 mg, 2.35 mmol), and K₂CO₃ (650 mg, 4.71 mmol) in DMF (10 mL) was stirred at ambient temperature for 3 h. The mixture was quenched with the addition of water and extracted with ethyl acetate. The combined organic layers were washed with water and brine, dried over sodium sulfate, and concentrated to dryness to afford the crude product. The crude product was purified by column chromatography (hexanes/ethyl acetate:2/1) to afford tert-butyl 6-((2-chloro-6-fluoropyrimidin-4-yl)amino)-2-azabicyclo[2.2.1]heptane-2-carboxylate (66.2) (600 mg, 74%) as a white solid.

¹HNMR (400 MHz, DMSO-d₆): δ 1.07-1.40 (m, 9H), 1.58-1.61 (m, 3H), 1.95-2.02 (m, 1H), 2.49 (s, 1H), 3.10-3.18 (m, 2H), 4.13-4.33 (m, 2H), 7.88 (d, 0.2H), 8.03-8.13 (m, 1H), 8.24 (d, 0.8H).

### 1-(6-((2-Chloro-6-fluoropyrimidin-4-yl)amino)-2-azabicyclo[2.2.1]heptan-2-yl)prop-2-en-1-one (66.3)

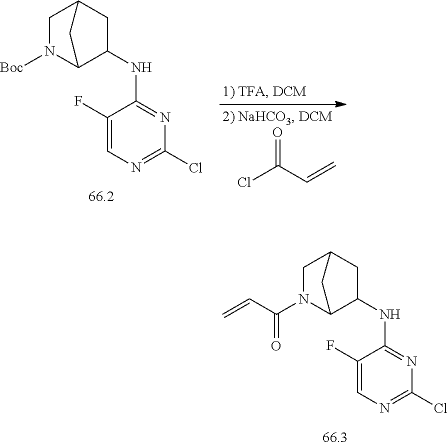

A suspension of tert-butyl 6-((2-chloro-6-fluoropyrimidin-4-yl)amino)-2-azabicyclo[2.2.1]heptane-2-carboxylate (66.2) (600 mg, 1.75 mmol) in DCM/TFA (10 mL/5 mL) was stirred at ambient temperature for 30 min. The reaction mixture was concentrated to afford the crude residue, and the crude residue was diluted with DCM/NaHCO₃ (10 mL/5 mL). Acryloyl chloride (142 mg, 1.58 mmol) was added to the resulting mixture, and the reaction mixture was stirred at ambient temperature for 10 min. The mixture was quenched with the addition of water and extracted with DCM. The combined organic layers were washed with water and brine, dried over sodium sulfate, and concentrated to dryness to afford the crude product. The crude product was purified by column chromatography (DCM/MeOH:10/1) to afford 1-(6-(2-chloro-6-fluoropyrimidin-4-yl)amino)-2-azabicyclo[2.2.1]heptan-2-yl)prop-2-en-1-one (66.3) (117 mg, 22%).

¹HNMR (400 MHz, DMSO-d₆): δ 1.54-1.58 (m, 1H), 1.67-1.79 (m, 2H), 2.04-2.05 (m, 1H), 2.53-2.58 (m, 1H), 3.27 (s, 0.5H), 3.31-3.34 (m, 1H), 3.47 (s, 0.5H), 4.29-4.37 (m, 1H), 4.52 (s, 0.7H), 4.67 (s, 0.3H), 5.28 (dd, 0.7H), 5.62 (dd, 0.3H), 5.91 (d, 0.2H), 5.95 (d, 0.5H), 6.00-6.07 (m, 1H), 6.47 (dd, 0.3H), 7.90 (d, 0.3H), 8.05, 8.09 (two doublets, 1H), 8.20 (d, 0.7H).

### 1-(6-((6-Fluoro-2-((2-methylisoindolin-5-yl)amino)pyrimidin-4-yl)amino)-2-azabicyclo[2.2.1]heptan-2-yl)prop-2-en-1-one (I-66)

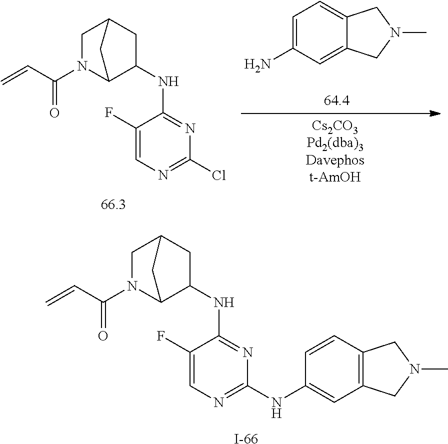

A mixture of 1-(6-((2-chloro-6-fluoropyrimidin-4-yl)amino)-2-azabicyclo[2.2.1]heptan-2-yl)prop-2-en-1-one (66.3) (50 mg, 0.17 mmol), 2-methylisoindolin-5-amine (32 mg, 0.22 mmol), Cs₂CO₃ (110 mg, 0.34 mmol), tris(dibenzylideneacetone)dipalladium(O) (15 mg, 0.017 mmol), and Davephos (7 mg, 0.017 mmol) in tert-amyl alcohol (10 mL) was stirred at 100° C. for 3 h. The mixture was quenched with the addition of water and extracted with ethyl acetate. The combined organic layers were washed with water and brine, dried over sodium sulfate, and concentrated to dryness to afford the crude product. The crude product was purified by HPLC to afford 1-(6-((6-fluoro-2-((2-methylisoindolin-5-yl)amino)pyrimidin-4-yl)amino)-2-azabicyclo[2.2.1]heptan-2-yl)prop-2-en-1-one (I-66) as a yellow solid (28 mg, 31%).

LCMS: 409.1 [M+1]⁺.

¹HNMR (400 MHz, CD₃OD): δ 1.55-1.65 (m, 1H), 1.77 (s, 1H), 1.88 (s, 1H), 2.24-2.31 (m, 1H), 2.71, 2.76 (two singlets, 1H), 3.17 (s, 3H), 3.38-3.55 (m, 1.5H), 3.64-3.67 (m, 0.5H), 4.48-4.68 (m, 3H), 4.84-4.95 (m, 3H), 5.50 (dd, 0.5H), 5.77 (dd, 0.5H), 6.17-6.26 (m, 1.5H), 6.55 (dd, 0.5H), 7.47 (t, 1H), 7.61-7.63 (m, 1H), 7.71 (d, 1H), 7.92 (dd, 1H).

### Example 67

### Preparation of (R)-1-(3-((5-fluoro-2-((2-(prop-2-yn-1-yl)isoindolin-5-yl)amino)pyrimidin-4-yl)amino)piperidin-1-yl)prop-2-en-1-one (I-67)

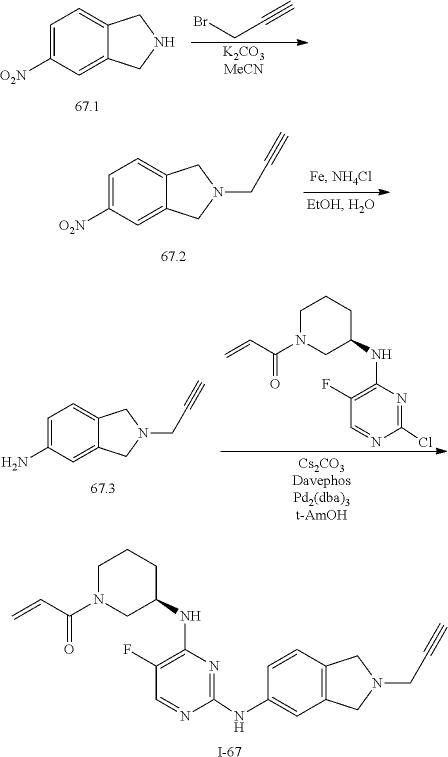

### 5-Nitro-2-(prop-2-yn-1-yl)isoindoline (67.2)

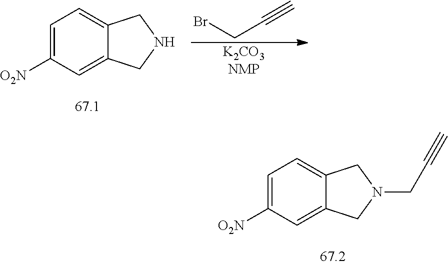

To a solution of 5-nitroisoindoline (67.1) (800 mg, 4.87 mmol) in N-methyl-2-pyrrolidone (NMP) (30 mL) was added 3-bromoprop-1-yne (1.0 g, 8.4 mmol) and K₂CO₃ (1.6 g, 11.6 mmol). The reaction mixture was stirred at ambient temperature for 3 h. TLC showed the reaction was complete. The reaction mixture was quenched with the addition of water and extracted with ethyl acetate. The combined organic layers were washed with water and brine, dried over sodium sulfate, and concentrated in vacuo to afford the crude product. The crude product was purified by column chromatography (hexane/ethyl acetate:1/1) to afford 5-nitro-2-(prop-2-yn-1-yl)isoindoline (67.2) as a yellow solid (438 mg, 44%).

¹HNMR (400 MHz, CDCl₃): δ 2.33 (t, 1H), 3.66 (d, 2H), 4.14 (s, 4H), 7.35 (d, 1H), 8.07 (d, 1H), 8.11 (dd, 1H).

### 2-(Prop-2-yn-1-yl)isoindolin-5-amine (67.3)

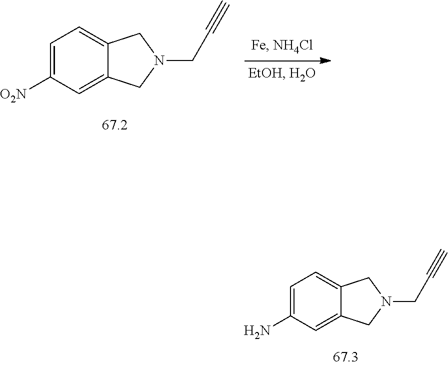

To a solution of 5-nitro-2-(prop-2-yn-1-yl)isoindoline (67.2) (400 mg, 1.98 mmol) in ethanol (15 mL) and water (15 mL) was added NH₄Cl (317 mg, 5.87 mmol) and Fe (332 mg, 5.94 mmol). The reaction mixture was stirred at 90° C. for 1 h. TLC showed the reaction was complete. The reaction mixture was filtered, and the filtrate was quenched with the addition of water and extracted with ethyl acetate. The combined organic layers were washed with water and brine, dried over sodium sulfate, and concentrated in vacuo to afford the crude product. The crude product was purified by column chromatography (hexane/ethyl acetate:1/2) to afford 2-(prop-2-yn-1-yl)isoindolin-5-amine (67.3) as a white solid (250 mg, 73%).

¹HNMR (400 MHz, CDCl₃): δ 2.27 (t, 1H), 3.59 (d, 4H), 3.96 (d, 4H), 6.52-6.55 (m, 2H), 6.97 (d, 1H).

### (R)-1-(3-((5-Fluoro-2-((2-(prop-2-yn-1-yl)isoindolin-5-yl)amino)pyrimidin-4-yl)amino)piperidin-1-yl)prop-2-en-1-one (I-67)

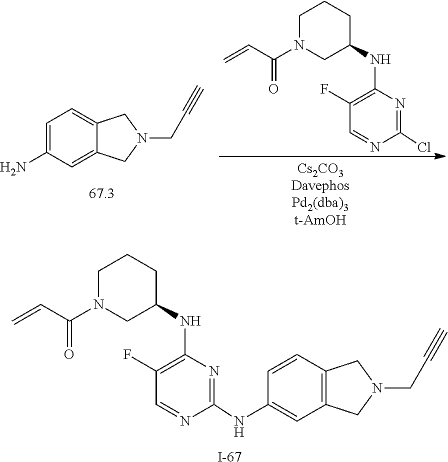

To a solution of 2-(prop-2-yn-1-yl)isoindolin-5-amine (67.3) (222 mg, 1.29 mmol) in tert-amyl alcohol (20 mL) was added (R)-1-(3-((2-chloro-5-fluoropyrimidin-4-yl)amino)piperidin-1-yl)prop-2-en-1-one (intermediate B, 368 mg, 1.29 mmol), Davephos (102 mg, 0.26 mmol), tris(dibenzylideneacetone)dipalladium(O) (102 mg, 0.11 mmol), and Cs₂CO₃ (842 mg, 2.58 mmol). The reaction mixture was stirred at 100° C. for 3 h under N₂. TLC showed the reaction was complete. The reaction mixture was quenched with the addition of water and extracted with ethyl acetate. The combined organic layers were washed with water and brine, dried over sodium sulfate, and concentrated in vacuo to afford the crude product. The crude product was purified by column chromatography (DCM/MeOH:20/1) to afford (R)-1-(3-((5-fluoro-2-((2-(prop-2-yn-1-yl)isoindolin-5-yl)amino)pyrimidin-4-yl)amino)piperidin-1-yl)prop-2-en-1-one (I-67) as a yellow solid (100 mg, 18%).

LCMS: 421.4 [M+1]⁺.

¹HNMR (400 MHz, DMSO-d₆): δ 1.38-1.42 (m, 1H), 1.61-1.67 (m, 1H), 1.81-1.84 (m, 1H), 1.97-2.00 (m, 1H), 2.68-2.83 (m, 1H), 2.99 (t, 0.5H), 3.12-3.16 (m, 0.5H), 3.19 (t, 1H), 3.52 (d, 2H), 3.84 (br, 4H), 4.00 (d, 2H), 4.17 (d, 0.5H), 4.45 (d, 0.5H), 5.50 (d, 0.5H), 5.71 (d, 0.5H), 6.08 (dd, 1H), 6.61 (dd, 1H), 6.84 (dd, 1H), 7.03 (d, 1H), 7.35 (d, 1.5H), 7.40 (d, 0.5H), 7.65 (two singlets, 1H), 7.89 (d, 1H); 9.01 (s, 1H).

### Example 68

### Omnia Assay Protocol for Potency Assessment Against BTK

The protocol below describes an assay to measure potency of compounds against the activated BTK enzyme. The mechanics of the assay platform are best described by the vendor (Life Technologies, Carlsbad, Calif.) on their website at the following URLs: www.lifetechnologies.com/us/en/home/life-science/drug-discovery/target-and-lead-identification-and-validation/kinasebiology/kinase-activity-assays/omnia-kinase-assays/omnia-kinase-principle.html and tools.lifetechnologies.com/content/sfs/manuals/omnia_kinase_assay_man.pdf

Briefly, 10× stocks of BTK (PV3363) from Life Technologies, 1.13×ATP (AS00lA) and Sox conjugated peptide substrates, Y5-Sox, (KZN305l) were prepared in 1× kinase reaction buffer consisting of 20 mM Tris, pH 7.5, 5 mM MgCl₂, 1 mM EGTA, 5 mM (3-glycerophosphate, 5% glycerol (10× stock, KB002A) and 0.2 mM DTT (DS00lA). 5 μL of each enzyme were pre-incubated in a Corning (#3574) 384-well, white, non-binding surface microtiter plate (Corning, N.Y.) for 30 min. at room temperature with a 0.5 μL volume of 50% DMSO or serially diluted compounds prepared in 50% DMSO. Kinase reactions were started with the addition of 45 μL of the ATP/Tyr-Sox peptide substrate Y5 mix and monitored every 71 seconds for 60 minutes at λₑₓ360/λₑₘ485 in a Synergy4 plate reader from BioTek (Winooski, Vt.). At the conclusion of each assay, progress curves from each well were examined for linear reaction kinetics and fit statistics (R2, 95% confidence interval, absolute sum of squares). Initial velocity (0 minutes to ˜30 minutes) from each reaction was determined from the slope of a plot of relative fluorescence units vs time (minutes) and then plotted against inhibitor concentration to estimate IC₅₀ from log [Inhibitor] vs Response, Variable Slope model in GraphPad Prism from GraphPad Software (San Diego, Calif.).

Reagent conditions for the assay are:

[BTK]=5 nM, [ATP]=40 μM, [Y5-Sox]=10 μM (ATP KMapp˜36 μM).

Table 6, under the column “IC₅₀”, shows the degree of inhibition of BTK activity by compounds of the invention in the BTK inhibition assay. The compound numbers correspond to the compound numbers in the examples. Compounds having an activity designated as “A” provided an IC₅₀<10 nM; compounds having an activity designated as “B” provided an IC₅₀ 10-100 nM; compounds having an activity designated as “C” provided an IC₅₀ of 101-1000 nM.

### Example 69

### BTK Ramos Cellular Assay

BTK activity in Ramos cells, in the presence of a compound of the invention, can be measured by 3 different methods: 1) inhibition of autophosphorylation of BTK; 2) inhibition of phosphorylation of the immediate BTK surrogate PLCγ2; and 3) BTK occupancy. All three of these methods are directly comparable to each other. In this instance, BTK occupancy was chosen and is described below.

Compounds of this invention were assayed in Ramos human Burkitt lymphoma cells. Ramos cells were grown in suspension in T175 flasks, spun down, resuspended to a concentration of 2×10⁶ cells per mL in serum-free media and incubated for 1 hour. Compound was added to Ramos cells to a final concentration of 500, 166.7, 55.6, 18.5, 6.2, 2.1, and 0.7 nM. Ramos cells were incubated with compound for 1 hour, washed with PBS and lysed in 1004 of BioRad lysis Buffer. Cell lysates were incubated with a biotinylated covalent probe (compound 1-215 disclosed in U.S. published application number 2010/0029610, incorporated by reference in its entirety) at a final concentration of 1 μM in a PBS, 0.05% Tween-20, 1% BSA solution for 1 h at room temperature. Standards and samples were transferred to a streptavidin-coated 96-well ELISA plate and mixed while shaking for 1 h at room temperature. The α-Btk antibody (BD 611116, 1:1000 dilution in PBS+0.05% Tween-20+0.5% BSA) was then added and incubated for 1 h at room temperature. After wash, goat α-mouse-HRP (1:5000 dilution in PBS+0.05% Tween-20+0.5% BSA) was added and incubated for 1 h at room temperature. The ELISA was developed with addition of tetramethyl benzidine (TMB) followed by Stop Solution and read at OD 450 nm. The standard curve (11.7-3000 pg/μL) was generated with human full-length recombinant Btk protein and plotted using a 4 parameter curve fit in Gen5 software. Uninhibited Btk detected from samples was normalized to μg total protein as determined by BCA protein analysis (Pierce Cat. 23225).

The results of this assay show the concentration of compound producing 50% occupancy of BTK. This level of occupancy empirically translates to a level of 50% inhibition of BTK activity (both as measured by autophosphorylation and by phosphorylation of PLCγ2). Thus, Table 6, under the column “EC₅₀,” shows the concentration of compounds of the invention that produce 50% inhibition of BTK activity. The compound numbers correspond to the compound numbers in the examples. Compounds having an activity designated as “A” provided an EC₅₀<10 nM; compounds having an activity designated as “B” provided an EC₅₀ 10-100 nM; compounds having an activity designated as “C” provided an EC₅₀ of 101-1000 nM; “nd” designates “not determined”.

TABLE 6

| Compound | IC₅₀ | EC₅₀ |
|---|---|---|
| I-1 | A | B |
| I-2 | A | A |
| I-3 | A | C |
| I-4 | A | B |
| I-5 | A | C |
| I-6 | A | C |
| I-7 | A | B |
| I-8 | A | A |
| I-9 | A | B |
| I-10 | A | B |
| I-11 | A | A |
| I-12 | A | B |
| I-13 | A | B |
| I-14 | A | B |
| I-15 | A | B |
| I-16 | A | A |
| I-17 | A | A |
| I-18 | A | A |
| I-19 | A | A |
| I-20 | A | A |
| I-21 | A | B |
| I-22 | A | B |
| I-23 | A | A |
| I-24 | A | C |
| I-25 | A | A |
| I-26 | A | A |
| I-27 | A | B |
| I-28 | A | B |
| I-29 | A | B |
| I-30 | A | B |
| I-31 | A | A |
| I-32 | A | A |
| I-33 | A | A |
| I-34 | A | A |
| I-35 | A | A |
| I-36 | A | A |
| I-37 | A | C |
| I-38 | A | A |
| I-39 | A | B |
| I-40 | A | A |
| I-41 | A | B |
| I-42 | A | A |
| I-43 | A | A |
| I-44 | A | B |
| I-45 | A | A |
| I-46 | A | A |
| I-47 | A | A |
| I-48 | A | A |
| I-49 | A | B |
| I-50 | A | B |
| I-51 | A | B |
| I-52 | A | B |
| I-53 | A | A |
| I-54 | A | B |
| I-55 | A | B |
| I-56 | A | B |
| I-57 | A | B |
| I-58 | A | A |
| I-59 | A | A |
| I-60 | A | B |
| I-61 | A | B |
| I-62 | A | C |
| I-63 | A | B |
| I-64 | A | A |
| I-65 | A | A |
| I-66 | C | nd |
| I-67 | A | B |

We claim:

1. A compound of formula I: 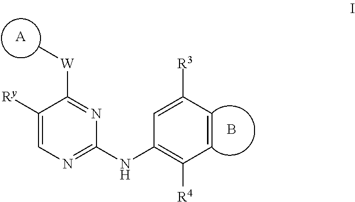
or a pharmaceutically acceptable salt thereof, wherein:
Ring A is a saturated 4-8 membered monocyclic or bridged heterocyclic ring having one —N(R¹)—, a saturated 7-11 membered spirofused heterocyclic ring having one —N(R¹)—, or a saturated 8-10 membered bicyclic heterocyclic ring having one —N(R¹)—, wherein Ring A is substituted with 0-3 R^{v} groups;
R¹ is -L-Y, wherein:
L is an optionally substituted bivalent C₂₋₈ unsaturated, straight or branched, hydrocarbon chain, wherein one, two, or three methylene units of L are optionally and independently replaced by cyclopropylene, —N(R)—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—; and
Y is hydrogen, halogen, —CN, C₁₋₆ aliphatic optionally substituted with oxo, halogen, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 groups independently selected from -Q-Z, oxo, —NO₂, halogen, —CN, and C₁₋₆ aliphatic, wherein:
Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —SO—, or —SO₂—; and
Z is hydrogen or C₁₋₆ aliphatic optionally substituted with oxo, halogen, or CN;
Ring B is a saturated 5-7-membered heterocyclo ring having 1-2 nitrogen atoms, wherein Ring B is substituted with 0-5 R^{x} groups;
W is —N(R²)CH₂— or —NH—;
R² is selected from hydrogen, C₁₋₆ aliphatic or —C(O)R;
R³ and R⁴ are each independently selected from hydrogen or halogen;
each R group is independently hydrogen or an optionally substituted group selected from C₁₋₆ aliphatic, phenyl, a 3-7 membered saturated or partially unsaturated carbocyclic ring, a 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
R^{y} is hydrogen, halogen, —CF₃, or C₁₋₄ aliphatic;
each R^{x} is independently oxo, halogen, —OR, —N(R)₂, —S(O)ₓR, —N(R)(CH₂)_{q}N(R)₂, —N(R)(CH₂)_{q}OR, —O(CH₂)_{q}OR, —O(CH₂)_{q}N(R)₂, an optionally substituted C₂₋₆ saturated, straight or branched, hydrocarbon chain wherein one or two methylene units are independently replaced by —O—, —N(R)— or —S(O)ₓ—, or an optionally substituted group selected from C₁₋₆ aliphatic, phenyl, a 3-7 membered saturated or partially unsaturated carbocyclic ring, a 4-7 membered heterocyclic ring having 1-2 heteroatoms independently selected from nitrogen, oxygen, or sulfur, or a 5-6 membered monocyclic heteroaryl ring having 1-4 heteroatoms independently selected from nitrogen, oxygen, or sulfur;
each R^{v} is independently selected from halogen or C₁₋₆ aliphatic;
q is 1 or 2; and
each x is 0, 1 or 2.

2. The compound according to claim 1, wherein Ring A is a saturated 4-8 membered monocyclic or bridged heterocyclic ring having one —N(R¹)—.

3. The compound according to claim 1, wherein Ring A is a saturated 7-11 membered spirofused heterocyclic ring having one —N(R¹)—.

4. The compound according to claim 2, wherein the compound is of formula II-a, II-b, II-c, II-d, II-e, II-f, II-g, II-h, II-i, II-j, II-k or II-l: 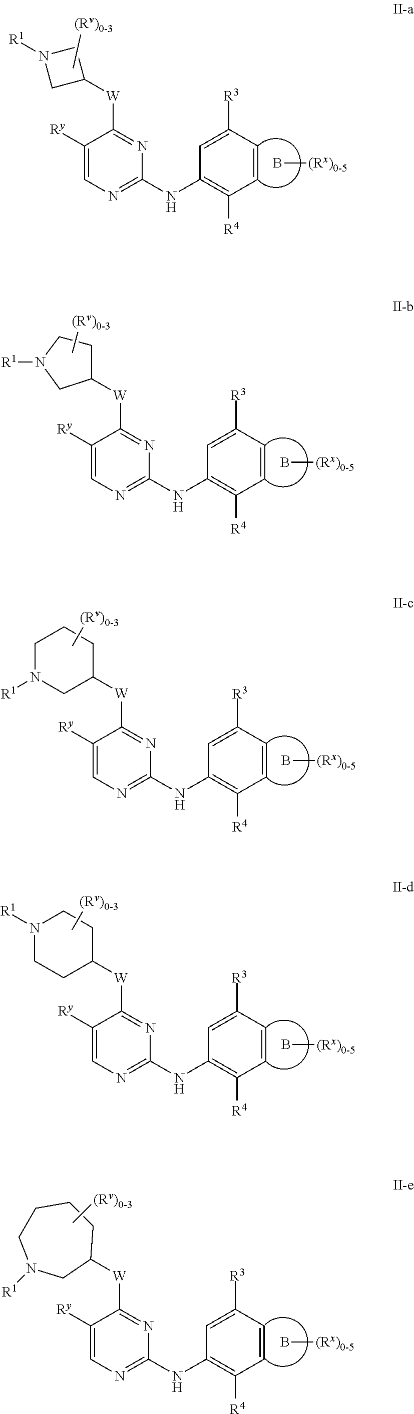 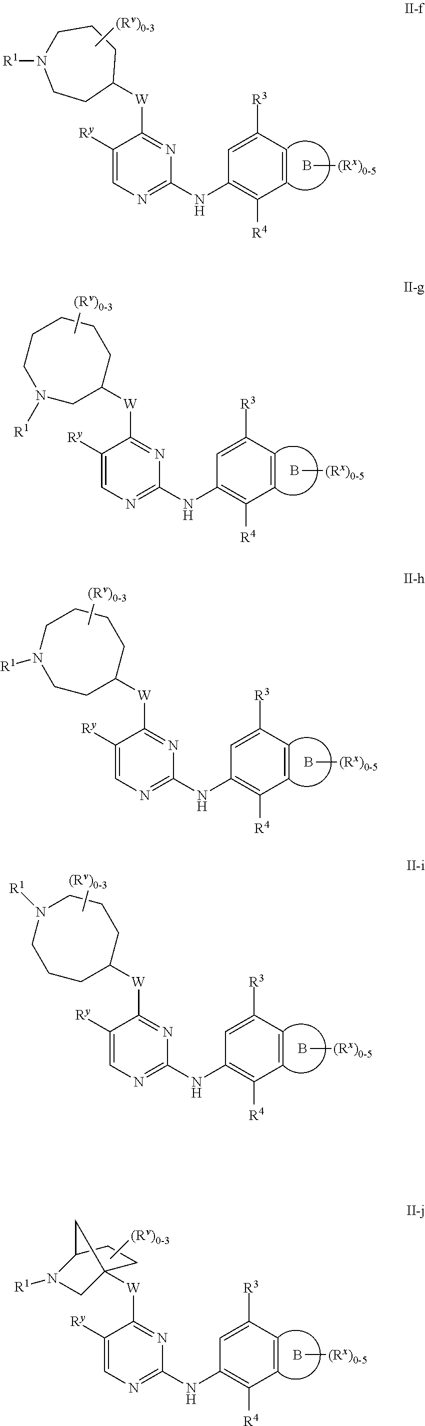 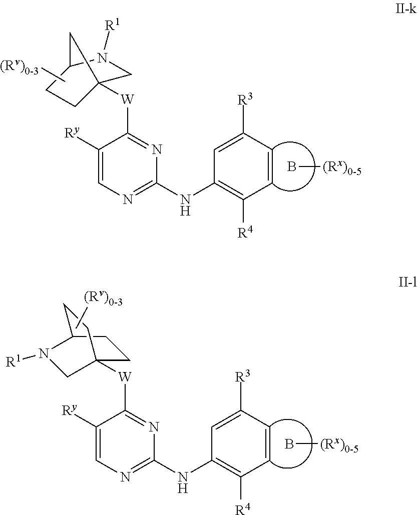
or a pharmaceutically acceptable salt thereof.

5. The compound according to claim 4, wherein the compound is of formula II-b-i, II-b-ii, II-c-i, II-c-ii, II-e-i, II-e-ii, II-f-i, II-f-ii, II-g-i, II-g-ii, II-h-i or II-h-ii: 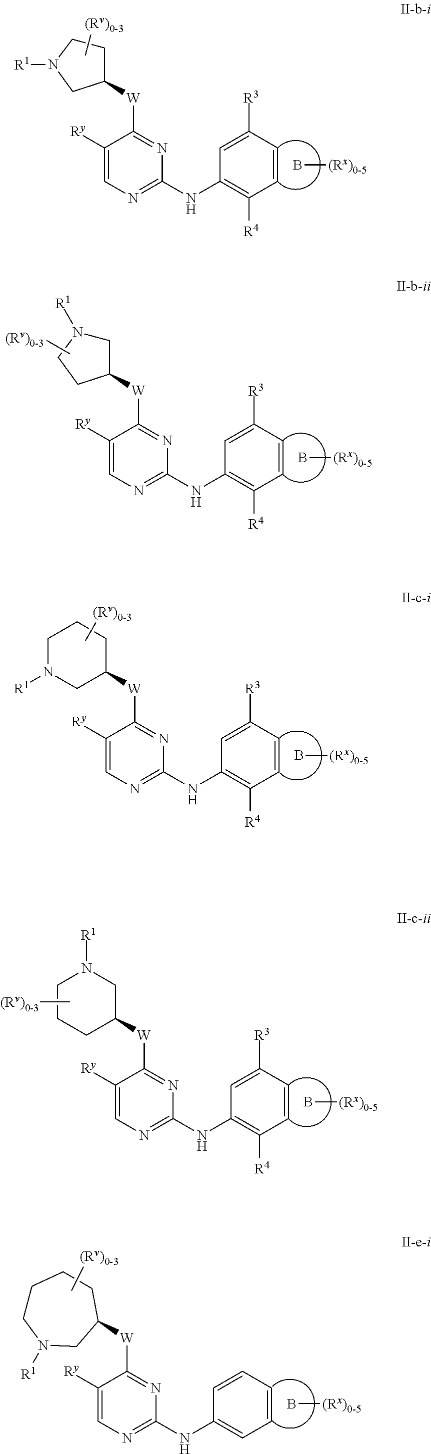 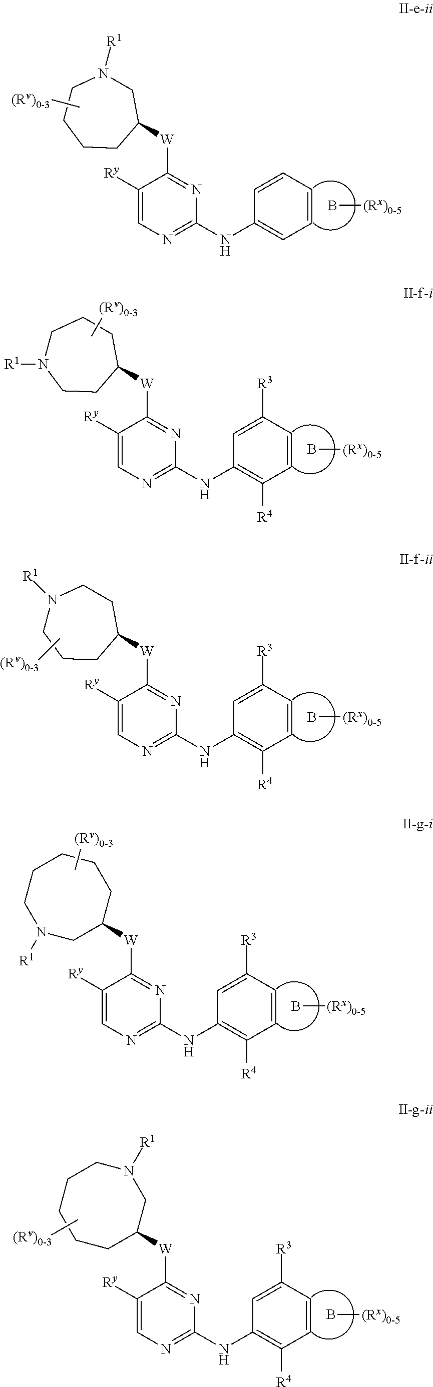 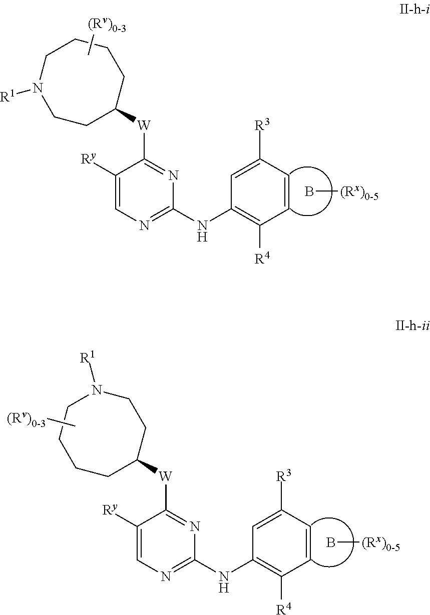
or a pharmaceutically acceptable salt thereof.

6. The compound according to claim 1, wherein Ring B is a 5-membered heterocyclo ring.

7. The compound according to claim 6, wherein Ring B is pyrrolidino ring.

8. The compound according to claim 1, wherein Ring B is a 6-membered heterocyclo ring.

9. The compound according to claim 8, wherein Ring B is a piperidino ring.

10. The compound according to claim 1, wherein the compound is of formula III-a, III-b, III-c, III-d, III-e, III-f, III-g or III-h: 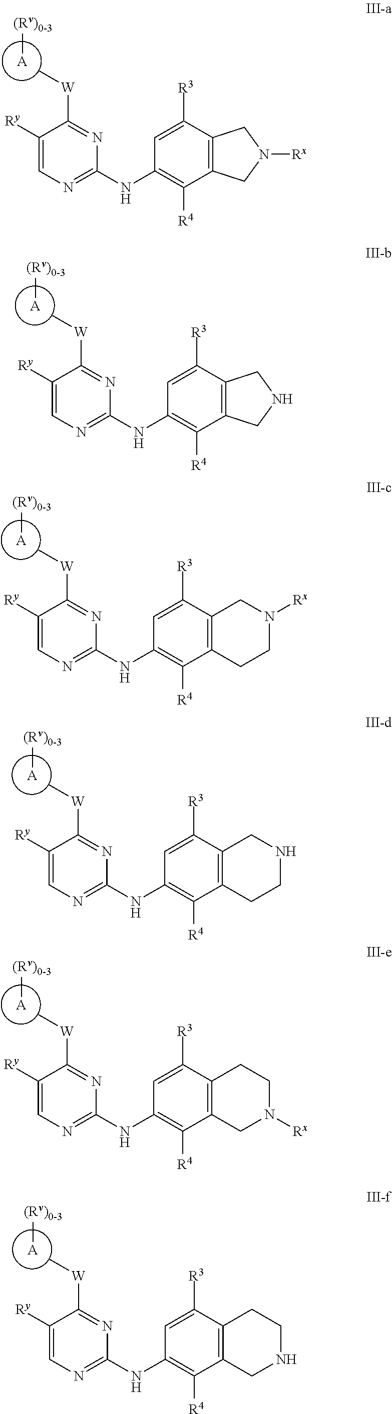 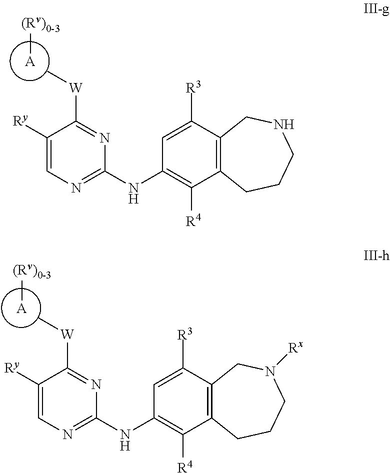
or a pharmaceutically acceptable salt thereof.

11. The compound according to claim 10, wherein Ring A is selected from

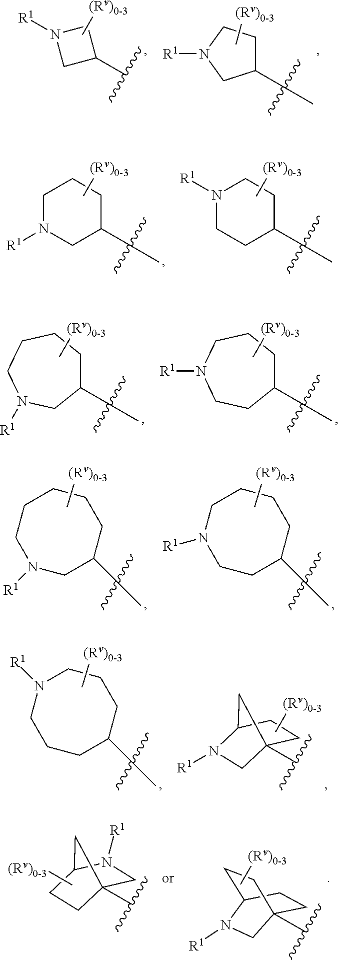

12. The compound according to claim 3, wherein Ring A is substituted with 0-3 R^{v} groups and is selected from azaspiro[2.4]heptane, azaspiro[3.3]heptane, azaspiro[2.5]octane, azaspiro[3.4]octane, azaspiro[3.5]nonane, azaspiro[4.4]nonane, azaspiro[4.5]decane, azaspiro[3.7]undecane, azaspiro[4.6]undecane, azaspiro[5.5]undecane.

13. The compound according to claim 12, wherein Ring A is substituted with 0-3 R^{v} groups and is selected from:

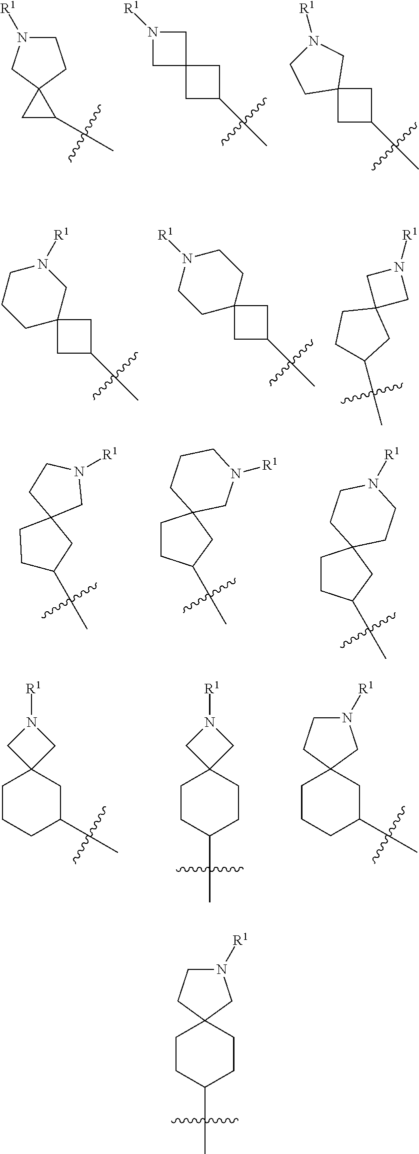

14. The compound according to claim 1, wherein R^{y} is halogen.

15. The compound according to claim 14, wherein R^{y} is fluoro.

16. The compound according to claim 1, wherein R² is hydrogen.

17. The compound according to claim 1, wherein R¹ is -L-Y, wherein:
L is an optionally substituted bivalent C₂₋₈ straight or branched, hydrocarbon chain wherein L has at least one double bond and one or two methylene units of L are optionally and independently replaced by —N(R)C(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—.

18. The compound according to claim 17, wherein:
L is an optionally substituted bivalent C₂₋₈ straight or branched, hydrocarbon chain wherein L has at least one double bond and at least one methylene unit of L is replaced by —C(O)—, —N(R)C(O)—, —C(O)NR—, —N(R)SO₂—, —SO₂N(R)—, —S—, —S(O)—, —SO₂—, —OC(O)—, or —C(O)O—, and one additional methylene unit of L is optionally replaced by cyclopropylene, —O—, —N(R)—, or —C(O)—; and
Y is hydrogen, halogen, —CN or C₁₋₆ aliphatic optionally substituted with oxo, halogen, NO₂, or CN.

19. The compound according to claim 18, wherein L is an optionally substituted bivalent C₂₋₈ straight or branched, hydrocarbon chain wherein L has at least one double bond and one methylene unit of L is replaced by —C(O)—, and one additional methylene unit of L is optionally replaced by cyclopropylene, —O—, —N(R)—, or —C(O)—.

20. The compound according to claim 18, wherein L is an optionally substituted bivalent C₂₋₈ straight or branched, hydrocarbon chain wherein L has at least one double bond and one methylene unit of L is replaced by —SO₂—

21. The compound according to claim 18, wherein L is substituted with —OH, —CN or halogen.

22. The compound according to claim 1, wherein L is —C(O)CH═CH—, —C(O)CH═C(F)—, —C(O)CH═C(CN)—, —CH(OH)CH═CH—, —CH(OH)C(F)═CH—, —CH(OH)C(CN)═CH—, —CH(OH)CH═C(F)—, or —CH(OH)CH═C(CN)—.

23. The compound according to claim 1, wherein R¹ is -L-Y, wherein:
L is an optionally substituted bivalent C₂₋₈ straight or branched, hydrocarbon chain wherein L has at least one triple bond and one or two additional methylene units of L are optionally and independently replaced by —C(O)—, —O—, N(R)—, —N(R)C(O)—, —C(O)NR—, —N(R)SO₂—, —SO₂N(R)—, —S—, —S(O)—, —SO₂—, —OC(O)—, or —C(O)O—.

24. The compound according to claim 23, wherein Y is hydrogen or C₁₋₆ aliphatic optionally substituted with oxo, halogen, NO₂, or CN.

25. The compound according to claim 24, wherein L is —C(O)C≡C—.

26. The compound according to claim 1, wherein R¹ is selected from:

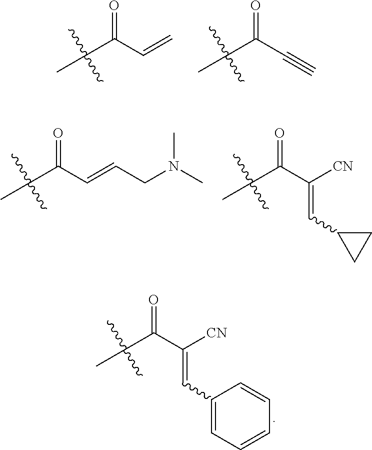

27. The compound according to claim 1, wherein W is —NH—.

28. The compound according to claim 1, wherein R^{v} is halogen.

29. The compound according to claim 28, wherein R^{v} is fluoro.

30. The compound according to claim 1, wherein R^{x} is an optionally substituted C₁₋₆ aliphatic.

31. The compound according to claim 30, wherein R^{x} is —CH₃, —CH(CH₃)₂, —C(CH₃)₃, or —CH₂C(CH3)₃.

32. The compound according to claim 30, wherein the C₁₋₆ aliphatic is substituted with oxo.

33. The compound according to claim 32, wherein R^{x} is —C(O)CH₃ or —CH₂C(O)NH₂.

34. The compound according to claim 30, wherein R^{x} is selected from 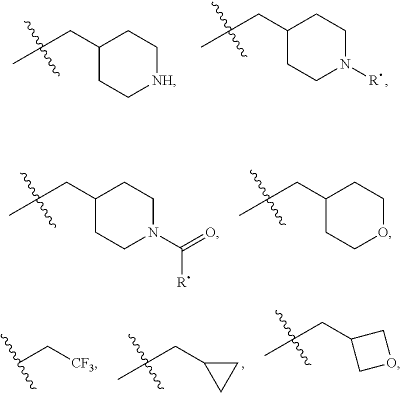
—CH₂CH₂F, —CH₂CH₂OH and —CH₂CH₂OCH₃,
wherein each R^{} is selected from hydrogen and C₁₋₆ aliphatic optionally substituted with halogen.

35. The compound according to claim 1, wherein R^{x} is an optionally substituted 4-7 membered heterocyclic ring having 1-2 heteroatoms independently selected from nitrogen, oxygen, or sulfur.

36. The compound according to claim 35, wherein R^{x} is selected from 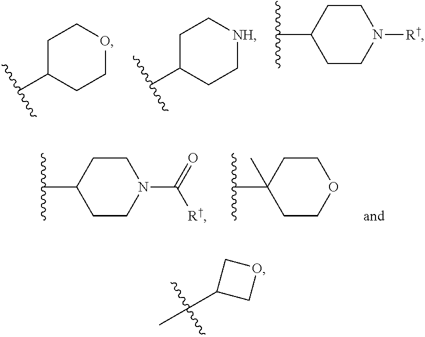
wherein each R^{†} is selected from hydrogen and C₁₋₆ aliphatic optionally substituted with halogen.

37. The compound according to claim 1, wherein R^{x} is an optionally substituted 3-7 membered saturated or partially unsaturated carbocyclic ring.

38. The compound according to claim 37, wherein R^{x} is selected from

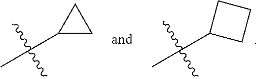

39. The compound according to claim 1, wherein R^{x} is optionally substituted phenyl.

40. The compound according to claim 39, wherein R^{x} is

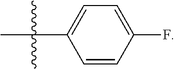 [SMILES: *c1ccc(F)cc1]

41. A method of inhibiting a B cell receptor, comprising contacting a cell with a compound according to claim 1.

42. A method of treating or lessening the severity of a B cell-mediated disorder, comprising administering to a patient in need thereof a compound according to claim 1.

43. A method of inhibiting a T cell receptor, comprising contacting a cell with a compound according to claim 1.

44. A method of treating or lessening the severity of a T cell-mediated disorder, comprising administering to a patient in need thereof a compound according to claim 1.
